(19) United States
(12) Patent Application Publication
(10) Pub. No.: US 2005/0244815 A1
(43) Pub. Date: Nov. 3, 2005

(54) Compositions and methods for viral resistance genes

(75) Inventors: Margo A. Brinton, Decatur, GA (US); Andrey A. Perelygin, Alpharetta, GA (US)

Correspondence Address: TROUTMAN SANDERS LLP, BANK OF AMERICA PLAZA, SUITE 5200, 600 PEACHTREE STREET , NE, ATLANTA, GA, 30308-2216, US

(73) Assignee: Georgia State University Research Foundation, Atlanta, GA (US)

(21) Appl. No.: 11/012,762

(22) Filed: Dec. 15, 2004

Related U.S. Application Data
(63) Continuation-in-part of application No. PCT/US03/19300, filed on Jun. 19, 2003.
(60) Provisional application No. 60/390,046, filed on Jun. 19, 2002.

(51) Int. Cl. C12Q 1/70; C12Q 1/68

(52) U.S. Cl. 435/5; 435/6

(57) ABSTRACT

The present invention is directed to methods and compositions for identifying viral resistance genes and for identifying individuals having resistant or susceptible genotypes. Once identified as either resistant or susceptible, appropriate actions, such as vaccination, can be undertaken by the individuals. In particular, methods and compositions for identifying genes associated with resistance or susceptibility for flaviviruses are disclosed.

### RELATED APPLICATIONS

This application is a continuation-in-part of PCT/US2003/19300, filed Jun. 19, 2003, which claimed priority to U.S. Provisional Patent Application No. 60/390,046, filed Jun. 19, 2002, each of which is herein incorporated in its entirety.

### TECHNICAL FIELD

This invention is directed to compositions and methods for viral resistance genes. In particular, the invention is directed to compositions and methods for identifying viral resistance genes and for identifying individuals having the resistance genes.

### BACKGROUND OF THE INVENTION

Viruses cause some of the most debilitating illnesses known in animals, including humans, and plants. Vaccination procedures have provided relief from some of the more deadly viruses, such as smallpox, measles, influenza and poliovirus. However, many viruses still cause much human and animal suffering, loss of work days, and sometimes death.

Not all individuals infected with a virus respond identically to the virus. Such individual variation in response to viral pathogens has been seen in both animals and humans. For example, when Australian rabbit populations were controlled by the introduction of a viral pathogen, 99% of the rabbits were killed, but 1% survived. In humans who were accidentally inoculated with hepatitis B virus, only a small percentage developed clinical hepatitis and only a small percentage of those individuals developed severe disease.

Such variation in response to infection, in the extent of the disease state and the ultimate outcome is presumed to be due to multiple factors. Some of these factors include genetic makeup, nutritional status, age, and immune competency. For particular viral pathogens, some of the factors have been suggested as being important, but there are no tests or assays that would enable one to predict an individual's response to exposure to the pathogen.

What is needed are compositions and methods for determining, prior to exposure to the viral pathogen how ill the individual will become if infected. Additionally, such methods could be used for determining which individuals should be vaccinated.

### SUMMARY OF THE INVENTION

The present invention comprises compositions and methods for identifying viral resistance/susceptibility genes and for identifying individuals having such viral resistance/susceptibility genes. In particular, compositions and methods for identifying flavivirus resistance/susceptibility genes are provided. Additionally, methods for testing body samples to determine the presence or absence of genes associated with viral resistance/susceptibility are provided.

Inbred mouse strains exhibit significant differences in their susceptibility to viruses in the genus Flavivirus, which includes human pathogens such as yellow fever, Dengue, and West Nile virus. A single gene, designated Flv, confers this differential susceptibility and maps to a region of mouse chromosome 5. A positional cloning strategy was used to identify twenty-two genes from the Flv interval including ten members of the 2′-5′ oligoadenylate synthetase gene family. One 2′-5′ oligoadenylate synthetase gene, Oas1b, was identified as Flv by correlation between genotype and phenotype in nine mouse strains. Susceptible mouse strains produce a protein lacking 30% of the C-terminal sequence as compared to the resistant counterpart due to the presence of a premature stop-codon. The Oas 1b gene differs from all of the other murine Oas genes by a unique four amino acid deletion in the P-loop located within a conserved domain thought to be involved in RNA binding. Expression of the resistant allele of Oas1b in susceptible embryofibroblasts resulted in partial inhibition of the replication of a flavivirus, but not of an alpha togavirus.

There are three types of Oas genes in mammals. The large three unit-containing Oas 3 gene, the two unit-containing Oas 2 gene and the single unit Oas 1 gene. Many mammals also contain single-unit Oas-like genes. The single unit Oas 1 genes have duplicated in mice; there are 8 mouse Oas1 genes. Only one of these, Oas1b, confers flavivirus resistance/susceptibility. In the human genome there is only a single OAS1 gene, but this gene produces multiple isoforms.

The present invention comprises the finding that resistance to infection by flaviviruses has been associated with variations in human OAS genes, particularly OAS1. If an G (G-allele) is present at nt position 12,349 (numbered beginning from the Atg-start codon in the genomic DNA), the p46 and p48 isoforms are produced. If a G to A transversion (A-allele) at nt position 12,349 is present, the p40, p48 and p52 isoforms are produced. The 346 N-terminal amino acids of these four proteins are identical but their C-terminal regions differ due to alternative splicing.

Data showed that the frequency of the A-allele in ethnic Russian populations who are known to be highly resistant to disease caused by the flavivirus, tick borne encephalitis virus, was significantly increased (up to 99%) as compared with the frequency in a group of patients with tick borne encephalitis virus-induced disease (59%). The GG homozygous individuals exhibited the most severe disease symptoms. No GG-homologous individuals were detected in the native Siberian populations. In contrast, the frequencies of the A-allele and the G-allele were similar in groups of healthy Russians and Russians infected with hepatitis C virus (a distantly related member of the flavivirus family from a different genus). Mutations in the human Oas2 and or Oas3 genes may also be relevant to virus resistance/susceptibility.

Methods for determining the individual genotypes have been developed. To determine the presence of the A and G alleles, samples of genetic material from individuals are obtained and the DNA is characterized for the presence or absence of the susceptibility/resistance polymorphism.

### FIGURES

FIG. 1 shows the physical and transcript maps of the mouse Flv interval.

FIG. 2A and B show the structures of the mouse Oas 1b gene and protein.

FIG. 3 is a Northern blot showing the constitutive expression of mouse Oas1b in different mouse tissues.

FIG. 4A is a graph and FIG. 4B is a table showing the effect of low level expression of the resistant Oas1b protein in susceptible C3H/He cells on the growth of a flavivirus, West Nile virus, and an alpha togavirus, Sindbis.

FIG. 5 is an un-rooted neighbor-joining, distance-based phylogenic tree of murine, rat and human Oas sequences.

FIG. 6A-H show multiple alignments of the protein sequences of the murine, rat and human 2′-5′ oligoadenylate synthetases.

FIG. 7 shows the relationship of the human OAS gene family members and the murine Oas gene family members. A comparison of the orthologous 2′-5′ oligoadenylate synthetase genes located on Homo sapiens autosome 12 (HSA12) and Mus musculus autosome 5 (MMA5) are shown. There are eight murine Oas 1 genes (from “a” through “h”) orthologous to a single human OAS1 gene.

FIG. 8 shows the position of a G to A transversion [indicated by r and located at nucleotide position 12,349 (numbered beginning from the ATG-start codon)] in human OAS1 genomic DNA and the amino acids present at the beginning and end of the C-terminal parts of the OAS1 isoforms, p40, p46, p52 and p48, that are generated depending on whether a G or A is present at nucleotide position 12,349.

FIG. 9 is a photograph of ethidium bromide stained PCR DNAs electrophoresed on a 2% agarose gel. The fragment patterns detected for humans with the different genotypes that determine viral resistance/susceptibility are shown. Lane 1—100 bp DNA ladder; Lane 2—AA homozygous DNA digested with Alu I; Lane 3—AG heterozygous DNA digested with Alu I; Lane 4—GG homozygous DNA digested with Alu I; Lane 5—undigested PCR product.

### DETAILED DESCRIPTION

The present invention is directed to compositions and methods for determining gene variations that confer resistance/susceptibility to viral pathogens and for determining the presence or absence of such genes in the genotypes of individuals. The determination of the viral resistance genotype of an individual is important in assessing the individual's response to a known viral pathogen, assessing the population's response to a viral pathogen and may be important in predicting the response of both the individual and the population's response to viral pathogens that enter the environment at a later time.

Global connections and travel are some of the causes of entry of viral pathogens into new geographic areas. For example, mobile populations and growing food imports make it more likely that diseases will continue to spread well beyond the borders where the diseases originated. For example, in the U.S., West Nile virus first appeared in New York in 1999 and has spread southward, northward and westward. Thousands of people have been infected. About 10% of these have developed moderate to severe clinical symptoms and several hundreds of people have died. Countless birds, horses and other animals have been infected and many have died. The virus is predicted to spread throughout the continental United States and to all regions of the Americas.

West Nile virus belongs to a family of disease-causing viruses known as flaviviruses, which are spread by insects, usually mosquitoes but also by ticks. The currently identified flaviviruses include, but are not limited to, tick-borne virus such as mammalian tick-borne virus groups including Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, subtypes European, Far Eastern, and Siberian, Louping ill virus, subtypes, Irish, British, Spanish, and Turkish, Seabird tick-borne virus group including Meaban virus, Saumarez Reef virus, Tyuleniy virus and also include mosquito-borne viruses such as the Aroa virus group including Aroa virus, Bussuquara virus, Iguape virus, Naranjal virus, the Dengue virus group including Dengue virus, Dengue virus type 1, Dengue virus type 2, Dengue virus type 3, Dengue virus type 4, Kedougou virus, and the Japanese encephalitis virus group including Cacipacore virus, Koutango virus, Japanese encephalitis virus, Alfuy virus, St. Louis encephalitis virus, Usutu virus, West Nile virus, Kunjin virus, Yaounde virus, the Kokobera virus group including Kokobera virus, Stratford virus, the Ntaya virus group including Bagaza virus, Ilheus virus, Rocio virus, Israel turkey meningoencephalomyelitis virus, Ntaya virus, Tembusu virus, the Spondweni virus group including Zika virus, Spondweni virus, the Yellow fever virus group including Banzi virus, Bouboui virus, Edge Hill virus, Jugra virus, Saboya virus, Potiskum virus, Sepik virus, Uganda S virus, Wesselsbron virus, Yellow fever virus and also include viruses with no known arthropod vector such as the Entebbe bat virus group including Entebbe bat virus, Sokoluk virus, Yokose virus, the Modoc virus group including Apoi virus, Cowbone Ridge Virus, Jutiapa virus, Modoc virus, Sal Vieja virus, San Perlita virus, the Rio Bravo virus group including Bukalasa bat virus, Carey Island virus, Dakar bat virus, Montana myotis leukoencephalitis virus, Phnom Penh bat virus, Batu Cave virus and Rio Bravo virus. Viruses that may also be included in this group include Tamana bat virus and cell fusing agent virus.

Flaviviruses that are important human pathogens include yellow fever virus, Japanese encephalitis virus, dengue virus, tick borne encephalitis virus and Saint Louis encephalitis virus. Most human infections are asymptomatic or mild, causing fever, headache and body aches, often accompanied by a skin rash and swollen lymph glands. If the virus crosses the blood-brain barrier, life-threatening encephalitis or meningitis is often the result. Polio-like paralysis and Parkinson's disease-like symptoms can also result from West Nile virus infection. West Nile is most often transmitted by insect vectors, although transmission can also occur through transfusion, transplantation and from mother to child during birth or during breast feeding.

West Nile virus primarily cycles between mosquitoes and birds. Other animals, such as humans or horses, are incidental hosts to which the virus is transmitted during the mosquito's blood meal. Many bird species are infected with West Nile virus, with crows, bluejays and sparrows being some of the most susceptible species. An increase in dead birds is often the first clue that West Nile is invading an area or that the virus is found there is large numbers.

Currently, there are no drugs to treat West Nile virus and no vaccines are available to prevent infection. This lack of treatment and vaccines is the case for many flaviviruses and other viral pathogens. While vaccines are often a goal for medical treatment, vaccines are not without some risk to the individuals being vaccinated. With the compositions and methods of the present invention, the genotypes of individuals in an infected area can be easily determined and this information used to decide what medical intervention may be necessary. If the population is comprised primarily of individuals who are resistant to infection, mass vaccination would be a waste of resources. Additionally, there may be a subset of the population that is highly susceptible and vaccination may cause harm in these individuals. Further, these highly susceptible individuals could take preventive measures, such as reducing exposure to conditions where insects are feeding, and thus avoid illness.

The methods and compositions of the present invention are useful for identifying individuals who are at risk of developing disease, and also for identifying individuals who are resistant to disease. Such individuals comprise mice, and also other species, including, but not limited to humans, horses, cattle, sheep, pigs, wild birds and chickens. It is very expensive to vaccinate farm animals yearly and if the genotypes of the animals are known, only the susceptible animals need be vaccinated. Wild bird populations could be restocked with resistant individuals.

Innate resistance to flavivirus-induced morbidity and mortality was first demonstrated in mice in the 1920's (1) and showed monogenic autosomal dominant inheritance (2). The alleles that determined resistance and susceptibility were designated Flv^{r} and Flv^{s}, respectively (3). Resistant mice are susceptible to infections with other types of viruses, but are resistant to all flaviviruses (4). The disease resistance conferred by the Flv^{r} allele has been demonstrated with a number of different flaiviviruses. Mosquito-borne flaiviruses tested include West Nile virus, dengue, St Louis encephalitis, yellow fever virus, Japanese encephalitis, Banzi, Ilheus, Murray Valley encephalitis, Kunjin, Alfuy and Kokobera. The tick-borne flaviviruses tested include louping ill and Russian Spring Summer encephalitis viruses. Flavivirus resistant and susceptible mouse strains were shown to be equally susceptible to viruses from other families, such as an arenavirus, three bunyaviruses, a picornavirus, two rhabdoviruses, seven alpha togaviruses, and two herpes viruses.

Resistant mice can be infected by flaviviruses, but the virus titers in their tissues are lower by 1,000- to 10,000-times than those in the tissues of susceptible animals and the spread of the infection in resistant mice is slower (5-6). Cell cultures derived from many different tissues of resistant mice also produce lower yields of virus; peak titers from resistant cultures are 100- to 1,000-times lower than from susceptible cultures (7-9). Previous studies indicate that the Flv gene product acts intracellularly on flavivirus replication.

The flavivirus resistant allele was demonstrated in wild Mus musculus domesticus populations in both the U.S. and Australia and flavivirus genetic resistance was also reported in other Mus species (11-13). Most commonly used inbred laboratory mouse strains were derived from a small number of progenitors and the majority of them have a homozygous flavivirus susceptible genotype. Only the Det, BSVR, BRVR, CASA/Rk, CAST/Ei, MOLD/Rk and PRI inbred strains have the resistant allele (10). The characteristics of a resistant-like allele (designated Flv^{r}-like) in CASA/Rk and CAST/Ei strains were similar to those of the PRI Flv^{r} allele. The MOLD/Rk animals carry an allele designated minor resistance Flv^{mr}, which can protect carriers from disease after infection with the attenuated 17D strain of yellow fever virus, but not from the virulent Murray Valley encephalitis virus (11).

The resistant allele from donor PRI mice was introduced onto the susceptible C3H/He background to produce the congenic inbred C3H.PRI-Flv^{r} (formerly C3H.RV) strain by a standard backcross protocol followed by brother-sister matings with selection at each generation for the resistance phenotype (13). These congenic strains also carry different alleles of the Ric gene, which controls susceptibility to Rickettsia tsutsugamushi and is located on mouse chromosome 5 (14). These data suggested linkage between the Flv and the Ric loci and the congenic strains were subsequently used to map the Flv locus on mouse chromosome 5 by linkage with the Ric and rd loci (15). Subsequently, twelve microsatellite markers from mouse chromosome 5 were genotyped relative to the Flv gene in 1325 backcross animals. Two of the microsatellite markers, D5Mit408, and D5Mit242, exhibited map distances with the Flv locus of 0.30 and 0.15 centiMorgans (cM), respectively, while one additional marker, D5Mit159, showed no recombination with Flv, indicating linkage of <0.15 cM (16).

To isolate the Flv gene a positional cloning strategy was used. The loci located near the D5Mit159 marker were first identified and then their sequences were compared in cells from congenic resistant and susceptible mice. The Flv gene was identified as mouse 2′-5′ oligoadenylate synthetase 1B (Oas1b) by these methods.

Abbreviations used herein include BAC, bacterial artificial chromosome: Flv, flavivirus resistance gene; Oas, 2′-5′ oligoadenylate synthetase; ORF, open reading frame; WNV, West Nile virus

Data depositions, and sequences disclosed herein by specific incorporation, include mouse sequences, AF217002 (SEQ ID 1), AF217003 (SEQ ID 2), AF261233 (SEQ ID 3), AF319547 (SEQ ID 4), AF328926 (SEQ ID 5), AF328927 (SEQ ID 6), AF418004 (SEQ ID 7), AF418005 (SEQ ID 8), AF418006 (SEQ ID 9), AF4188007 (SEQ ID 10), AF88008 (SEQ ID 11), AF418009 (SEQ ID 12), AF418010 (SEQ ID 13), AF453830 (SEQ ID 14), AF459815 (SEQ ID 15), AF459816 (SEQ ID 16), AF478457 (SEQ ID 17), AF480417 (SEQ ID 18), AF481733 (SEQ ID 19), AF481734 (SEQ ID 20), AY055829 (SEQ ID 21), AY055830 (SEQ ID 22), AY055831 (SEQ ID 23), AY057107 (SEQ ID 24), AY196696 (SEQ ID 25), AY196697 (SEQ ID 26), AY196698 (SEQ ID 27), AY196699 (SEQ ID 28), AY196700 (SEQ ID 29), AY221507, (SEQ ID 30), AY196701 (SEQ ID 31), AY227756 (SEQ ID 32), AY230746 (SEQ ID 34), AY237116 (SEQ ID 33), bovine sequence AY243505 (SEQ ID 36), AY250706 (SEQ ID 35), equine sequence AY321355 (SEQ ID 37) Human genomic OAS1 sequence (SEQ ID 38) (derived from the GenBank sequence AC00455 (Homo sapiens 12q24.1 PAC RPCI1-71H24), positions 120325-133280, presented here as the reverse complement of the GenBank sequence):

aaccaacagcagtccaagctcagtcagcagaagagataaaagcaaacaggtctgggaggcagttctgttgccactctctctcctgtca
atgatggatctcagaaataccccagccaaatctctggacaagttcattgaagactatctcttgccagacacgtgtttccgcatgcaaatca
accatgccattgacatcatctgtgggttcctgaaggaaaggtgcttccgaggtagctcctaccctgtgtgtgtgtccaaggtggtaaagg
tgagtccaggcctgcctggccaggggaggggtggctgaatgtgcaagagttgagattgagaatgagagagagagagagagagaa
gcaaaaacctagaacccagggtgcaaatgtgagtacagagagctgagatcttctgggatggtggtttcttatttatccacacagcatgtt
aaaatagattctggggtgaaatcctacatccctattattaacaagtgaccctcccccctacttcccgctgaagtttatgaaccactgtcctg
ggcgatgcccatttcagaaatagggaactgaatcccagctctggtaaacagtttgctaattcgtggccaggctaggggctcaccatttct
gcagtgaagaatcatatgttttgaaagcaaatagcacctgctggctgcaagaccttgagcaagtcacttaactactctgtgttccaatttcc
tcagccataatccccaatactgttgcagtcttgccagtgcaccttaatgtagcagcttctcactgaattagtacccaaggttctttgtcctgc
atccaagaaaattaaggaacatggacacaaacgtgagcttggagcaaaagttcagtaagcaaaagaagaaagctgtctccactgtgg
agagggaagtctgagtggattgccagattgcagctgaatgcaaaaaacttttataagaaaccactctcctccctgtaactgtttgagaaa
ctttttatcagtaaagctgtgcaacttcccttaccttatgcagctgtgggtatatctctaggcaagcataaagcgctgcttctcttgtatgtata
actgtggatttgttttaggtaagtcccactccctgcgccagtttcaggcaggccgctcctccagggcccagccttgaccatttacctaact
gatttttcctctactttccctcaatacctcatagggccgtgtagattaagtaaaatagtaagtgtgaaccacccagcataagctagtcctgg
gcatcgtaaaggacaatgggaaaagaacacagatcctggaagaaggcccccaggtttgaattgtatttgccacctactagctgggtga
tggggctgatatattatctcactgagcatccattttcccatctgtaaaatgggaactaatgataatggcatccaaatcatagcatcattgtga
gcattataggagtttaagacatgcaatgccttcagaacagtggctagtgctccataatgttagtgattgctcctgtcattttatttagggagg
tttgcctcactaagcatcaattattatttttgtcgtctttttcagggtggctcctcaggcaagggcaccaccctcagaggccgatctgacgct
gacctggttgtcttcctcagtcctctcaccacttttcaggatcagttaaatcgccggggagagttcatccaggaaattaggagacagctg
gaagcctgtcaaagagagagagcattttccgtgaagtttgaggtccaggctccacgctggggcaacccccgtgcgctcagcttcgtac
tgagttcgctccagctcggggagggggtggagttcgatgtgctgcctgcctttgatgccctgggtgagagctcccagcttctttttctccc
tcttcccatttctgagcagaaatctcccacagtttgagagctttttgccccaacagggcatctctctaaagcagggtgggaggagatctta
ggatctgtcccggggcaagaatgaatacggtcatgatctatcacaggagagacattaaacagcaaattggcataatgtggggacaaa
gacatttcttacagaacatctgcaaggcttactggttctgtttaaggcaaaatgtgtgaattttatctttctaaaatcaggcagcaaagatgt
ggcttaaagttcatgttactctcatctttgtcccaacatgagatctcatcaaacgtatgcagcacgttgggagatagatatttataatttgcag
gaacatttggacaggaagtgtaacctctcagaggctcccttgccacatcaggagaattggtaaaaccacactacctgtatcatatcattat
tttaagtgataaatgatcatctacattcagctctgatgagtaataggtgttcaaaaataggaacttccagccaagtgtggtggctcatgctt
gtaattccaacacttttggaggctgaggcaggagggtcgcttgagcccaggagttcaagaccagcctgggcagcaaagtgaaacctc
atctctactaaaaattttaaaacattagccaagtgtggtggtacatgcctgtggtcgcagttattcaggacgctgagactgaacgatcaca
tgaggccagccaaggattcgaggtgtcagtgagccacgaatgtaccactgcactccatcctaggcacagagcaagagcaagaccct
gtctcaatcaatcagtcaatcagtcaaaactatgaatttcccagctgtatatgaaggcacctcaaaacaccacagtgaactcacagagg
gacacggaatagtttagattttaattttttgagggaaatgcgatgacatctgtcacacaccgcacaaacggctactattaaactgaacttac
tgattagtggctactaattaatagttggtcattaagcagtaattagtgattaattatcaagtaattaggacttaattaaaggaactgtcacagtt
tcctttagtcctagggcagccatgaaaaaaaaaatgctgactctccaaagacaccagggtatgagaaagttttggattctctcctttgtgc
catctcctgtgttgggggctgaagtacaatggttgtaaaagacaagagggagaaggctggtcacagtggctcacgcctgtaatctcag
cactttgggaggccaaagtggggggatcacttgaagtcaggaattcaagaccagcctggccaacatggtgaaatctcacatctactaa
aaatacaaaaattagctgggcgtggtggtgtgtgcctgtaatcacagctactcgggaggctgaggcaggagaattgcttgaacccagg
agatggaggttgcaargagccaagatcatgccattgcactccagcctgggcaacagagtgagactccatctcgaaaaaaagaaaaaa
gaaaagaatataaggagtgattaaaaaagaaaagaaaagaaaactaagtagggtgaaacaatagatagccatgggggttagggagc
ttttttagacagggtcgtgagggagggtccctgagcctgagtggcgagaaggagtgagccttggggagatctggaggttctgggaag
aggaatggcaagtgcagaggccctgaagcagcaatgaccatggcacatttgaggaagagagaaaaagtcagagaagtagaaagtg
ggcaaaggaagcaagacaggaggtgaggtgggagaggttccagagaccagatcacaccagacatcattggccaccataagatcttt
gggttttaaaattccagatgttatgggatgcaggaagcagcatgatcagcagcattctctaggtgccaggttgagaacaggctgtgggg
gaacctgtaaagaggttgctgccatagttccggcgagtgacggtggtggcttggatggggtgatggcagtggagagggcaggaggg
aggatcaggaatggacctcaagacttcccagccctgggtctgctgcacttttcaatcaaaccccatggccagggagattgtcccctcag
agtgactgaaggaaattcagagaagagctgacacctaagttgtagattttgcccgaacaggtcagttgactggcggctataaacctaac
ccccaaatctatgtcaagctcatcgaggagtgcaccgacctgcagaaagagggcgagttctccacctgcttcacagaactacagaga
gacttcctgaagcagcgccccaccaagctcaagagcctcatccgcctagtcaagcactggtaccaaaatgtatggccctcccaccag
gcctggtgggtcctgtctcgactgggagcagaggaggggtggggggaggagagaaagaagggagtgaagggaagaggagggg
gagtggtggagggaaatagagggatggaaaaaggagagaaaggaaaaagaggtggagagaggagcctgcaacagaagggaga
atgaaagggaaggaagagagaaaggaagggattttggtgttctgttcactgctgtatccccagaacttaaaacagagcctggtgcata
ataggtgtaaataactgttgaataaatgaatcaatgctacatacacacacgcacgcacacacacacagagagagagtcaaccacactc
ttcagaaggtggataagttaaaacaagagtttcaaacaaatatatgttcagatgccctttcctcccacttactggctggctggccttaagta
agcaacttaacctttctgttctttctgctttcttatctgcaacgagtagcatgccatagctagagtaacacggcatatagttggtcctgataaa
tgtagcatattttagccaccataggagtacacataataaaagctaacatgtagtatgtgcttagcttatctatgttttgtggatgtgatacaatt
ttctgttcacttttaaatgccctgcatcttagtcaattttaacagtgattctgtaagttagataaggttaggcattattattaaatccattttacacc
aagagaaacttgggtcaaaaagagaaactcctgggtcacatggctcattcggccaataagtagcagaagtaaaatttgaatttggctgg
gcgcggtagctcacaccagtaatcccagcactttgggaagccaaggcaggtagattgcttgagcccaggagttcaagactagcctga
gcaacatggcaaaacctcgtctctacaaaataaactaaaaatttagccaggtgtgatggtgagcacctgtagccccagctactgggtag
gctgaggtgggaggatcgcttgagcctgggaggaggaggttgcagtaagtcaggattgcactactgccctccagcctgtgagacag
agcaagatcttctctcaaacaaacaaacaaacaaacaaacaaaaactcgaatttgggtctattgacttaagagtttgcctgataataatag
gcattcaatgtatatttcttgaatgaacgaatgaatgaaaataatcaggaataaactttccaatttaaaagtaacacctctaggtaaaaaaa
agacaatcatttagttgccagacttctaagtgtttgctgttctatgaattgtaatcatggagcctgagcattgtagaatttacaaaagcagttc
ctgacaaaagcagcactgcccccagggacatattgaaaattaatgagggtgtttttggtaaccatggtgatgggaggacatgggtgcta
cttatatttagtggaaagaagacaagaatgctagttattgtacaatgatcaagagagtcctgcacagccaagaattgtctttttctttctttctt
gatgctgttctcctttaaaacaagacaagattaacaataatttaactccactaaccaccatcatcaccacctccaacttatatgctacatttct
tgtatatttcaagtctgtttatattttcaagtgcctcgaagtattattgttttatagccaaatgtttagttaatctgctcacagatttaccactttctt
cactattcattctgtcttacacctctaacattccatctggggtaattttcctaaatgatcatgcatcctttgggatttcttttgatgatggtctattg
gtagtaaactctctcagttattgtttgtctgaaaatgtcatgcttttgccttcattgttgaagggtgcttttgctgggtggtcatttcagtatattg
aatatatcattccatcttccagtgtcatcattaaaaagtcagttgccagtctaactgcagctcttttataagtaacctgtcttattcttctggctg
catgtaaaagttttctctttgtctttgattttgtttagcttcaatctgctgtgtcttaatgatgggttcctattgtttgtcctgattgggattccgttaa
gattcctgaatctgtgggtagatatctttaatcagttttgaaacttctcagccattcttctaaaatattgattctccttcattctctcctcaccttct
agaattccaattaaatgtatgttagaccctgctctatctttcatatctctatactctcttctgtgtttttcatccttttgtctatttttccatgctttattc
tgaatagttccttctaatctaccttccaattaactaattttctctttagctatatctaatttgctgtaattaattacagttgccatttttatcctaaaatt
tctatttcatatttttgtatctgccatggtacttcttatggcttttaattccctgctaactatttaaagttcttattttatcctgtgaatatgatattccta
gttattttatttttaatttttattatttgttaatcttatgttttatttacacttcttttctgtgacatgagcacacacagattcatgtgtatacatatatgg
ctctgatacctctcctttcctgtcctcattcaaaccactgatcacagagagaggactatttttttttatttttaatttttctatttcaataggtttttgg
gggaacaggtggtgtttggttacatgaataagttctttagtggtgattttggtgcacccatcacccaaacagtgtacattgtacccaatgtg
taatct
tttaacccttgccacaccccaccctttccccgcagtccgcaaagtcccatgtatcattcttatgcctttgcttcctcatagcttagctcccac
atatgagtgagaacatacaatgtttggttttccattcctgagttatttaattaaaataatagtatccaattccatccaggttgctgtgaatgcca
ttattttgttcctttttatggttgagtagtattccatggtgtgtttgtgtgtgtataacatttttctttatccactcattgattgatgggcatttggct
ggttccatatttttgcaattgcaaattgtgctgttataaacatgtgtgtgcaagtatcttttttgtataatgacttcttttcctctgggtagatacct
agtagtgggattgctggatcaaatggtagatctacttttagttctataaggaatctccacactgttttccatagtggttgtatgagtttacattc
ccaccaatggtgtaaaagtgttcccttttcaccacatccacaccaacatctattattgtttgattttttattatgaccattcttgcaggagtgag
gtggtatcacattgtggttttgatttgcatttccctgataattagggatgttgagcatttttccatatgcttgttggtatttgttttttttttttttttttca
ttattatactttaagttttagggtacatgtgcacaatgtgcaggttagttacatatgtatacatgtgccatgctggtgtgctgcacccattaac
ccgtcatttagcattaggtatatctcctaatgctatccctccccaattccccccaccccgcttgttggtatttgtatatcttcatttgagaattct
ctgttcatgtccttagcccactttttgatgagattttttttttcttgctgattcgtttgagttctttgtagattctggatattagttggatgtatagatt
gtgaagattttctcccattctgtgggttgtctgttaactctgctaattatttcttttgctttgcagaagctttttagtttaattaagtcccatctattta
tctttgtttttgttgcatttgcttttgggttcttggtcatgaagtctttgcctaagccaatgtgtaggagggtttttccaatattatcttctagaatct
ttatggtttcaggtcttagatttaagtatttgatcgattttgagttgaattttgtataaggggagagagaaggattcagtttcattcttctacatg
caacttgccaattatcctaggaccatttgttgaatagggtgtcctttccccattttatgtttttgtttggtttgtcaaagatcagttggctgtaagt
gtttggctttatttctgggttatctattctgttccatttgtctacgtgactatttttataccagtaccatgttgttttggtgactatggccttacagta
tagtttgaagtctgataatgtaatgcctccagatttgttctttttacttagtcttgctttggctatgtgagctcttttttggtgccatatgaattttag
gattgttttttctagttctgtgaagaatgatggtggtattttgatgggaattgcattgaatttgtagattgtttttgggagtatggtcattttcaca
atattgattctacccattcatgagcatgggatgtgtttccatttgtttgtgtcatctatgattttctttcagcaatgttttgtagttttccttgtagag
ttcctagttattttaaagtctgtgttcggtctttcagcatttaaagtttgtaggtttattactatttctcttctttctgttggtcataactcttagtgtttt
gtttccttgtgtgcctggttacatatgtgctggtcattgtatttgaaaattatgtgtgaaataatttgaggttttggattatgtatattcctccaga
aagaatttcatttgcttctgtgcatttcttaggaacattacaagtccttcttctcagttaattttcgtagtatctttatcagataggtgctattacaa
ccactcacttagcagatgaaaatcatgaggctctgagagtctaagtcatctacttagaattggacaatggtgaagccaggattcaaaccc
acatcaataagaatccagcgctcttaacaaggggccagtacacttttttaaaaaataaaaggctagatagtaaatattttagactttgtgga
ctgcacagcctctgttgcaactactcaaccctgcctttgtagcatgaatgcagtcataaactatacataaatgaatgagcctggattcgttc
caaggaaactttataaaaacaggtggcaggctggatttggcccatgagaagtgtagtttacacaaaagttgagcaaaccaatttttttctg
attgtttttcctcttctcagtgtaagaagaagcttgggaagctgccacctcagtatgccctggagctcctgacggtctatgcttgggagcg
agggagcatgaaaacacatttcaacacagcccagggatttcggacggtcttggaattagtcataaactaccagcaactctgcatctact
ggacaaagtattatgactttaaaaaccccattattgaaaagtacctgagaaggcagctcacgaaacccaggtatgctatccccacatgg
cttagctcccctatgtaaatgaacacctggatacaggtacagtgccttggaaatggaggaggtgggagggctccccacttagtgagaa
tctcctgttgcccatcattgtactgggcattttactactgccatctgttttaaacacctacctccaaccctgtgaggcaggcactatgccaatt
attttacaggtgagtaaactgaggttctgagaggtaaggagcttgtccaacccttaacagaaaatgagtaaaatagctgcagtttgaact
gaaataagaacagcagcaacaacaatgatagtaattgctcccaggtattgaaagcttgttgtaagactaacacatgctaatataatagta
aaaattattagcaatattactgatatgtatgttatgttctagtcgctgtgctgagcatttcatataactgggctttttctatcctcacagcatagc
ctttgagataggtatgtggaactattcccattttacagataagaatcctgaggcttagagagttcaagtgacctacccaagggcacatcac
tgataaagggcagaggtgggattcaaacccacatctgtcaggtgcaagtgcaaggctccttctcctcatgctcactgcctgctggggaa
tagggcactggggacataccccagggagcccttcctcatgttctgagtcccagttcatcccatgctgctattttgctctcccaggagcatc
tggactccctagacagagccccagcttctcacctgtccctctctaaatgctgctctgcaggcctgtgatcctggacccggcggacccta
caggaaacttgggtggtggagacccaaagggttggaggcagctggcacaagaggctgaggcctggctgaattacccatgctttaag
aattgggatgggtccccagtgagctcctggattctgctggtgagacctcctgcttcctccctgccattcatccctgcccctctccatgaag
cttgagacatatagctggagaccattctttccaaagaacttacctcttgccaaaggccatttatattcatatagtgacaggctgtgctccata
ttttacagtcattuggtcacaatcgagggtttctggaattttcacatcccttgtccagaattcattcccctaagagtaataataaataatctcta
acaccatttattgactgtctgcttcgggctcaggttctgtcctaagccctttaatatgcactctctcattaaatagtcacaacaatcccatgag
gcatttttaaaaattttttattattttagattcagagggcacatgtgccatttgttacacagctatattgtgtaatggtggggtttgggcctctatt
gatcctgtcgcccaaatagtgaacagagtacccaaaaagaattttttcaacctttgcctttctcccttcctcctccctgttggagtccctagt
gtctattgttcccatctttagcagatgttaagtatttgattttctgtttctgggttaattcacttcggataatggcctccagctgcaaccatgattt
cattctttcttatggctgcataatactccatggtgtagatataccacactttctttatccagttcacactgatgggcacttaagttgattccatg
actttgctattgtgaatcgtactgcgataaacatacgagtgccggtgtcttttgatagaatgatttctttacctttgggtagataccgagtagt
gggattgctgggttgaatggacattctacttttagttatttgaaaagtcccatgaggcatgttttctatcattcccatcttacagatgagacaa
aggctcagagaggtgaggtcacttgctcaaggacatcagctaacaagtggtggaaatggaattcaagctcagtggactctaaagcca
gtgctcatgtcactgtgctaaacagcctgccttgtcacatccccacctctcatctgaccaatgggagactctgagcagctgagtgacttg
ggttgtcacacagctaaacaggggcaaaggacccagtcttggatctttccacctccaagcaggaatctgtctgattccaggggattgat
gatgttgcagatggctaggaagcagactccaggatggaatttagtatgcaggatgttctgggggagagccactggaaccagcactca
gggaaaggggggaagaaaggataggaaggaagcatgaaagagaatagggagaagtgaacagggatgcagagcgaatgccagtt
tcagccaactccaaggacagccctggagctggaatggcctttagagctgccccatggtgacagaggtggccaggcttctataccccta
cgtggatcactcactgtgcttgggcaccttgggaaagggcatggctttgagcaaaaggctctctgcagctgaggcaacccctaaaagg
gctgacggctgaagtctgtctgctgaccactgtcccagcagctggggcttgttagtccttcctcaaagggggatccagatggcatgtca
cagtgtctaccgtaaatgctcactgaatccagctgcaatgcaggaagactccctgatgtgatcatgtgtctcaccctttcaggctgaaag
caacagtgcagacgatgagaccgacgatcccaggaggtatcagaaatatggttacattggaacacatgagtaccctcatttctctcata
gacccagcacactccaggcagcatccaccccacaggcagaagaggactggacctgcaccatcctctgaatgccagtgcatcttggg
ggaaagggctccagtgttatctggaccagttccttcattttcaggtgggactcttgatccagagaggacaaagctcctcagtgagctggt
gtataatccaggacagaacccaggtctcctgactcctggccttctatgccctctatcctatcatagataacattctccacagcctcacttca
ttccacctattctctgaaaatattccctgagagagaacagagagatttagataagagaatgaaattccagccttgactttcttctgtgcacct
gatgggagggtaatgtctaatgtattatcaataacaataaaaataaagcaaataccattta

The sequence of the OAS1 PCR fragment for the G-allele (the G nucleotide is indicated in upper case): (SEQ ID 39)

cagatggcatgtcacagtgtctaccgtaaatgctcactgaatccagctgcaatgcaggaagactccctgatgtgatcatgtgtctcaccc
tttcaGgctgaaagcaacagtgcagacgatgagaccgacgatcccaggaggtatcagaaatatggttacattggaacacatgagtac
cctcatttctctcatagacccagcacactccaggcagcatccaccccacaggcagaagaggactggacctgcaccatcctctgaatgc
cagtgcatcttgggggaaagggctccagtgttatctggaccagttccttcattttcaggtgggactcttgatccagagaggacaaagctc
ctcagtgagctggtgtataatccaggacagaacccaggtctcctgactcctggccttctatgccctctatcctatcatagataacattctcc
acagcctcacttcattccacctattctctgaaaatattccctgagagagaacagagagatttagataagagaatgaaattccagccttgac
tttcttctgtgcacctgatgggagggtaatgtctaatgtattatcaataacaataaaaataaagcaaataccatttattgggtgtttattaactt
caaggcacagagccaagaagtacagatgcatatctaggggtattgtgtgtgtatatacattgattcaacaagaaatatttattgagcactt
actatgtgccaagcatagctctgg

The sequence of the OAS1 PCR fragment for the A-allele (the A nucleotide is indicated in upper case): (SEQ ID 40)

cagatggcatgtcacagtgtctaccgtaaatgctcactgaatccagctgcaatgcaggaagactccctgatgtgatcatgtgtctcaccc
tttcaAgctgaaagcaacagtgcagacgatgagaccgacgatcccaggaggtatcagaaatatggttacattggaacacatgagtac
cctcatttctctcatagacccagcacactccaggcagcatccaccccacaggcagaagaggactggacctgcaccatcctctgaatgc
cagtgcatcttgggggaaagggctccagtgttatctggaccagttccttcattttcaggtgggactcttgatccagagaggacaaagctc
ctcagtgagctggtgtataatccaggacagaacccaggtctcctgactcctggccttctatgccctctatcctatcatagataacattctcc
acagcctcacttcattccacctattctctgaaaatattccctgagagagaacagagagatttagataagagaatgaaattccagccttgac
tttcttctgtgcacctgatgggagggtaatgtctaatgtattatcaataacaataaaaataaagcaaataccatttattgggtgtttattaactt
caaggcacagagccaagaagtacagatgcatatctaggggtattgtgtgtgtatatacattgattcaacaagaaatatttattgagcactt
actatgtgccaagcatagctctgg

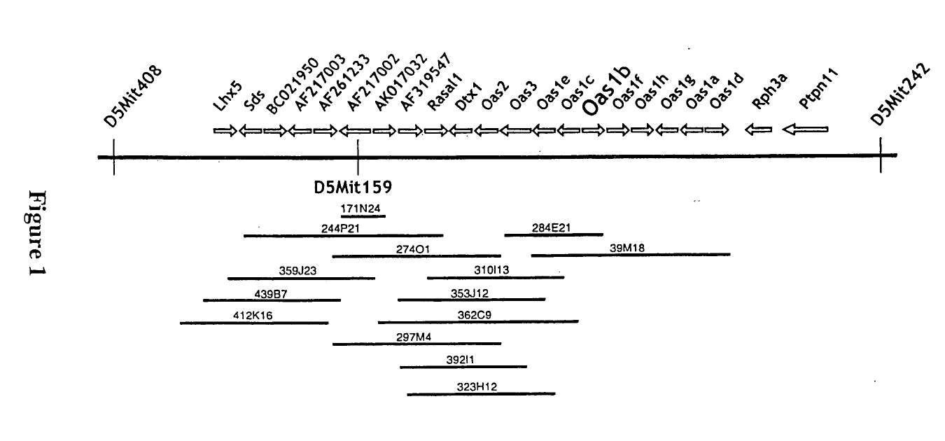

FIG. 1 shows physical and transcript maps of the Flv interval. Genes are represented by their accepted abbreviations or the GenBank accession numbers of their transcripts. The arrows represent the direction of gene transcription. The centromere is oriented toward the left of the figure. The Oas1b gene is indicated in bold. The flanking microsatellite markers are shown inside vertical rectangles, the D5Mit159 marker is shown inside a horizontal rectangle. The horizontal bars beneath the genes represent the BAC clones listed by the library name.

Twenty-two candidate genes for the Flv gene which controls resistance or susceptibility to flavivirus disease were identified. Full-length cDNAs were amplified by RT-PCR from congenic flavivirus resistant (C3H.PRI-Flvr) and susceptible (C3H/He) mouse strains for each gene identified in the Flv region using the primers listed in Table 2, sequenced and compared. The sequences of the majority of the genes in the two mouse strains were either identical or very similar (with only a few silent substitutions). In contrast, two genes, Na+/Ca2+-exchanger and Oas1b, were polymorphic and differed by several missense mutations. The Na+/Ca2+-exchanger cDNA from the C3H.PRI-Flv^{r} and C3H/He mouse strains differed by five non-synonymous substitutions (data not shown). cDNAs for this gene were subsequently sequenced from two additional susceptible (BALB/c and C57BL/6) and one additional resistant (BRVR) mouse strains. A random distribution of substitutions in the Na⁺/Ca²⁺-exchanger cDNAs were observed between the five mouse strains studied.

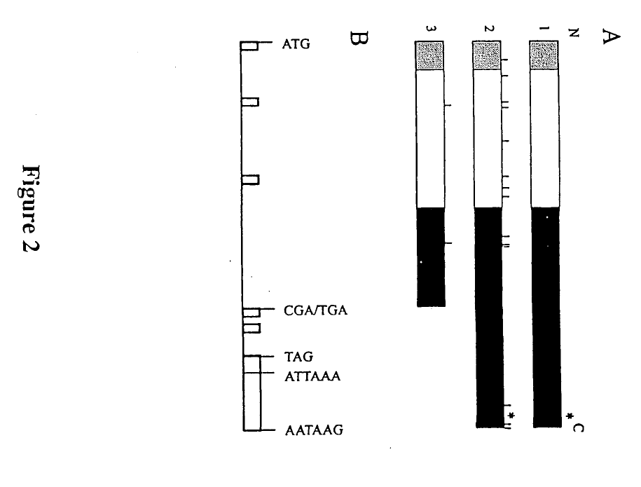

A total of 31 substitutions in Oas1b cDNA were found between the congenic C3H.PRI-Flv^{r} and C3H/He mouse strains. Most of these substitutions were silent, but the C820T transversion, in the susceptible C3H/He strain resulted in a premature stop-codon. The C3H/He Oas1b gene product therefore lacked 30% of its C-terminal sequence as compared to the C3H.PRI-Flv^{r} product (FIG. 2A). Two additional non-synonymous mutations resulted in a threonine to alanine substitution at position 65 and an arginine to glutamine substitution at position 190 in the susceptible C3H/He gene product.

FIG. 2 illustrates the structures of the Oas 1b gene and protein. A. Domain architecture of Oas1b proteins. The N-terminal domain (˜30 aa) (shown in gray) and the C-terminal domain (shown in black) are specific to the Oas protein family (generated with the ProDom tool). The nucleotidyltransferase domain (Pfam 01909) is shown in white. The CFK tetramerization motif is indicated by an asterisk. (1) Products of the Flv^{r} and Flv^{r}-like alleles. (2), Product of the Flv^{mr} allele. (3), Product of the Flv^{s} allele. Positions of amino acid substitutions between the Flv^{mr} and the Flv^{r} proteins are shown as vertical bars. B. Exon-intron structure of the mouse Oas1b gene. Exons are shown as open boxes. The positions of the start (ATG) and stop (TAG) codons, the substitution (CGA/TGA) that results in a premature stop codon and the two potential polyadenylation sites are indicated by vertical lines.

Comparison of Oas1b genomic (AC015535) and cDNA (AF328926) sequences revealed six exons. Based on the results of the 5′ RACE experiments, the size of the first exon was determined to be 243 bp in length and included 64 bp of the 5′ non-coding region (NCR). The lengths of the second, third, fourth and fifth exons were 277, 185, 233 and 154 bp, respectively. The fourth exon of Oas1b in the susceptible strain contained a premature stop-codon (FIG. 2B). All exon-intron boundaries contained conventional splicing sites. In the resistant strain, the sixth exon included the last 102 bp of the ORF and the 3′ NCR, which contained two potential polyadenylation signals separated by about 2 kb.

The individual exons of the Oas1b genes from eight additional mouse strains were next amplified from genomic DNA and sequenced. The Oas 1b gene encodes an identical full-length protein in all resistant strains (BRVR, C3H.PRI-Flv^{r}, CASA/Rk, and CAST/Ei), whereas the homologous gene from all susceptible strains (129/SvJ, BALB/c, C3H/He, C57BL/6, and CBA/J) encodes an identical truncated protein. The flavivirus susceptibility phenotype correlated with the Oas1b genotype in all nine mouse strains studied.

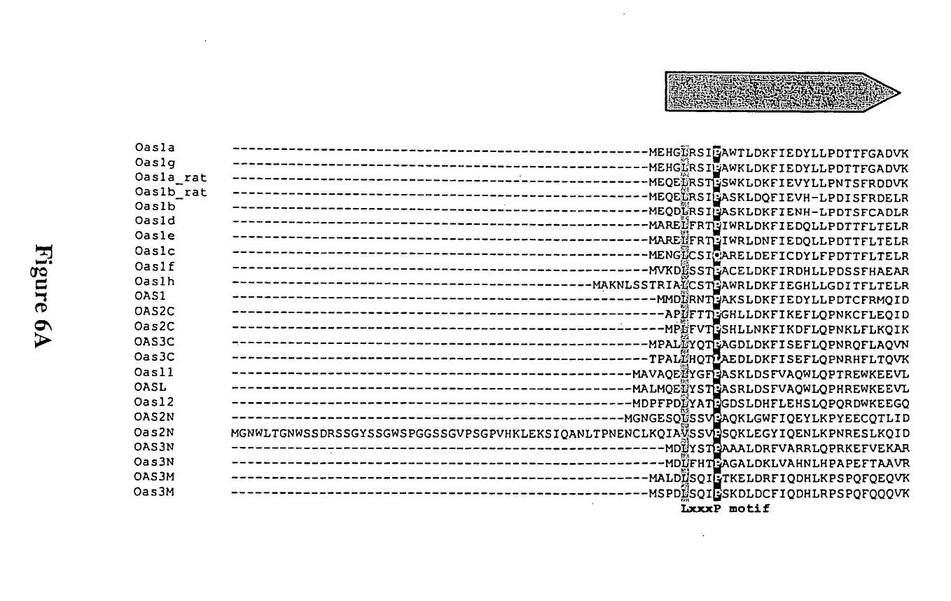

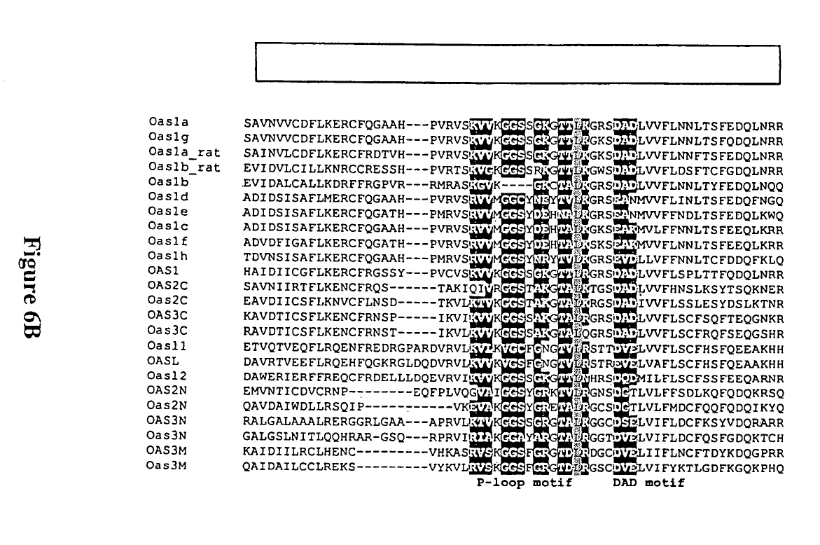

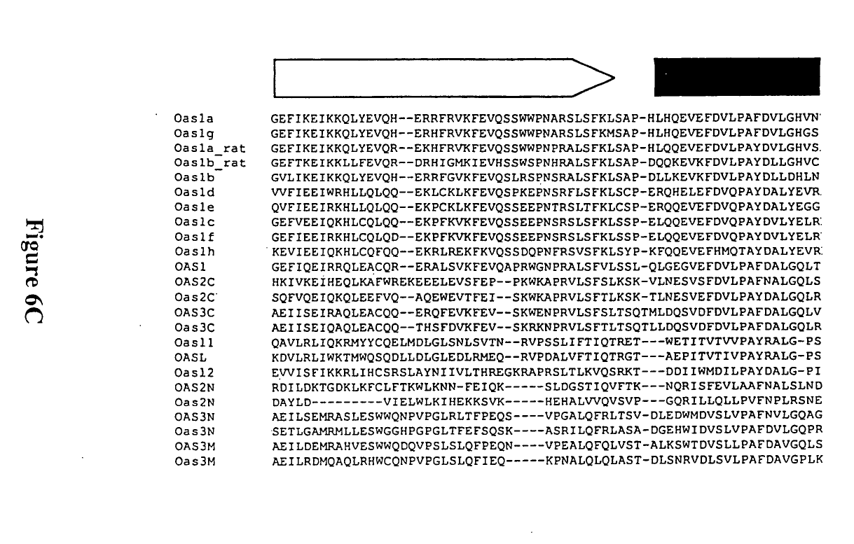

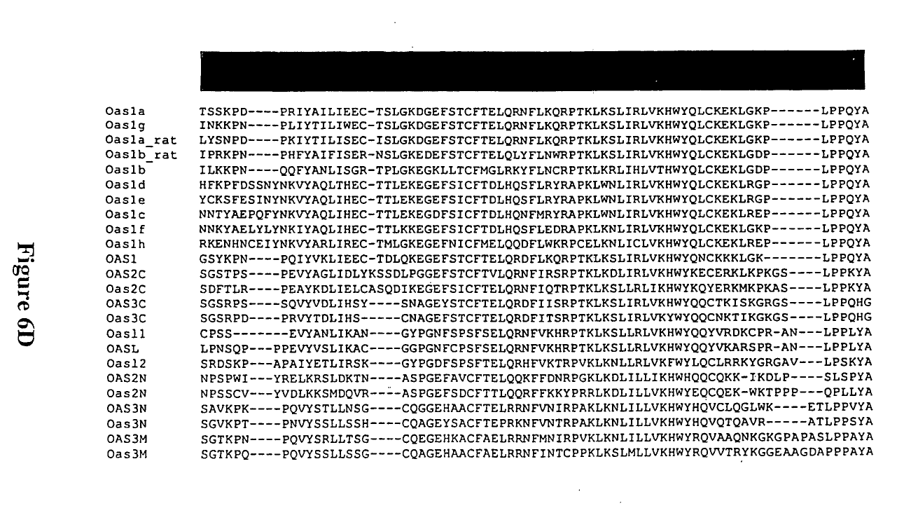

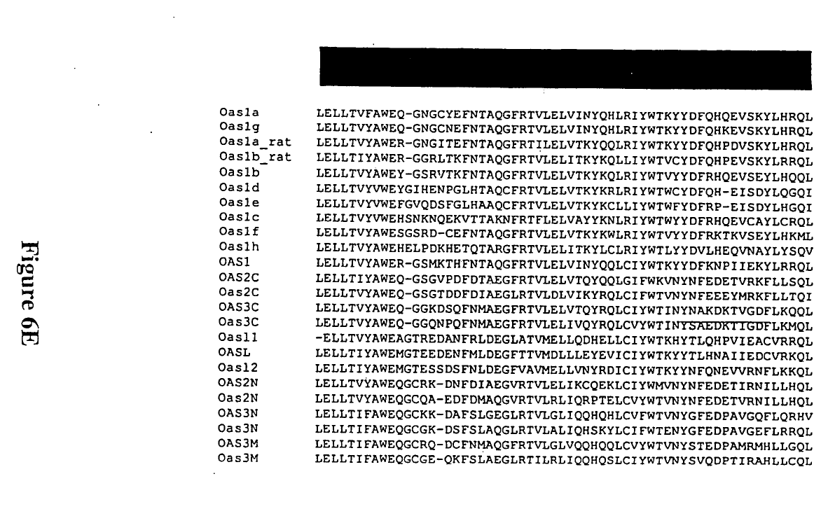

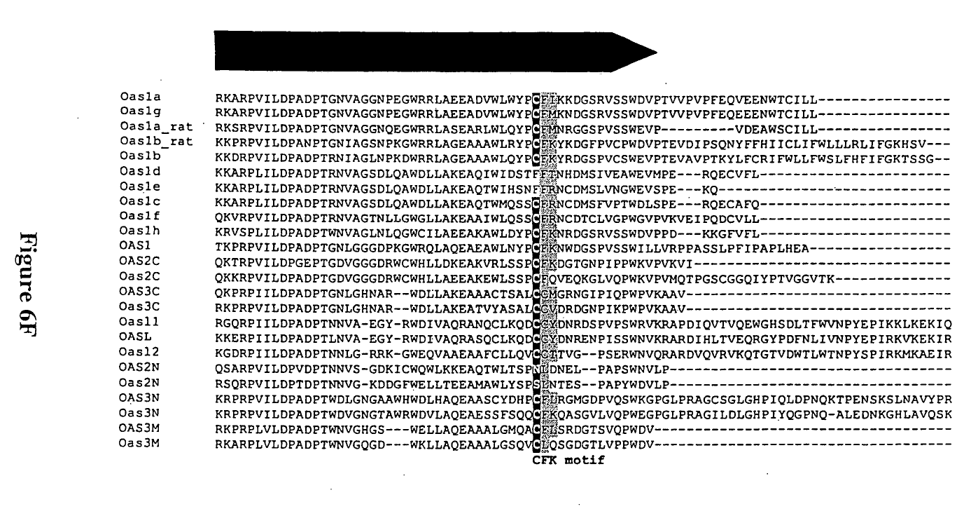

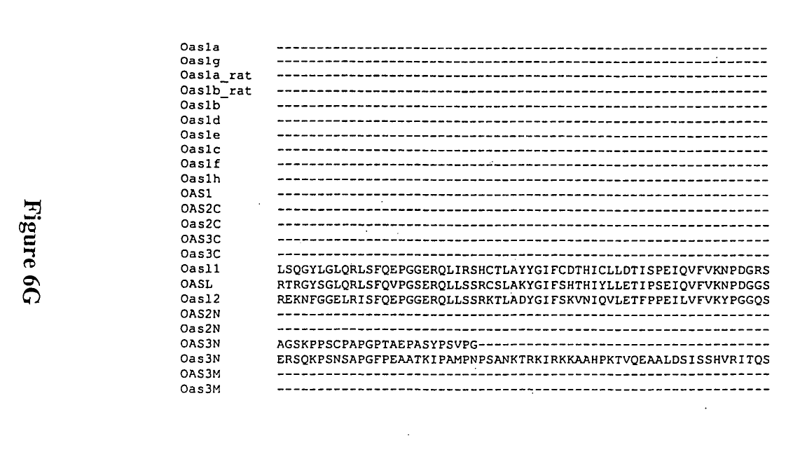

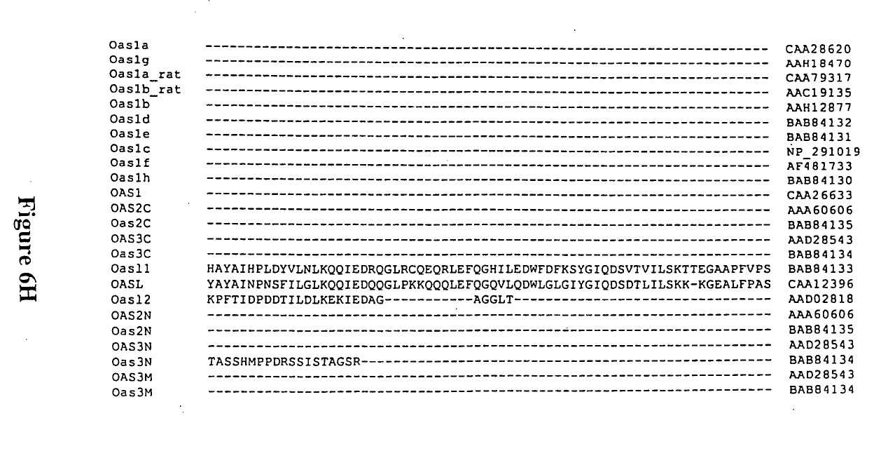

The Oas1b protein contains three domains (FIG. 2A). The N- and the C-terminal domains are unique to the 2′-5′ oligoadenylate synthetase family, whereas the central domain has a distinct nucleotidyltransferase fold. Several motifs were previously detected in murine 2′-5′ oligoadenylate synthetases (FIG. 6). FIG. 6 shows alignment of the protein sequences of the murine, rat and human 2′-5′ oligoadenylate synthetases. Conserved positions within known functional motifs are colored according to the physico-chemical properties of amino acid residues: hydrophobic residues are highlighted in yellow, charged residues are shown in a white or red background and small residues are shown in a white or blue background. The domain structure is shown above the alignment and corresponds to that shown in FIG. 2A. An N-terminal LxxxP motif is required for 2′-5′ oligoadenylate synthetase activity (30), whereas the P-loop motif is responsible for dsRNA binding (31). It has also been shown that a DAD Mg²⁺ binding motif is required for normal functioning of the murine 2′-5′ oligoadenylate synthetase (32). Although the LxxxP and DAD motifs were conserved in the products of both the resistant and susceptible alleles of the Oas1b gene, the P-loop motif contained a four amino acid deletion that was not found in the other murine 2′-5′ oligoadenylate synthetases (FIG. 6). A C-terminal CFK motif appears to be critical for tetramerization of the small form of mouse 2′-5′ oligoadenylate synthetase (33). The truncated susceptible Oas1b protein does not contain the CFK motif and so could not form the tetramer structure required for 2′-5′ oligoadenylate synthetase activity.

Although the Oas1b cDNA sequence from the MOLD/Rk strain (intermediate Flv phenotype) also encodes a full-length protein, it differs from the proteins of the other resistant strains by 14 amino acid substitutions, F26L, S45F, G63C, T65A, S83Y, C103Y, F110C, H118Q, P176L, S183L, I184T, T322A, G347A and M350T, distributed randomly across the protein (FIG. 2A). The MOLD/Rk Oas1b protein sequence contains alanine at position 65 similar to the proteins encoded by the susceptible strains. The MOLD/Rk Oas1b sequence differs by two substitutions, L26F and R206H, from the recently released sequence, AAH12877, derived from the CZECH II mouse strain, which has an unknown Flv phenotype. Both MOLD/Rk and CZECH II contain the same four amino acid deletion in the P-loop motif found in all Oas1b proteins.

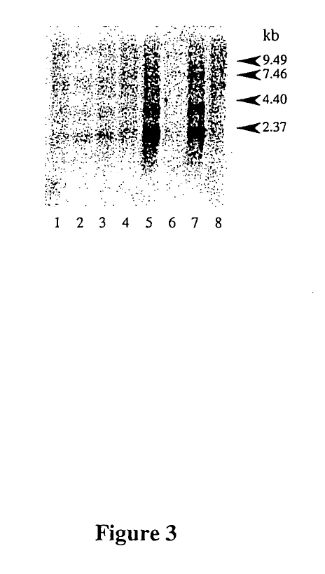

Investigations of the constitutive expression of the Oas1b gene in different mouse tissues were conducted. Although alpha/beta-interferon treatment up-regulated the transcription of the murine Oas1b gene (data not shown), constitutive expression of this locus was detected by Northern blotting in all 14 murine tissues tested. Two transcripts of the expected sizes, 2 kb and 4 kb (FIG. 3), were identified using a labeled cDNA probe derived from the 3′ NCR of Oas1b (cDNA positions 1384-1691 bp). The highest levels of constitutive expression were detected in lung and spleen (FIG. 3), thymus, placenta and uterus (data not shown).

FIG. 3 shows the constitutive expression of mouse Oas1b in different mouse tissues. A labeled Oas1b probe derived from the 3′ NCR of Oas1b was used to probe a BALB/c Northern blot (Stratagene) containing poly-A⁺ RNA (2 μg/lane) extracted from: 1, heart; 2, kidney; 3, liver; 4, lung; 5, skeletal muscle; or 6, spleen.

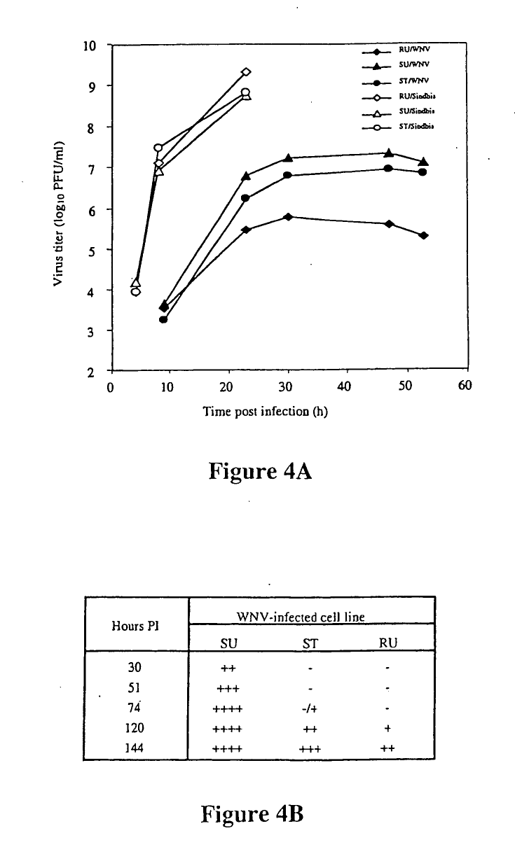

The effect of expression of C3H.PRI-FLV^{r} proteins in C3H/He cells on flavivirus replication was investigated. Since the Flv^{r} allele is dominant, its expression in susceptible cells was expected to have a dominant negative effect on flavivirus replication. C3H/He cells were transfected with the mammalian expression vector pEF6/V5-His-TOPO containing either the Oas1b or the Na⁺/Ca²⁺-exchanger cDNA from C3H.PRI-Flv^{r}. Stable cell lines were established by selection and cloning of transfected cells. The growth of the flavivirus, WNV, in susceptible C3H/He cell lines expressing either the Na⁺/Ca²⁺-exchanger or the Oas1b protein from resistant C3H.PRI-Flv^{r} was compared to that in untransfected C3H/He and C3H.PRI-Flv^{r} cells. No differences were observed either in the yields of WNV or in the time of appearance of cytopathic effect (CPE) between cell lines expressing the Na⁺/Ca²⁺-exchanger protein and untransfected C3H/He cells (data not shown). However, in C3H/He cell lines expressing a low level of the resistant Oas1b protein, viral titers were lower than those observed in untransfected cells, but not as low as in untransfected C3H.PRI-Flv^{r} cells (FIG. 4A).

FIG. 4 shows the effect of the low level expression of the resistant Oas1b protein in C3H/He cells on the growth of a flavivirus, West Nile virus, and an alpha togavirus, Sindbis. A. Virus growth curves. Cells were infected with either West Nile or Sindbis virus at a MOI of 0.5. Samples of culture fluid were taken at the indicated times and titered by plaque assay on BHK cells. RU, untransfected resistant C3H.PRI-Flv^{r} cells; SU, untransfected susceptible C3H/He cells; ST, susceptible C3H/He cells stably transfected with Oas1b cDNA from resistant C3H.PRI-Flv^{r} cells. B. Time course of the development of cytopathic effect (CPE) after infection of SU, RU and ST cells with West Nile virus. −, no obvious CPE; +, rounding or detachment of about 25% of the cells in the monolayer; ++, rounding or detachment of about 50% of the cells in the monolayer; +++, rounding or detachment of about 75% of the cells in the monolayer; ++++, complete destruction of the monolayer.

The appearance of CPE in C3H.PRI-Flv^{r} cells after WNV infection was significantly delayed as compared to that in C3H/He cells. The appearance of CPE was also delayed in C3H/He cells expressing the resistant Oas1b (FIG. 4B). In contrast, the growth and the time of appearance of CPE of an alpha togavirus, Sindbis, were similar in the three types of cells. The recombinant Oas1b protein contained both C-terminal 6×His and V5 tags which may have interfered with the activity of the 2′-5′ oligoadenylate synthetase by reducing the efficiency of tetramer formation. Surprisingly, cell lines expressing intermediate levels (8×) of resistant Oas1b protein showed lower levels of WNV suppression, while those expressing high levels (20×) of the protein showed no suppression (data not shown). The reasons for a negative correlation between suppression of WNV replication and the level of resistant protein expressed are currently not understood. Experiments to produce and test knock-in mice are underway.

The N-terminal 346 amino acids of OAS1 represent one functional unit, while OAS2 and OAS3 contain two and three functional units, respectively (19, 34-36). The murine Oas sequences obtained previously by different laboratories were named without knowledge of the entire gene family and designated by different symbols. A proposed simplified nomenclature for the murine Oas gene family is shown in Table 1. The single functional unit sequences were designated Oas1a through Oas1h, whereas the two- and three-unit sequences were designated Oas2 and Oas3, respectively (FIG. 1 and Table 1).

The 2′-5′ oligoadenylate synthetase-like genes, OASL and Oasl2 have recently been cloned from humans (36-37) and mice (38), respectively. A Celera database search revealed an additional murine gene, Oasl1, which was located close to Oasl2 and was about 6 Mb upstream the Oas2 locus. All of the human and mouse 2′-5′ oligoadenylate synthetase-like genes contained C-terminal ubiquitin-like domains. Although the Oas-like 1 (Oasl1) gene was mapped outside of the Flv interval, the cDNA of this gene was cloned and sequenced so that the comparative analysis of 2′-5′ oligoadenylate synthetase motifs included all of the known family members.

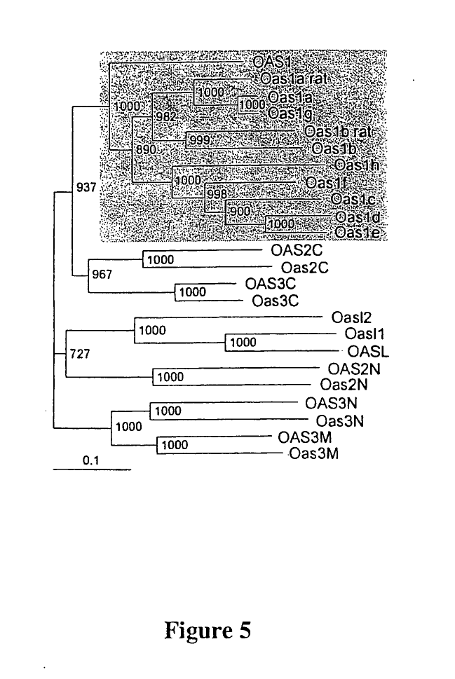

Available protein sequences for murine, rat and human 2′-5′ oligoadenylate synthetases were aligned and a phylogenetic tree was constructed (FIG. 5). The known rat proteins, AAC19135 and CAA79317, are encoded by two genes orthologous to murine Oas1b and Oas1a, respectively. Six additional rat Oas1 sequences have now been seqenced and are attached. All rodent Oas1 sequences cluster with the single human ortholog, OAS1. This clustering was fully supported by bootstrap analysis. The existence of eight apparent Oas1 paralogs in mice likely resulted from a series of gene duplication events. The one-to-many orthologous relationship between human and murine genes is unique to Oas1 and was not observed for other members of the Oas family (FIG. 5). FIG. 5 shows unrooted neighbor-joining, distance-based phylogenic tree of murine, rat and human Oas sequences. Human genes are designated by capital letters, while only the first letter is capitalized for the mouse genes. The sequences of the Oas2 and OAS2 proteins were divided into N- and C-terminal domains according to (19). The sequences of Oas3 and OAS3 proteins were divided into N-terminal (N), middle (M) and C-terminal (C) domains. The indicated bootstrap values were obtained with 1000 pseudoreplicates. The Oas1 cluster is shown on a gray background. The bar indicates the number of substitutions per site.

Twenty-two loci, including thirteen novel genes [a Ca²⁺-channel gene (AF217002), an unknown mRNA (AF217003), an ATP-dependent helicase (AF319547), a serine dehydratase (AF328927), a Na⁺/Ca²⁺ exchanger (AF217002), the Oas1b (AF328926), the Oas1d (AY055829), the Oas1e (AY055830 and AY055831), the Oas1f (AF481733), the Oas1g (AF480417), the Oas2 (AF418010), the Oas3 (AF453830), and the Oasl1 (AY057107)] were detected in a region of mouse chromosome 5 during positional cloning of the Flv gene. The D5Mit159 microsatellite sequence used for the initial BAC library screening was detected in the second intron of the Ca²⁺-channel gene (AF217002). By correlation of a polymorphism in the Oas1b gene with the susceptibility phenotypes of nine strains of flavivirus resistant and susceptible mice, the Flv gene was identified as Oas1b, a member of the 2′-5′ oligoadenylate synthetase gene family. 2′-5′ oligoadenylate synthetases bind dsRNA or particular secondary structures within single-stranded RNA (ssRNA) and catalyze the synthesis of 2′-5′ oligoadenylates (2-5A) from ATP (39). A major function of 2-5A is to bind and activate a latent endoribonuclease, RNase L, responsible for the degradation of viral and cellular ssRNAs (40). 2′-5′ oligoadenylate synthetases are also involved in other cellular processes such as apoptosis, cell growth and differentiation, regulation of gene expression, DNA replication and RNA splicing (19).

Data obtained with the three types of human 2′-5′ oligoadenylate synthetases, OAS1, OAS2, and OAS3 indicate that OAS3 functions as a monomer, while OAS2 and OAS1 are enzymatically active only as a homodimer and a homotetramer, respectively (19). The Oas1b genes from resistant mice encode full-length proteins, while those from susceptible mice encode C-terminally truncated proteins. Since the C-terminal region of the single-unit proteins is required for tetramerization, which is crucial for 2′-5′ oligoadenylate synthetase activity, it is likely that the Oas1b proteins produced by susceptible mice are not active. The OAS1, OAS2 and OAS3 genes are differentially induced by interferons alpha, beta and gamma in various tissues (19). Although the expression of the mouse Oas 1b gene was up-regulated after incubation with alpha/beta-interferon, it was found to be constitutively expressed at low levels in both resistant and susceptible cells (data not shown). These results are consistent with the previous observation that flavivirus resistance was not diminished in resistant mice after injection of anti-alpha/beta interferon antibody (41).

The effect of the Flv gene product is virus-specific, since it suppresses the replication of the members of the genus Flavivirus, but has no effect on the replication of other types of viruses. The functions of 2′-5′A and the latent endoribonuclease, RNase L are both non-specific. The Oas1b proteins from both resistant and susceptible mice differ from other 2′-5′ oligoadenylate synthetases by one unique change, a four amino acid deletion within the P-loop motif. The P-loop region is involved in RNA recognition and binding and may allow the Oas1b protein to specifically recognize and bind a specific conserved RNA structure unique to flavivirus RNAs. In support of this hypothesis, Urosevic and co-authors (42) reported that the OR156 strain of Murray Valley encephalitis virus, which had a 62-nucleotide deletion in its 3′non-coding region (43), replicated more efficiently in resistant mice than did strains of this flavivirus with full-length RNAs. Results from previous sucrose gradient analyses (10) and recent RNase protection experiments (data not shown) indicate that the levels of genomic flavivirus RNA, but not anti-genomic RNA, are preferentially reduced in flavivirus infected resistant cells as compared to susceptible cells. Also, more flavivirus dsRNA and less viral ssRNA were detected in the brains of resistant mice as compared to those of susceptible animals (44). Since genomic RNA is found free in the cytoplasm, it would be more susceptible to digestion by RNase L than would anti-genomic RNA, which is only present in double-stranded replication intermediate RNA structures. Since the Oas1b proteins from both resistant and susceptible mice have the same four amino acid deletion in the P-loop motif, both proteins would be expected to bind specifically to flavivirus RNAs, but RNA binding would only activate the full-length resistant Oas1b protein. It is currently not known whether the 2′-5′ oligoadenylate synthetase activity alone is sufficient to confer the flavivirus resistant phenotype or whether as yet uncharacterized activities of the Oas1b protein also contribute. Even though the Flv^{mr} proteins also had the same deletion in the P-loop motif, the additional amino acid substitutions in these proteins were apparently responsible for the reduced level of resistance observed in this strain. This suggests that additional regions of the Oas1b may also be functionally important for susceptibility. Functional studies with a recombinant Oas1b protein are currently underway to address these questions. The advantage provided to mice and possibly to other rodents by the large number of Oas1 genes is currently not understood. Some of the mouse Oas1 gene products are not active synthetases, including Oas 1b. Preliminary data suggest that an inactive synthetase such as Oas1b can enhance the enzymatic activity of an active synthetase. One mechanism by which this could occur would be via the formation of heterotetramers. The formation of different heterotetramers could regulate activity levels. The Oas1 gene products may also interact with other cell proteins to accomplish additional as yet unknown functions.

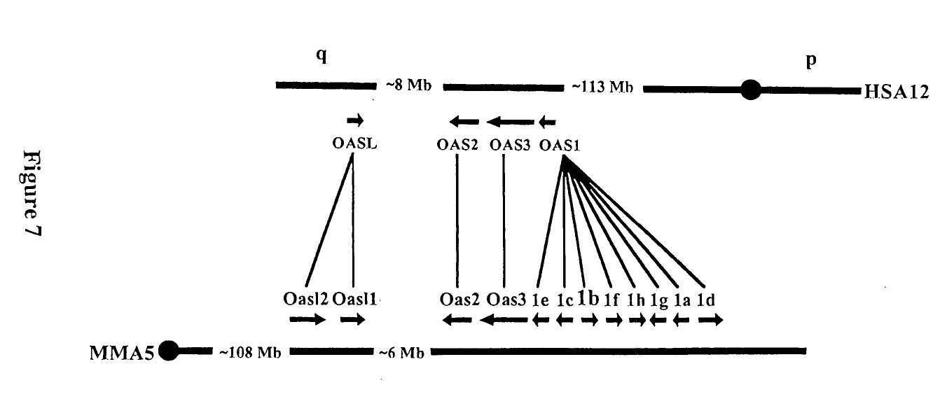

Prior to the current invention, inherited flavivirus resistance appeared to be restricted to Mus species. Rats have multiple Oas1 genes and an Oas1b ortholog (AF068268), but resistance to flaviviruses has not yet been studied in rats. Humans have only a single OAS1 gene, but this gene produces multiple isoforms via alternative splicing (FIG. 7).

Human OAS proteins, OAS1, OAS2, OAS3 and OAS-like, are 2′,5′-oligoadenylate synthetases. The members of this family of interferon-induced proteins function in the antiviral action pathways of interferon but also have functions in gene regulation, apoptosis and development. When activated by double-stranded RNA, these proteins polymerize ATP into 2′,5′-linked oligomers with the general formula pppA(2′p5′A)ₙ. This mixture of oligonucleotides is known as 2-5A and currently it is believed that 2-5A binds and activates a latent endoribonuclease responsible for the degradation of viral and cellular RNAs.

Resistance to infection by flaviviruses has been associated with variations in human OAS genes, particularly OAS1. The current invention also contemplates that mutations in other OAS genes may also be important for virus resistance/susceptibility.

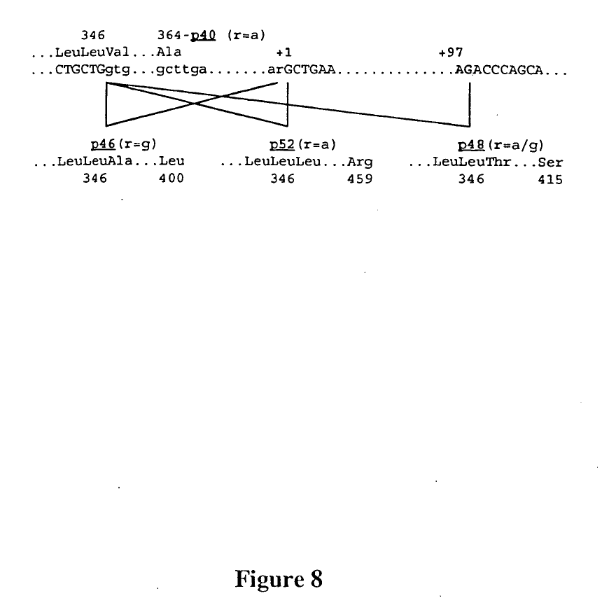

When the G is present (G-allele) in the human OAS1 gene at nt position 12,349 (numbered beginning from the ATG-start codon in the genomic DNA) (FIG. 8; Celera SNP accession number hCV2567433) the OAS1 transcripts encoding the p46 and p48 isoforms are produced as a result of alternative splicing events. The conventional splicing acceptor “ag” at the end of intron 5 is utilized for processing of the p46 mRNA. Utilization of a different splice acceptor located 96 nucleotides downstream from the conventional intron 5 acceptor results in the production of the p48 mRNA.

When an A is present (A-allele) at OAS1 nucleotide position 12,349, the splice acceptor site is mutated to a non-functional “aa” and the p40 mRNA is produced by read-through into intron 5 (FIG. 8). An additional splice acceptor (aG, where G is the +1 nucleotide located at the beginning of the conventional exon 6) can alternatively be utilized to produce p52 mRNA designated hCT31628 in the Celera human transcript database. The p48 transcript can also be produced by individuals with the A-allele. The 346 N-terminal amino acids of the p40, p46, p48, and p52 are identical but their C-terminal regions differ due to alternative splicing. Each of the isoforms has a unique C-terminus translated from different ORFs. Stop-codons for the ORFs encoded by p46, p48 and p52 transcripts are located in exon 6 at positions +163/+165, +303/+305, and +341/+343, respectively. The stop-cod the p40 transcript is located at position +54/+57 in intron 5.

Data from a study of human populations indicated that the different alleles of the OAS1 gene are important for determining viral resistance/susceptibility. The frequency of the A-allele in ethnic Russian populations who are known to be highly resistant to disease caused by the flavivirus, tick borne encephalitis virus, was significantly increased (up to 99%) as compared with the frequency in a group of non-ethnic patients with tick borne encephalitis virus-induced disease (59%). The GG homozygous individuals exhibited the most severe disease symptoms. No GG-homologous individuals were detected in the native Siberian populations. In contrast, the frequencies of the A-allele and the G-allele were similar in groups of healthy Russians and Russians infected with hepatitis C virus (a distantly related member of the flavivirus family from a different genus. The data is summarized in Table 1.

TABLE 1
Human Viral Resistance Genotypes

| | | Frequencies and numbers of genotypes | | | | | Frequencies of alleles | |
|---|---|---|---|---|---|---|---|---|
| Population | N | AA | | AG | | GG | A | G |
| Russians | 134 | 0.69 | (93) | 0.28 | (37) | 0.03 (4) | 0.83/0.71* | 0.17/0.29* |
| Altains | 30 | 0.70 | (21) | 0.30 | (9) | 0.00 (0) | 0.85 | 0.15 |
| Chukchi | 114 | 0.85 | (97) | 0.15 | (17) | 0.00 (0) | 0.93 | 0.07 |
| Tuvinians | 42 | 0.98 | (41) | 0.02 | (1) | 0.00 (0) | 0.99 | 0.01 |
| TBE | 22 | 0.36 | (8) | 0.45 | (10) | 0.19 (4) | 0.59 | 0.41 |
| patients | | | | | | | | |

*Data from two genotypings.

Linkage with additional polymorphisms located in exon 6 has been observed (Table 2).

TABLE 2
Linkage disequilibrium of A/G mutations in the intron 5 acceptor
site (hCV2567433) with additional SNPs in exon 6 of the OAS1.

| hCV2567433 | rs3177979 | rs1051042 | hCV2567429 | hCV2567429 + 1nt |
|---|---|---|---|---|
| AA | AA | CC | AA | AG |
| GG | GG | GG | GG | GG |

The G-allele linked mutations in exon 6 would cause the following amino acids changes: 352A and 361R in p46 and 397G in p48. The A-allele linked mutations in exon 6 would cause the following amino acid changes: 397K/R in p48 and 361R and 429K in p52. Some or all of these additional changes may also be functionally relevant. Data also shows that mutations in other human OAS genes may be relevant in determining viral resistance/susceptibility (Table 3).

TABLE 3
Frequencies of genotypes and alleles in OAS2 and OAS3 SNPs.

| | | OAS2 SNP rs15895 | | | | | OAS3 SNPs rs2285932 | | | | |
|---|---|---|---|---|---|---|---|---|---|---|---|
| | | Genotypes | | | Alleles | | Genotypes | | | Alleles | |
| Population | N | AA | AG | GG | A | G | CC | CT | TT | C | T |
| Altaians | 30 | 0.00 | 0.43 | 0.57 | 0.13 | 0.87 | 0.70 | 0.30 | 0.00 | 0.85 | 0.15 |
| TBE patients | 18 | 0.12 | 0.44 | 0.44 | 0.33 | 0.67 | 0.33 | 0.44 | 0.23 | 0.56 | 0.44 |

The methods of the present invention comprise the identification of resistant genotypes. By sequencing a human resistance/susceptibility gene, such as the OAS1 gene, specific sequences were identified that were cleaved by a specific restriction endonuclease. A method for identifying the genotype of an individual comprises cleaving a nucleic acid sample from an individual with one or more specific endonucleases that are known to differentiate between viral resistant genotypes. The pattern is observed after separating the cleaved nucleic acid segments by electrophoresis on a gel and used to determine the genotype of the source of the nucleic acid sample.

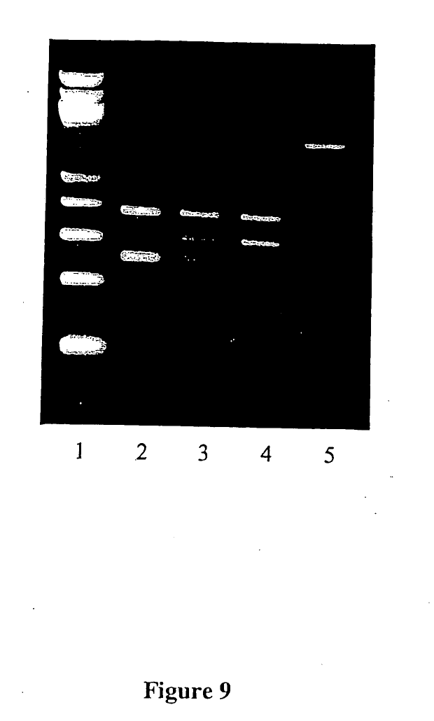

About 20 ng of human genomic DNA was amplified by PCR for each sample using the HO1-F forward primer (5′ CAGATGGCATGTCACAGTGTCTAC 3′) and the HO1-R reverse primer (5′ CCAGAGCTATGCTTGGCACATAGT 3′) in a total volume of 25 microliters. After amplification, 5 microliters of restriction mix containing 3 microliters of 10× restriction buffer, 10 units of Alu I endonuclease, and 1 microliter of distilled water were added directly to each PCR tube and incubated for 1 hour at 37° C. Restriction reactions were resolved on 2% agarose gels, stained with ethidium bromide and the gels were photographed under UV light. Other visualization or measurement methods are contemplated by the present invention. There are four AluI-recognition sites in the PCR product derived from the G-allele. AluI digestion of this product generates two large restriction fragments of 378 bp and 306 bp as well as three short (invisible) restriction fragments of 46 bp, 12 bp, and 8 bp, respectively. In PCR DNA containing the A-allele, an additional AluI restriction site is present which would divide the 306 bp fragment into a large 255 bp fragment and a short (invisible) 51 bp fragment. Therefore, the AluI restriction pattern of the G-allelic variant contains a unique visible fragment of 306 bp, while that of the A-allelic variant contains a unique visible fragment of 255 bp on the agarose gel (FIG. 9). There are no isoschizomers for AluI. There are currently no additional known restriction enzymes specific for the sequence around the mutation. Additional techniques that could be used for genotyping of this mutation are: 1). Direct sequencing of region containing the mutation; 2). A SNP assay using single nucleotide extension by Real Time PCR; 3). Microchip hybridization. The methods are well known to those skilled in the art.

Compositions of the present invention comprise endonucleases, solutions and buffers necessary for cleaving of DNA samples. Additionally, reference DNA samples of resistant and susceptible genes are included. Preferred compositions are found in kits for testing the genotypes of individuals. Other compositions included in the present invention comprise constructs and vectors comprising the relevant sequences, cell lines derived from individuals that have different alleles, which affect their virus resistance/susceptible phenotype, which could be used for comparing the efficacy of candidate antiviral agents or strategies under different host-virus conditions. Additionally, the present invention comprises compositions comprising cell lines transformed by the relevant sequences comprising the resistant or susceptible variants.

The present invention comprises methods and compositions for determining viral resistance/susceptibility by indentifying the genotype of the human or animal. Alleles of the OAS genes are one set of indentifiable genes that determine viral resistance/susceptibility. Identifying these alleles in a human or animal, either alone or in combination with other genes, determines the resistance/susceptibility to viral infection, particularly flavivirus infection. For example, seven single nucleotide polymorphisms (SNPs) were genotyped within the human locus encoding interferon-inducible double stranded RNA dependent protein kinase (PRKR). Two of these SNPs, rs4648174 and rs2287350, showed complete linkage disequilibrium (always segregated together) in 122 Russian DNA samples tested. However, in 44 DNA samples from TBEV-induced disease patients, the genotype distribution of these two SNPs in 20 patients with febrile disease (fever) differed significantly (χ²=12.4; P=0.002) from that in 24 patients with severe disease (meningitis, encephalitis and/or poliomyelitis). These data suggest that variation in additional innate immune system genes, such as PRKR, could also be associated with the severity of flavivirus-induced disease in humans. The present invention comprises methods and compositions for identifying OAS alleles and other genes for determining the extent of flavivirus infection, severity of viral disease, and susceptible/resistant populations, among other aspects of viral disease.

Methods of the present invention are not limited to the viruses described herein, but include methods for determining the genotype of individuals for a resistant or susceptible response to any virus for which an interferon response is made by the body. Preferred methods comprise determining the genotype of an individual, particularly for OAS alleles, including OAS1, OAS2, OAS3 and OAS-like alleles, using selective endonuclease characterization of the individual's DNA.

Sequences Disclosed Herein:

| (SEQ ID 1)LOCUS AF217002 2742 bp mRNA linear ROD 25-JUN-2002 | |
|---|---|
| DEFINITION Mus musculus calcium channel mRNA, complete cds. | |
| ACCESSION AF217002 | |
| translation=“MAVSLDDDVPLILTLDEAESAPLPPSNSLGQEQLPSKNGGSHSI | |
| HNSQVPSLVSGADSPPSSPTGHNWEMNYQEAAIYLQEGQNNDKFFTHPKDARALAAYL | |
| FVHNHFFYMMELLTALLLLLLSLCESPAVPVLKLHTYVHATLELFALMVVVFELCMKL | |
| RWLGFHTFVRHKRTMVKTSVLVVQFIEAIVVLVRQTSHVRVTRALRCIFLVDCRYCGG | |
| VRRNLRQIFQSLPPFMDILLLLLFFMIIFAILGFYLFSTNPSDPYFSTLENSIVNLFV | |
| LLTTANFPDVMMPSYSRNPWSCVFFIVYLSIELYFIMNLLLAVVFDTFNDIEKHKFKS | |
| LLLHKRTAIQHAYGLLASQRRPAGISYRQFEGLMRFYKPRMSARERFLTFKALNQSNT | |
| PLLSLKDFYDIYEVAALQWKAKRNRQHWFDELPRTAFLIFKGINILVNSKAFQYFMYL | |
| VVAVNGVWILVETFMLKGGNFTSKHVPWSYLVFLTIYGVELFMKVAGLGPVEYLSSGW | |
| NLFDFSVTAFAFLGLLALTLNMEPFYFIVVLRPLQLLRLFKLKKRYRNVLDTMFELLP | |
| RMASLGLTLLTFYYSFAIVGMEFFNGRLTPNCCNTSTVADAYRFINHTVGNKTKVEEG | |
| YYYLNNFDNILNSFVTLFELTVVNNWYIIMEGVTSQTSHWSRLYFMTFYIVTMVVMTI | |
| IVAFILEAFVFRMNYSRKSQDSEVDSGIVIEKEMSKEELMAVLELYREERGTSSDVTR | |
| LLDTLSQMEKYQQNSMVFLGRRSRTKSDLSLKMYQEEIQEWYEEHAREQEQQKLRGSV | |
| PGPAAQQPPGSRQRSQTVT” | |
| BASE COUNT 568a 819c 727g 628t | |
| ORIGIN | |
| 1 | aaggctggcg cagctgccgc tgtggcagcg gtgaggcggc ggtggcggct gctgaggctc |
| 61 | cgcgctgggg atattggcgg cggcaactgc gggctgagct acgctgtgca gacccagtgc |
| 121 | acagtgcggg atcccgggac ggcgcgtacc ttagaagatg cctctgatgg aacaggctct |
| 181 | gggaagcttc cccggccccg tggctttgaa caggagctca agccggaggc agtttaaagc |
| 241 | cctggccgtt gtatcctgag gaccgcaggt caggagaaga tggctgtaag tttagatgac |
| 301 | gatgtgccgc tcatcctgac cttggacgag gctgagagtg ctccgctgcc tccttcgaac |
| 361 | agcctgggcc aagagcagct gcccagcaaa aatgggggca gccacagcat ccacaactcc |
| 421 | caggtcccca gtctggtctc cggagcggac agccccccct ccagtcccac cggacacaac |
| 481 | tgggagatga attatcaaga ggcggcaatc tacctccagg aaggtcagaa caacgacaag |
| 541 | ttcttcaccc accccaagga tgccagagcg ctggcggcct acctcttcgt ccacaaccac |
| 601 | ttcttctaca tgatggagct gctcacggcc ctgctcctgc tgctgctgtc gctgtgcgag |
| 661 | tcccccgctg tccccgtgct caagctgcac acttacgtcc acgccacgct ggaactcttt |
| 721 | gccctcatgg tggtggtatt tgaactctgc atgaaattgc ggtggctggg cttccacacg |
| 781 | ttcgtccggc acaaacgtac catggtcaag acgtccgtcc tcgtggtgca gttcatcgag |
| 841 | gccattgtgg tgctggttcg gcagacgtcc cacgtgcggg tgacccgggc actacgctgc |
| 901 | attttcctgg tggactgtcg ctactgtggc ggtgtacggc gcaacctgcg gcagatcttc |
| 961 | cagtctctcc cacccttcat ggacatcctc ctgttgctgc tcttctttat gatcatcttt |
| 1021 | gccatcctcg gtttctactt attctccaca aatccttccg acccctactt cagcaccctg |
| 1081 | gagaacagca tcgtcaacct gttcgttctc ctgaccacag ccaactttcc agatgtcatg |
| 1141 | atgccctcct actcccggaa cccctggtcc tgcgtcttct tcattgtata cctctccatt |
| 1201 | gagctgtact tcatcatgaa cctgctcctg gccgtggtgt tcgacacctt caacgacatt |
| 1261 | gaaaagcaca agttcaagtc tttgctgctg cacaaacgga ccgccatcca gcatgcctac |
| 1321 | ggcctgcttg ccagccaacg gaggccggct ggcatctcct acaggcagtt cgaaggctta |
| 1381 | atgcgcttct acaagccccg gatgagtgca agggaacgct tcctgacttt caaggccttg |
| 1441 | aaccagagca acacgcctct gctcagcctg aaggacttct atgatattta cgaagtcgct |
| 1501 | gctctgcagt ggaaggcaaa gagaaacaga cagcattggt ttgatgagct cccccggaca |
| 1561 | gccttcctca tcttcaaagg gattaacatc cttgtgaatt ccaaggcctt ccagtatttc |
| 1621 | atgtacttgg tggtggctgt caacggtgtc tggatcctgg tggagacatt catgttgaaa |
| 1681 | ggtgggaatt tcacctcaaa gcatgtgccc tggagttacc tcgtgtttct taccatctat |
| 1741 | ggagttgaac tgttcatgaa ggtggctggc ctgggccctg tggagtacct gtcctctgga |
| 1801 | tggaacctgt tcgatttctc ggtcacggca ttcgccttcc tgggactgct cgcactgacg |
| 1861 | ctcaacatgg aacccttcta tttcattgtg gtcctgcgtc cccttcagct gctgaggtta |
| 1921 | tttaaactga agaaacgcta ccgcaacgtg ttggacacca tgtttgagct gctgccgcgg |
| 1981 | atggccagcc ttggcctcac gctgctcacc ttctactatt ccttcgccat cgtgggcatg |
| 2041 | gagttcttca acgggcggct gacccccaac tgctgcaaca ccagcacagt ggccgacgcc |
| 2101 | taccggttca tcaaccacac tgtgggcaat aagaccaagg tagaggaagg ctactactat |
| 2161 | ctcaacaact ttgacaacat cctcaacagc ttcgtgacct tgtttgagct caccgttgtc |
| 2221 | aacaattggt acatcatcat ggaaggcgtc acctcgcaga cgtcccactg gagccgcctg |
| 2281 | tacttcatga ccttttacat agtgaccatg gtggtgatga ccattatcgt ggccttcatc |
| 2341 | ctggaggcct tcgtcttccg catgaactac agccgcaaga gccaggactc ggaagtggac |
| 2401 | agtggcatcg tcatcgagaa ggaaatgtcc aaggaggagc ttatggccgt cctggagctt |
| 2461 | tatcgtgagg agcgaggcac ctcctctgac gtgacccggc tgctggacac cctctctcag |
| 2521 | atggagaaat accagcaaaa ttccatggtg tttctgggac ggcgatcgag aaccaaaagt |
| 2581 | gacctgagtc tgaagatgta ccaggaggag atccaggagt ggtacgagga gcatgcccgg |
| 2641 | gaacaggagc agcagaagct caggggcagc gtgcccggcc ctgcagccca gcagccccct |
| 2701 | ggcagtcgcc agcgctccca gactgtcacc tagctgggtt tc |
| // | |
| (SEQ ID 2)LOCUS AF217003 3569 bp mRNA linear ROD 25-JUN-2002 | |
| DEFINITION Mus musculus unknown mRNA. | |
| ACCESSION AF217003 | |
| /translation=“MAAPVDGSSGGWAARALRRALALTSLTTLALLASLTGLLLSGPAGALP | |
| TLGPGWQRQNPDPPVSRTRSLLLDAASGQLRLEDGFHPDAVAWANLTNAIRETGWA | |
| YLDLSTNGRYNDSLQAYAAGVVEASVSEELIYMHWMNTVVNYCGPFEYEVGYCEKL | |
| KNFLEANLEWMQREMELNPDSPYWHQVRLTLLQLKGLEDSYEGRLTFPTGRFTIKPLG | |
| FLLLQISGDLEDLEPALNKTNTKPSLGSGSCSALIKLLPGGHDLLVAHNTWNSYQNML | |
| RIIKKYRLQFREGPQEEYPLVAGNNLVFSSYPGTIFSGDDFYILGSGLVTLETTIGNKN | |
| PALWKYVQPQGCVLEWIRNVVANRLALDGATWADVFKRFNSGTYNSQWMIVDYKAFL | |
| PNGPSPGSRVLTILEQIPGMVVVADKTAELYKTTYWASYNIPYFETVFNASGLQALVA | |
| QYGDWFSYTKNPRAKIFQRDQSLVEDMDAMVRLMRYNDFLHDPLSLCEACNPKPNAEN | |
| AISARSDLNPANGSYPFQALHQRAHGGIDVKVTSFTLAKYMSMLAASGPTWDQCPPFQ | |
| WSKSPFHSMLHMGQPDLWMFSPIRVPWD” | |
| BASECOUNT 675a 1133c 945g 816t | |
| ORIGIN | |
| 1 | acctgccctc gcgatggcgg cccccgtgga tgggagctcc ggcggctggg cggcccgggc |
| 61 | gctacggcgg gcactggcgc tgacctccct gaccacactg gccttgctgg cctcgctgac |
| 121 | cgggctgttg ctgagcggcc cggcgggcgc tctccctacc ctggggcccg gctggcagcg |
| 181 | ccaaaatccg gacccgccgg tctcccgcac ccgctcgctg ctgctggacg ccgcgtcggg |
| 241 | ccagctgcgc ctggaggatg gcttccaccc cgacgcggtg gcctgggcca acctcaccaa |
| 301 | cgccatccgc gagaccgggt gggcctatct ggacctgagc acaaatggca ggtacaatga |
| 361 | cagcctgcag gcctatgcag ctggtgtggt ggaggcctct gtgtctgagg agctcatcta |
| 421 | catgcactgg atgaacacgg tggtcaacta ctgcggcccc ttcgaatacg aagtcggcta |
| 481 | ctgtgagaag cttaagaact tcctggaggc caacctggag tggatgcaga gggaaatgga |
| 541 | gcttaacccg gactctccgt actggcacca ggtgcggctg accctcctgc agctgaaagg |
| 601 | cctggaggac agctatgaag gccgtttaac cttcccaact gggaggttca ccatcaaacc |
| 661 | cttggggttc ctcctgctgc agatctctgg agacctggaa gacctagagc cagccctgaa |
| 721 | taagaccaac accaagcctt ccctgggctc cggttcatgc tctgccctca tcaagctgct |
| 781 | gcctggcggg catgacctcc rggtggcgca caacacgtgg aactcctacc agaacatgtt |
| 841 | acgcatcatc aagaagtaca ggctgcagtt ccgggagggg ccgcaagagg agtaccccct |
| 901 | ggttgctggc aacaacttgg ttttctcgtc ctacccgggc accatcttct ccggagatga |
| 961 | cttctacatc ctgggcagtg gcctggtcac cctggagacc accattggca acaagaaccc |
| 1021 | agccctgtgg aagtacgtgc agccccaggg ctgtgtgctg gagtggatac gaaatgtcgt |
| 1081 | ggccaaccgc ctggcctrgg acggggccac ctgggcagac gtcttcaagc ggttcaacag |
| 1141 | cggcacgtac aatagccagt ggatgattgt ggactacaag gcattcctcc ccaacggacc |
| 1201 | cagccctgga agccgggtgc tcactatcct agaacagatc ccgggcatgg tggtggtggc |
| 1261 | tgacaagact gcagagctct acaaaacgac ctactgggct agctacaaca tcccgtactt |
| 1321 | tgagactgta ttcaacgcta gtgggctgca ggccctggtg gcccagtatg gagattggtt |
| 1381 | ctcttacact aagaaccctc gagccaagat cttccagagg gaccagtcac tggtggagga |
| 1441 | catggacgcc atggtccggc tcatgaggta caatgatttc ctccatgacc ctctgtcatt |
| 1501 | gtgtgaagcc tgcaacccaa agcctaatgc ggagaatgcc atctctgccc gctctgacct |
| 1561 | caaccccgcc aatggctcct acccatttca ggccctgcat cagcgcgccc atggtggcat |
| 1621 | tgatgtgaag gtgaccagct ttaccctggc caagtacatg agcatgctgg cggccagtgg |
| 1681 | ccccacgtgg gaccagtgtc ctccgttcca gtggagcaaa tcgccattcc acagcatgct |
| 1741 | gcacatgggc cagcccgacc tctggatgtt ctcacccatc agggtgccat gggactgaga |
| 1801 | gagagtccgc ctccgcctag ctgccttcat tttgtgtggc cagtgggtca tacacctgcc |
| 1861 | gtccacccct cgggcttctg tcttcactag actctggtcc tagcggcttc cttcgcaagg |
| 1921 | acacaaccca gtgggctcag agttgcctct gtccctgagc cctctgcccc ttcatggctc |
| 1981 | atcctccctg tccctgtcac cagcaggctg gggcttatgc ttggctgtgg gcctggtggg |
| 2041 | atccggggca cacattctcc tagtgctggt ccctcagcat atgtgtgaac tgacagggga |
| 2101 | cattatggtt gtcactgctg gcctgtgggc ccatcgcctc agaaggcagc cctgtgccct |
| 2161 | tctgggcagc tcttctaagt gcaggagctt gagaacaaaa ccaaagtttc tggctgcttg |
| 2221 | tagctggagg gccttgagtc ttctttcagc aggaggaatg gaccgtcacc ccacacttct |
| 2281 | acccctactc ccagccaagc ctgcccctgg cctcctggta ggtgtctctg gctgtgtgct |
| 2341 | ccggtcagcg aagcccaggc tgtgcttctg ttaaacaagc cttgtggggc ggcaccacac |
| 2401 | cctgtctgtc catggctgtc tccttccatc tgtcctttct ctagtccacg tacctactgt |
| 2461 | tcacctgcat caccagcccc ctgcccgtcc atcccctctc ctgtccaccc taccgtcttt |
| 2521 | tcactcagtc agcggtccac acacctaccc gtccacttgc ctgccttttc atctgaacgt |
| 2581 | cctcacctct cacccaccca tccacctgcc cgtccgtcag tctgtccacc cagaactgca |
| 2641 | catccctttt cattttggtt ttgctctgta actcagtgtg gccttgaact tgaatgcctt |
| 2701 | gtggtccaca gtcatcctct ggttattgta tctgctggcc gccttcaccc tcacatgctg |
| 2761 | ggtttccatg tacaggccgc cgtgccttga ttctgctttt catcagccca ctcatctctc |
| 2821 | tgtccattta tccctgttag tccttctgct cagcatctgt ctcaagtgct gtattctggg |
| 2881 | tcctagcctg gcccaacctg ctgaccacta agaccctaaa tcctagaccc agagacccac |
| 2941 | atgaaaccaa gtcccctgtg tggggcccag agagctcaca gcctgtgaac cagaggccct |
| 3001 | aagatgtgtg ggtagcttta cccgagccag tccgggcctg ccatgtgttc tgagtttcgt |
| 3061 | tggtagtcag gggttggttt cctgtcaggt tcactccacc ttccctgctc tgcagatgcc |
| 3121 | tgcacggggt tggttttcca gacaccaggg tggggccgct ggtgtctcag gatggccttg |
| 3181 | tcctgtccct aaggacttca tattagcatg tggtggcctt gcctgtgcat ctcccagctg |
| 3241 | ctttgtgacc ccaccctaga tccctgtgtg gcttagctct ggtcctctct cctgacttac |
| 3301 | ggatgtgtgg gttccaccag tcaatccact gggagaccca agaccccaga ggggagcacc |
| 3361 | actcctttga gagccaggga cgatgtggct gggtgtaaag ggagactgaa caaggcaagg |
| 3421 | aagctaacgc ctgtctgccc ggcactcaga agatgaggca ggaggatctc cagtttgaga |
| 3481 | ccagcctggg ctgcacatag accctcatgt caagaacaaa caaagctcaa aagatctttg |
| 3541 | ctctataaat atatatttat tttttattt |
| // | |
| (SEQ ID 3)LOCUS AF261233 2868 bp mRNA linear ROD 25-JUN-2002 | |
| DEFINITION Mus musculus sodium/calcium exchanger protein mRNA, complete cds. | |
| ACCESSION AF261233 | |
| /translation=“MASRWLALLWAPVFLCVALLLETASGTGDPSTKAHGHIQFSAGS | |
| VNQTAMADCRAVCGLNTSDRCDFVRRNPDCRSEAGYLDYLEGIFCYFPPNLLPLAITL | |
| YVFWLLYLFLILGVTAAKFFCPNLSAISTNLKLSHNVAGVTFLAFGNGAPDIFSALVA | |
| FSDPRTAGLAIGALFGAGVLVTTVVAGGITILHPFMAASRPFLRDIAFYMVAVFLTFT | |
| ALYLGRITLAWALGYLGLYVFYVVTVIICTWVYQRQRSRSLVHSISETPELLSESEED | |
| QMSSNTNSYDYGDEYRPLLLGQETTVQILIQALNPLDYRKWRTQSISWRVLKVVKLPV | |
| EFLLLLTVPVVDPDKDDRNWKRPLNCLQLVISPLVLVLTLQSGVYGLYEIGGLLPVWA | |
| VVVIVGTALASVTFFATSNREPPRLHWLFAFLGFLTSALWINAAATEVVNILRSLGVI | |
| LRLSNTVLGLTLLAWGNSIGDAFSDFTLARQGYPRMAFSACFGGIIFNILVGVGLGCL | |
| LQIIRNHVVEVKLEPDGLLVWVLASALGLSLIFSLVSVPLQCFQLSKAYGLCLLLFYV | |
| CFLVVVLLTEFGVIHLKKA” | |
| BASE COUNT 505a 800c 829g 734t | |
| ORIGIN | |
| 1 | ctccgggcca ggactctagg ggcggaaggt tgtggcgctg gccatccggc tagaggaaga |
| 61 | ctccgaggtc gcggatccag gccccgcccg aggcactaga gcagccagcc cgtgagcaga |
| 121 | gagggctctg gtcaggcctc aaggggccca tggcaagcag atggctggct ctgctctggg |
| 181 | ctcctgtctt cctctgtgtg gctctgttac tggagacggc gtctgggacc ggagacccat |
| 241 | ccacaaaagc ccatggacac atccagtttt cagctggaag tgtcaaccag actgccatgg |
| 301 | cggattgccg agccgtgtgt ggcctgaaca catctgatcg ctgtgacttt gtcaggagga |
| 361 | atccggactg ccgcagcgag gcgggctacc tggactacct tgagggcatc ttctgctact |
| 421 | tcccccccaa cctcctccct ctggccatca ccctctacgt tttctggctg ctttacctct |
| 481 | ttctgatcct gggagtcacc gcggccaagt tcttctgccc taacctgtca gccatctcca |
| 541 | ctaacctcaa actctcccac aacgtggcag gtgtcacctt cctggccttt ggaaatggcg |
| 601 | ctccagacat cttcagtgct ttagtggctt tcrcagaccc acgtactgcc ggcctggcca |
| 661 | tcggggctct gtttggtgca ggggtgctgg tcaccactgt ggtggctgga ggcatcacca |
| 721 | tcctgcatcc cttcatggct gcctccaggc ccttcctcag ggacatcgct ttctacatgg |
| 781 | tggctgtgtt cctaaccttc actgcactct atcttggcag gatcacgctg gcgtgggcgc |
| 841 | tgggttacct gggcctctac gtgttctacg tggtcacggt catcatctgc acttgggtct |
| 901 | accaacggca gcgaagcagg tctctggtcc actccatatc ggagacacca gagttgctgt |
| 961 | ctgagtcaga ggaggaccag atgtcttcca acaccaacag ctatgactat ggagatgagt |
| 1021 | accggcctct gttgctgggt caggagacca ctgtccagat cctgatccaa gccctgaatc |
| 1081 | ccttggacta caggaagtgg agaactcagt cgatatcctg gagggtcctg aaggtagtca |
| 1141 | agttacctgt ggagttcttg ttgctgctca cagtaccggt tgtggaccct gacaaggacg |
| 1201 | atcggaattg gaaacggcca ctcaactgtc tgcagctggt catcagcccc ctggtcctgg |
| 1261 | tcctgaccct gcagtcgggg gtctatggcc tctatgagat tggcggtctc cttcctgtct |
| 1321 | gggctgtggt ggtgatcgtg ggcacagcgc tggcttcagt gaccttcttt gccacgtcta |
| 1381 | acagagaacc ccctagactg cactggctct ttgctttcct gggtttcctg accagtgccc |
| 1441 | tgtggatcaa tgcagccgcc acagaggtgg tgaacatctt acggtccctg ggtgtgatcc |
| 1501 | tccgcctgag caacaccgtc ctagggctga ccctcctggc ctggggaaac agcattggag |
| 1561 | atgccttctc agatttcacg ctggcccgcc aaggataccc tcggatggcc ttctccgcct |
| 1621 | gtttcggggg catcatcttc aacatcctgg ttggtgtggg gctgggctgc ttgctgcaga |
| 1681 | tcatcaggaa ccacgttgtg gaggtgaagc tggagccaga cggattactg gtgtgggtgc |
| 1741 | tggccagtgc cctgggcctc agcttgatct tctccctggt ctccgtgccg cttcagtgtt |
| 1801 | tccagctcag caaggcttac ggcctctgcc tcctcctctt ctacgtctgt ttccttgttg |
| 1861 | tggtcctgct cacagagttt ggggtgattc acctgaagaa ggcgtgactg aagctgcttg |
| 1921 | gcctagaggt gtgggggcga ttctgctagc ctcctgaggg ggaggtgtgg ggagggggac |
| 1981 | cctctgtggt ccccgtggat ctcctgagaa gatagtcact ggcagagctc tgcagggtga |
| 2041 | gaaggtcctg actgccggca cctaacagcc ttagtgtggg gatctggagg ctggctttgc |
| 2101 | tggggacaat cccgggtagg aatgatggga tctaaatgac accggaggct ctggggggag |
| 2161 | ggcagccttt cagtcagccc ccatgcctgc tgggctctgg gcagccctgc agttccctct |
| 2221 | ctggctcttc cactctctgt ggggtcctgc gtacctacag ggtggcctga aaacagactc |
| 2281 | cacacgtgga aacaagactg ggtttctcag cttccgtgtc aagtcagcca gaggaaagag |
| 2341 | gtcgagttga cagcagcagg cactgccctt cctagttggt ggctgccatg ttggactgtg |
| 2401 | gatctaaaac acttctagag ctttgtggtc caaactctgg ctctcccgtc tgcaaaacag |
| 2461 | gagccagcat gggctctgtg cctacctcaa ggggagctgg gggttggggg gactgaccct |
| 2521 | ccccagggag gtcttacaag tagtgtgacc agtcttttgt ttgtttggtt ggttggttgt |
| 2581 | tttttggaga cagggtttct ctgtgtagcc ctggctgtcc tggaactcac tctgtagacc |
| 2641 | aggctggcct tgaactcaga aatctgcctg cctttgcctc ccaagtgctg ggactaaagg |
| 2701 | tgtgtgccac cactgtccag cttgtgacca gccttttaaa agtgccactc actccctgac |
| 2761 | tgccttcagc tgtaattaag agacttgact gttggggttt ttttttgttt gtttgttttt |
| 2821 | tgtttttgtt ttttgttgtt tccaaaaata aaagatgagt tatttcac |
| // | |
| (SEQ ID 4)LOCUS AF319547 3324 bp mRNA linear ROD 25-JUN-2002 | |
| DEFINITION Mus musculus ATP-dependent RNA-helicase (Ddx) mRNA, | |
| Ddx-RV allele, complete cds. | |
| ACCESSION AF319547 | |
| /translation=“MAAGRRVGPGPPSRPTMAPWKKKRLRKRRTGASQGRDSDSDDGE | |
| FEIQAEDDARARKLGPGRALPSFPTSECVSDVEPDTREMVRAQNKKKKKSGGFQSMGL | |
| SYPVFKGIMKKGYKVPTPIQRKTIPVILDGKDVVAMARTGSGKTACFLLPMFERLKAR | |
| SAQTGARALILSPTRELALQTMKFTKELGKFTGLKTALILGGDKMEDQFAALHENPDI | |
| IIATPGRLVHVAVEMNLKLQSVEYVVFDEADRLFEMGFAEQLQEIIGRLPGGHQTVLF | |
| SATLPKLLVEFARAGLTEPVLIRLDVDSKLNEQLKTSFLLVREDTKAAVLLYLLQNVV | |
| RPQDQTVVFVATKHHAEYLTELLMGQGVSCAHIYSALDQTARKINLAKFTHNKCSTLI | |
| VTDLAARGLDIPLLDNVINYSFPAKGKLFLHRVGRVARAGRSGTAYSLVAPDEVPYLL | |
| DLHLFLGRSVTLARPCEEPSVADAVGRDGVLGRVPQSVVDDEDSSLQTAMGASLDLQG | |
| LHRVANNAQQQYVRSRPAPSPESIKRAKELDLAELGLHPLFSSCFEEGELQRLRLVDS | |
| IKNYRTRTTIFEINASSKDPSSQMMRAKRQRDRKAVASFQQRRQERQEGPADPAPQR | |
| ELPQEEEEEMVETVEGVFTEVVGQKRPRPGPSQGAKRRRMETRQRDQEFYVPYRPK | |
| DFDSERGLSVSGAGGAFEQQVAGAVLDLMGDEAQNMSRGQQQLKWDRKKKRFVGQSGQEDK | |
| KKIKTESGRFISSSYKRDLYQKWKQKQKIDDRDSEEEGPSNQRGPGPRRGGKRGRSQG | |
| TSQPRASSVPAGRMRSELKTKEQILKQRRQAQKQRFLQRGGLKQLSARNRRRAQELRQ | |
| GAFGRGAPSRKGKMRKRM” | |
| polyA signal 3303 . . . 3308 | |
| /gene=“Ddx” | |
| BASE COUNT 725a 973c 1023g 603t | |
| ORIGIN | |
| 1 | agttaccaca tctctcacag cttggcattc gcgcagttag taggtccctt aagcatctat |
| 61 | cacggttcag ttgacacatt ccgcccccac cagttggtat gtccctcagg cctcgcctct |
| 121 | gcccggctgg tacaccactt aggccccgcc cccgttctgt tggttcctcc ttctggcttc |
| 181 | gcccattgat aaagccatgt tgacgctccg cccctgcgaa gttggttggt ttcctcaggc |
| 241 | ccggccccgc cctacgggtg aaacctggat tctcgacgcc gctcttgcgt ctcacaggct |
| 301 | ccgcccccgc gcagtcgacg cgtcccttag gccccgccct cttccgggtc taagagcccg |
| 361 | gcccgcatgg ctgctggcag acgtgtggga cctggcccgc cgtcgcgtcc caccatggcg |
| 421 | ccgtggaaga agaagaggct gcggaaacgc cgaactgggg cttcccaagg ccgcgacagc |
| 481 | gactcggatg acggcgagtt cgagatccag gcggaggatg acgcccgggc gaggaagctg |
| 541 | ggccctggca gagccttgcc ctcatttcct acctcagagt gcgtatcaga tgtggagccc |
| 601 | gacactcggg agatggtgcg agcccagaac aagaaaaaga agaagtctgg aggcttccag |
| 661 | tccatgggcc tgagttaccc tgtgttcaag gggatcatga aaaagggcta caaggtgccg |
| 721 | acgcccatcc agaggaagac catccccgtg atcttggatg gcaaggatgt ggtggccatg |
| 781 | gcccggacag gcagtggcaa gacggcctgc ttcctcctcc cgatgtttga gcggctgaag |
| 841 | gcacgcagtg cacagacggg ggctcgagcc ctcatcctct cacccacccg ggagctggcc |
| 901 | ctgcagacca tgaagttcac taaagagcta ggcaagttca ccggcctcaa gactgccttg |
| 961 | atcctgggtg gagacaaaat ggaagaccag tttgcagccc tgcacgagaa ccctgacata |
| 1021 | atcattgcca cccctgggcg tctggtgcat gtggctgtgg agatgaactt gaagctgcag |
| 1081 | agtgtggagt atgtggtgtt cgatgaagca gacaggctct ttgaaatggg ctttgctgag |
| 1141 | cagctacagg agatcatagg ccgccttcct gggggccacc agacggtgct gttctcagct |
| 1201 | acactgccca agctgctggt ggaatttgca cgggcaggcc tcacagagcc cgtgctcatc |
| 1261 | cgcctggacg tagactccaa gctcaatgag cagctcaaga cctccttcct ccttgtgcgc |
| 1321 | gaagacacca aggctgccgt gctcctctac ctgctgcaga atgtcgttcg gccccaggac |
| 1381 | cagactgtgg tgtttgtagc cacaaagcac catgcggagt acctcacaga gttgctgatg |
| 1441 | ggccagggtg tgagttgcgc ccacatctat agtgccttgg accagacggc ccgcaagatc |
| 1501 | aacttggcca agttcacaca caacaaatgt tccaccctca tcgtgactga cctggccgcc |
| 1561 | cggggcctgg acatcccact gctggacaac gtcatcaact acagcttccc tgccaagggc |
| 1621 | aagctcttcc tgcaccgagt gggccgtgtg gcccgagcag gccgaagtgg cacagcctat |
| 1681 | tctttggtgg ccccagacga ggtcccctac ctgcttgacc tacacctgtt cctgggccgc |
| 1741 | tctgtcaccc tggcccgtcc ttgtgaggag ccttcagtgg cagatgcggt tggcagggac |
| 1801 | ggagtgctgg gtcgcgtgcc ccagagtgta gtggatgatg aggacagcag cctgcagact |
| 1861 | gccatggggg catccctgga tcttcagggc ctgcaccgcg tggccaacaa cgctcagcag |
| 1921 | cagtatgtgc gctcacggcc agcgccctcg cctgagtcca tcaagagagc caaggagctg |
| 1981 | gacctggcag agctgggctt gcacccactc ttcagctcat gctttgagga gggagagctc |
| 2041 | caacgcctga ggctggtgga cagcatcaag aactatcgca cgcgcacaac catctttgag |
| 2101 | atcaatgcct ccagcaagga cccaagcagc caaatgatgc gtgccaagcg gcagagggac |
| 2161 | cggaaagctg ttgccagttt ccagcagcgg cgccaggaaa ggcaggaagg cccagctgac |
| 2221 | ccagcccccc agagggagct gcctcaggag gaggaggagg agatggttga gactgtagag |
| 2281 | ggtgtcttca cagaagtcgt gggccagaaa cggccaaggc cgggacccag ccaaggagcc |
| 2341 | aagaggcgga ggatggagac ccgtcagcga gaccaggagt tttatgtccc ctaccggccc |
| 2401 | aaggatttcg acagtgagcg ggggctgagt gtcagtgggg ctggaggggc ctttgagcag |
| 2461 | caggtggctg gtgcagtcct ggacctgatg ggggatgaag cacagaacat gagccggggg |
| 2521 | cagcagcagc tcaagtggga ccggaagaag aagcggtttg tggggcagtc aggccaagaa |
| 2581 | gacaagaaaa agatcaagac agagagcggc cggtttatca gcagctctta caagcgggat |
| 2641 | ctctaccaga agtggaagca gaagcagaaa attgatgacc gggactccga ggaagaaggg |
| 2701 | ccatccaacc agcgaggccc tgggccccgc agaggtggaa agcgaggtcg tagtcaaggc |
| 2761 | acatcccagc cccgagcttc cagtgtaccc gcaggccgca tgcgctcgga actcaagacc |
| 2821 | aaggagcaaa tcctcaagca gcgccggcaa gctcagaagc agcgcttcct gcagcgaggg |
| 2881 | ggcctgaagc agctttcagc acgcaaccga cgccgagccc aggagctgcg ccagggcgcc |
| 2941 | tttggccggg gtgctccctc caggaagggc aagatgagga aaaggatgtg aggagccaga |
| 3001 | cgcagccctg gggcttcctg gtagccccgg gtgtggacgt cagggactat gtccatgtgc |
| 3061 | tgttggaaga tccttccaca ggcgctgctc tgtgaggagt agtgccatat ggccacagag |
| 3121 | caacagctgc ttttgactgg gacattgggt gacctctgaa aggatgcata ggagtctagc |
| 3181 | tatgcaaagc aggcagaccc aagtcctgac cctgcaagtc acagcggctt ctggttccac |
| 3241 | accttcagga ttcagagtca gggccgtgtg gatgcctctg acccagcatt gagttttaat |
| 3301 | gtaataaact ttactgcctc tagt |
| // | |
| (SEQ ID 5)LOCUS AF328926 1858 bp mRNA linear ROD 25-JUN-2002 | |
| DEFINITION Mus musculus 2′-5′ oligoadenylate synthetase | |
| 1B (Oas1b) mRNA, complete cds. | |
| ACCESSION AF328926 | |
| /translation=“MEQDLRSIPASKLDKFIENHLPDTSFCADLREVIDALCALLKDR | |
| SFRGPVRRMRASKGVKGKGTTLKGRSDADLVVFLNNLTSFEDQLNQQGVLIKEIKKQL | |
| CEVQHERRCGVKFEVHSLRSPNSRALSFKLSAPDLLKEVKFDVLPAYDLLDHLNILKK | |
| PNQQFYANLISGRTPPGKEGKLSICFMGLRKYFLNCRPTKLKRLIRLVTHWYQLCKEK | |
| LGDPLPPQYALELLTVYAWEYGSRVTKFNTAQGFRTVLELVTKYKQLRIYWTVYYDFR | |
| HQEVSEYLHQQLKKDRPVILDPADPTRNIAGLNPKDWRRLAGEAATWLQYPCFKYRDG | |
| SPVCSWEVPTEVGVPMKYLFCRIFWLLFWSLFHFIFGKTSSG” | |
| BASE COUNT 430a 475c 501g 452t | |
| ORIGIN | |
| 1 | ctgcttcagc gagcctagga gacacaggac ctgctggctg cagaggtatt agctggacct |
| 61 | aggatggagc aggatctgag gagcatcccg gcctcgaagc ttgataagtt catagagaac |
| 121 | catctcccgg acaccagctt ctgtgctgac ctcagagaag tcatagatgc cctgtgtgct |
| 181 | ctcctgaagg acagatcctt ccggggcccc gtccgccgaa tgagggcctc taaaggggtc |
| 241 | aagggcaaag gcaccacact caagggcagg tcagacgctg acctggtggt gttccttaac |
| 301 | aatctcacca gctttgagga tcagttaaac caacagggag tgttgattaa ggaaattaag |
| 361 | aaacagctgt gcgaggttca gcatgagaga cgttgtggag tgaagtttga ggtccacagt |
| 421 | ttaaggagtc ccaactcccg ggctctgagc ttcaagctga gcgcccccga cctgctgaag |
| 481 | gaggtgaagt ttgatgtgct gccagcctat gatttactgg atcatcttaa catcctcaag |
| 541 | aagcctaacc aacaattcta cgccaatctc atcagtgggc gtaccccgcc ggggaaggag |
| 601 | ggcaagttat cgatctgctt tatggggctt cggaagtact tcctgaactg tcgcccaacc |
| 661 | aagctgaagc gcctcatccg cctggtcacg cactggtacc aactgtgtaa ggagaagctg |
| 721 | ggggacccgc tgcccccaca gtatgccctg gagctgctca cagtctatgc ctgggagtat |
| 781 | gggagtcgag taactaaatt caacacagcc cagggcttcc gaaccgtctt ggaactggtc |
| 841 | accaagtaca aacagcttcg aatctactgg acagtgtatt atgactttcg acatcaagag |
| 901 | gtctctgaat acctgcacca acagctcaaa aaagacaggc ctgtgatctt ggaccccgct |
| 961 | gacccaacaa ggaacatagc tggtttgaac ccaaaggact ggcggcgtct agcaggagag |
| 1021 | gctgccacct ggctgcaata cccatgcttt aagtacaggg acggttcccc agtgtgctcc |
| 1081 | tgggaggtgc cgacggaggt tggagtgcca atgaagtatc tcttttgtcg tattttctgg |
| 1141 | ttattgtttt ggtctttgtt tcatttcatc tttgggaaga cttcatctgg atagcccaga |
| 1201 | gtgtcttgga tattgccatc ctcctgcctt agcgctggca tgactgcagc gtaggcctgt |
| 1261 | tatgctctgc ctcccctcca tcctcaagtg gacaagaact gggcatgtgt tttcctgtga |
| 1321 | gcccagtggg acctgtccag gaggctccag agtcaggggc atgtcctgct ctgctacagg |
| 1381 | gccttgaccc agagaagaca ggaaggtgcc caaagcccaa gagagggagg gtccaacctg |
| 1441 | tgatcagact ccaggcttct gtcccctgtc ctcaacccct gcacagacag cctttctcac |
| 1501 | agcatgcttt atctgtcttg tcccccaaca gtgttctctg ggagacaaga gattcagaag |
| 1561 | gagaatatga tggtttgtat atggttggcc cagggaatgg cactgttagg agatgtggcc |
| 1621 | atgttggaat gggtgtggcc ttgtgggtgt gggctttctc ttgtcttagc tgcctggaag |
| 1681 | tcagtatgct gctagcagcc ttcaaatgaa gatgtagaac tctcagctcc tcctgcacca |
| 1741 | tgcctgcctg gacgttgcca tgctcttgcc ttggtgataa tggactgaac ttctgaacct |
| 1801 | gtaagccaac cccaattaaa tgttgttttt ataaaaattg ccttggtcat ggtgtctg |
| // | |
| (SEQ ID 6)LOCUS AF328927 1482 bp mRNA linear ROD 25-JUN-2002 | |
| DEFINITION Mus musculus serine dehydratase (Sds) mRNA, complete cds. | |
| ACCESSION AF328927 | |
| /translation=“MEGALAERVGAEPFHRVTPLLESWALSQVAGMPVFLKYENVQIA | |
| GSFKIRGIGHFCQQMAKRGCRHLVCSSGGNAGIAAAYSARKLGIPVTIVLPEGTSVQV | |
| VRRLEGEGAEVQLTGKVWDEANVKAQELATRDGWVNVSPFDHPLIWEGHASLVRELKE | |
| SLGTPPGAVVLAVGGGGLLAGVTAGLLEVGWQHVPIVAMETRGAHSFNSALQAGRPVT | |
| LPDITSVAKSLGAKTVAARTLECAKECEVLSEVVEDREAVSAVQRFLDDERMLVEPAC | |
| GAALAAIYSGILWRLQAEGRLSSALASVVVIVCGGNNISSQQLQELKTSWAALKISGT | |
| PPKFLDTWWVIKGPRFQWSCPLPSR” | |
| polyA signal 1451 . . . 1456 | |
| /gene=“Sds” | |
| BASE COUNT 296a 396c 487g 303t | |
| ORIGIN | |
| 1 | gaagaccttg acagaaaacc tcacacccca agggcacatg cagaagaggc cttcctgatt |
| 61 | ctgtctcacg tggcttcgtt cttagatgag cccaggtcgt tcatgagcca tgagctgcgt |
| 121 | aaggaagaca gagaggattg aataccccca cgggttcatc ttggtcattt tttgttgtga |
| 181 | gttctcacca ggaccccaga atcaggagct gtccctttaa caaggaggag gggccaggcc |
| 241 | ctggtagccg gaagctgatc tggtagccag tgcgtccagg ttggtcctgg ctgggctgtc |
| 301 | cttcaggaag gtggtcagcg cgctgctgga atggaggggg ccttggcaga acgcgtcggg |
| 361 | gcggagcctt tccacagggt cacgcccctg ctggagagct gggcgctgtc tcaggtggca |
| 421 | ggcatgccgg tcttcctcaa atatgagaat gtgcagatag ctggctcctt taagattcgg |
| 481 | ggcatcggac atttctgcca gcagatggcc aagaggggat gcagacatct ggtgtgctcc |
| 541 | tcagggggca atgcgggcat tgcggctgca tactcggctc gtaagctggg catccccgtc |
| 601 | accatcgtgc tcccagaggg cacctccgtg caggtggtga ggcggctcga gggggaaggg |
| 661 | gccgaggtcc agctgactgg gaaagtctgg gatgaagcca atgtaaaagc acaagaactg |
| 721 | gccacaaggg atggctgggt gaacgtctcc ccgtttgacc atccccttat atgggaaggc |
| 781 | catgccagcc tagtgcggga gctgaaggag tcactaggga cccctccagg tgccgtggtg |
| 841 | ctggccgtgg ggggcggagg gctcctggca ggtgtgactg ctggcctgct ggaggtgggc |
| 901 | tggcagcatg tgcccatcgt tgccatggag acccgcgggg cgcacagttt caattcggcc |
| 961 | ttgcaggcag gcaggccggt caccctgcca gacatcacca gtgtagccaa gagcctcgga |
| 1021 | gccaagacgg tggctgcacg gaccttggag tgtgcaaagg agtgtgaggt cctctctgag |
| 1081 | gtggtagaag accgggaggc tgtcagcgct gtgcagaggt tcctggacga tgagcgcatg |
| 1141 | ctggtggaac ctgcctgcgg tgccgccctg gccgccatct actcgggcat cctgtggagg |
| 1201 | cttcaggctg agggccgcct gagttctgcc ctagcttccg ttgtggtcat cgtgtgcggt |
| 1261 | ggcaacaaca ttagtagcca acagcttcag gagctgaaaa ccagctgggc tgcattgaaa |
| 1321 | atctcaggaa ctcccccaaa gttcctggac acctggtggg tcatcaaggg acctcggttc |
| 1381 | cagtggtcct gccctcttcc ttccaggtag ccctcctggg ttgctctcag tggctccctg |
| 1441 | ctgtccagtg aataaacctg actgagctga aaaaaaaaaa aa |
| // | |
| (SEQ ID 7)LOCUS AF418004 1837 bp mRNA linear ROD 25-JUN-2002 | |
| DEFINITION Mus musculus C3H/He 2′-5′ oligoadenylate synthetase | |
| 1B (Oas1b) mRNA, complete cds. | |
| ACCESSION AF418004 | |
| /translation=“MEQDLRSIPASKLDKFIENHLPDTSFCADLREVIDALCALLKDR | |
| SFRGPVRRMRASKGVKGKGTALKGRSDADLVVFLNNLTSFEDQLNQQGVLIKEIKKQL | |
| CEVQHERRCGVKFEVHSLRSPNSRALSFKLSAPDLLKEVKFDVLPAYDLLDHLNILKK | |
| PNQQFYANLISGRTPPGKEGKLSICFMGLQKYFLNCRPTKLKRLIRLVTHWYQLCKEK | |
| LGDPLPPQYALELLTVYAWEYGSRVTKFNTAQGF” | |
| BASE COUNT 428a 476c 491g 442t | |
| ORIGIN | |
| 1 | ctgcttcagc cagcctagga gacacaggac ctgctggctg cagaggtaaa agctggacct |
| 61 | aggatggagc aggatctgag gagcatcccg gcctcgaagc ttgataagtt catagagaac |
| 121 | catctcccgg acaccagctt ctgtgctgac ctcagagaag tcatagatgc cctgtgtgct |
| 181 | ctcctgaagg acagatcctt ccggggcccc gtccgccgaa tgagggcctc taaaggggtc |
| 241 | aagggcaaag gcaccgcgct caagggcagg tcagacgctg acctggtggt gttccttaac |
| 301 | aatctcacca gctttgagga tcagttaaac caacagggag tgttgattaa ggaaattaag |
| 361 | aaacagctgt gcgaggttca gcatgagaga cgttgtggag tgaagtttga ggtccacagt |
| 421 | ttaaggagtc ccaactcccg ggctctgagc ttcaagctga gcgcccccga cctgctgaag |
| 481 | gaggtgaagt ttgatgtgct gccagcctat gatttactgg atcatcttaa catcctcaag |
| 541 | aagcctaacc aacaattcta cgccaatctc atcagtgggc gtaccccgcc ggggaaggag |
| 601 | ggcaagttat cgatctgctt tatggggctt cagaagtact tcctgaactg tcgcccaacc |
| 661 | aagctgaagc gcctcatccg cctggtcacg cactggtacc aactgtgtaa ggagaagctg |
| 721 | ggggacccgc tgcccccaca gtatgccctg gagctgctca cagtctatgc ctgggagtat |
| 781 | gggagtcgag taactaaatt caacacagcc cagggcttct gaaccgtctt ggaactggtc |
| 841 | accaagtaca aacagcttca aatctactgg acagtgtatt atgactttcg acatcaagag |
| 901 | gtctctgaat acctgcacca acagctcaaa aaagacaggc ctgtgatctt ggaccccact |
| 961 | gacccaacaa ggaacatagc cggtttgaac ccaaaggact ggaggcgtct agcaggagag |
| 1021 | gctgccgcct ggctgcaata cccatgcttt aagtacaggg acggttcctc agtgtgctcc |
| 1081 | tgggaggtgc cgacggaggt tgcagtgcca acgaagtatc tcttttgtcg tattttctgg |
| 1141 | ttattgtttt ggtctttgtt tcatttcatc tttgggaaga cttcatctgg atagcccaga |
| 1201 | gtgtcttgga tattgccatc ctcctgcctt agcgctggca tgactgcagc gtaggcctgt |
| 1261 | tatgccctgc ctcccttcca tcctcaagtg gacaagaact gggcatgtgt tttcctgtga |
| 1321 | gcccagtggg acctgtccag gatgctccag agtcagacgc atgtcctgct ctgctgcagg |
| 1381 | gccttgaccc agagaagaca ggaaggtgcc caaagcccaa gagagggagg gtccaacctg |
| 1441 | tgatcagact ccaggcttct gtcccctgcc ctcaacccct gcacagacag cctttctcac |
| 1501 | agcctgcttt atctgccttg tcccccaaca gtgttctctg ggagacaaga gattcagaag |
| 1561 | gagaatatta tggtttgtat atggttggcc cagggaatgg cactgttagg aggtgtggcc |
| 1621 | atgttggagt gggtgtggcc ttgtgggtgt gggctttctc ttgtcttagc tgcctggaag |
| 1681 | tcagtattct gctagcagcc ttcagatgaa gatgtagaac tctcagctcc tcctgcacca |
| 1741 | tgcctgcctg gacgttgcca tgctcttgcc ttggttataa tggactgaac gtctgaacct |
| 1801 | gtaagccaac cccaattaaa tgttgttttt ataaaaa |
| // | |
| (SEQ ID 8)LOCUS AF418005 1837 bp mRNA linear ROD 25-JUN-2002 | |
| DEFINITION Mus musculus BRVR 2′-5′ oligoadenylate synthetase | |
| 1B (Oas1b) mRNA, complete cds. | |
| ACCESSION AF418005 | |
| /translation=“MEQDLRSIPASKLDKFIENHLPDTSFCADLREVIDALCALLKDR | |
| SFRGPVRRMRASKGVKGKGTTLKGRSDADLVVFLNNLTSFEDQLNQQGVLIKEIKKQL | |
| CEVQHERRCGVKFEVHSLRSPNSRALSFKLSAPDLLKEVKFDVLPAYDLLDHLNILKK | |
| PNQQFYANLISGRTPPGKEGKLSICFMGLRKYFLNCRPTKLKRLIRLVTHWYQLCKEK | |
| LGDPLPPQYALELLTVYAWEYGSRVTKFNTAQGFRTVLELVTKYKQLRIYWTVYYDFR | |
| HQEVSEYLHQQLKKDRPVILDPADPTRNIAGLNPKDWRRLAGEAATWLQYPCFKYRDG | |
| SPVCSWEVPTEVGVPMKYLFCRIFWLLFWSLFHFIFGKTSSG” | |
| BASE COUNT 431a 472c 493g 441t | |
| ORIGIN | |
| 1 | ctgcttcagc cagcctagga gacacaggac ctgctggctg cagaggtaaa agctggacct |
| 61 | aggatggagc aggatctgag gagcatcccg gcctcgaagc ttgataagtt catagagaac |
| 121 | catctcccgg acaccagctt ctgtgctgac ctcagagaag tcatagatgc cctgtgtgct |
| 181 | ctcctgaagg acagatcctt ccggggcccc gtccgccgaa tgagggcctc taaaggggtc |
| 241 | aagggcaaag gcaccacact caagggcagg tcagacgctg acctggtggt gttccttaac |
| 301 | aatctcacca gctttgagga tcagttaaac caacagggag tgttgattaa ggaaattaag |
| 361 | aaacagctgt gcgaggttca gcatgagaga cgttgtggag tgaagtttga ggtccacagt |
| 421 | ttaaggagtc ccaactcccg ggctctgagc ttcaagctga gcgcccccga cctgctgaag |
| 481 | gaggtgaagt ttgatgtgct gccagcctat gatttactgg atcatcttaa catcctcaag |
| 541 | aagcctaacc aacaattcta cgccaatctc atcagtgggc gtaccccgcc ggggaaggag |
| 601 | ggcaagttat cgatctgctt tatggggctt cggaagtact tcctgaactg tcgcccaacc |
| 661 | aagctgaagc gcctcatccg cctggtcacg cactggtacc aactgtgtaa ggagaagctg |
| 721 | ggggacccgc tgcccccaca gtatgccctg gagctgctca cagtctatgc ctgggagtat |
| 781 | gggagtcgag taactaaatt caacacagcc cagggcttcc gaaccgtctt ggaactggtc |
| 841 | accaagtaca aacagcttcg aatctactgg acagtgtatt atgactttcg acatcaagag |
| 901 | gtctctgaat acctgcacca acagctcaaa aaagacaggc ctgtgatctt ggaccccgct |
| 961 | gacccaacaa ggaacatagc tggtttgaac ccaaaggact ggcggcgtct ageaggagag |
| 1021 | gctgccacct ggctgcaata cccatgcttt aagtacaggg acggttcccc agtgtgctcc |
| 1081 | tgggaggtgc cgacggaggt tggagtgcca atgaagtatc tcttttgtcg tattttctgg |
| 1141 | ttattgtttt ggtctttgtt tcatttcatc tttgggaaga cttcatctgg atagcccaga |
| 1201 | gtgtcttgga tattgccatc ctcctgcctt agcgctggca tgactgcagc gtaggcctgt |
| 1261 | tatgctctgc ctcccctcca tcctcaagtg gacaagaact gggcatgtgt tttcctgtga |
| 1321 | gcccagtggg acctgtccag gaggctccag agtcaggggc atgtcctgct ctgctacagg |
| 1381 | gccttgaccc agagaagaca ggaaggtgcc caaagcccaa gagagggagg gtccaacctg |
| 1441 | tgatcagact ccaggcttct gtcccctgtc ctcaacccct gcacagacag cctttctcac |
| 1501 | agcatgcttt atctgtcttg tcccccaaca gtgttctctg ggagacaaga gattcagaag |
| 1561 | gagaatatga tggtttgtat atggttggcc cagggaatgg cactgttagg agatgtggcc |
| 1621 | atgttggaat gggtgtggcc ttgtgggtgt gggctttctc ttgtcttagc tgcctggaag |
| 1681 | tcagtatgct gctagcagcc ttcaaatgaa gatgtagaac tctcagctcc tcctgcacca |
| 1741 | tgcctgcctg gacgttgcca tgctcttgcc ttggtgataa tggactgaac ttctgaacct |
| 1801 | gtaagccaac cccaattaaa tgttgttttt ataaaaa |
| // | |
| (SEQ ID 9)LOCUS AF418006 1837 bp mRNA linear ROD 25-JUN-2002 | |
| DEFINITION Mus musculus MOLD/Rk 2′-5′ oligoadenylate synthetase | |
| 1B (Oas1b) mRNA, complete cds. | |
| ACCESSION AF418006 | |
| translation=“MEQDLRSIPASKLDKFIENHLPDTSLCADLREVIDALCALLKDR | |
| FFRGPVRRMRASKGVKGKCTALKGRSDADLVVFLNNLTYFEDQLNQQGVLIKEIKKQL | |
| YEVQHERRFGVKFEVQSLRSPNSRALSFKLSAPDLLKEVKFDVLPAYDLLDHLNILKK | |
| PNQQFYANLISGRTPLGKEGKLLTCFMGLRKYFLNCRPTKLKRLIRLVTHWYQLCKEK | |
| LGDPLPPQYALELLTVYAWEYGSRVTKFNTAQGFRTVLELVTKYKQLRIYWTVYYDFR | |
| HQEVSEYLHQQLKKDRPVILDPADPTRNIAGLNPKDWRRLAGEAAAWLQYPCFKYRDC | |
| SPVCSWEVPTEVAVPTKYLFCRIFWLLFWSLFHFIFGKTSSG” | |
| BASE COUNT 425a 470c 491g 451t | |
| ORIGIN | |
| 1 | ctgcttcagc cagcctagga gacacaggac ctgctggctg cagaggtaaa agctggacct |
| 61 | aggatggagc aggatctgag gagcatcccg gcctcgaagc ttgataagtt catagagaat |
| 121 | catctcccgg acaccagctt gtgtgctgac ctcagagaag tcatagatgc cctgtgtgct |
| 181 | ctcctgaagg acagattctt ccggggcccc gtccgccgaa tgagggcctc taagggggtc |
| 241 | aagggcaaat gcaccgcgct caagggcagg tcagacgctg acctggtggt gttccttaac |
| 301 | aatctcacct actttgagga tcaattaaac caacagggag tgttgattaa ggaaattaag |
| 361 | aaacagctgt acgaggttca gcatgagaga cgttttggag tcaagtttga ggtccagagt |
| 421 | ttaaggagtc ccaactcccg ggctctgagc ttcaagctga gcgcccccga cctgctgaag |
| 481 | gaggtgaagt ttgacgtgct gccagcctat gatttactgg atcatcttaa catcctcaag |
| 541 | aagcctaacc aacaattcta cgccaatctc atcagtgggc gtaccccgct ggggaaggag |
| 601 | ggcaagttat tgacctgctt tatggggctt cggaagtact tcctgaactg tcgcccaacc |
| 661 | aagctgaagc gcctcatccg cctggtcacg cactggtacc aactgtgtaa ggagaagctg |
| 721 | ggggacccgc tgcccccaca gtatgccctg gagctgctca cagtctatgc ctgggagtat |
| 781 | gggagtcgag taactaaatt caacacagcc cagggcttcc gaaccgtctt ggaactggtt |
| 841 | accaagtaca aacagcttcg aatctactgg acagtgtatt atgactttcg acatcaagag |
| 901 | gtctctgaat acctgcacca acagctcaaa aaagacaggc ctgtgatctt ggaccccgct |
| 961 | gatccaacaa ggaatatagc tggtttgaac ccaaaggact ggcggcgtct agcaggagag |
| 1021 | gctgccgcct ggctgcaata cccatgcttt aagtacaggg acggttcccc agtgtgctcc |
| 1081 | tgggaggtgc cgacggaggt tgcagtgcca acgaagtatc tcttttgtcg tattttctgg |
| 1141 | ttattgtttt ggtctttgtt tcatttcatc tttgggaaga cttcatctgg atagcccaga |
| 1201 | gtgtcttgga tattgccatc ctcctgcctt agcgctggca tgactgcagt gtaggcctgt |
| 1261 | tatgccctgc ctcccctcca tcctcaagtg gacaagaact gggcatgtgt tttcctgtga |
| 1321 | gcccagtggg acctgtccag gatgctccag agtcagacgc atgtcctgct ctgctgcagg |
| 1381 | gccttgaccc agagaagaca ggaaggtgcc caaagcccaa gagagggagg ttccaacctg |
| 1441 | tgatcagact ccaggcttct gtcccctgcc ctcaacccct gcacagacag cctttctcac |
| 1501 | agcctgcttt atctgtcttg tcccccaaca gtgttctctg ggagacaaga gattcagaag |
| 1561 | gagaatatga tggtttgtat atggttggcc cagggaatgg cactgttagg aggtgtggcc |
| 1621 | atgttggagt gggtgtggcc ttgtgtgtgt gggctttctc ttgtcttagc tgcctggaag |
| 1681 | tcagtatgct gctagcagcc ttcagatgaa gatgtagaac tctcagctcc tcctgcacca |
| 1741 | tgcctgcctg gacgttgcca tgctcttgcc ttggtgataa tggactgaac ttctgaacct |
| 1801 | gtaagccaac tccaattaaa tgttgttttt ataaaaa |
| // | |
| (SEQ ID 10)LOCUS AF418007 1587 bp mRNA linear ROD 25-JUN-2002 | |
| DEFINITION Mus musculus MOLC/Rk 2′-5′ oligoadenylate synthetase | |
| 1B (Oas1b) mRNA, complete cds. | |
| ACCESSION AF418007 | |
| /translation=“MEQDLRSIPASKLDKFIENHLPDTSFCADLREVIDALCALLKDR | |
| FFRGPVRRMRASKGVKGKCTALKGRSDADLVVFLNNLTYFEDQLNQQGVLIKEIKKQL | |
| YEVQHERRFGVKFEVQSLRSPNSRALSFKLSAPDLLKEVKFDVLPAYDLLDHLNILKK | |
| PNQQFYANLISGRTPPGKEGKLLICFMGLRKYFLNCRPTKLKRLIRLVTHWYQLCKEK | |
| LGDPLPPQYALELLTVYAWESGSRDCEFNTAQGFRTVLELVTKYKRLRIYWTVYYDFR | |
| KTKVSEYLHKLLQKDRPVILDPADPTRNIAGLNPKDWRRLAGEAAAWLQYPCFKYRDG | |
| SPVCSWEVPTEVAVPTKYLFCRIFWLLFWSLFHFIFGKTSSG” | |
| BASECOUNT 372a 414c 428g 373t | |
| ORIGIN | |
| 1 | ctgcttcagc cagcctagga gacacaggac ctgctggctg cagaggtaaa agctggacct |
| 61 | aggatggagc aggatctgag gagcatcccg gcctcgaagc ttgataagtt catagagaat |
| 121 | catctcccgg acaccagctt ctgtgctgac ctcagagaag tcatagatgc cctgtgtgct |
| 181 | ctcctgaagg acagattctt ccggggcccc gtccgccgaa tgagggcctc taagggggtc |
| 241 | aagggcaaat gcaccgcgct caagggcagg tcagacgctg acctggtggt gttccttaac |
| 301 | aatctcacct actttgagga tcaattaaac caacagggag tgttgattaa ggaaattaag |
| 361 | aaacagctgt acgaggttca gcatgagaga cgttttggag tcaagtttga ggtccagagt |
| 421 | ttaaggagtc ccaactcccg ggctctgagc ttcaagctga gcgcccccga cctgctgaag |
| 481 | gaggtgaagt ttgacgtgct gccagcctat gatttactgg atcatcttaa catcctcaag |
| 541 | aagcctaacc aacaatttta cgccaatctc atcagtgggc gtaccccgcc ggggaaggag |
| 601 | ggcaagttat tgatctgctt tatggggctt cggaagtact tcctgaactg tcgcccaacc |
| 661 | aagctgaagc gcctcatccg cctggtcacg cactggtacc aactgtgtaa ggagaagctg |
| 721 | ggggacccgc tgcccccaca gtacgccctg gagctgctca cagtgtacgc ctgggaaagt |
| 781 | gggagtcgag actgtgaatt caacacagcc cagggcttcc gaactgtctt ggaactggtc |
| 841 | accaagtaca agcggcttcg aatctactgg acagtgtatt atgactttag aaagacgaag |
| 901 | gtctctgaat acctgcacaa actgctccaa aaagacaggc ctgtgatctt ggaccccgct |
| 961 | gatccaacaa ggaatatagc tggtttgaac ccaaaggact ggcggcgtct agcaggagag |
| 1021 | gctgccgcct ggctgcaata cccatgcttt aagtacaggg acggttcccc agtgtgctcc |
| 1081 | tgggaggtgc cgacggaggt tgcagtgcca acgaagtatc tcttttgtcg tattttctgg |
| 1141 | ttattgtttt ggtctttgtt tcatttcatc tttgggaaga cttcatctgg atagcccaga |
| 1201 | gtgtcttgga tattgccatc ctcctgcctt agcgctggca tgactgcagt gtaggcctgt |
| 1261 | tatgccctgc ctcccctcca tcctcaagtg gacaagaact gggcatgtgt tttcctgtga |
| 1321 | gcccagtggg acctgtccag gatgctccag agtcagacgc atgtcctgct ctgctgcagg |
| 1381 | gccttgaccc agagaagaca ggaaggtgcc caaagcccaa gagagggagg ttccaacctg |
| 1441 | tgatcagact ccaggcttct gtcccctgcc ctcaacccct gcacagacag cctttctcac |
| 1501 | agcctgcttt atctgtcttg tcccccaaca gtgttctctg ggagacaaga gattcagaag |
| 1561 | gagaatatga tggtttgtat atggttg |
| // | |
| (SEQ ID 11)LOCUS AF418008 1587 bp mRNA linear ROD 25-JUN-2002 | |
| DEFINITION Mus musculus MOLF/Ei 2′-5′ oligoadenylate synthetase | |
| 1B (Oas1b) mRNA, complete cds. | |
| ACCESSION AF418008 | |
| translation=“MEQDLRSIPASKLDKFIENHLPDTSFCADLREVIDALCALLKDR | |
| FFRGPVRRMRASKGVKGKCTALKGRSDADLVVFLNNLTYFEDQLNQQGVLIKEIKKQL | |
| YEVQHERRFGVKFEVQSLRSPNSRALSFKLSAPDLLKEVKFDVLPAYDLLDHLSILKK | |
| PNRQLYANLISGRTPPGKDPKLSICFMGLRKYFLNCRPTKLKRLIRLVTQWYQLCKEK | |
| LGDPLPPQYALELLTVYAWESGSRDCEFNTAQGFRTVLELVTKYKRLRIYWTVYYDFR | |
| KTKVSEYLHKLLQKDRPVILDPADPTRNIAGLNPKDWRRLAGEAAAWLQYPCFKYRDG | |
| SPVCSWEVPTEVAVPTKYLFCRIFWLLFWSLFHFIFGKTSSG” | |
| BASE COUNT 372a 417c 427g 371t | |
| ORIGIN | |
| 1 | ctgcttcagc cagcctagga gacacaggac ctgctggctg cagaggtaaa agctggacct |
| 61 | aggatggagc aggatctgag gagcatcccg gcctcgaagc ttgataagtt catagagaat |
| 121 | catctcccgg acaccagctt ctgtgctgac ctcagagaag tcatagatgc cctgtgtgct |
| 181 | ctcctgaagg acagattctt ccggggcccc gtccgccgaa tgagggcctc taagggggtc |
| 241 | aagggcaaat gcaccgcgct caagggcagg tcagacgctg acctggtggt gttccttaac |
| 301 | aatctcacct actttgagga tcaattaaac caacagggag tgttgattaa ggaaattaag |
| 361 | aaacagctgt acgaggttca gcatgagaga cgttttggag tcaagtttga ggtccagagt |
| 421 | ttaaggagtc ccaactcccg ggctctgagc ttcaagctga gcgcccccga cctgctgaag |
| 481 | gaggtgaagt ttgacgtgct gccagcctat gatttactgg atcatcttag catcctcaag |
| 541 | aagcctaacc gacaattata cgccaatctc atcagtgggc gtaccccgcc ggggaaggac |
| 601 | cccaagttat cgatctgctt tatggggctt cggaagtact tcctgaactg tcgcccaacc |
| 661 | aagctgaagc gcctcatccg cctggtcacg caatggtacc aactgtgtaa ggagaagctg |
| 721 | ggggacccgc tgcccccaca gtacgccctg gagctgctca cagtgtacgc ctgggaaagt |
| 781 | gggagtcgag actgtgaatt caacacagcc cagggcttcc gaactgtctt ggaactggtc |
| 841 | accaagtaca agcggcttcg aatctactgg acagtgtatt atgactttag aaagacgaag |
| 901 | gtctctgaat acctgcacaa actgctccaa aaagacaggc ctgtgatctt ggaccccgct |
| 961 | gatccaacaa ggaatatagc tggtttgaac ccaaaggact ggcggcgtct agcaggagag |
| 1021 | gctgccgcct ggctgcaata cccatgcttt aagtacaggg acggttcccc agtgtgctcc |
| 1081 | tgggaggtgc cgacggaggt tgcagtgcca acgaagtatc tcttttgtcg tattttctgg |
| 1141 | ttattgtttt ggtctttgtt tcatttcatc tttgggaaga cttcatctgg atageccaga |
| 1201 | gtgtcttgga tattgccatc ctcctgcctt agcgctggca tgactgcagt gtaggcctgt |
| 1261 | tatgccctgc ctcccctcca tcctcaagtg gacaagaact gggcatgtgt tttcctgtga |
| 1321 | gcccagtggg acctgtccag gatgctccag agtcagacgc atgtcctgct ctgctgcagg |
| 1381 | gccttgaccc agagaagaca ggaaggtgcc caaagcccaa gagagggagg ttccaacctg |
| 1441 | tgatcagact ccaggcttct gtcccctgcc ctcaacccct gcacagacag cctttctcac |
| 1501 | agcctgcttt atctgtcttg tcccccaaca gtgttctctg ggagacaaga gattcagaag |
| 1561 | gagaatatga tggtttgtat atggttg |
| // | |
| (SEQ ID 12)LOCUS AF418009 1587 bp mRNA linear ROD 25-JUN-2002 | |
| DEFINITION Mus musculus MOLG/Dn 2′-5′ oligoadenylate synthetase | |
| 1B (Oas1b) mRNA, complete cds. | |
| ACCESSION AF418009 | |
| translation=“MEQDLRSIPASKLDKFIENHLPDTSFCADLREVIDALCALLKDR | |
| FFRGPVRRMRASKGVKGKCTALKGRSDADLVVFLNNLTYFEDQLNQQGVLIKEIKKQL | |
| YEVQHERRFGVKFEVQSLRSPNSRALSFKLSAPDLLKEVKFDVLPAYDLLDHLNILKK | |
| PNQQFYANLISGRTPPGKEGKLSICFMGLRKYFLNCRPTKLKRLIRLVTHWYQLCKEK | |
| LGDPLPPQYALELLTVYAWESGSRDCEFNTAQGFRTVLELVTKYKRLRIYWTVYYDFR | |
| KTKVSEYLHKLLQKDRPVILDPADPTRNIAGLNPKDWRRLAGEAAAWLQYPCFKYRDG | |
| SPVCSWEVPTEVAVPTKYLFCRIFWLLFWSLFHFIFGKTSSG” | |
| BASE COUNT 372a 415c 428g 372t | |
| ORIGIN | |
| 1 | ctgcttcagc cagcctagga gacacaggac ctgctggctg cagaggtaaa agctggacct |
| 61 | aggatggagc aggatctgag gagcatcccg gcctcgaagc ttgataagtt catagagaat |
| 121 | catctcccgg acaccagctt ctgtgctgac ctcagagaag tcatagatgc cctgtgtgct |
| 181 | ctcctgaagg acagattctt ccggggcccc gtccgccgaa tgagggcctc taagggggtc |
| 241 | aagggcaaat gcaccgcgct caagggcagg tcagacgctg acctggtggt gttccttaac |
| 301 | aatctcacct actttgagga tcaattaaac caacagggag tgttgattaa ggaaattaag |
| 361 | aaacagctgt acgaggttca gcatgagaga cgttttggag tcaagtttga ggtccagagt |
| 421 | ttaaggagtc ccaactcccg ggctctgagc ttcaagctga gcgcccccga cctgctgaag |
| 481 | gaggtgaagt ttgacgtgct gccagcctat gatttactgg atcatcttaa catcctcaag |
| 541 | aagcctaacc aacaatttta cgccaatctc atcagtgggc gtaccccgcc ggggaaggag |
| 601 | ggcaagttat cgatctgctt tatggggctt cggaagtact tcctgaactg tcgcccaacc |
| 661 | aagctgaagc gcctcatccg cctggtcacg cactggtacc aactgtgtaa ggagaagctg |
| 721 | ggggacccgc tgcccccaca gtacgccctg gagctgctca cagtgtacgc ctgggaaagt |
| 781 | gggagtcgag actgtgaatt caacacagcc cagggcttcc gaactgtctt ggaactggtc |
| 841 | accaagtaca agcggcttcg aatctactgg acagtgtatt atgactttag aaagacgaag |
| 901 | gtctctgaat acctgcacaa actgctccaa aaagacaggc ctgtgatctt ggaccccgct |
| 961 | gatccaacaa ggaatatagc tggtttgaac ccaaaggact ggcggcgtct agcaggagag |
| 1021 | gctgccgcct ggctgcaata cccatgcttt aagtacaggg acggttcccc agtgtgctcc |
| 1081 | tgggaggtgc cgacggaggt tgcagtgcca acgaagtatc tcttttgtcg tattttctgg |
| 1141 | ttattgtttt ggtctttgtt tcatttcatc tttgggaaga cttcatctgg atagcccaga |
| 1201 | gtgtcttgga tattgccatc ctcctgcctt agcgctggca tgactgcagt gtaggcctgt |
| 1261 | tatgccctgc ctcccctcca tcctcaagtg gacaagaact gggcatgtgt tttcctgtga |
| 1321 | gcccagtggg acctgtccag gatgctccag agtcagacgc atgtcctgct ctgctgcagg |
| 1381 | gccttgaccc agagaagaca ggaaggtgcc caaagcccaa gagagggagg ttccaacctg |
| 1441 | tgatcagact ccaggcttct gtcccctgcc ctcaacccct gcacagacag cctttctcac |
| 1501 | agcctgcttt atctgtcttg tcccccaaca gtgttctctg ggagacaaga gattcagaag |
| 1561 | gagaatatga tggtttgtat atggttg |
| // | |
| (SEQ ID 13)LOCUS AF418010 3897 bp mRNA linear ROD 25-JUN-2002 | |
| DEFINITION Mus musculus 2′-5′ oligoadenylate synthetase | |
| 2 (Oas2) mRNA, complete cds. | |
| ACCESSION AF418010 | |
| /translation=“MGNWLTGNWSSDRSSGYSSGWSPGGSSGVPSGPVHKLEKSIQAN | |
| LTPNENCLKQIAVSSVPSQKLEGYIQENLKPNRESLKQIDQAVDAIWDLLRSQIPVKE | |
| VAKGGSYGRETALRGCSDGTLVLFMDCFQQFQDQIKYQDAYLDVIELWLKIHEKKSVK | |
| HEHALVVQVSVPGQRILLQLLPVFNPLRSNENPSSCVYVDLKKSMDQVRASPGEFSDC | |
| FTTLQQRFFKKYPRRLKDLILLVKHWYEQCQEKWKTPPPQPLLYALELLTVYAWEQGC | |
| QAEDFDMAQGVRTVLRLIQRPTELCVYWTVNYNFEDETVRNILLHQLRSQRPVILDPT | |
| DPTNNVGKDDGFWELLTEEAMAWLYSPSLNTESPAPYWDVLPMPLFVTPSHLLNKFIK | |
| DFLQPNKLFLKQIKEAVDIICSFLKNVCFLNSDTKVLKTVKGGSTAKGTALKRGSDAD | |
| IVVFLSSLESYDSLKTNRSQFVQEIQKQLEEFVQAQEWEVTFEISKWKAPRVLSFTLK | |
| SKTLNESVEFDVLPAYDALGQLRSDFTLRPEAYKDLIELCASQDIKEGEFSICFTELQ | |
| RNFIQTRPTKLKSLLRLIKHWYKQYERKMKPKASLPPKYALELLTVYAWEQGSGTDDF | |
| DIAEGFRTVLDLVIKYRQLCIFWTVNYNFEEEYMRKFLLTQIQKKRPVILDPADPTGD | |
| VGGGDRWCWHLLAEEAKEWLSSPCFQVEQKGLVQPWKVPVPRDLKTSDMVGVFTTGGI | |
| LWQDQGFLSFV” | |
| polyA signal 3878 . . . 3883 | |
| /gene=“Oas2” | |
| BASE COUNT 978a 1030c 940g 948t 1 others | |
| ORIGIN | |
| 1 | gaccagctag caacgatggg aaactggctg actggaaact ggtcatctga caggtcatct |
| 61 | ggctattcat ctggctggtc acctggtggg tcttcagggg tgccctccgg gccagtgcac |
| 121 | aagttagaaa agtctatcca ggcaaacctc acacccaacg aaaactgtct gaagcagatt |
| 181 | gcggtgtcct cggtgccatc gcagaagcta gaagggtata tccaggaaaa cctcaaacct |
| 241 | aacagagaat ctctgaagca gatagaccag gccgtggatg ccatctggga cctgctgcgc |
| 301 | agtcagatcc ctgtgaagga agtggctaag ggtggctcct atggccggga aacagcccta |
| 361 | agaggctgct ccgatggtac ccttgttctc ttcatggact gcttccaaca gttccaggat |
| 421 | cagataaaat accaagatgc ataccttgac gtcattgaac tgtggctgaa aatccatgag |
| 481 | aagaagtcag taaagcatga acatgccctt gtagtacaag tgtctgtacc agggcagaga |
| 541 | atactcctgc aattacttcc agtcttcaat cctctacgct ccaatgagaa tcccagctcc |
| 601 | tgtgtctatg tggatctcaa aaaatccatg gatcaagtaa gagcctcacc aggggagttc |
| 661 | tcagactgct tcaccacact gcagcagcgg tttttcaaga aatatccccg aagactgaag |
| 721 | gatttgatcc tattggtcaa gcactggtat gaacagtgcc aggagaagtg gaaaacaccc |
| 781 | ccacctcagc cattgctgta cgcactggaa ctgctcactg tgtatgcctg ggaacagggc |
| 841 | tgccaagctg aagacttcga catggcacaa ggcgtcagga ccgtgctgcg acttatccag |
| 901 | cggccgacag agctgtgtgt ctactggaca gtcaattaca actttgagga tgagacagtc |
| 961 | cggaacatcc ttctgcacca gctcaggtcc caaagaccag tcatcttgga tccaactgac |
| 1021 | ccaaccaata atgtgggcaa agatgatggg ttctgggagc tactgacaga ggaagctatg |
| 1081 | gcctggctgt actctcccag cctgaatact gagtcacctg caccatattg ggatgttctg |
| 1141 | cccatgccac ttttcgtcac tccaagccac ttactgaaca agttcatcaa ggactttctc |
| 1201 | cagcccaaca agctcttcct aaagcagatc aaggaagctg ttgacattat atgttccttc |
| 1261 | cttaaaaatg tctgcttctt gaattctgac accaaagtcc tgaagaccgt caagggagga |
| 1321 | tccactgcca aaggcacagc tctgaagcgg ggatcagatg ctgacattgt tgtgttcctc |
| 1381 | tcctcgctgg agagttacga ctctctaaaa accaaccgct cccagttcgt ccaggagatc |
| 1441 | cagaagcagt tagaagaatt cgtgcaggcg caggagtggg aggtgacgtt tgagatttca |
| 1501 | aaatggaagg ctcccagagt gctgagtttt accttgaaat ccaagactct caatgaaagt |
| 1561 | gtcgagttcg atgtccttcc cgcctatgat gcactaggtc aactgcggtc tgacttcacc |
| 1621 | ctcaggcccg aagcctacaa ggatctcatt gagctgtgtg catcacagga catcaaagaa |
| 1681 | ggagagtttt ctatctgttt tactgagctg cagagaaact tcattcaaac ccggcccacc |
| 1741 | aaactgaaga gtctactccg cctgatcaag cactggtaca aacagtatga aaggaagatg |
| 1801 | aagccaaaag catctttacc cccaaagtac gccctggagc tgctcaccgt gtatgcctgg |
| 1861 | gagcagggca gtggcacaga tgactttgac attgctgaag gcttccggac cgtcctggac |
| 1921 | ctggttataa aataccggca gctctgcatc ttctggacag tcaattacaa ctttgaagag |
| 1981 | gaatacatgc ggaagttcct actgacccag atccagaaaa agaggcctgt aatcctggat |
| 2041 | ccagcagatc ccacaggcga tgtgggagga ggtgaccgct ggtgctggca tcttctagct |
| 2101 | gaagaagcga aggagtggct gtcctcccct tgtttccaag tggagcaaaa aggcctggta |
| 2161 | cagccttgga aagtgccagt acctagagat ctaaagacaa gtgacatggt gggagtgttc |
| 2221 | actacaggag ggatcttgtg gcaggaccag ggctttttgt catttgtcta ggtaatgcag |
| 2281 | acccccggaa gctgtggagg tcagatctac cccactgtgg gtggagttac taagtaggag |
| 2341 | tccattcagc tctggaagac gcttctggag tgatctggca aagactcaga ctgtgttaga |
| 2401 | aaagggagcc tggttcagtc ctctctggca ggctcgcacc tctattcttc cttcttggaa |
| 2461 | tcaagacatg ggattatcct tcctcctccc ccagggtctc acagcacagg ccctgctctg |
| 2521 | tgtgagtgac ctccttcaga gacacttgcc ccatgcagct cgatgggttc tggttttgtc |
| 2581 | tgtattctgt gcagttattt tcctgcctcc tgctctgtta gtctctagtc agcagctcca |
| 2641 | gactcaccct gtgtcactaa ggttaaggcc ctccctagcc cttcagcatt gtcaatccca |
| 2701 | actagccctc ggagtcttcc attgtgcgtc tttgcctgtc tctttccctg tccctgtgga |
| 2761 | tacagagatg taccatccat ccagcagcta gccaactccc ctccctccac ctctgctgtt |
| 2821 | aaaacccttt ctcttgggga aatgtaaaca atatctacct ctcttaatgt cccaggacaa |
| 2881 | actaagctgc atttctccct tccctgagaa gccaaagctt ccctgattga gcttrgctgc |
| 2941 | tcacaggaga ggggttacag gcctttgaag ctggccacac tagaagatct gcacccagct |
| 3001 | agatgggtgc agatggcttc cctggggctg cataaagaga acccctcccc tcatctttcc |
| 3061 | tcctgtatcc tctagcccct ctcagagatc ctgtgcaatc agggcagaat agcatgcagc |
| 3121 | tggttgaaac cacttgctaa ataactcagg tgagggtccc ataaccttcc cagcccacct |
| 3181 | cccttccaag agtgaagata acagtcaaca agcccagctg tgatgttcat tgataagcag |
| 3241 | gctctggtgg actcctaaag atggtgccag tgtggctcag tgaatagccc tgcataacat |
| 3301 | tttacacaca ccaaatgctg gttgatatct cttgctggct gcccagggag ccttcacccc |
| 3361 | agggctttaa ctgcacagag acatgaggtc taagcccttc gcatccccaa gtaaggctga |
| 3421 | gccttttttc tgcctgtgct tgctctgatg cattgaggat catgcctggc cactgtgcaa |
| 3481 | cttttaagca gagccgtgca acatcccagg gagttgactt ctatgtaaac accttcatcc |
| 3541 | atttctgatg tatgctttga ggtggctcag gctgggctag cccagcccag acagaaatcc |
| 3601 | taggcatgtg attagaggat cagaaccctt ctggcccttc ttcaggggag agatggggct |
| 3661 | gaaggtgggg ttcaaatctc atgccgagtg atggaacccg acatccctag gtgctaaggc |
| 3721 | cccaccaaat tctctggata aggaagttcc aggaatcttt actgataaac atcccaatgt |
| 3781 | atcaacaagg tagactctga cctccatggg acagaagatc ctgggtcagt cccctccctg |
| 3841 | gggactctgc agttggctgt tcatttatat gcttcataat aaatggtttc tttgtgt |
| // | |
| (SEQ ID 14)LOCUS AF453830 4708 bp mRNA linear ROD 25-JUN-2002 | |
| DEFINITION Mus musculus 2′-5′ oligoadenylate synthetase | |
| 3 (Oas3) mRNA, complete cds. | |
| ACCESSION AF453830 | |
| /translation=“MDLFHTPAGALDKLVAHNLHPAPEFTAAVRGALGSLNITLQQHR | |
| ARGSQRPRVIRIAKGGAYARGTALRGGTDVELVIFLDCFQSFGDQKTCHSETLGAMRM | |
| LLESWGGHPGPGLTFEFSQSKASRILQFRLASADGEHWIDVSLVPAFDVLGQPRSGVK | |
| PTPNVYSSLLSSHCQAGEYSACFTEPRKNEVNTRPAKLKNLILLVKHWYHQVQTQAVR | |
| ATLPPSYALELLTIFAWEQGCGKDSFSLAQGLRTVLALIQHSKYLCIFWTENYGFEDP | |
| AVGEFLRRQLKRPRPVILDPADPTWDVGNGTAWRWDVLAQEAESSFSQQCFKQASGVL | |
| VQPWEGPGLPRAGILDLGHPIYQGPNQALEDNKGHLAVQSKERSQKPSNSAPGFPEAA | |
| TKIPAMPNPSANKTRKIRKKAAHPKTVQEAALDSISSHVRITQSTASSHMPPDRSSIS | |
| TAGSRMSPDLSQIPSKDLDCFIQDHLRPSPQFQQQVKQAIDAILCCLREKSVYKVLRV | |
| SKGGSFGRGTDLRGSCDVELVIFYKTLGDFKGQKPHQAEILRDMQAQLRHWCQNPVPG | |
| LSLQFIEQKPNALQLQLASTDLSNRVDLSVLPAFDAVGPLKSGTKPQPQVYSSLLSSG | |
| CQAGEHAACFAELRRNFINTCPPKLKSLMLLVKHWYRQVVTRYKGGEAAGDAPPPAYA | |
| LELLTIFAWEQGCGEQKFSLAEGLRTILRLIQQHQSLCIYWTVNYSVQDPTIRAHLLC | |
| QLRKARPLVLDPADPTWNVGQGDWKLLAQEAAALGSQVCLQSGDGTLVPPWDVTPALL | |
| HQTLAEDLDKFISEFLQPNRHFLTQVKRAVDTICSFLKENCFRNSTIKVLKVVKGGSS | |
| AKGTALQGRSDADLVVFLSCFRQFSEQGSHRAEIISEIQAQLEACQQTHSFDVKFEVS | |
| KRKNPRVLSFTLTSQTLLDQSVDFDVLPAFDALGQLRSGSRPDPRVYTDLIHSCSNAG | |
| EFSTCFTELQRDFITSRPTKLKSLIRLVKYWYQQCNKTIKGKGSLPPQHGLELLTVYA | |
| WEQGGQNPQFNMAEGFRTVLELIVQYRQLCVYWTINYSAEDKTIGDFLKMQLRKPRPV | |
| ILDPADPTGNLGHNARWDLLAKEATVYASALCCVDRDGNPIKPWPVKAAV” | |
| polyA signal 4690 . . . 4695 | |
| /gene=“Oas 3” | |
| BASE COUNT 1031a 1322c 1248g 1107t | |
| ORIGIN | |
| 1 | gaaactctac tgagagtacc ggtcaacatg gacctgttcc acacgccagc cggagctctg |
| 61 | gataagctgg tggcccacaa cctgcaccca gcccctgagt tcacagcagc cgtacggggt |
| 121 | gctctggggt cgctaaacat caccctacag cagcacagag cccgagggtc acagagacca |
| 181 | agagtgataa ggattgccaa gggaggagcc tatgcccggg gcacagctct cagaggtggc |
| 241 | accgatgtcg aactcgtcat cttcctcgac tgcttccaga gctttggtga ccagaagacc |
| 301 | tgtcactcag agaccctggg tgccatgcga atgttgctgg agtcctgggg gggccacccc |
| 361 | gggcctggcc tgacttttga gttttctcag tcaaaggcgt ccaggatctt acagtttcgt |
| 421 | ctggcatcgg cagacggaga acactggata gatgttagcc tggtgcctgc ctttgatgtc |
| 481 | ctaggacagc cccgctctgg agtcaagccg acacccaacg tgtactcctc cctccttagc |
| 541 | agccactgcc aggccgggga gtactcagcc tgcttcactg agccccgaaa gaactttgtg |
| 601 | aacactcgcc cagccaagct taagaactta atcctgctgg tcaaacactg gtaccaccag |
| 661 | gtgcagacac aggccgtgag ggccacactg ccccccagct acgccctaga gctgcttacc |
| 721 | atctttgcct gggagcaggg ctgtgggaag gacagcttca gcctggccca agggctccgg |
| 781 | accgtcctgg ccttgatcca acacagcaag tacctctgca ttttctggac ggaaaactat |
| 841 | ggcttcgagg accctgcagt tggagagttc ttgcgaaggc agcttaagag acccaggccc |
| 901 | gtgatcctgg atccagctga tccaacgtgg gacgtgggca acgggacagc ctggcgctgg |
| 961 | gatgtgctgg cccaggaggc tgagtccagc tttagccagc agtgcttcaa gcaggcctca |
| 1021 | ggagtccttg tgcagccttg ggaggggccg ggcctgccac gggctgggat cttggatttg |
| 1081 | ggccacccaa tctatcaagg gcctaaccag gcccttgaag acaacaaagg ccaccttgct |
| 1141 | gttcagtcaa aggaaaggag ccaaaaacct tccaattcag ctccaggatt tccagaagca |
| 1201 | gccaccaaga tccctgctat gcccaaccca agtgccaata aaacccgcaa gatccgcaag |
| 1261 | aaagcagctc acccaaagac tgtccaggaa gcagcattgg atagtatctc aagtcatgtt |
| 1321 | cggatcaccc agagcacagc atcctcacac atgcctcctg accgctctag catctccacc |
| 1381 | gctgggtcac ggatgagccc agatctgtca cagatcccca gcaaggatct agactgcttc |
| 1441 | atccaggacc accttaggcc gagtccccag ttccagcagc aggtgaagca ggccatcgac |
| 1501 | gccatcttgt gctgcctccg ggagaagagt gtatacaaag tcttgagggt cagcaagggc |
| 1561 | ggctctttcg gccgtggcac agacctcagg ggcagctgcg atgtggaact tgtcatcttt |
| 1621 | tataaaaccc tcggggactt caagggccag aagcctcacc aggcagagat cctgcgtgac |
| 1681 | atgcaggccc agctacgaca ctggtgtcag aaccccgtgc ctggactgag cctccagttt |
| 1741 | attgaacaga agcccaacgc tctgcaactc cagctggcgt ccaccgacct cagcaaccgg |
| 1801 | gtggacctca gtgtgctgcc tgcttttgat gctgtggggc cgctgaagtc cggcaccaaa |
| 1861 | cctcagcccc aggtgtactc ctcgctcctc agcagcggct gccaggctgg ggagcacgca |
| 1921 | gcctgcttcg cagagcttcg aaggaacttc ataaacactt gccctcccaa acttaagagc |
| 1981 | ctgatgctac tggtcaaaca ctggtaccgc caggttgtca ctcgatataa aggaggagag |
| 2041 | gcggcaggtg atgctccgcc cccagcctac gccctggagc tcctgaccat ctttgcctgg |
| 2101 | gaacaaggct gtggagagca aaagttcagc ctggctgaag gcctgcggac catcctgagg |
| 2161 | ctgatccaac agcaccagtc gctttgtatc tactggacgg tcaactacag tgtgcaggac |
| 2221 | ccgaccatca gagcacatct tctctgccag cttcggaaag ccaggcctct agtcctggac |
| 2281 | cctgcagatc ccacctggaa cgtgggccag ggcgactgga agctattagc tcaggaggca |
| 2341 | gctgcccttg ggtcacaagt ctgccttcag agtggggatg ggactctggt gccaccctgg |
| 2401 | gatgtgacgc cagccctcct tcaccagacc ctagctgagg acctggacaa attcatcagt |
| 2461 | gaattcctcc agcccaaccg ccacttcctg actcaagtga agagagccgt ggacaccata |
| 2521 | tgttccttcc tgaaagaaaa ctgcttccgg aactctacca tcaaggtgct caaggtggtc |
| 2581 | aagggtgggt cttctgccaa aggcacggct ctacaaggac gctcagatgc cgacctggtg |
| 2641 | gtgtttctca gctgcttccg ccagttctct gagcaaggca gccatcgggc agagatcatc |
| 2701 | tcggagatcc aggctcagct ggaggcgtgt cagcagacgc acagcttcga tgtcaagttt |
| 2761 | gaggtctcca agaggaagaa cccccgagtg ctcagcttca cgctgacatc ccagacgctg |
| 2821 | ctggaccaaa gcgtggactt tgacgtcctg ccagcctttg atgctctcgg ccagctgagg |
| 2881 | tccggctctc ggcctgatcc ccgggtctac acagacctca tccacagctg cagtaatgca |
| 2941 | ggagagttct ctacctgctt cacagagctg cagagggact tcattacctc ccgtcccacc |
| 3001 | aaactcaaga gcctgatccg gctggtgaaa tactggtacc aacagtgtaa caagaccatc |
| 3061 | aaggggaagg gttccttgcc tccccagcac gggctggagc tcctaactgt gtacgcctgg |
| 3121 | gagcaaggtg gccagaatcc ccagttcaac atggcggagg gcttccgcac tgttctggag |
| 3181 | ctgattgtcc agtaccggca gctctgcgtc tattggacca tcaactacag cgcagaagac |
| 3241 | aagaccatcg gtgacttcct gaagatgcag cttcggaagc ccaggcctgt catcctggac |
| 3301 | ccagctgacc cgacaggcaa cctgggccac aacgctcgct gggatctgct tgccaaggag |
| 3361 | gctaccgtgt acgcatctgc cctgtgctgc gtggacaggg atggcaatcc catcaagcca |
| 3421 | tggccggtaa aggccgctgt gtgaagtcta gagagatcag tggtcaccat tgatagaaag |
| 3481 | tgacaccagc cctcagcaag tgatactcag agtatctgag tgtgtgtgtg tgtgtgttgt |
| 3541 | atttatctgt atgtgtgtat ttgtggtatg tctgtgtgcc tatatgaggg tgtgtctatg |
| 3601 | tgcgtgtctg tgtatctgtg ggtatctata tgtgtctgta tatatgtatg tgtgtgtgtg |
| 3661 | tgtgtgtgtg tattcatgta tgtgtgtctg tttgtgtata gtgtgtctat aggtgactct |
| 3721 | gtgtgtctgt gtatctgtga gtatctatat gtgtctgtct gtatgtaaat gtgtgtatgt |
| 3781 | atgtgtgttc atatgtctgt gtgtgtgtct atatctgtgt atctttgggt atctatatgt |
| 3841 | gtctatctat atgtaaatgt atgtatgtac ttatgttcat gtgtatctgt gtgaatgtct |
| 3901 | gtgtgtttat gtgtagtgta tctgtaagtg tatctgtatg tctatagatg tattatgtct |
| 3961 | ttgtgtgtcg acatgtctgt gtgtatgtat gtttgtatgt gtatgttata tatgtatata |
| 4021 | tgcatgtatg tgcttcctca caccatctcc cttctgccca cctgcccacc catagccctc |
| 4081 | cctttcttcc cactgtttac ccacctggtg gggcttcatt gacctcaacc atgatcatcc |
| 4141 | cggtgtccct gactcccaca ctagacaccc taggaaccag acatctctag atcttctagt |
| 4201 | ctgctgttca tctaccatgg gctctgcccc aacttccaca gccccaccca ggagtgcctc |
| 4261 | agccctgcca agaagccata ctcctccctg gcatctctct gccccttgag cctgtgtata |
| 4321 | tccctctgcc tacagagacc caccagctga ggtccaacta tgttcctgta ctggctggtt |
| 4381 | ttgtgtgtca acttgacata ggctggagtt atcagagaaa ggagcttcag ttggggaaat |
| 4441 | gcctccatga gatccagctg tggggcattt tctcagttgg tgatcaaaga ggagggccca |
| 4501 | ttgtgggtgg tgccatccct gggctggtag tcttgagttc tataagagat caagctgagc |
| 4561 | aagccagggg aagcaagcca ataagaaaca tccctccatg gcctctgcat cagctcctgc |
| 4621 | ttcctgacct gtttgagttc cagttctgac ttcctttagt gatgaacagc aatgtggaag |
| 4681 | tgtaagctga ataaaccctt tcctcccc |
| // | |
| (SEQ ID 15)LOCUS AF459815 1914 bp mRNA linear ROD 25-JUN-2002 | |
| DEFINITION Mus musculus 2′-5′ olygoadenylate synthetase | |
| 1c (Oas1c) mRNA, Oas1c-RV allele, complete cds. | |
| ACCESSION AF459815 | |
| /translation=“MENGLCSIQARELDEFICDYLFPDTTFLTELRADIDSISAFLKE | |
| RCFQGAAHPVRVSRVVMGGSYDEHTALKGKSEAKMVLFFNNLTSFEEQLKRRGEFVEE | |
| IQKHLCQLQQEKPFKVKFEVQSSEEPNSRSLSFKLSSPELQQEVEFDVQPAYDVLYEL | |
| RNNTYAEPQFYNKVYAQLIHECTTLEKEGDFSICFTDLHQNFMRYRAPKLWNLIRLVK | |
| HWYQLCKEKLREPLPPQYALELLTVYVWEHSNKNQEKVTTAKNFRTFLELVAYYKNLR | |
| IYWTWYYDFRHQEVCAYLCRQLKKARPLILDPADPTRNVAGSDLQAWDLLAKEAQ | |
| TWMQSSCFRNCDMSFVPTWDLSPERQECAFQ” | |
| BASE COUNT 495a 486c 489g 444t | |
| ORIGIN | |
| 1 | aaacactcct ggcctcagga tggagaatgg tctctgcagc atccaagcca gggagctgga |
| 61 | cgagttcata tgtgattacc tctttcctga caccaccttc cttactgagc tcagagcaga |
| 121 | catcgactcc ataagtgctt tcctgaagga gagatgcttc caaggtgccg cccatcctgt |
| 181 | gagggtctcc agggttgtga tgggcggctc ctatgatgaa cacactgcac tcaagggcaa |
| 241 | gtcagaggcc aaaatggtgt tgttctttaa caatctcacc agctttgagg agcagttaaa |
| 301 | gcgacgggga gagttcgttg aggaaattca gaaacacctg tgtcagctgc agcaagagaa |
| 361 | accatttaaa gtgaagtttg aagtgcagag ctcagaggag cccaactcca ggtctctgag |
| 421 | cttcaagctg agctcccccg agctccagca ggaggtggaa tttgatgtgc agccagccta |
| 481 | tgatgtccta tatgaactga gaaacaacac gtatgctgaa ccccaattct acaacaaagt |
| 541 | ctacgcccaa ctcatccatg agtgcaccac cctggagaag gagggcgatt tctccatctg |
| 601 | cttcaccgac ctccatcaga acttcatgag gtatcgtgcg cccaagctct ggaacctcat |
| 661 | ccgtctggtc aagcactggt atcaactgtg taaggagaag ctgagggagc cgctgccccc |
| 721 | acagtacgcc ctggagctgc tcactgtcta tgtatgggaa cattcgaata aaaatcaaga |
| 781 | aaaagtaacc acagccaaga acttccggac cttcttagaa ctggtcgcct attacaagaa |
| 841 | tcttcgaatc tactggacat ggtattatga cttccgacat caagaggtct gtgcctacct |
| 901 | gtgcagacag ctcaaaaaag ccaggcctct gatcctggat ccagcagacc caacaaggaa |
| 961 | cgtggctggt tcagacttac aggcatggga cctgctggca aaggaggctc agacctggat |
| 1021 | gcagtcctct tgctttagaa actgtgatat gtcctttgtg cccacctggg atttgtcgcc |
| 1081 | agagagacaa gaatgtgcct tccagtgagc agtgcagcgc ttgctctgaa ggctccagag |
| 1141 | tcaggggcat accttcctct gctgcaagac cttgacctag agaggacagg atggtgctaa |
| 1201 | aggctccagt gaggggcatc cagcctgtga tcagactcca ggcttctgat ccctgactgc |
| 1261 | ccatggatag ccttcctcac aggctgcttc gtctgcctta gcttccaaca gtgttctctg |
| 1321 | ggagtcagac tgtgatggac agagaagaac gcaagctcga cttccatctg tccacctgtt |
| 1381 | gggaggttct gtccaacagt ggctgattgt catcaacaaa ccacagcaag ccatggggga |
| 1441 | gggtgcactc tgagagaagg aacctttaag tacacttgtg tgtctgtgtg tttaaggatg |
| 1501 | tggtgtgtcc atatgcaact agaaaccttg agcacgtgtt acaagctcca catgggccca |
| 1561 | ggtaattgcc agaaaggggt ggacagagca aaaccaaact gttacacgta ttgatgttgg |
| 1621 | gtagcttggg atccttctag atctctgatg caagaaaccc agactagaat ccatggctcc |
| 1681 | tgctgtccat tctcctgtga caaaatttta ggccttcccc atcccacaca gaaactgttc |
| 1741 | tccaaccaca catgaccctg gagccctggg aatctggcca gcgtgcatcg tggtgcactg |
| 1801 | attctgcagc atgcaggctg aggtccacag cagtgtggga aagtaaaact atgtgcaatt |
| 1861 | tgtgaccagt gatgacttga aagcttagct gtctgtgtga gggtgagatt tgaa |
| // | |
| (SEQ ID 16)LOCUS AF459816 1911 bp mRNA linear ROD 25-JUN-2002 | |
| DEFINITION Mus musculus 2′-5′ olygoadenylate synthetase | |
| 1c (Oas1c) mRNA, Oas1c-He allele, complete cds. | |
| ACCESSION AF459816 | |
| /translation=“MENGLCSIQARELDEFICDYLFPDTTFLTELRADIDSISAFLKE | |
| RCFQGAAHPVRVSRVVMGGSYDEHTALKGKSEAKMVLFFNNLTSFEEQLKRRGEFVEE | |
| IQKHLCQLQQEKPFKVKFEVQSSEEPNSRSLSFKLSSPELQQEVEFDVQPAYDVLYEL | |
| RNNTYAEPQFYNKVYAQLIHECTTLEKEGDFSICFTDLHQNFMRYRAPKLWNLIRLVK | |
| HWYQLCKEKAEEPLPPQYALELLTVYVWEHSNKNQEKVTTAKNFRTFLELVAYYKNLR | |
| IYWTWYYDFRHQEVCAYLCRQLKKARPLILDPADPTRNVAGSDLQAWDLLAKEAQ | |
| TWMQSSCFRNCDMSFVPTWDLSPERQECAFQ” | |
| BASE COUNT 494a 487c 487g 443t | |
| ORIGIN | |
| 1 | aaacactcct ggcctcagga tggagaatgg tctctgcagc atccaagcca gggagctgga |
| 61 | cgagttcata tgtgattacc tctttcctga caccaccttc cttactgagc tcagagcaga |
| 121 | catcgactcc ataagtgctt tcctgaagga gagatgcttc caaggtgccg cccatcctgt |
| 181 | gagggtctcc agggttgtga tgggcggctc ctatgatgaa cacactgcac tcaagggcaa |
| 241 | gtcagaggcc aaaatggtgt tgttctttaa caatctcacc agctttgagg agcagttaaa |
| 301 | gcgacgggga gagttcgttg aggaaattca gaaacacctg tgtcagctgc agcaagagaa |
| 361 | accatttaaa gtgaagtttg aggtgcagag ctcagaggag cccaactcca ggtctctgag |
| 421 | cttcaagctg agctcccccg agctccagca ggaggtggaa tttgatgtgc agccagccta |
| 481 | tgatgtccta tatgaactga gaaacaacac gtatgctgaa ccccaattct acaacaaagt |
| 541 | ctacgcccaa ctcatccatg agtgcaccac cctggagaag gagggcgatt tctccatctg |
| 601 | cttcaccgac ctccatcaga actttatgag gtatcgtgcg cccaagctct ggaacctcat |
| 661 | ccgtctggtc aagcactggt atcaactgtg taaggagaaa gctgaggagc cgctgccccc |
| 721 | acagtacgcc ctggagctgc tcactgtcta tgtatgggaa cattcgaata aaaatcaaga |
| 781 | aaaagtaacc acagccaaga acttccggac cttcttagaa ctggtcgcct attacaagaa |
| 841 | tcttcgaatc tactggacat ggtattatga cttccgacat caagaggtct gtgcctacct |
| 901 | gtgcagacag ctcaaaaaag ccaggcctct gatcctggat ccagcagacc caacaaggaa |
| 961 | cgtggctggt tcagacttac aggcatggga cctgctggca aaggaggctc agacctggat |
| 1021 | gcagtcctct tgctttagaa actgtgatat gtcctttgtg cccacctggg atttgtcgcc |
| 1081 | agagagacaa gaatgtgcct tccagtgagc agtgcagcgc ttgctctgaa ggctccagag |
| 1141 | tcaggggcat accttcctct gctgcaagac cttgacctag agaggacagg atggcactca |
| 1201 | aggctccagt gaggggcatc cagcctgtga tcagactcca ggcttctgat ccctgactgc |
| 1261 | ccatggatag ccttcctcac aggctgcttc atctgcctta gcttccaaca gtgttctctg |
| 1321 | ggagtcagac tgtgatggac agagaagaac gcaagctcga cttccatctg tccacctgtt |
| 1381 | gggaggttct gtccaacagt ggctgattgt catcaacaaa ccacagcaag ccatggggga |
| 1441 | gggtgcactc tgagagaagg aacctttaag tacacttgtg tgtctgtgtg tttaaggatg |
| 1501 | tggtgtgtcc atatgcaact agaaaccttg agcacgtgtt acaagctcca catgggccca |
| 1561 | ggtaattgcc agaaaggggt ggacagagaa aaaccaaact gttacacgta ttgatgttgg |
| 1621 | gtagcttggg atccttctag atctctgatg caagaaaccc agactagaat ccatggctcc |
| 1681 | tgctgtccat tctcctgtga caaaatttta ggccttcccc atcccacaca gaaactgttc |
| 1741 | tccaaccaca catgaccctg gagccctggg aatctggcca gcgtgcatcg tggtgcactg |
| 1801 | attctgcagc atgcaggctg aggtccacag cagtgtggga aactatgtgc aatttgtgac |
| 1861 | cagtgatgac ttgaaagctt agctgtctgt gtgagggtga gatttgaagc a |
| // | |
| (SEQ ID 17)LOCUS AF478457 4380 bp mRNA linear PRI 25-JUN-2002 | |
| DEFINITION Homo sapiens ATP-dependent RNA helicase mRNA, complete cds. | |
| ACCESSION AF478457 | |
| /translation=“MAADKGPAAGPRSRAAMAQWRKKKGLRKRRGAASQARGSDSEDG | |
| EFEIQAEDDARARKLGPGRPLPTFPTSECTSDVEPDTREMVRAQNKKKKKSGGFQSMG | |
| LSYPVFKGIMKKGYKVPTPIQRKTIPVILDGKDVVAMARTGSGKTACFLLPMFERLKT | |
| HSAQTGARALILSPTRELALQTLKFTKELGKFTGLKTALILGGDRMEDQFAALHENPD | |
| IIIATPGRLVHVAVEMSLKLQSVEYVVFDEADRLFEMGFAEQLQEIIARLPGGHQTVL | |
| FSATLPKLLVEFARAGLTEPVLIRLDVDTKLNEQLKTSFFLVREDTKAAVLLHLLHNV | |
| VRPQDQTVVFVATKHHAEYLTELLTTQRVSCAHIYSALDPTARKINLAKFTLGKCSTL | |
| IVTDLAARGLDIPLLDNVINYSFPAKGKLFLHRVGRVARAGRSGTAYSLVAPDEIPYL | |
| LDLHLFLGRSLTLARPLKEPSGVAGVDGMLGRVPQSVVDEEDSGLQSTLEASLELRGL | |
| ARVADNAQQQYVRSRPAPSPESIKRAKEMDLVGLGLHPLFSSRFEEEELQRLRLVDSI | |
| KNYRSRATIFEINASSRDLCSQVMRAKRQKDRKAIARFQQGQQGRQEQQEGPVGPAPS | |
| RPALQEKQPEKEEEEEAGESVEDIFSEVVGRKRQRSGPNRGAKRRREEARQRDQEFYI | |
| PYRPKDFDSERGLSISGEGGAFEQQAAGAVLDLMGDEAQNLTRGRQQLKWDRKKKRFV | |
| GQSGQEDKKKIKTESGRYISSSYKRDLYQKWKQKQKIDDRDSDEEGASDRRGPERRGG | |
| KRDRGQAGASRPHAPGTPAGRVRPELKTKQQILKQRRRAQKLHFLQRGGLKQLSARNR | |
| RRVQELQQGAFGRGARSKKGKMRKRM” | |
| polyA signal 4351 . . . 4356 | |
| BASE COUNT 935a 1293c 1361g 791t | |
| ORIGIN | |
| 1 | ccttctgcgt tcccagcgcg cggcccgaat ggcggccgac aagggcccgg cggctggacc |
| 61 | tcggtcgcga gctgccatgg cccagtggag gaagaagaaa gggctccgga agcgccgagg |
| 121 | cgcggcctcc caggcccgcg gcagcgactc ggaggacggc gagtttgaga tccaggcgga |
| 181 | agatgacgcc cgggcccgga agctgggacc tggaagaccc ctgcccacct tccccacctc |
| 241 | ggaatgcacc tcggatgtgg agccggacac ccgggagatg gtgcgtgccc agaacaagaa |
| 301 | gaagaagaag tctggaggct tccagtccat gggcctgagc tacccggtgt tcaaaggcat |
| 361 | catgaagaag gggtacaagg tgccaacacc catccagagg aagaccatcc cggtgatctt |
| 421 | ggatggcaag gacgtggtgg ccatggcccg gacgggcagt ggcaagacag cctgcttcct |
| 481 | cctcccaatg ttcgagcggc tcaagaccca cagtgcccag accggggccc gcgccctcat |
| 541 | cctctcgccg acccgagagc tggccctgca gaccctgaag ttcactaagg agctaggcaa |
| 601 | gttcactggc ctcaagactg ccctgatcct gggtggagac aggatggaag accagtttgc |
| 661 | agccctgcac gaaaatcccg acataattat tgccacgccc ggacggttgg tgcatgtggc |
| 721 | tgtggaaatg agcctgaagc tgcagagtgt ggaatacgtg gtgttcgatg aagctgaccg |
| 781 | gctttttgaa atgggtttcg cagagcagct gcaggagatc atcgcccgcc tccccggggg |
| 841 | ccaccagacg gtgctgttct ccgccacgct gcccaaactg ctggtggaat ttgcccgggc |
| 901 | tggcctcacg gagcccgtgc tcatccggct tgacgtggat accaagctca acgagcagct |
| 961 | gaagacctcc ttcttcctcg tgcgggagga caccaaggct gccgtgctgc tccacctgct |
| 1021 | gcacaacgtg gtgcggcccc aggaccagac cgtggtgttt gtggccacga agcaccacgc |
| 1081 | cgagtacctc actgagctgc tgacgaccca gcgggtgagc tgcgcccaca tctacagtgc |
| 1141 | cctagacccg acagcccgca agatcaatct cgccaaattc acgcttggca agtgctccac |
| 1201 | tctcattgtg actgacctgg ccgcccgagg cctggacatc ccgctgctgg acaatgtcat |
| 1261 | caactacagc ttccccgcca agggcaaact cttcctgcac cgcgtgggcc gtgtggctcg |
| 1321 | ggctggccga agtggcacag cctactcctt ggtggcccct gatgaaatcc cctacctgct |
| 1381 | ggatctgcac ctgttcctgg gccgctccct caccctcgcc cgacccctca aggagccctc |
| 1441 | aggtgtggcc ggtgtggatg gcatgctggg tcgggtgcca cagagtgtgg tggacgagga |
| 1501 | ggacagtggt ctgcagagca ccctggaggc atcgctggag ctacggggcc tggcccgcgt |
| 1561 | tgctgataac gcccagcagc agtatgtgcg ctcacgcccg gcgccctcgc ctgagtccat |
| 1621 | caagagggcc aaggagatgg accttgtggg gctgggcctg caccccctct tcagctcgcg |
| 1681 | ttttgaggag gaggagctgc agcggctgag gctggtggac agcataaaga actaccgctc |
| 1741 | ccgggcgact atctttgaga tcaacgcctc cagccgagac ctgtgcagcc aggtgatgcg |
| 1801 | cgccaagcgg cagaaggacc gcaaggccat cgcccgcttc cagcagggac agcaggggcg |
| 1861 | gcaggagcag caggagggcc cagtgggccc agccccgagc cgcccagcac tgcaggagaa |
| 1921 | gcagcctgag aaggaggagg aggaggaggc gggagagagt gtggaggaca ttttctcaga |
| 1981 | ggtcgtgggc cggaagcggc agcggtcagg acccaacagg ggagccaaga ggcggaggga |
| 2041 | ggaggcccgg cagcgggacc aggaattcta catcccctac cggcccaagg actttgacag |
| 2101 | cgagcggggc ctgagcatca gcggggaagg gggagccttt gagcagcagg cagctggcgc |
| 2161 | tgtcctggac ttgatggggg atgaagccca gaacctgacg aggggccggc agcagctcaa |
| 2221 | gtgggaccgt aagaagaagc ggtttgtggg acagtcagga caggaagaca agaagaagat |
| 2281 | taagacagag agcggccgct acatcagcag ctcctacaag cgagacctgt atcagaagtg |
| 2341 | gaaacagaaa cagaaaattg atgatcgtga ctcggacgaa gaaggggcat ctgaccggcg |
| 2401 | aggcccagag cgaagaggtg ggaagcgaga ccgtggccaa gcaggtgcat cccggcccca |
| 2461 | cgccccaggc acccctgcag gccgagtccg cccggaactc aagaccaagc agcagatcct |
| 2521 | gaagcagcgg cgccgggccc agaagctgca cttcctgcag cgtggtggcc tcaagcagct |
| 2581 | ctctgcccgc aaccgccgcc gcgtccagga gctgcagcag ggcgccttcg gccggggtgc |
| 2641 | ccgctccaag aagggcaaga tgcggaagag gatgtgagga ccaggaccca gccccgtggc |
| 2701 | tccttgattg gccttagggt gggcatcagc agacgttccc gtgcaccact gtgtgcctgg |
| 2761 | ccctgtgctg ggcactgggg gcactccctg caggagccat catctgtgaa aaggagcact |
| 2821 | gtatggccac agaagggcag cagctgcgtc agcctaagac agagacattt gaacagggcc |
| 2881 | ttgaagggtg tgcaggagtt cgccagcaaa gccaggcagg ccaagacttg agttggcaac |
| 2941 | tcagctgctg ctgcttccat gtgttctggg ttcagaggtc atggctgcac cggtcagagc |
| 3001 | cctgagtgcc tcagggtttg gcaatggaat ttttaatgta ataaatcttt attgagcact |
| 3061 | gctggtggcc aggagtgcgg tctacttggg gaactggaat ggagagaccc aggtactaaa |
| 3121 | atcccagcta acgtggcaga ggagttgcgg gtctcctgag ggtgagttct gctgccttgt |
| 3181 | ccatttagcg atgaggaaag tgaagctcag agcacaaacc aggtgccaga ggcgggagtt |
| 3241 | ggtccccctt cctcccactg gacatggttg cagctgggag tgggctgggg gaggggaaca |
| 3301 | ggatgcccag cccagggggc aaggacacag ctgctcccct ctggctatga agaggttaac |
| 3361 | gcggcccctc cacacctgga ggtcagaacc tggcctgtcc tctgtcttct tgccacccac |
| 3421 | cccctgtttg aggttctgag aaggtcaagg gcagccccag cagctggatt ctcaggctgg |
| 3481 | gcccctcacc tggcagagtc catagtggag ggggccttgg tgatctctca tctagcatgg |
| 3541 | accctgttct gagacctgac aaagagtttt ttttcatgcc ccaaccaccc tggcagggag |
| 3601 | ggcctggtct gatctcattt tagaggcagt tgccccacac atagcccctt gaccttccca |
| 3661 | tcacctcctc atcagggcct gcatttatgg agtgcttgct atgtgcccct catggcaggc |
| 3721 | ccacagcact ctgaacaggc acagccctcc catcttccca atgaggaaac ggattcagga |
| 3781 | agagccactc agtccacacc ccattggaaa tgtgggccct cctatctcag ggtctctcag |
| 3841 | gggttccctc tcccgctgct cacctgtgtc agggctagga ggcaggggct gcaggctcag |
| 3901 | cctgacccag gccggccagc atggcagaga ggtcctgcat gaacagcttc acctgggggg |
| 3961 | gcccagcacg aagtcacatc ccagccccag agtcactgtc cagcccccac ccctcaacac |
| 4021 | gcgggggagg ccgaaaggta gggcggggac tggagatccc ctcattaaaa gaacagtgat |
| 4081 | gatggtggtt cccagaggtg gtgactgaga tcctaaaccg ttctgggttt tgaaagcctc |
| 4141 | aggccaacct tcccaactgc tgcgtgagca gacaccttca cagcttcctc gctgctgtca |
| 4201 | cctgcactat ccaattagta ttttcattta catcaatcag ctttattttc ttgtaactgg |
| 4261 | atcagtcata ttcattggtt tgtgacctac tcttatctcc gtgggtggct ctccttttgt |
| 4321 | tttaattaac ttctttatga atatgaactt aataaatacc atggatccat tgtaaaaact |
| // | |
| (SEQ ID 18)LOCUS AF480417 1993 bp mRNA linear ROD 26-JUN-2002 | |
| DEFINITION Mus musculus 2′-5′ olygoadenylate synthetase | |
| 1G (Qas1g) mRNA, complete cds. | |
| ACCESSION AF480417 | |
| /translation=“MEHGLRSIPAWTLDKFIEDYLLPDTTFGADVKSAVNVVCDFLKE | |
| RCFQGAAHPVRVSKVVKGGSSGKGTTLKGRSDADLVVFLNNLTSFEDQLNRRGEFIKE | |
| IKKQLYEVQHERRFRVKFEVQSSWWPNARSLSFKLSAPHLHQEVEFDVLPAFDVLGHG | |
| SINKKPNPLIYTILIWECTSLGKDGEFSTCFTELQRNFLKQRPTKLKSLIRLVKHWYQ | |
| LCKEKLGKPLPPQYALELLTVYAWEQGNGCNEFNTAQGFRTVLELVINYQHLRIYWTK | |
| YYDFQHKEVSKYLHRQLRKARPVILDPADPTGNVAGGNPEGWRRLAEEADVWLWYPCF | |
| MKNDGSRVSSWDVPTVVPVPFEQVEENWTCILL” | |
| BASE COUNT 493a 495c 537g 468t | |
| ORIGIN | |
| 1 | gccaggctgg gagacccagg aagctccaga cttagcatgg agcacggact caggagcatc |
| 61 | ccagcctgga cgctggacaa gttcatagag gattacctcc ttcccgacac cacctttggt |
| 121 | gctgatgtca aatcagccgt caatgtcgtg tgtgatttcc tgaaggagag atgcttccaa |
| 181 | ggtgctgccc acccagtgag ggtctccaag gtggtgaagg gtggctcctc aggcaaaggc |
| 241 | accacactca agggcaggtc agacgctgac ctggtggtgt tccttaacaa tctcaccagc |
| 301 | tttgaggatc agttaaaccg acggggagag ttcatcaagg aaattaagaa acagctgtac |
| 361 | gaggttcagc atgagagacg ttttagagtc aagtttgagg tccagagttc atggtggccc |
| 421 | aacgcccggt ctctgagctt caagctgagc gccccccatc tgcatcagga ggtggagttt |
| 481 | gatgtgcttc cagcctttga tgtcctgggt catggtagta tcaataagaa gcctaatccc |
| 541 | ttaatctaca ccatcctcat ctgggaatgt acctccctgg ggaaggatgg cgagttctct |
| 601 | acctgcttca cggagctcca gcggaacttc ctgaagcagc gcccaaccaa gctgaagagt |
| 661 | ctcatccgcc tggtcaaaca ctggtaccaa ctgtgtaagg agaagctggg gaagccactg |
| 721 | cccccacagt atgccctgga gctactcact gtctatgcct gggaacaggg gaatggatgt |
| 781 | aatgagttca acacagccca gggcttccgg accgtcttgg aactggtcat caattatcag |
| 841 | catcttcgaa tctactggac aaagtattat gactttcaac acaaggaggt ctccaaatac |
| 901 | ctgcacagac agctcagaaa agccaggcct gtgatcctgg acccagctga cccgacaggg |
| 961 | aatgtggctg gtgggaaccc agagggctgg aggcggttgg ctgaagaggc tgatgtgtgg |
| 1021 | ctgtggtacc catgttttat gaaaaatgat ggttcccgag tgagctcctg ggatgtgccg |
| 1081 | acggtggttc ctgtaccttt tgagcaggtg gaggagaact ggacatgtat cctgctgtga |
| 1141 | gcacagcagc acctgcccag gagactgctg gtcaggggca tttgctgctc tgctgcaggc |
| 1201 | ccatgaccca gtgagggagg gccccacctg gcatcagact ccgtgcttct gatgcctgcc |
| 1261 | agccatgttt gactcctgtc caatcacagc cagccttcct caacagattc agaaggagag |
| 1321 | gaaagaacac acgcttggtg tccatctgtc cacctgttgg aaggttctgt ctgacaaagt |
| 1381 | ctgatcaaca ataaaccaca gcaggtgccg tcatggtgtg tgaactctga ggagtgggcc |
| 1441 | atacaagaac agtgcaggtg tgtgagcgtg tgtgtgccca tgcacatgcg tgtgtgtctt |
| 1501 | cacggttcaa ctagatgcat ttagtgagca cttactacat atgctacatg attcagatgt |
| 1561 | tcagcagtgg ttagagcaaa gcctaactgc taggcttttt gatgcaagtt ggattgggat |
| 1621 | ccttccaggt ctcttcttac acatacacac aagagaggaa cccttggttt cttctgccca |
| 1681 | tgaccccaag acaagattct agccctgccc tatctgacac agaaacagtt ccctggccac |
| 1741 | acatggacat ggaacactga gactgtggcc tgtgctctca gggtgccctt gagtggctac |
| 1801 | aacatgcagg ctgggggccc ataggtatga tgaaaataaa aggtacctgg aatttttgac |
| 1861 | acatgtaact ttgaaacagg gtcattggta gcaacgatca gctttatcac atttagttaa |
| 1921 | atcacaatga ttgtggtttc ctttctgaga catgaatttg atgtgacaca cgctgtcgtg |
| 1981 | gaactcacag gaa |
| // | |
| (SEQ ID 19)LOCUS AF481733 1476 bp mRNA linear ROD 25-JUN-2002 | |
| DEFINITION Mus musculus 2′-5′ olygoadenylate synthetase | |
| 1F (Oas1f) mRNA, complete cds. | |
| ACCESSION AF481733 | |
| translation=“MVKDLSSTPACELDKFIRDHLLPDSSFHAEARADVDFIGAFLKE | |
| RCFQGATHPVRVSRVVMGGSYDEHTALKSKSEAKMVVFLNNLTSFEEQLKRRGEFIEE | |
| IRKHLCQLQDEKPFKVKFEVQSSEEPNSRSLSFKLSSPELQQEVEFDVQPAYDVLYEL | |
| RNNKYAELYLYNKIYAQLIHECTTLKKEGEFSICFTDLHQSFLEDRAPKLKNLIRLVK | |
| HWYQLCKEKLGKPLPPQYALELLTVYAWESGSRDCEFNTAQGFRTVLELVTKYKWLRI | |
| YWTVYYDFRKTKVSEYLHKMLQKVRPVILDPADPTRNVAGTNLLGWGLLAKEAAIWLQ | |
| SSCFRNCDTCLVGPWGVPVKVEIPQDCVLL” | |
| polyA signal 1459 . . . 1464 | |
| /gene=“Oas1f” | |
| BASE COUNT 378a 391c 385g 322t | |
| ORIGIN | |
| 1 | tcagcaaaca cttcctggcc ataaaatggt gaaggatctt agcagcaccc cagcctgtga |
| 61 | gctggacaag ttcatacgtg atcatctcct tcccgattcc agcttccatg ctgaggccag |
| 121 | agcagacgtg gacttcatag gtgctttcct gaaggagaga tgcttccaag gtgccaccca |
| 181 | ccctgtgagg gtctccaggg ttgtgatggg cggctcctac gacgaacaca ctgcactcaa |
| 241 | gagcaagtca gaggctaaaa tggtggtgtt ccttaacaat ctcaccagct tcgaggagca |
| 301 | gttaaagcga cggggagagt tcattgagga aattcggaaa cacctgtgtc agctgcagga |
| 361 | tgagaaacca tttaaagtga agtttgaggt gcagagctca gaggagccca actccaggtc |
| 421 | tctgagcttc aagctgagct cccctgagct ccagcaggag gtggaatttg atgtgcagcc |
| 481 | agcctatgat gtcctgtatg aactgagaaa caacaagtat gctgaactct acttgtacaa |
| 541 | caaaatctac gcccaactca tccatgagtg caccacacta aagaaggagg gcgagttctc |
| 601 | catctgcttc accgacctcc atcagagctt cctggaggat cgtgcaccca agctgaagaa |
| 661 | cctcatccgt ttggtcaagc actggtatca actgtgtaag gagaagctgg ggaagccgct |
| 721 | gcccccacag tatgccctgg agctgctcac agtgtacgcc tgggaaagtg ggagtagaga |
| 781 | ctgcgaattc aacacagccc agggcttccg aactgtcttg gaactggtca ccaagtacaa |
| 841 | gtggcttcga atctactgga cagtgtatta tgactttaga aagacgaagg tctctgaata |
| 901 | cctgcacaaa atgctccaaa aagtcaggcc tgtgatcctg gaccctgctg acccaacaag |
| 961 | gaacgtggct ggtaccaacc tactaggctg ggggctgttg gcaaaagaag ctgccatctg |
| 1021 | gctgcagtcc tcctgcttta ggaactgtga tacgtgcctc gtgggcccct ggggtgtgcc |
| 1081 | ggtgaaggtc gagattccac aggactgtgt ccttctatga gcaccaaagc acctgccagg |
| 1141 | atgctcaaga gtcagggata tgagatcctt gctctgctgc aggcccttga accagagaag |
| 1201 | ggggaaagct gctcacggcc ccaatcaggg agggtccaac ctgtgatcag actccaggct |
| 1261 | tctgacccct gccttctcac ccctgcatcc ggtcctatca cagatagcct tcttcgaagc |
| 1321 | ctgctttatc tgccttatcc accaacagtg tcctccggga gatgagagat tcagaattca |
| 1381 | gaaggggagg caggaactca agcttgactt ccacctgtcc acctgttggg aggttctgtc |
| 1441 | caatgtctga tgcacaataa taaatcacag agagcc |
| // | |
| (SEQ ID 20)LOCUS AF481734 13943 bp DNA linear ROD 25-JUN-2002 | |
| DEFINITION Mus musculus 2′-5′ olygoadenylate synthetase | |
| 1b (Flv) gene, Flv-C3H.PRI-Flvr allele, complete cds. | |
| ACCESSION AF481734 | |
| translation=“MEQDLRSIPASKLDKFIENHLPDTSFCADLREVIDALCALLKDR | |
| SFRGPVRRMRASKGVKGKGTTLKGRSDADLVVFLNNLTSFEDQLNQQGVLIKEIKKQL | |
| CEVQHERRCGVKFEVHSLRSPNSRALSFKLSAPDLLKEVKFDVLPAYDLLDHLNILKK | |
| PNQQFYANLISGRTPPGKEGKLSICFMGLRKYFLNCRPTKLKRLIRLVTHWYQLCKEK | |
| LGDPLPPQYALELLTVYAWEYGSRVTKFNTAQGFRTVLELVTKYKQLRIYWTVYYDFR | |
| HQEVSEYLHQQLKKDRPVILDPADPTRNIAGLNPKDWRRLAGEAATWLQYPCFKYRDG | |
| SPVCSWEVPTEVGVPMKYLFCRIFWLLFWSLFHFIFGKTSSG” | |
| polyA signal 12974 . . . 12979 | |
| /gene=“Flv” | |
| polyA site 12996 | |
| /gene=“Flv” | |
| BASE COUNT 3434a 3551c 3318g 3640t | |
| ORIGIN | |
| 1 | catccttttt ttttttttcc attcatttct tcatctgttt tagggttttt tccccctcac |
| 61 | actctggagt gaatccagct gtagtgtagc ccaggatgga ccagcctcca ctatccatcc |
| 121 | tcctgcatca gcctctgcct gctgaactgc tagggccacc cacaatgctg tttttcttcc |
| 181 | ctgttttcct gcatgtgaag ggctaatgac ctagaagccc caggcagtct gagtgactcc |
| 241 | ctgcaggagg agatatggtg ctgatgtcag tgtgcagtga ggatcaggga ggccaacggg |
| 301 | gtaagcaccc caccaaaatc caccagcttg gggaagcagg actaggtctg cagaccccca |
| 361 | aaggtcccat ttccacccta atcccctgga gctccctgtg cccggtgagc agtctccact |
| 421 | agcaatggct tccttcctgg agatgcataa aggccaatcc tgctcttgca gaaggccaga |
| 481 | ctctttattt cctcatcgaa gtacatcctg gttaatgctc gggtgtgcac atgtgagtat |
| 541 | acacttgttt gtgtatttgt gtgcatatgt gcacaggcaa gctcaggagt gtatttagtg |
| 601 | tgtgcatgcc ctgtgcgtgt gcatgtgaat gtgtgtgtgt gtgtgtgtgt gtgtgtgttt |
| 661 | gggacctgcc atgtcagttc tcagaattgg aactggaagc acagcccgag ctcttaaact |
| 721 | ctgagccctc tctctgccct ctaaacaaga gatgagaaga tggaggcagg ggttggaggg |
| 781 | catcaaactg ttttgatgag gccttgggta tgtcacccaa aggtgatttt caaccttgcc |
| 841 | agggccctga aagacagctc attttaatag tcactgctga cctgtactgt ccccagcttg |
| 901 | gtggtacggc ctgtacttca cccttgggga tgcccagacc ccaccctcac ccagaccccc |
| 961 | tgcaccacag gggaaaatgt agggtgtgag taggagaggg ggcttctggg aagtctgagc |
| 1021 | tttctgttag aattgtctgt aatcctagac ctgcaagtcc agaggtaaag gaaaaacaaa |
| 1081 | acctggggag gtgctgttct taaaaagaaa aaaaaaaaaa aaaagcagtt ccgcctaaaa |
| 1141 | cgtttgcagg agatggaagc cgagctcagg agccaccagc caccttctgt ggtttcctga |
| 1201 | ttccgtttcc cttcttctac gtgcacagat tgttcctaaa acgtaccgtg taacagcctt |
| 1261 | ttttccagaa gaaatcccga gaaagccagg tcagtttcct tacacattct tccctgggcc |
| 1321 | ggatcttaag aaagctcagg cttggatggg gaggtacctg ttcagaagcc ctaacgccat |
| 1381 | tggctgctcg ggcctggatg atttgcatat ccgcgccctt cccgggaaat ggaaactgaa |
| 1441 | agtcccattt ctgcttcagc gagcctagga gacacaggac ctgctggctg cagaggtatt |
| 1501 | agctggacct aggatggagc aggatctgag gagcatcccg gcctcgaagc ttgataagtt |
| 1561 | catagagaac catctcccgg acaccagctt ctgtgctgac ctcagagaag tcatagatgc |
| 1621 | cctgtgtgct ctcctgaagg acagatcctt ccggggcccc gtccgccgaa tgagggcctc |
| 1681 | taaaggggtc aaggtgagcc ttcctcagcc tgagctggcc gagatgaggt gggacaggac |
| 1741 | tttcagaagc caggctgcaa ccctgatccc tcctcttaat tctgatcaca gctggcgatg |
| 1801 | ggttcttccc cccaagtccc acatctgtat tggagaagga gcctcagcta cagtttatgt |
| 1861 | tccccactcc caggccatgt ccatttcaga gtcggggaaa ctgaggccca gaatggcaaa |
| 1921 | gcagttttct ggaaagtgga ggggcaggtg gtggggcagt ggtaatacaa cctttgcact |
| 1981 | gctgtgtgac ttatggtaac ttaactacct tctccatgtc cagtgttcca cgccccagga |
| 2041 | atgataaggt cacatagact aagtcaaatg tgacacggag gacagaccag gactcgcctg |
| 2101 | ctatgtgact ggattgatgg accctctctg gactcggttc ccgggctgtc tgtcaaatca |
| 2161 | catttcatga cactggtact taactctcag actctgtgaa gggtgcagaa atgaagttgt |
| 2221 | tttcttgttt tcagttgact gtatttctac atttttagtt gtgagtgggg ctctgtatgt |
| 2281 | gttagtgcag gtttggtcag aattgaaaag ggggtgtcag atcccttaga gctgcagtta |
| 2341 | cataggtggc tctgagctgg cctgcttggg tgctgggaac taaactgagg tcatcagcag |
| 2401 | gaacagcagg cagttttagc tactgaccca cctctccatc cggagatgtg acaatttgta |
| 2461 | atgccctctc tcaacagtag ctgatgctta tgtgtgttag tgatgattct aagcatttta |
| 2521 | tttagggagg ctttgttcag catgaattct tctttttatg atatttgttt tacagacttg |
| 2581 | gatcaccttt attgtttttt tcagtttctc tatgatgtta tatgttattc tatatcacac |
| 2641 | aaggccatct tctctttaat gatgtcactt gcgcatattt cctcccagct aacaatgcca |
| 2701 | accatgggac tatggctaca ttctcatata tctgtattaa tttagtcata gtctccttcc |
| 2761 | aatctctact atcctttacc tcccaccaga ccctccccag agattcactc tctgatggta |
| 2821 | tgtcacatgt gtgcagctaa tgtgggcact gcatgagtgc tactgtaccc ttcccaaact |
| 2881 | cttctgtgct tgtttcattt agacctctcc cccactccca tacacagccc ccccccacac |
| 2941 | acacacacac ttgaacacac agagactcca cagatgaaca tggatatttt ccttcacggt |
| 3001 | gtggtttatc ttctgtatca agaggagatc cagcaccctc tattttccta catagattct |
| 3061 | gatgtcactc ttcagggctg actaatattc tcctgcacat ctagcagctt gctccatcca |
| 3121 | tccaaccatc catccatcca tccatccatc tatcaccttg gtgatggact ccagttcagc |
| 3181 | atctggacct cccagcatgg ctctccctgt cctttgtcat tctctttctt gtctttcagg |
| 3241 | gcaaaggcac cacactcaag ggcaggtcag acgctgacct ggtggtgttc cttaacaatc |
| 3301 | tcaccagctt tgaggatcag ttaaaccaac agggagtgtt gattaaggaa attaagaaac |
| 3361 | agctgtgcga ggttcagcat gagagacgtt gtggagtgaa gtttgaggtc cacagtttaa |
| 3421 | ggagtcccaa ctcccgggct ctgagcttca agctgagcgc ccccgacctg ctgaaggagg |
| 3481 | tgaagtttga tgtgctgcca gcctatgatt tactgggtaa ggcagcctgc cagagagcct |
| 3541 | cagctcaccc ttctgcatgc cttcatcctc ccttctagtt ccacctctgt gtgtgtgcac |
| 3601 | atgtgtgttg cgtaggtgtt ctcttgtgtg tgggtgtgat ccggaaccat ggctaactca |
| 3661 | tctcaagagt catccatctt ttttgagacc ttgtctctcc ctggttcaga attcacactt |
| 3721 | gagcctgact ggctgggaag agccccaggg accctcagag tctccccctt ctcagttctg |
| 3781 | agattacaat cctacaccac caacactggc tttttctctg agttctggga ttccgacggg |
| 3841 | ggtcctcatg cttgcgagcc aagcacgtta cagactgagc tatctcccca gcccacacct |
| 3901 | tccatccctg aactgaaaac cattatcatt tgagaaattc aactaaagag aatgtctctt |
| 3961 | taaagctgcg ctggggagga gggattttgg aaatttggta gcggggagac agaatccatc |
| 4021 | ccacagggac attgagcagc ctgctggctc acttagggga gggagtcaca ctagatgctc |
| 4081 | cctcagcata ccagtcctac tcacggctag ctgtgggttt tgtcttctta aacatgagac |
| 4141 | agtgtagact tcactgaaaa taacaaactc gcgaattttc tccctcagag ctgttcagag |
| 4201 | acctggggtg tctgcaggag agtttgcaca ggacgcagat ctcatcggag ctctggcccc |
| 4261 | tcagtaaaac tgcagatgca gacacccaac tcacacatgc acacacattt tataacatta |
| 4321 | cacattaaaa gatcttatct tttgacaaaa atctcaatta ttcaatttta ccaatgaaga |
| 4381 | cttgcaaagc cagatactgt ggtaaaatcc tgctagctca gggaggcaga gaaagcacct |
| 4441 | agctgacctt cctcctgagc cagaatgttc cagaatgcat ccccttctca tgctatctca |
| 4501 | taacctcttt caaatggaat gtccctcccc tctacttcct gtgggtctct ctatccctcc |
| 4561 | ttctgactcc ctcttactct ctgtggttcc ttcttagtaa tcccatgttc gcttctggtc |
| 4621 | agctggttgc ttgctctgcc tcttgatcta tgattggctt tatttaatcc tgtttacaat |
| 4681 | attcaagcag aaagctcctg gattaagggt gtgtgctaag gctgagccac cacacctaaa |
| 4741 | accagttttt tccaataaac aactcaatct cagggttcac agtgtgatca aatatcctgc |
| 4801 | aacaatcttt taccaggcct gatacagcat gcgtcgccaa aattacagat tgtccaactc |
| 4861 | aggaaacact cctgagagca cagtgggatc ttgggggagc ttagagaata cttgaggctt |
| 4921 | tacatttggg ggaagcacag caccccctgt tggccactgt gtgaacttct gctgttaact |
| 4981 | tagtgacaag gccactagcc cttggcttag gagagccgtg gaaacagtcc agagtctcag |
| 5041 | gcagcaggga ctgagagggc cagcctggga gtaggaccag ggcagcctgc tgtgggaagc |
| 5101 | tgcaagaggc ttagaacctt gttcagtgga gggggatcag agtgggtgac cagaaatgat |
| 5161 | ctgtgacagc aggtcccaca gaagcctgtc tgggtcatgg atttcctcag cctcagcagc |
| 5221 | tttccagtgg tgaggggctc taccacagaa ggactgcctg tgggcaacag ccaaagagag |
| 5281 | gatcatagcc acagccctat agaagatgac ataaagcaat gagccctatt gtaactagac |
| 5341 | cctgacacta atctagacta gcccataacc tccatggtca ctgagtgaca ggcttgtcga |
| 5401 | agctccatcc gtgagtgact ttgccctgtg tcagcatcac agtctgatgg tgtggtctgc |
| 5461 | tgcatggcac acatgtgcac acacctaagg cacagcctgc ctagactact caaatgtgca |
| 5521 | cacacaccta tacacacatc tattccatag tctgctgctc ttctactcta taagtgcaca |
| 5581 | ggacacacac acacaaacac acacacaccc taacactgcc atatcttgtt ccatctggac |
| 5641 | aaacaaaacc acatgatatt aacttatcac tacaagaaat cacccaccga aaacacacag |
| 5701 | tggctgggga gacagctcag tggataaggg cgattgctgc tcaagcatga gtacctgagt |
| 5761 | tcaaatcccc aatatccacc taaacttagg aagtgccaca cattgacctg gatacctggt |
| 5821 | cactatgggg gcaaaagaca ggagattctg taagacctgc aggattccag ccaagctcca |
| 5881 | catttacaga cagactctga cgcaaagaaa catactgaca aactataaag aagagacacc |
| 5941 | ttaggtcctc tcctgtctct gaacatatca ggaacactcg ccatgcatac acaatataga |
| 6001 | gcagagactc tgtcagtgtg agacgtgata tgtgaatgca gccgactgct ccacagtgaa |
| 6061 | ctttgacaca tacatgagtg cacaactttg acaagaacta tgcagaagat actggaacca |
| 6121 | gacgtggttg gccattatct gatgatcacg tgtgtgtgac cacagtgtgc tggttcctcc |
| 6181 | cacctctgtc tccaggcctg caagaaagac cctcactaga gcaaactggg agcagggact |
| 6241 | ttgtagtgca cagcatgtgt gcagtcctaa ggagtggtgg ggcagatggg tatatgcgga |
| 6301 | gcgatgcctg ctggggaaaa acgctgcctg ttcgctgaac cccataggct gtgattctgg |
| 6361 | acctggggag actgaggaac ctcagagagg ggctgagctg ttccactgaa tgaaatcttg |
| 6421 | tttacagatc atcttaacat cctcaagaag cctaaccaac aattctacgc caatctcatc |
| 6481 | agtgggcgta ccccgccggg gaaggagggc aagttatcga tctgctttat ggggcttcgg |
| 6541 | aagtacttcc tgaactgtcg cccaaccaag ctgaagcgcc tcatccgcct ggtcacgcac |
| 6601 | tggtaccaac tggtaaggca ttggcctggt cactgtgcgc ttcatctgta cagagtgtag |
| 6661 | acagggggca gagaaaatgt gtactctagt tcggcgtcta ttgaggtaat aactctarca |
| 6721 | catgaccaaa acgctcctgg gaaggaaggg tattcttatg tttacacatc taaggcacac |
| 6781 | tccatcattg agggaagtca gaggaataac tgaagcagag gccatggagg aattctgctc |
| 6841 | accgtttgct gtccatgacg cactcagcgg ccatttctac acagcccaag gccacattcc |
| 6901 | attcatggct ccaaccacag tggtctggtc ccacccacat caatcagtga ttagtaacat |
| 6961 | gcccataaac tacccaagcc agtcagatgg atctgcattt tactgagcaa gatggactcc |
| 7021 | taccagagga ggctagctgg tgtttagtgg acaaaaagta agcagcacaa ttgacccctt |
| 7081 | ttcaacatgg cacacaaaca aaccacagtt gaaccataac ctttcccttc ttgcttgtcc |
| 7141 | ctaagatctc atgtcgctat cacaccatag aacacaactt aggtttaaag tccccaaatt |
| 7201 | aaaaatcaaa cctaaagaaa ctcccaacac ataaaaagac tagtctctca atgctggcct |
| 7261 | catataaaat taaaacacaa gttaaatact ttcttactgc aagagggaag aaccaagtca |
| 7321 | ctgtcacaat ctaattaaaa cacaaccaaa attcaacaga acaaaaggct cagtatctga |
| 7381 | tatctgggac tcatagttct ctgagctttt tgggctctgc catccacagc acacacagca |
| 7441 | ggtctcaggc tcaggccagc tttgttctca cagtgctctg ttcctgacag tcatctcctg |
| 7501 | ctcagggcaa ggccagtatc ttggggtttc cactgccatt gaggctgcac tttcactaat |
| 7561 | ggcttctcct ggcctctcgc agtgccaggc ctcagctgct ctccaaggcc ccttcagtcc |
| 7621 | agcagttctc atgggacttg aaatctggga gacatctctg cagttccagg tgtgattgac |
| 7681 | acttggagat gcagacttgg gacccctgga ctacagctga tagagtccat ggtctgccaa |
| 7741 | agaatgatgt cctgcactca aattgtatgc aaaagcaaag agcgttttta ttctgtggag |
| 7801 | tccagcatgc tgggatctat cattaaccaa gatggagatg cccagatgag atcacaggct |
| 7861 | caatttaaag cacattagtg gaattccatg gaagagtagg tgaccttttt cttgattggt |
| 7921 | tggctctatt ctagggacat tcctgaatca ttactggggg gctagaaact gtcacttggg |
| 7981 | gaagcctgga aactgttgct gactctgttc tttccctagg ccaggtggca gggaagcttc |
| 8041 | tgatggctgg gcagtttctg attggatgct ctaggcctgg ggtttttcca ataaatgact |
| 8101 | tgcctggact tgtacagttc tgagaaacac aaattcaggc ctagtctcct gatatgccag |
| 8161 | tttgaagccc attatggaat caccctggct cagtccactt acacctactc ttgctgacta |
| 8221 | tgaagaaact ctttccaaaa gatttgcacc ccccccccca cacacaccca tggtgcaggt |
| 8281 | ttcttcttta tcagagcaga ttttcagccc caactgatga gaaattactg actcttaatg |
| 8341 | taaacacagc acacaaacag cccagatggg tttttgcttt tctctgaaac ttcactccag |
| 8401 | cctctgtcac ctacctgcct ctcagcactc ctgtcttcca agttcccaca aacagcctgg |
| 8461 | taggctctca gctctcaaca gctctcccac ccaaagttcc ataatccttt ctcaatcctc |
| 8521 | ccaaggagca acatggcagg tcagtcacag cagcgcccct cctccctagg accagtttct |
| 8581 | gtcctagttt gctctgactg ctgtggtaaa gatgctgcaa agtaaattga ggtggaaatg |
| 8641 | gattgtctgg ctcacgttcc tgcatcacac tccatcactg agggaagcag aggcaggaac |
| 8701 | tgaagctgag gccatggagg aactctgcta gctgccggag acccttgggc gatcagtctg |
| 8761 | atttttacaa acaactcagg aacacctgcc cagggcaggc aatgcccaca atgggcgggg |
| 8821 | cccttctaca ctgatcaagg aaatgcccca caggcttgac tccaggccag gctgatggag |
| 8881 | gcattttctc agttgtgggt ccatcttctc cagatgattc tgtgtcactt tggcaaaaag |
| 8941 | aatcaaaatt agcaaaagca gtgagaaaaa caatgtaaaa aaaaaaaaaa agtgggtgga |
| 9001 | ggggaatcag aggggagggg atgagagaga agcaggggtg agtggagatg gggggagatg |
| 9061 | atgctgtagg aggggcaagg agtggggtgg gggatctgag aggagggagg attttccttg |
| 9121 | cctgttctag gtgatttctt ctagtccact ttccaatccg ctaactctct cttcagctgt |
| 9181 | gtctgattca ctgttccatg gcttctgctt catttgtctt tggatttttc tcccattgtt |
| 9241 | ggcaaatcct tcatgtcact tttcatactt tgattttcct gtcttgtcaa gctatgtttt |
| 9301 | taaatgtgtg atttcttatt atatcctgag tgtttttatg tgtatgtgtt tatacctatt |
| 9361 | cttggaaaca cacgtgtatg tgtaggcatg tgtgtccatt aggatttgag ggcagagtgt |
| 9421 | gagagcaggt tctgagtcac gccccacctt gtgtaatcag tcagggtttc acacgtgaca |
| 9481 | ctccgtgtgc tagattagca gttctggaca gtcatacagc ccgggggagc cccatcttta |
| 9541 | tccccttgaa cagggatgag gagtcagctt cctcaagagc ctgacaacac atagatgctg |
| 9601 | gggatctgac ctccagttct catccctgca tggggaggga ggcgctttac ctctgagccc |
| 9661 | tttatctccc tggccccttc ccagttactt cagagaccac aagcgaactt ctacagtgag |
| 9721 | tactccttat gtcctgcccc accctcactc ctagctgtca ctcatggtgt cctgattcct |
| 9781 | cctgggcctg ttagttacgt gctcagtctg tggagcacac agtgtccatc tcaaccgctc |
| 9841 | agctcttggc tatagcacaa gaaggcatag actgaattca agtgactggg tgtgacagtg |
| 9901 | tgacaataac cctgtcatga catcatcagg tggcagctga attgagcctg tgagaagcag |
| 9961 | ccccacacac aaccaccagg aaccccgttt tttttctcat tgcctttctc ttctcagtgt |
| 10021 | aaggagaagc tgggggaccc gctgccccca cagtatgccc tggagctgct cacagtctat |
| 10081 | gcctgggagt atgggagtcg agtaactaaa ttcaacacag cccagggctt ccgaaccgtc |
| 10141 | ttggaactgg tcaccaagta caaacagctt cgaatctact ggacagtgta ttatgacttt |
| 10201 | cgacatcaag aggtctctga atacctgcac caacagctca aaaaagacag gtagtctgtc |
| 10261 | cccacatgcc tgtgatcccc gtggagtgtc aggctgcact tgtgtttaaa gggggaggtg |
| 10321 | ggagctctgt gtctatgcag catcccctgt actgagcact gtcctgctgt cacctgattg |
| 10381 | acatgaggtc ctacagccct gggaggcagg ggtgggctct cctgtctgca gatgaggaga |
| 10441 | ccaaggctct gacattgtcc caacccaatc agcaaaggaa aggggggggg gacatggagt |
| 10501 | ggggactgag attttccaac agtgacagca tcataaaggc tgtgaggcac caaagacctg |
| 10561 | aggtggacaa attacctgta aataatacag tgctatgtca ccctagggag tgacatgtct |
| 10621 | tcattctgtg ttgtgttcag tttccacacc ctattctttg agcctggctg gtccaacact |
| 10681 | cttcatgtca cagatcagag accctgaggg atctgtccca ggacacacag ctactaaatc |
| 10741 | atgcccaagg atccctccag gcttctatat gctgtggggc agggacaaaa aaaaaatctt |
| 10801 | tgaaaaggag ctctctgctg ggctcctccc ccacccctgt gcagtggttc ctgggtcagt |
| 10861 | tgacagcagc tcgtttccct ctcttcatgt tgctctgcag gcctgtgatc ttggaccccg |
| 10921 | ctgacccaac aaggaacata gctggtttga acccaaagga ctggcggcgt ctagcaggag |
| 10981 | aggctgccac ctggctgcaa tacccatgct ttaagtacag ggacggttcc ccagtgtgct |
| 11041 | cctgggaggt gccggtgaga acgtgtcacc atcatcttca ttccacaccc cccccccttc |
| 11101 | tcttcatcta cctggagaat cttcccacac agatgtggtc tccgcactcc tgtggaggcc |
| 11161 | attggctctc cttattccac agccatctta gcgctattca cagatttcat ggactcacag |
| 11221 | gatggctcag tgggtaaagg tgcttgctgc caacacagat gacctgggtt tcatcccagg |
| 11281 | atctcctatg gtagaaggag agaatcaaat ccccaaagtt atcctctgac cttcatctct |
| 11341 | gctgttacac acagggacat gagtgtgtgc atacacacac atacacatac acacacacat |
| 11401 | gtcactacac ccacccaccc acctatccac aggttgcccc caaccaccac cacatagaca |
| 11461 | gacacacaca aaaatacaca aacacagagg ttgcccacac ccattcacac aaagacacac |
| 11521 | acacagacac acacacacag acacacctac acacacacac accatcatca tcatcatcat |
| 11581 | catcatcatc cacaaataga taacacacat acatagactt ggagacctga agccactggc |
| 11641 | tcggggacac tcagatagag tgaagaacgg ctgggacccc gtggactctg aagcccctgt |
| 11701 | caggtccatg aagcagcctg tcttccctgt ctctatcatg tggccaatgg gactcagctg |
| 11761 | ggctctatca cagcacagag gcacagaggg acctgcactt ggacctttct cctcctgggg |
| 11821 | cagggcctgc ctgatgctag ggacccagtg atgccatgtg tgccctccag atggcgttta |
| 11881 | gcatggctgt agtaatgctg gaaccatacc ccagggcaga atgggagttt gatgggtgtg |
| 11941 | gggatggtga tttgggaggt gggaggggcg agtcagactg cagtggcctt tagagctgtc |
| 12001 | cctctggcta tgcccactct caattgatgg atctgtcagt ggatgagagg ggacaagtgg |
| 12061 | gaggcatggc ttcagggaag agctgtatat atcctcaagg gcctaggccc tgtttagcct |
| 12121 | actgttgacc ttctcagcag cctgaccaag gggcccaggt ggcctgtcac agaactcatc |
| 12181 | ttcagggctc agttgcatcc ccttcccagt aagaatgtag gctcctgaca tgatcgtgtt |
| 12241 | ttctattaca gacggaggtt ggagtgccaa tgaagtatct cttttgtcgt attttctggt |
| 12301 | tattgttttg gtctttgttt catttcatct ttgggaagac ttcatctgga tagcccagag |
| 12361 | tgtcttggat attgccatcc tcctgcctta gcgctggcat gactgcagcg taggcctgtt |
| 12421 | atgctctgcc tcccctccat cctcaagtgg acaagaactg ggcatgtgtt ttcctgtgag |
| 12481 | cccagtggga cctgtccagg aggctccaga gtcaggggca tgtcctgctc tgctacaggg |
| 12541 | ccttgaccca gagaagacag gaaggtgccc aaagcccaag agagggaggg tccaacctgt |
| 12601 | gatcagactc caggcttctg tcccctgtcc tcaacccctg cacagacagc ctttctcaca |
| 12661 | gcatgcttta tctgtcttgt cccccaacag tgttctctgg gagacaagag attcagaagg |
| 12721 | agaatatgat ggtttgtata tggttggccc agggaatggc actgttagga gatgtggcca |
| 12781 | tgttggaatg ggtgtggcct tgtgggtgtg ggctttctct tgtcttagct gcctggaagt |
| 12841 | cagtatgctg ctagcagcct tcaaatgaag atgtagaact ctcagctcct cctgcaccat |
| 12901 | gcctgcctgg acgttgccat gctcttgcct tggtgataat ggactgaact tctgaacctg |
| 12961 | taagccaacc ccaattaaat gttgttttta taaaaattgc cttggtcatg gtgtctgttc |
| 13021 | acagcagtaa aaccctaaga cagggaggga ggaactcaag cttgaattgc atatgttcgc |
| 13081 | acgtcgggag gttctgtata tcaataacaa caaaccacag caagccatga gtgtgtgtgt |
| 13141 | atgtgtgtgg gtgtgggtct ctctttgtgt ctgtacctgt gtgtgtataa ggatgtgtgt |
| 13201 | gtgtatctct gtgtctgcat ttgtgtataa gaatgtgtgt gtatctgtgc atgtgtgtgt |
| 13261 | atgtggaatt gtgtgtgttt gtatctctct gtgtctgcat gtgtgtgtag gagggtgtat |
| 13321 | gtgtgtgcat ctgtgtatgt atatgtgttt gtctttctgt gtgtgtgtct ctgtgtgtgt |
| 13381 | gtgtctgcag attcatggtc aagtagctgc acttattaca ttctccatgt gggtcccagc |
| 13441 | accaagtcct cagtggtgga cagagcagaa ccaaactgat acaacttttg atgtcaggta |
| 13501 | gcctgggatc cttccaggtc tcttcttgca cataaacttg agacaggaat tcttagttcc |
| 13561 | ttctgtgttc ccaagcaaca ataatttctc ttgggaacaa ccccctttta gctatcttaa |
| 13621 | agcacatagt gtcttttcct ggtctgtttt gttttgctct ctctttctgg cttagagtca |
| 13681 | gatgaccgtt tgatctgaga tagagatttg aggagactaa acaggctgct taggttggga |
| 13741 | gaggagggtc ataatccaac tccagagcca gctttataat aatgctgaac agcataaaat |
| 13801 | aatgcttcag tgttattcat gcccaaaaga cagcatagct gagtacctgc aaaatggact |
| 13861 | caaggactgc agggagtgac tagagcaaac agagatcaaa ggacactcaa agatctgaga |
| 13921 | tttagcagga cttaaacatc agg |
| // | |
| (SEQ ID 21)LOCUS AY055829 1695 bp mRNA linear ROD 25-JUN-2002 | |
| DEFINITION Mus musculus 2′-5′ olygoadenylate synthetase | |
| 1D (Oas1d) mRNA, complete cds. | |
| ACCESSION AY055829 | |
| translation=“MARELFRTPIWRLDKFIEDQLLPDTTFLTELRADIDSISAFLME | |
| RCFQGAAHPVRVSRVVMGGCYNEYTVLKGRSEANMVVFLINLTSFEDQFNGQVVFIEE | |
| IWRHLLQLQQEKLCKLKFEVQSPKEPNSRFLSFKLSCPERQHELEFDVQPAYDALYEV | |
| RHFKPFDSSNYNKVYAQLTHECTTLEKEGEFSICFTDLHQSFLRYRAPKLWNLIRLVK | |
| HWYQLCKEKLRGPLPPQYALELLTVYVWEYGIHENPGLHTAQCFRTVLELVTKYKRLR | |
| STFFTNHDMSIVEAWEVMPERQECVFL” | |
| BASE COUNT 438a 432c 432g 393t | |
| ORIGIN | |
| 1 | gcagtcagca aacactcctg gcctcaggat ggcgagggaa ctcttcagaa ccccaatctg |
| 61 | gaggctggat aagttcatag aggatcaact ccttcctgac accaccttcc ttactgagct |
| 121 | cagagcagac atcgactcca taagtgcttt cctgatggag agatgcttcc agggtgccgc |
| 181 | ccatcctgtg agggtctcca gggttgtgat gggtggctgc tacaatgaat acactgtgct |
| 241 | caagggcagg tctgaggcca acatggtggt gttccttatc aatctcacaa gctttgagga |
| 301 | tcagttcaac ggacaggtag tgttcattga ggaaatttgg agacacctac tccagttgca |
| 361 | gcaagagaaa ctatgtaaac tcaagtttga ggtccagagc ccaaaggagc ccaactccag |
| 421 | gtttctgagc ttcaagctga gctgccccga gcgccagcat gagttggaat ttgatgtgca |
| 481 | gccagcctat gatgccctgt atgaagtaag acacttcaag ccctttgact ccagtaacta |
| 541 | caacaaagtc tacgcccaac tcacccatga gtgcaccaca ctggagaagg agggcgagtt |
| 601 | ctccatctgc ttcaccgacc tccatcagag cttcctgagg tatcgtgcgc ccaagctctg |
| 661 | gaacctcatc cgtttggtca agcactggta tcaactgtgt aaggagaagc tgagggggcc |
| 721 | gctgcctcca cagtacgccc tggagctgct cacagtctac gtctgggaat acgggatcca |
| 781 | cgaaaaccct ggactccata cagcccagtg cttccgcact gtcttagaac ttgtcaccaa |
| 841 | gtacaaacgg cttcgaatct actggacatg gtgttatgac tttcaacacg agatctctga |
| 901 | ctacctgcag ggacagatca aaaaagccag gcctctgatc ctggatccag cagacccaac |
| 961 | aaggaatgtg gctggttcag acttacaggc atgggacctg ctggcaaagg aggctcagat |
| 1021 | ctggatagat tcgactttct ttacgaacca tgatatgtcc attgtggaag cctgggaagt |
| 1081 | gatgccagag agacaagaat gtgtcttcct gtgagcaccc ccagcatctg cctaggagac |
| 1141 | tccagagtca ggggcatgtc ctcctcttct gtaagacctt gacctagaga ggacagacag |
| 1201 | gatggcactc aaggctccag cgaggggcat ccaacctgtg atcagactcc aggcttctga |
| 1261 | tccctgcctg cccatggaca gccttcctca caggctgctt cgtctgcctt agcttccaac |
| 1321 | agtgttctct gggagtcaga ctgtgatgga cagagaagaa cgcaagctcg acttccatct |
| 1381 | gttcacctgt tgggaggtta tgtccaatag tggctgatca tcatcaacaa accacagcaa |
| 1441 | gccatgaggg ggggtgcact ctgagggagg agtcctcaga ccacacagaa acttttcagc |
| 1501 | agtgcatgtg gccctggagc cctgggaatc tggccagtgt tcatcaaggt gcactgtttc |
| 1561 | tgcaacatgc aggctgggtt tatggtagtg caggaaaata aaattgcatg cattttaaaa |
| 1621 | tttatgattt taaaacttag gggtgtgtgt gtatgagatt tgaagcacta aattaaagca |
| 1681 | aaacgcattg aatta |
| // | |
| (SEQ ID 22)LOCUS AY055830 1336 bp mRNA linear ROD 25-JUN-2002 | |
| DEFINITION Mus musculus 2′-5′ olygoadenylate synthetase | |
| 1E, 45kD isoform (Oas1e) mRNA, complete cds; alternatively spliced. | |
| ACCESSION AY055830 | |
| /translation=“MARELFRTPIWRLDNFIEDQLLPDTTFLTELRADIDSISAFLKE | |
| RCFQGATHPMRVSRVVMGGSYDEHNALKGRSEANMVVFFNDLTSFEDQLKWQQVFIEE | |
| IRKHLLQLQQEKPCKLKFEVQSSEEPNTRSLTFKLCSPERQQEVEFDVQPAYDALYEG | |
| GYCKSFESINYNKVYAQLIHECTTLEKEGEFSICFTDLHQSFLRYRAPKLWNLIRLVK | |
| HWYQLCKEKLRGPLPPQYALELLTVYVWEFGVQDSFGLHAAQCFRTVLELVTKYKCLL | |
| IYWTWFYDFRPEISDYLHGQIKKARPLILDPADPTRNVAGSDLQAWDLLAKEAQTWIH | |
| SNFFRNCDMSLVNGWEVSLPTVFSGSQAVMDREERKLDFHLSTCWEVLSNSG” | |
| BASE COUNT 335a 342c 349g 310t | |
| ORIGIN | |
| 1 | tcaggatggc gagggaactc ttcagaaccc caatctggag gctggataac ttcatagagg |
| 61 | atcaactcct tcctgacacc accttcctta ctgagctcag agcagacatc gactccataa |
| 121 | gtgctttcct gaaggagaga tgcttccaag gtgccaccca ccctatgagg gtctccaggg |
| 181 | ttgtgatggg aggctcctat gatgaacaca atgcactcaa gggaaggtca gaggccaaca |
| 241 | tggtggtgtt ctttaatgat ctcaccagct ttgaggacca gttaaagtgg cagcaagtgt |
| 301 | tcattgaaga aattcggaaa cacctgctcc agttgcagca agagaagcca tgtaaactca |
| 361 | agtttgaggt gcagagctca gaggagccca acaccaggtc tctgaccttc aagctgtgct |
| 421 | cccccgagcg ccagcaggag gtggaatttg atgtgcagcc agcctatgat gctctgtatg |
| 481 | aagggggata ctgcaagtcc tttgaatcca ttaactacaa caaagtctac gcccaactca |
| 541 | tccatgagtg caccaccctg gagaaggagg gcgagttctc catctgcttc accgaccttc |
| 601 | atcagagctt cctgaggtat cgtgcgccca agctctggaa cctcatccgt ctggtcaagc |
| 661 | actggtatca actgtgtaag gagaagctga ggggaccgct gcctccacag tatgccctgg |
| 721 | agctgctcac agtctacgtc tgggaatttg gggtccaaga cagctttgga ctccatgcag |
| 781 | cccagtgctt ccgaacggtc ttagaactgg tcaccaagta caaatgcctt ctaatctact |
| 841 | ggacatggtt ttatgacttt cgacctgaga tctctgacta cctgcacgga cagatcaaaa |
| 901 | aagccaggcc tctgatcctg gatccggcag acccaacaag gaacgtggct ggttcagact |
| 961 | tacaggcatg ggacctgctg gcaaaggagg ctcagacctg gatacattca aattttttta |
| 1021 | ggaactgtga tatgtccctt gtgaatggct gggaagtgtc gcttccaaca gtgttctctg |
| 1081 | ggagtcaggc tgtgatggac agagaagaac gcaagctcga cttccatctg tccacctgtt |
| 1141 | gggaggttct gtccaatagt ggctgatcgt catcatcaaa tcacagcaag ccatggggga |
| 1201 | gggtgcactc tgagggagtc ctcagaccac acagaaactt ttcagcagtg catgtggccc |
| 1261 | tggcaccctg ggaatctggc cagtgttcat caaggtgcac tgtttctaca acatgcaggc |
| 1321 | cgggtttatg gcagtt |
| // | |
| (SEQ ID 23)LOCUS AY055831 1559 bp mRNA linear ROD 25-JUN-2002 | |
| DEFINITION Mus musculus 2′-5′ olygoadenylate synthetase | |
| 1E, 42kD isoform (Oas1e) mRNA, complete cds; alternatively spliced. | |
| ACCESSION AY055831 | |
| translation=“MARELFRTPIWRLDNFIEDQLLPDTTFLTELRADIDSISAFLKE | |
| RCFQGATHPMRVSRVVMGGSYDEHNALKGRSEANMVVFFNDLTSFEDQLKWQQVFIEE | |
| IRKHLLQLQQEKPCKLKFEVQSSEEPNTRSLTFKLCSPERQQEVEFDVQPAYDALYEG | |
| GYCKSFESINYNKVYAQLIHECTTLEKEGEFSICFTDLHQSFLRYRAPKLWNLIRLVK | |
| HWYQLCKEKLRGPLPPQYALELLTVYVWEFGVQDSFGLHAAQCFRTVLELVTKYKCLL | |
| IYWTWFYDFRPEISDYLHGQIKKARPLILDPADPTRNVAGSDLQAWDLLAKEAQTWIH | |
| SNFFRNCDMSLVNGWEVSPEKQ” | |
| BASE COUNT 385a 403c 405g 366t | |
| ORIGIN | |
| 1 | tcaggatggc gagggaactc ttcagaaccc caatctggag gctggataac ttcatagagg |
| 61 | atcaactcct tcctgacacc accttcctta ctgagctcag agcagacatc gactccataa |
| 121 | gtgctttcct gaaggagaga tgcttccaag gtgccaccca ccctatgagg gtctccaggg |
| 181 | ttgtgatggg aggctcctat gatgaacaca atgcactcaa gggaaggtca gaggccaaca |
| 241 | tggtggtgtt ctttaatgat ctcaccagct ttgaggacca gttaaagtgg cagcaagtgt |
| 301 | tcattgaaga aattcggaaa cacctgctcc agttgcagca agagaagcca tgtaaactca |
| 361 | agtttgaggt gcagagctca gaggagccca acaccaggtc tctgaccttc aagctgtgct |
| 421 | cccccgagcg ccagcaggag gtggaatttg atgtgcagcc agcctatgat gctctgtatg |
| 481 | aagggggata ctgcaagtcc tttgaatcca ttaactacaa caaagtctac gcccaactca |
| 541 | tccatgagtg caccaccctg gagaaggagg gcgagttctc catctgcttc accgaccttc |
| 601 | atcagagctt cctgaggtat cgtgcgccca agctctggaa cctcatccgt ctggtcaagc |
| 661 | actggtatca actgtgtaag gagaagctga ggggaccgct gcctccacag tatgccctgg |
| 721 | agctgctcac agtctacgtc tgggaatttg gggtccaaga cagctttgga ctccatgcag |
| 781 | cccagtgctt ccgaacggtc ttagaactgg tcaccaagta caaatgcctt ctaatctact |
| 841 | ggacatggtt ttatgacttt cgacctgaga tctctgacta cctgcacgga cagatcaaaa |
| 901 | aagccaggcc tctgatcctg gatccggcag acccaacaag gaacgtggct ggttcagact |
| 961 | tacaggcatg ggacctgctg gcaaaggagg ctcagacctg gatacattca aattttttta |
| 1021 | ggaactgtga tatgtccctt gtgaatggct gggaagtgtc gccagagaaa caataatgtg |
| 1081 | tcttccagtg agcagtgtag cacttgccta gaaggctcca gagtcaggat catgtgctcc |
| 1141 | tccgctgtaa gactttgacc tagagaggac aggatggtgc tcatgtctcc agcgaggggt |
| 1201 | atccaacctg tgattagact ccaggcttct gatccctgcc tgcccatgga tagccttcct |
| 1261 | cacaggctgc ttcatctgcc ttagcttcca acagtgttct ctgggagtca ggctgtgatg |
| 1321 | gacagagaag aacgcaagct cgacttccat ctgtccacct gttgggaggt tctgtccaat |
| 1381 | agtggctgat cgtcatcatc aaatcacagc aagccatggg ggagggtgca ctctgaggga |
| 1441 | gtcctcagac cacacagaaa cttttcagca gtgcatgtgg ccctggcacc ctgggaatct |
| 1501 | ggccagtgtt catcaaggtg cactgtttct acaacatgca ggccgggttt atggcagtt |
| // | |
| (SEQ ID 24)LOCUS AY057107 2041 bp mRNA linear ROD 25-JUN-2002 | |
| DEFINITION Mus musculus 2′-′ olygoadenylate synthetase-like | |
| 1 (Oas11) mRNA, complete cds. | |
| ACCESSION AY057107 | |
| /translation=“MAVAQELYGFPASKLDSFVAQWLQPTREWKEEVLETVQTVEQFL | |
| RQENFREDRGPARDVRVLKVLKVGCFGNGTVLRSTTDVELVVFLSCFHSFQEEAKHHQ | |
| AVLRLIQKRMYYCQELMDLGLSNLSVTNRVPSSLIFTIQTRETWETITVTVVPAYRAL | |
| GPSCPSSEVYANLIKANGYPGNFSPSFSELQRNFVKHRPTKLKSLLRLVKHWYQQYVR | |
| DKCPRANLPPLYALELLTVYAWEAGTREDANFRLDEGLATVMELLQDHELLCIYWTKH | |
| YTLQHPVIEACVRRQLRGQRPIILDPADPTNNVAEGYRWDIVAQRANQCLKQDCCYDN | |
| RDSPVPSWRVKRAPDIQVTVQEWGHSDLTFWVNPYEPIKKLKEKIQLSQGYLGLQRLS | |
| FQEPGGERQLIRSHCTLAYYGIFCDTHICLLDTISPEIQVFVKNPDGRSHAYAIHPLD | |
| YVLNLKQQIEDRQGLRCQEQRLEFQGHILEDWFDFKSYGIQDSVTVILSKTTEGAAPF | |
| VPS” | |
| polyA signal 2013 . . . 2018 | |
| /gene=“Oas11” | |
| BASE COUNT 469a 591c 551g 430t | |
| ORIGIN | |
| 1 | gtgtagccat ggcagtcgcc caggagctgt acggcttccc ggcctccaag ctggactcct |
| 61 | ttgtggctca gtggctgcag ccaaccagag agtggaaaga agaggtcctg gagaccgtgc |
| 121 | agacagtgga gcagttcctg aggcaggaga atttccgtga agatcgtggc ccggctcggg |
| 181 | atgtgcgcgt gctcaaggta ctcaaggtag gctgctttgg gaatggcacc gtgctcagga |
| 241 | gcactacaga cgtggagctg gtcgtgttcc tgagctgttt ccacagcttc caggaagaag |
| 301 | ccaagcacca tcaggctgtc ctgagactga tacagaaaag gatgtactac tgccaggagc |
| 361 | tgatggacct tgggctcagt aacctgagtg tgactaacag agtacccagt agtctcatct |
| 421 | tcacgatcca gaccagggag acctgggaga ccatcactgt caccgttgtg cccgcctaca |
| 481 | gagccctggg cccttcctgt cccagctccg aggtctacgc aaatctgatc aaggctaatg |
| 541 | ggtacccagg aaatttctct ccatccttca gcgagctgca gcgaaacttc gtgaagcatc |
| 601 | ggccgacgaa gctgaagagc ctccttcggt tggtcaaaca ctggtaccag cagtatgtga |
| 661 | gagacaagtg cccccgggcc aacctgcccc ctctctatgc cctggagctg ctcactgtct |
| 721 | atgcctggga agcgggcacc cgggaggatg ccaacttcag gctggatgaa ggcctcgcca |
| 781 | cggtgatgga gctgctccag gatcatgagc tcctctgtat ctactggacc aagcactaca |
| 841 | cgctgcagca cccggtcatc gaggcctgtg tcaggagaca gctcagggga caaaggccta |
| 901 | tcatcctgga cccagcagac cccaccaaca atgtggcaga aggctacaga tgggacatag |
| 961 | tggctcagcg ggccaaccag tgtctgaaac aggactgttg ctatgacaac agggacagcc |
| 1021 | ccgtccccag ctggagggtg aagagagcac ccgatatcca ggtgaccgtg caggagtggg |
| 1081 | ggcactcgga tttaaccttc tgggtgaacc cttatgaacc cataaagaaa ctgaaagaga |
| 1141 | aaatccaact gagccagggc tacttgggcc tgcagcgtct gtcctttcag gagcccggcg |
| 1201 | gagaacgtca gctcatcaga agccattgca cgctcgccta ctacggaatc ttctgcgaca |
| 1261 | cccacatctg cctgctggac accatctccc ctgagatcca ggtctttgtg aaaaacccgg |
| 1321 | atggcaggag ccacgcctat gcgatccacc cgcttgatta tgtcctgaac ctgaagcagc |
| 1381 | agatagaaga caggcagggc cttcgatgcc aggagcagcg cctggagttc cagggccaca |
| 1441 | tcctggagga ctggtttgac tttaaatcct atggcatcca agacagtgtc acagtcatcc |
| 1501 | tgtccaagac gacggagggg gcagctccat ttgtgcccag ctagcttcct ctgtcggtgg |
| 1561 | ctctgcctgt tttattgtct catcctagac tcagcctagt tgcctctcct tcccgtcctc |
| 1621 | tgcccggatg gtccacgtct tcagtacctt gccagcaggg agtcagaggg ggtgtgagaa |
| 1681 | gtcgtgtaca gccagacact cttgtgtgac aatggaattc tgcagtcccc tgggaagtca |
| 1741 | tgccaggacc tctgccttcc tcgtggcctc actgtcaaga ctgtgtcagt gaatagctgg |
| 1801 | cctcacagac tattctcaca tgttcagaga aagccaaacc atcttcctaa ccaattacag |
| 1861 | ggaccctgct tgaggttgtc ccacctccaa attcttccca gtgacctcca tcagggcggc |
| 1921 | tctgaagcct tcccctgtgt ccccaaccac ttctgcctgc cttcgactat ccaaggcaag |
| 1981 | gtaggagggg atcaagttcc tttcaaatgg agaataaaaa agccattgtt tcttcccaga |
| 2041 | t |
| // | |
| (SEQ ID 25)LOCUS AY196696 1442 bp mRNA linear ROD 20-MAY-2003 | |
| DEFINITION Rattus norvegicus 2′-5′ oligoadenylate synthetase | |
| 1A mRNA, complete cds. | |
| ACCESSION AY196696 | |
| translation=“MEKDLKSTPASELDKFIQDHLLPDTTFRDEVRADIDFICTFLKE | |
| KCFHGAALKVSKVVKGGSSGKGTTLQGKSDADLVVFLNNLTSFEDQLKRRGEVIEKIR | |
| KHLCQLQQEKQFKLKFEFQTPEQANSRSLSFKLSSPQLQHVVKFDVLPAYDVLGHVNI | |
| NSKPNAQIYASLIRKCTDLNKEGAFSTCFTELQRNFLKRRPTKLKSLIRLVKHWYQLC | |
| KEKLGDSLPPQYALELLTVHAWERGNGLTEFNTAQGFRTVLELVTKYQQLRIYWTMYY | |
| DFQHLDVSKYLYRQLKKPRPVILDPADPTGNVAGGNQEGWRRLASEARLWLQCPCFMN | |
| RDGSPVSSWEVQTEVPVCF” | |
| BASE COUNT 366a 380c 387g 309t | |
| ORIGIN | |
| 1 | gctatggata taagggcagc caactccaga ggcaaggctg cagtcagcaa acatttgtgg |
| 61 | cctcaggatg gagaaggatc tcaagagtac tccagcctcg gagctggaca agttcataca |
| 121 | ggatcacctt cttcctgaca ccacattccg tgatgaggtt agagcagaca ttgacttcat |
| 181 | atgtactttc ctgaaggaga aatgcttcca cggtgccgcc ttgaaggtct ctaaggttgt |
| 241 | gaagggtggc tcctcaggca aaggaaccac gctacagggc aagtcagatg cagacctggt |
| 301 | ggtgttcctt aacaatctca ccagctttga ggatcagtta aagcgacggg gagaggtcat |
| 361 | tgagaaaatt cggaaacacc tgtgccagtt gcagcaagag aaacagttta aactgaagtt |
| 421 | tgagttccag accccagagc aggccaactc caggtctctg agcttcaagc tgagctcgcc |
| 481 | ccagctccag catgtggtga agtttgatgt gctaccagcc tatgatgtcc tgggtcatgt |
| 541 | taacatcaac agcaagccta acgcccaaat ctatgccagt ctcatcagga agtgcaccga |
| 601 | cctgaataaa gagggcgcgt tttctacctg cttcacggag ctccagagga acttcctgaa |
| 661 | gcggcgccca accaagctga agagtctcat ccgcctggtc aagcactggt accaactgtg |
| 721 | taaggagaag ctgggggatt cgctgccccc acagtatgcc ctggagctgc tcacggtcca |
| 781 | tgcctgggaa cgtggaaatg gacttactga gttcaacaca gctcagggct tccggacagt |
| 841 | cttggaactg gtcacaaagt accagcagct tcgaatctac tggactatgt attatgactt |
| 901 | tcaacaccta gatgtctcca aatacctata cagacagctc aaaaaaccca ggcctgtgat |
| 961 | cctggaccct gctgacccaa cagggaacgt ggctggtggg aaccaagaag gctggcggcg |
| 1021 | gttggcctca gaggcgaggc tgtggctgca gtgcccatgt tttatgaaca gggatggttc |
| 1081 | cccagtgagc tcctgggaag tgcagacgga ggttcctgta tgtttctagc aggtggatga |
| 1141 | ggcctggtca tgcatcctgc tgtgaaccca gcagcaccag cccaggaggc tccggagtca |
| 1201 | ggggcacgtg ctgctctgct gcaggacctt gacacagtga gggagggccc cactcgggat |
| 1261 | cacagtccat gcttctgatg cccgcccgcc atgtttgaat actgtccaat cacagatagc |
| 1321 | cttcctcaac agattcagaa ggggcggaaa gaactcaagc ttgacttcca tctgaccgtc |
| 1381 | cacctgttgg gaggttctgt ccaaccatgt ctgtcaacaa caataaagta caccaggtgc |
| 1441 | ca |
| // | |
| (SEQ ID 26)LOCUS AY196697 1680 bp mRNA linear ROD 20-MAY-2003 | |
| DEFINITION Rattus norvegicus 2′-5′ oligoadenylate synthetase | |
| 1D mRNA, complete cds. | |
| ACCESSION AY196697 | |
| translation=“MGHGLSSISASELDKFIEVYLLPNTSFGADVKLAINVVCDFLKE | |
| RCFRGAAHPVRVSKVVKGGSSGKGTTLKGKSDADLVVFLNNLTSFEDQLNRRGEFIKE | |
| IKKQLYEVQRERHFGVKFEVQSSWWPNPRALSFKLSAPHLQQEVEFDVLPAYDVLGHV | |
| SIYSMPDPQIYASLIRKCMYLGKEGEFSTCFTELQRNFLKRRPTKLKSLIRLVKYWYH | |
| LCKEKLGKPLPPQYALELLTVYAWERGNGFVDFETAQGFRAVLELIIKYQELRIYWTT | |
| YYNFQHQEVSNYLHTQLTRIRPVILDPADPTGNIAGSNPEGWRRLAGEAAAWLRYPCF | |
| KYKDGSPVCPWDVPMEVDVPYQEDHFFRNFCLFFLFLFLFIFWRVSCV” | |
| BASE COUNT 410a 438c 441g 391t | |
| ORIGIN | |
| 1 | aggctgcaga agcaaatgct ccggaccaat catggggcac ggactcagca gtatctcagc |
| 61 | ctcggagctg gacaagttca tagaggttta cctccttcca aacaccagct ttggtgctga |
| 121 | cgtcaaatta gcgatcaatg tcgtgtgtga tttcctgaag gagagatgct tccgaggtgc |
| 181 | tgcccaccca gtgagggtct ccaaggtggt gaagggtggc tcctcaggca aaggcaccac |
| 241 | actcaagggc aagtcagacg ctgacctggt ggtgttcctt aacaatctca ccagctttga |
| 301 | ggatcagtta aacagacggg gagagttcat caaggaaatt aagaaacagc tgtatgaggt |
| 361 | tcaacgtgaa agacattttg gagtgaagtt tgaggtccag agttcatggt ggcccaaccc |
| 421 | ccgggctctg agcttcaagc tgagtgcacc acacctccaa caggaggtgg agtttgatgt |
| 481 | gcttccagcc tatgatgtcc taggtcatgt aagcatctac agcatgcctg acccccaaat |
| 541 | ctatgccagt ctcatcagga agtgcatgta cctggggaag gagggcgagt tctctacctg |
| 601 | cttcacggag ctccagagga acttcctgaa gcggcgccca accaagctga agagtctcat |
| 661 | ccgcctggtc aagtactggt accatctgtg taaggagaag ctggggaagc cgctgccccc |
| 721 | acagtacgcc ctggagctgc tcacggtcta tgcctgggag cgtggaaatg gatttgtcga |
| 781 | ttttgagaca gcccagggct tccgggcagt cttggaactg atcataaagt accaggagct |
| 841 | tagaatctac tggacaacct attataactt tcagcaccaa gaggtctcca actacctgca |
| 901 | cacacagctc acaagaatca ggcctgtgat cctggacccg gctgacccaa caggaaacat |
| 961 | tgctggttcg aacccagagg gctggaggcg actagcagga gaggctgctg cctggctgcg |
| 1021 | gtacccatgc tttaagtaca aggacggttc cccagtgtgt ccctgggatg tgccgatgga |
| 1081 | ggttgacgtg ccgtaccagg aggatcactt ttttcgtaat ttttgtctat tttttttgtt |
| 1141 | tttgttcctt ttcatatttt ggagggtttc ttgtgtatag tgcaggctct cgtgtatatc |
| 1201 | gtcatcctcc tgcctcggtg ctggcatgac tgcagagtcc gcctgatgtg ccctggattc |
| 1261 | cctccatcct caagtggaca agactgtgca tctgtcgtcc tgtgagccca gcaggacctg |
| 1321 | cccaggaggc tccagagtca gtcatggctt tctgtgctgc aggcccttga cccagagagg |
| 1381 | gaaggaaggt tcccaagacc ccagtgaggg agggtccaac ctgtgatcag actctggtct |
| 1441 | tctgacccct gccttcctac tcctgcatcc tgtcccatca cagacagccc tcctcacagc |
| 1501 | ctgcttcatc tgccttgtcc tccaacagtg ctctcttggg agacaagaga ttcagaaggg |
| 1561 | gaggcaggaa ctcgagcttg acttccacct gtccacctgt tgggagttct gtccaatgtg |
| 1621 | tgaccaacga caataaacca tagcaagcaa aaaaaaaaaa aaaaaaaaaa aaaaaaaaaa |
| // | |
| (SEQ ID 27)LOCUS AY196698 1712 bp mRNA linear ROD 20-MAY-2003 | |
| DEFINITION Rattus norvegicus 2′-5′ oligoadenylate synthetase | |
| 1C mRNA, complete cds. | |
| ACCESSION AY196698 | |
| translation=“MSRRLARTPIWRLDRFIEDHLLPDTTFLTEVRADIDSICAFLTE | |
| RCFQGATHPVRVSRIVKGGWFNKYTMLKGRSEATLVVFLNNLTCFEDQLKRREEFTEE | |
| IWKHLCQLQQEKQFKLNFEVQSTEQPDSRTLSIKLSSPQLQQEVEFDVQPAYDVLYEL | |
| RDKDDFNPDNYREIYARLIRESTTLEMEGEFSVCFTDLHQNFLKYRAPKLWNIIRLVK | |
| HWYQLCKKRLKYPLPPQYALELLTVYAWERGLEDRPALHTGHCFRSVLDLITEYKKLR | |
| IYWTWCYEFKKETFDYLSRQIKKDRPVILDPADPTRNVAGSNIQSWHVLAKEAAVWAR | |
| SSFFRNSDMTFVNGWKVSAQKQECVYQ” | |
| BASE COUNT 458a 427c 452g 375t | |
| ORIGIN | |
| 1 | gcagtcagca cacattcctg acctcaggat gtcgaggcga ctcgccagaa ccccaatctg |
| 61 | gaggctggac agattcatag aggatcacct ccttcctgac accaccttcc ttactgaggt |
| 121 | cagagcagac attgactcta tatgtgcttt cctgactgag agatgcttcc aaggtgccac |
| 181 | ccaccctgtg agggtctcta ggattgtgaa gggtggctgg ttcaataaat acaccatgct |
| 241 | taagggaagg tcagaggcca ccctggtggt gttccttaac aatctcacct gctttgagga |
| 301 | tcagttaaag cgacgagaag agttcactga ggaaatttgg aaacacctgt gccagttgca |
| 361 | gcaagagaaa cagtttaaac tgaattttga ggtccagagc acagaacagc ccgactccag |
| 421 | gactctgagc atcaagctga gctcgcccca gctccagcag gaggtggagt ttgatgtgca |
| 481 | gccagcctat gatgtcctgt atgagctaag agacaaggac gactttaacc cggataacta |
| 541 | cagggaaatc tatgcccgcc ttatccgtga gagcaccacc ctggagatgg agggcgagtt |
| 601 | ctccgtctgc ttcacggacc ttcatcagaa cttcctgaag tatcgtgcac ccaagctgtg |
| 661 | gaatatcatc cgtctggtca agcactggta tcaactgtgt aagaagaggc tgaagtatcc |
| 721 | gctgccccca cagtacgccc tggagctgct cacggtctat gcctgggagc gtgggctgga |
| 781 | agaccgccct gcactacata caggccactg tttccgaagt gtcttagatc tgatcacaga |
| 841 | gtacaagaag cttcgaatct actggacatg gtgctatgaa ttcaaaaaag agacctttga |
| 901 | ctatctgagc agacagataa aaaaagacag gcccgtgatc ctggaccctg ctgacccaac |
| 961 | aaggaacgtg gctggttcaa acatccaatc ctggcacgtg ctggcaaaag aagctgcggt |
| 1021 | ctgggcgcgg tcgtctttct ttaggaacag tgatatgacc tttgtcaatg gctggaaagt |
| 1081 | gtcggcacag aaacaagaat gtgtctacca gtgagcacca cagcccttgc ccaggaggct |
| 1141 | tctgaagtca gggccatatg ctcttctgct gcaagacctt gtcctgaaga gaacagtttg |
| 1201 | gcgcaccagg gtccagtgag gggcatccaa cctgtgatca gactccaggc ttctattccg |
| 1261 | tcctgcccat gggacagcct tcctcacagc ctgcttcacc tgccttatcc cccacagtgt |
| 1321 | tctttgggag tcagactccg aaggaaagag aagagctcaa acgtggcttc tgtctgtcca |
| 1381 | cctgtgggga ggctctgtcc acaaacatgt ctgatcatga acaatcgacc acagcaatcc |
| 1441 | ctgagggagt gtgcactcgg aggggtgagt cctcagccca cataggaacg gctcagcaga |
| 1501 | ggagcgtggc ccgggaacgc tggggatctg gccagtgtgc atcgaggtgc actgttgggc |
| 1561 | aaagtgtgag ctggggttac ggcagtgcag ggaaataaaa ttgtatgcac tttaaaactt |
| 1621 | aggaccttaa aacttagatg tgtgtgcgtg tgaggatgag atttgaagcc ctgaattaaa |
| 1681 | gtaaaatgca aaaaaaaaaa aaaaaaaaaa aa |
| // | |
| (SEQ ID 28)LOCUS AY196699 1518 bp mRNA linear ROD 20-MAY-2003 | |
| DEFINITION Rattus norvegicus 2′-5′ oligoadenylate synthetase | |
| 1E mRNA, complete cds. | |
| ACCESSION AY196699 | |
| translation=“MPWKLANTPIWRLDRFIQDHLLPDTTFLTEVRADIDFICAFLTE | |
| RCFQGATHPVRVSRIVMGGCYDEYSMLKGRSEATLVVFLNNLTCFEDQLKRREEFIEE | |
| IWKHLCQLQQEKQFKLNFEFQTSEQANSRSLSIKLSSPQLQQEVEFDVQPAYDVLYAW | |
| RYNKYLDPRIYNKIYASLIRESTNLDKEGEFSVCFTELQQHFLKHPGHKMWNLIRLVK | |
| YWYQLWKEKLGDLLPPQYALELLTVHAWELGIENTCELYIARGFRSVLELIIKYRCLL | |
| IFWTLCYDFNHNEVSEYLNKQLQKDRPVILDPADPTRNVAGSNLQAWHLLAEEAGAWV | |
| QSSFFRNSDMSLVHSWKVPPEKQTCVIL” | |
| BASE COUNT 375a 404c 382g 357t | |
| ORIGIN | |
| 1 | tcaggatgcc gtggaaactt gccaataccc ccatctggag gctggacaga ttcatacagg |
| 61 | atcacctcct tcctgacacc accttcctta ctgaggtcag agcagacatt gactttatat |
| 121 | gtgctttcct gactgagaga tgcttccaag gtgccaccca ccctgtgagg gtctctagga |
| 181 | ttgtgatggg cggctgctac gatgaatact ccatgctcaa gggaaggtca gaggccaccc |
| 241 | tggtggtgtt ccttaacaat ctcacctgct ttgaggatca gttaaagcga cgagaagagt |
| 301 | tcattgagga aatttggaaa cacctgtgcc agttgcagca agagaaacag tttaaactga |
| 361 | attttgagtt ccagacctca gagcaggcca actccaggtc tctgagcatc aagctgagct |
| 421 | cgccccagct ccagcaggag gtggagtttg atgtgcagcc agcctatgat gtcctgtatg |
| 481 | cttggagata caacaagtac cttgatccca gaatctacaa caaaatctac gccagcctca |
| 541 | tccgtgagag caccaacctg gataaggagg gcgagttctc cgtctgcttc acagagctcc |
| 601 | aacaacactt cctgaagcat cctggacata agatgtggaa tctcatccgc ctggtcaagt |
| 661 | actggtatca actgtggaag gagaagctgg gggatttgct gcccccacag tacgccctgg |
| 721 | agctgctcac ggtccatgcc tgggaactgg ggattgaaaa cacctgtgag ctatacatag |
| 781 | cccggggctt ccgaagtgtc ttagaactga tcatcaaata tcggtgtctc ctaatcttct |
| 841 | ggacattgtg ttatgacttt aatcacaatg aggtctctga gtacctgaac aaacaactcc |
| 901 | aaaaagacag gcccgtgatc ctggaccctg ctgacccaac aaggaacgtg gctggttcaa |
| 961 | acctccaggc ctggcacctg ctggcagaag aggctggggc ctgggtgcag tcgtctttct |
| 1021 | ttaggaacag cgatatgtcc cttgttcaca gttggaaagt gccgccagag aaacaaacat |
| 1081 | gtgtcatcct gtgagcccag caggacctgc ccaagaggct tcggagtcag ggccatgtgc |
| 1141 | tcttctgctg caagaccttg ccctggagag aacagttcgg tgcaccaggc tccagtaagg |
| 1201 | ggcatccaac ctgtgatcag actccaggct tctgatcccg tcctgccctg cccagggaca |
| 1261 | gccttcctca cagcctgctt cacccgcctt atcccccaca gtgttctctg ggagtcggac |
| 1321 | tctgaaggaa agggacaaac ttaaccttga cttccacctt ttcacctgtt gtgaagctca |
| 1381 | gtccaacaat gtctgattat ctatgataag ccacagcaag ccattgcggg ggggtgtgca |
| 1441 | ctctgaggga tggggcctta gaagtacgtg tatgactttt catgctctac tagaaacatt |
| 1501 | gagtacttgt tgcaagct |
| // | |
| (SEQ ID 29)LOCUS AY196700 1619 bp mRNA linear ROD 20-MAY-2003 | |
| DEFINITION Rattus norvegicus 2′-5′ oligoadenylate synthetase | |
| 1F mRNA, complete cds. | |
| ACCESSION AY196700 | |
| /translation=“MVNLSSTPACELDRFIKDHLPADTSFHAELRADIDFICAFLKER | |
| CFQGAAHPVRVSRVVMGEHTMLKGRSEANLVVFLNDLPSFEDQLNLQGEFIEEIRKRL | |
| CQLQQEKTLQVKLEVQSSEQPSSKSLSFTLSSPQLQQEVEFDVQPAYDVLFALRNNHK | |
| PDPQIYTKTYAYLISVCTTLKKEGEFSTCFMELRQNFLKHREPKLKSLIRLVKHWYQL | |
| CKEKLGKPLPPQYALELLTVYAWESGSRDCEFNTAQGFRTVLELVTKYQWLRIYWTLY | |
| YDFRNKKVSDYLHKQLKKTRPVILDPADPTRNVAGSNPLCWRLLAKEAASWLQCPCFR | |
| TCDMSLVHSWEVLTKVEFPQECVLL” | |
| BASE COUNT 409a 462c 388g 359t 1 others | |
| ORIGIN | |
| 1 | ggctgcagtc agcaaacact cccggcctta aaatggtgaa cctcagcagc accccagcct |
| 61 | gtgagctgga caggttcata aaggatcacc tccctgctga caccagcttc catgctgagc |
| 121 | tcagagcaga cattgacttc atatgtgctt tcctgaagga gagatgcttc caaggtgccg |
| 181 | ctcaccctgt gagggtctcc agggttgtga tgggcgaaca caccatgctt aagggtaggt |
| 241 | cagaagccaa cctagtggtg ttccttaacg atctccccag ctttgaggat cagttgaatc |
| 301 | tacagggaga gttcattgag gaaattcgga aacgactgtg tcagctgcag caagagaaaa |
| 361 | cgttacaagt gaagcttgag gtccagagct cagagcagcc cagctccaag tctctgagct |
| 421 | tcacgctgag ctcgccccag ctccagcagg aggtggagtt tgatgtgcag ccagcctatg |
| 481 | atgtcctgtt tgccctaaga aacaaccaca agcccgaccc ccaaatctac accaaaacct |
| 541 | acgcctacct catcagtgtg tgcaccactc tgaagaagga gggcgagttc tccacctgct |
| 601 | tcatggagct ccggcaaaac ttcctgaagc atcgggaacc caagctgaag agcctcatcc |
| 661 | gtctggtcaa gcactggtat caactgtgta aggagaagct ggggaagccg ctgcccccac |
| 721 | agtacgccct ggagctgctc acggtctatg cctgggaaag tgggagtaga gactgtgaat |
| 781 | tcaacacagc ccagggcttc cgaaccgtct tggaactggt caccaagtat cagtggcttc |
| 841 | gaatctactg gacattgtat tatgacttta gaaacaagaa ggtctctgat tacctacaca |
| 901 | aacagctcaa aaaaaccagg cctgtgatcc tggacccggc tgacccgaca agaaatgtgg |
| 961 | ctggttcaaa ccccctctgc tggcgactgt tggcaaaaga agctgctagc tggctgcagt |
| 1021 | gcccatgctt taggacctgt gatatgtccc tcgtgcactc ctgggaagtg ctgacaaagg |
| 1081 | tcgagtttcc acaggaatgt gtccttctat gagcaccaaa gcacctgccc aggatgctca |
| 1141 | agagtcaggg gcatgacctc tgctgcaggc ccttgaccta gagaggagag gaagctcccc |
| 1201 | aaaaccccaa caagggaggg tccaacctgt gatcagactc tggtcttctg acccctgcct |
| 1261 | tcctactcct gcatcctgtc ccatcacaga cagccctcct cacagcctgc ttcatctgcc |
| 1321 | ttgtcctcca acagtgctct cttgggagac aagagattca gaaggggagg caggaactcg |
| 1381 | agcttgactt ccacctgtcc acctgttggg agttctgtcc aatgtgtgac caacgacaat |
| 1441 | aaaccatagc aagccatgag gatatgtgaa cattctgaag gatgtgtgtt ctcctccctc |
| 1501 | tccctctccc cttccctacc taccctgtcc tgcctcacat acgttttctt acctctacnt |
| 1561 | gcatatgaca tgatagtata tttaagtgat cccaaaagtt ccaccagaga actactaaa |
| // | |
| (SEQ ID 30)LOCUS AY221507 1469 bp mRNA linear ROD 02-JUN-2003 | |
| DEFINITION Rattus norvegicus 2′-5′ oligoadenylate synthetase | |
| 1G (Oas1g) mRNA, complete cds. | |
| ACCESSION AY221507 | |
| translation=“MEQELRSTPSWKLDKFIEVYLLPNTSFRDDVKSAINVLCDFLKE | |
| RCFRDTVHPVRVSKVVKGGSSGKGTTLKGKSDADLVVFLNNFTSFEDQLNRRGEFIKE | |
| IKKQLYEVQREKHFRVKFEVQSSWWPNPRALSFKLSAPHLQQEVEFDVLPAYDVLGHV | |
| SLYSNPDPKIYTILISECISLGKEGEFSTCFTELQRNFLKQRPTKLKSLIRLVKHWYQ | |
| LCKEKLGKPLPPQYALELLTVYAWERGNGITEFNTAQGFRTILELVTKYQQLRIYWTK | |
| YYDFQHPDVSKYLHRQLRKSRPVILDPADPTGNVAGGNQEGWRRLASEARLWLQYPCF | |
| MNRGGSPVSSWEVPTEVPVPSEQVDEAWSCILL” | |
| polyA_signal 1428 . . . 1433 | |
| /gene=“Oas1g” | |
| BASE COUNT 385a 388c 392g 304t | |
| ORIGIN | |
| 1 | gttccaagtt caaccaggct gcgagacaca ggacctgcag gctgcagagg caaaagctcc |
| 61 | ggaggtcatg gagcaggaac tcaggagcac cccgtcctgg aagctggaca agttcataga |
| 121 | ggtttaccrc cttccaaaca ccagcttccg tgatgatgtc aaatcagcta tcaatgtcct |
| 181 | gtgtgatttc ctgaaggaga gatgcttccg agatactgtc cacccagtga gggtctccaa |
| 241 | ggtggtgaag ggcggctcct caggcaaagg caccacactc aagggcaagt cagacgctga |
| 301 | cctggtggtg ttccttaaca atttcaccag ctttgaggat cagttaaaca gacggggaga |
| 361 | gttcatcaag gaaattaaga aacagctgta tgaggttcag cgtgaaaaac attttagagt |
| 421 | gaagtttgag gtccagagtt catggtggcc caacccccgg gctctgagct tcaagctgag |
| 481 | tgcaccacac ctccaacagg aggtggagtt tgatgtgctt ccagcctatg atgtcctagg |
| 541 | tcatgttagc ctctacagca atcctgatcc caagatctac accatcctca tctccgaatg |
| 601 | tatctccctg gggaaggagg gcgagttctc tacctgcttc acggagctcc agaggaactt |
| 661 | cctgaagcag cgcccaacca agctgaagag tctcatccgc ctggtcaagc actggtacca |
| 721 | actgtgtaag gagaagctgg ggaagccgct gcccccacag tacgccctgg agctgctcac |
| 781 | ggtctatgcc tgggaacgtg gaaatggaat tactgagttc aacacagctc agggcttccg |
| 841 | gacaatcttg gaactggtca caaagtacca gcagcttcga atctactgga caaagtatta |
| 901 | tgactttcaa cacccagatg tctccaaata cctacacaga cagctcagaa aatccaggcc |
| 961 | tgtgatcctg gaccctgctg acccaacagg gaacgtggct ggtgggaacc aagaaggctg |
| 1021 | gcggcggttg gcctcagagg cgaggctgtg gctgcagtac ccatgtttta tgaacagggg |
| 1081 | tggttcccca gtgagctcct gggaagtgcc gacagaggtt cctgtgcctt cagagcaggt |
| 1141 | ggatgaggcc tggtcatgca tcctgctgtg aacccagcag caccagccca ggaggctccg |
| 1201 | gagtcagggg cacgtgctgc tttgctgcag gaccttgaca cagtgaggga gggccccact |
| 1261 | cgggatcaca gtccatgctt ttgatgcccg cccgccatgt ttgaatactg tccaatcaca |
| 1321 | gacagccttc ctcaacagat tcagaagggg cggaaagaac tcaagcttga cttccatctg |
| 1381 | accgtccacc tgttgggagg ttctgtccaa ccatgtctgt caacaacaat aaagtacagc |
| 1441 | aggtgccaaa aaaaaaaaaa aaaaaaaaa |
| // | |
| (SEQ ID 31)LOCUS AY196701 1701 bp mRNA linear ROD 20-MAY-2003 | |
| DEFINITION Rattus norvegicus 2′-5′ oligoadenylate synthetase | |
| 1H mRNA, complete cds. | |
| ACCESSION AY196701 | |
| translation=“MEKDLKSTPAWKLDKFIQNHLLADTTFVTEAKADIEFLCDFLTE | |
| RCFQDASHPVRVSRIVMGGCYDEYSMLKGRSEATLVVFFNNLTRFEDQLKRREELIEE | |
| IWKHLCQLQHEKQFKLKFEVQSSEQDNYSSLSIKLSSPQLQQEVEFDVQPAYDVLYEL | |
| KEKLELDCEFYNKIYARLIRECITLRKEGEFSVCFMELQQKFLWNRPEDLRNLLVLVK | |
| HWYQLCKEKLGDSLPPQYALELLTVHAWENEIPAKYGAQTARGFQSVLELIIKYTCLR | |
| VYWTFYYDILHQDVSSYLHKQLRKERPVILDPADPTRNVAGLNIDGWCELAKEAEAWL | |
| KYPCFRHIDETFVGSWEVPPEKQGGVFL” | |
| BASE COUNT 440a 421c 460g 380t | |
| ORIGIN | |
| 1 | gcagtcagca aacattcctg gcctcaggat ggagaaggat ctcaagagta ctccagcttg |
| 61 | gaagctggac aagttcatac agaatcacct ccttgctgac accaccttcg ttactgaggc |
| 121 | caaggcagac atagagttcc tatgtgattt cctgactgag agatgcttcc aagatgcctc |
| 181 | ccaccctgtg agggtctcta ggattgtgat gggcggctgc tatgatgaat actccatgct |
| 241 | caagggaagg tcagaggcca ccctggtggt gttctttaac aatctcacca gatttgagga |
| 301 | tcagttaaag cgacgggaag agctcattga agaaatttgg aaacacctgt gccagttgca |
| 361 | gcacgagaaa cagtttaaac tgaagtttga ggtccagagc tcagagcagg acaactacag |
| 421 | ctctctgagc atcaagctga gctcgcccca gctccagcag gaggtggagt ttgatgtgca |
| 481 | gccagcctat gatgtcctgt atgaactgaa agaaaaactg gagcttgact gtgagttcta |
| 541 | caacaaaatc tatgcccggc tcatccgtga gtgcatcacc ctgaggaagg agggcgagtt |
| 601 | ctccgtctgc ttcatggagc tccagcaaaa gttcctgtgg aatcgtccag aagacctgag |
| 661 | gaatctcctc gtactggtca agcactggta tcaactgtgt aaggagaagc tgggggattc |
| 721 | gctgccccca cagtacgccc tggagctgct cacggtccat gcctgggaaa acgaaattcc |
| 781 | agccaaatat ggagcacaga cagctcgggg tttccagagt gtcttagaac tgatcattaa |
| 841 | gtacacctgt cttcgagtct actggacatt ttattacgac attctacacc aggatgtctc |
| 901 | cagctacctg cacaaacagc tccgtaaaga aaggcctgtg atcctggacc ctgctgaccc |
| 961 | aacaaggaac gtggctggtt tgaacataga tggctggtgt gagctggcaa aagaggcaga |
| 1021 | agcctggctg aagtacccgt gctttaggca catcgatgag acctttgtgg gctcctggga |
| 1081 | agtgccgcca gagaaacaag gaggtgtctt cctgtgagca ccacagccct tgcccaggag |
| 1141 | gctctggagt caggggcatt cactcctctg ctgcaagacc ttgtcctgca gagaacagtt |
| 1201 | tggtacacca ggctccagtg aggggcatcc aacctgtgat cagactctag gcttctgatc |
| 1261 | ccgtcctgcc catgggacag ccttcctcac agcctgcttc acctgcctta tcctccacag |
| 1321 | tgctctctgg gagtcagact ccgaaggaaa gagaagagct caaacttggc ttctgtctgt |
| 1381 | ccacctgtgg ggaggctctg tccacaaaca tgtctgatca tgaacaattg accacagcaa |
| 1441 | tccctgaggg agtgtgcact cggaggggtg agtcctcagc ccacatagga acggctcagc |
| 1501 | agaggagcgt ggcccggaac gctggggatc tggccagtgt gcatcaaggt gcactgttgg |
| 1561 | gcaaagtgtg agctggggtt acggcagtgc agggaaataa gattatgtac actttaaaac |
| 1621 | ttaggacctt aaaacgtatg tgtgtgcatg tgaggatgag atttgaagcc ctgaattaaa |
| 1681 | gtaaaatgca aaggactaaa c |
| // | |
| (SEQ ID 32)LOCUS AY227756 1980 bp mRNA linear ROD 06-JUN-2003 | |
| DEFINITION Rattus norvegicus 2′-5′ oligoadenylate synthetase-like | |
| 1 protein (Oas11) mRNA, complete cds. | |
| ACCESSION AY227756 | |
| /translation=“MAVAQELYSFPASKLDSFVAQWLQPTREWKEEVLETVQTVEQFL | |
| RQENFRGERGPAQDVRVLKVLKVGCFGNGTVLRSTTDVELVVFLSCFHSFQEEAKHHQ | |
| AVLRLIQKRMSYCRDLLDLGLSNLSVIEEVPSSLIFTIQTRETWEPITVTIVPAFRAL | |
| GPSCPNSAEVYVNLIKANGYPGNFSPSFSELQRSFVKHRPTKLKSLLRLVKHWYQQYV | |
| RDKCPRANLPPLYALELLTVYAWEAGTQEDSNFRLDEGLATVMELLQDHELLCIYWTK | |
| YYTLQHPVIERFVRRQLKGERPIILDPADPTHNVAQGYRWDIVAQRASQCLKQDCCYD | |
| DRDAPVPSWTVKRAPYIQVTVQQWGHPDLILWVNPYEPIKKLKEKIRLSRGYSGLQRL | |
| SFQEPGGQRQLIRSQCSLAYYGIFCDTQICLLDTISPEIQVFVKNPDGGSHAYALHPL | |
| DFVLSLKQQIEDRQGLQSQEQQLEFQGRVLEDWFDFKSYGIQDSITIILSRKREGKAP | |
| SAPS” | |
| polyA_signal 1953 . . . 1958 | |
| /gene=“Oasl1” | |
| BASE COUNT 477a 586c 510g 407t | |
| ORIGIN | |
| 1 | gcgcagacat ggcagtagcc caggagcttt acagcttccc agcctccaag ctggactcct |
| 61 | ttgtggctca gtggctgcag ccaaccagag aatggaagga ggaggtcctg gagacggtgc |
| 121 | agacagtgga gcagttcctg aggcaggaga acttccgtgg agaacgtggc ccggcccagg |
| 181 | atgtacgagt gctcaaggta ctcaaggtag gctgctttgg gaatggcaca gtactcagga |
| 241 | gtaccacaga cgtggagctg gtggtgttcc tgagctgttt ccacagcttc caggaagagg |
| 301 | ccaaacacca ccaggctgtt ctgagactga tacagaaaag gatgtcttac tgccgggacc |
| 361 | tgctggatct cgggctcagt aacctgagtg tgattgaaga agtgcccagt agtctcatct |
| 421 | tcactatcca gaccagggag acctgggagc ccatcactgt caccatcgtg cccgccttca |
| 481 | gagccctggg accttcctgt cccaactccg ccgaggtcta tgtgaatctg atcaaggcta |
| 541 | acggctaccc cggaaatttc tctccttcct tcagcgagct acagaggagc ttcgtgaagc |
| 601 | ataggccgac taagctgaag agcctcctac ggttggtcaa acactggtac cagcagtacg |
| 661 | tgagagacaa gtgcccccgg gccaacctgc cccccctcta tgccctggag ctgctaactg |
| 721 | tctacgcgtg ggaagcaggt acgcaggagg attcgaactt caggctggat gaaggtctcg |
| 781 | ccactgtcat ggagttgctc caggatcatg aactcctgtg catctactgg accaagtact |
| 841 | acaccctgca acacccagtc attgagcgct tcgtcaggag acagctcaaa ggagaaaggc |
| 901 | ccattatcct ggacccagca gaccccaccc acaacgtggc gcaaggctac aggtgggata |
| 961 | tagttgctca gcgcgccagc cagtgtctga aacaggactg ttgctatgac gacagggacg |
| 1021 | cccccgtccc cagctggact gtgaagagag caccatatat ccaggtgacc gtgcagcagt |
| 1081 | ggggtcaccc ggatttaatc ctctgggtga acccttatga acccataaag aagctgaaag |
| 1141 | agaaaatccg actgagccgg ggctactccg gcctgcagcg cctgtccttt caggagcccg |
| 1201 | gcggccaacg gcagctcatc agaagccaat gctcgcttgc ctactacgga atcttctgcg |
| 1261 | acactcagat ctgcctgctg gacaccatct cccccgagat ccaggtcttt gtgaaaaacc |
| 1321 | cggatggtgg aagccacgcc tacgccatcc acccacttga cttcgtcctg agcctgaagc |
| 1381 | agcagatcga agataggcag ggccttcaaa gccaggagca gcagctggag ttccagggcc |
| 1441 | gcgtcctgga agactggttt gactttaaat cctatggcat ccaagatagt atcacgatca |
| 1501 | tcctatccag gaagagggag gggaaagccc catctgcgcc cagctagctt cctctgcctc |
| 1561 | ttttgctatc tcatcctaaa gtcagcctag tcacccctcc ttccggtcct cagccgggat |
| 1621 | gatcccagca gggagccaga agggaatact gccagacgct cttgtgtgac aatgaaactc |
| 1681 | tgcagtcacc tgtgaaatca caccaggacc tctacgctct caagactggg tcagtgaatg |
| 1741 | gccgtcccac aaataaacta ttctcgcttg ttcttgggaa gccaaacgat cttcctaacc |
| 1801 | aatcaaatgg accctgcttc aggttgttcc cccacacaca ccagcaacct ccatcaggtt |
| 1861 | ggatctgaag ccttcccctg tgctcccaac cacttctgcc tgcctcagcc tatccaaggc |
| 1921 | aaggtagggt atcaagttcc tttcaaatgg agaataaaca acctttgttt cctcccagat |
| // | |
| (SEQ ID 33)LOCUS AY237116 1750 bp mRNA linear ROD 12-JUN-2003 | |
| DEFINITION Rattus norvegicus 2′-5′ oligoadenylate synthetase | |
| 2-like protein (Oas12) mRNA, complete cds. | |
| ACCESSION AY237116 | |
| /translation=“MDTLPDLYGTVGDSLDYFLEHSLQPQRDWKEEGKDAWERIERFF | |
| REKCFCDELLLDQEVRVLKVVKGGSSGKGTALNHRSDQDMILFLSCFSSFKQQARDRK | |
| AVIDFIKSKLIHCRKSLAYNITVRQHKEGKRTPRSLTLEIQSRKSNDIICMDILPAYN | |
| ALGSFSRDCKPEPEIYENLIRCKGYPGDFSPSFAELQRHFVKSRPVKLKNLLRLVKFW | |
| HLKYLRHKYRRAVLPSKYALELLTIYAWEMGTDSSDNFNLDEGFVAVMELLRDYQDIC | |
| IYWTKYYDFQNEVVRNFLKEQLKGDRPIILDPADPTNNLGRRGKWELVAKEATYCLLQ | |
| LCCVTADRWNVQVSIAHYLRGARDVQVTVKQTGREEWILLTNPHSPIRKLKAKIKKRM | |
| NLCGELRISFQEPGGERQPLSGRKTLSDYGIFSKVNIRVMETFPPEIQVFVRYPGGQN | |
| KPFAIDPDATILSLWEKIEEDGGPCTEDWVLLFEGEELDDDDNLAELQIKDCDTIQLS | |
| RVS” | |
| BASE COUNT 485a 415c 468g 382t | |
| ORIGIN | |
| 1 | ttctcccagc attgctgagc agaagcacag aagattcaat ccagaggaca ggctgatcca |
| 61 | gcagagatgg atacattacc cgacctgtat gggaccgttg gggacagtct agactacttc |
| 121 | ctggaacaca gccttcagcc ccaaagggac tggaaagagg aaggaaagga tgcctgggag |
| 181 | agaattgaga ggttctttcg ggaaaagtgc ttctgtgatg agctgctcct ggaccaagaa |
| 241 | gtcagggtgc ttaaagtggt aaagggaggc tcctcaggaa aggggacggc gctgaaccac |
| 301 | agatctgacc aggacatgat tctgttcctg agctgttttt ccagtttcaa acagcaggca |
| 361 | agagaccgga aggccgtcat agacttcatt aagagcaagc tgattcattg taggaaaagc |
| 421 | ctggcctaca acatcactgt ccgtcaacac aaagaaggca aaaggacccc tcgctccctg |
| 481 | accctagaga tccagtccag gaagagcaat gacataattt gcatggatat tctccctgct |
| 541 | tacaatgcct tgggatcctt ttccagagac tgcaaaccag aacctgaaat ctacgagaat |
| 601 | ctgataaggt gtaagggcta ccctggcgac ttctcgccaa gtttcgcaga gttacagaga |
| 661 | cattttgtga aaagtcgccc ggttaaactg aaaaacctcc tacggttggt gaagttctgg |
| 721 | cacctgaagt acctgaggca taaatataga agagcagtgt tgccctcaaa atatgcactg |
| 781 | gagttgctga ccatctatgc ttgggagatg ggtacagaca gcagtgataa tttcaatctg |
| 841 | gatgaagggt ttgtagccgt catggagctc ctcagggact accaagacat ctgcatctac |
| 901 | tggaccaagt actatgattt ccaaaatgag gtcgtcagga acttcctgaa ggaacagctt |
| 961 | aagggcgacc ggcctatcat tctagaccca gctgacccca ccaacaacct aggaagaaga |
| 1021 | ggaaaatggg aactggtggc caaagaagct acttactgcc tgctacagtt gtgttgtgtg |
| 1081 | actgcagacc gctggaatgt tcaggtatct atcgcgcact acctccgagg agcgagggac |
| 1141 | gttcaggtga cagtgaaaca aacaggaagg gaagagtgga ttctcttgac aaacccccac |
| 1201 | agccccatca ggaagttgaa ggcaaagatc aagaagagaa tgaacctctg tggggagctg |
| 1261 | cgtatctcct tccaggagcc gggaggggag aggcagccgc tgagtggccg gaaaaccctg |
| 1321 | tcggattatg gaattttctc taaggtgaac atccgggtga tggagacctt tcctcctgag |
| 1381 | atccaggtct ttgtgaggta tcccggtggc cagaacaagc cttttgccat cgaccctgat |
| 1441 | gctaccatct taagcctgtg ggagaaaatt gaggaagatg gaggcccatg tacggaggat |
| 1501 | tgggtactac tgtttgaggg tgaggagctg gacgatgatg acaaccttgc agagcttcag |
| 1561 | atcaaggact gtgacaccat ccagctcagc agggtctcct agtctgcctc cccacatcac |
| 1621 | ccctttactc tgacatattc ctcctgtagc ttaaacatca tcatatccgg ttggggattt |
| 1681 | ggctcagtgg tagagcgctt gcctagcaac cgcaaggccc tgggttcggt ccccagctcc |
| 1741 | gaaaaaaaaa |
| // | |
| (SEQ ID 34)LOCUS AY230746 2311 bp mRNA linear ROD 05-JUN-2003 | |
| DEFINITION Rattus norvegicus 2′-5′ oligoadenylate synthetase | |
| 2 (Oas2) mRNA, complete cds. | |
| ACCESSION AY230746 | |
| /translation=“MGNWMPGWSSSGSLGVPPMPVQKLEKSVQVNLEPDEKCLSQTEV | |
| SSVPSQKLEEYIQANLKPDEESLKQIDQAVDAISDLLCSEVMIDVLKVVKGGSYGRKT | |
| VLRDCSDGTLVLFTGLFKQFQDQKKYQDKLLDLIEQRLKSHEKYKKSVKRKLSLLEVQ | |
| VSIPGQSILLQLLPTFNPLCISENPSAQVYQNLKRSMDQVKASPGEFSDCFTTLQQRF | |
| FEKYPGRLKDLILLVKHWYKQLQDKWIIPSPPPLLYALELLTVYAWEQGCQTKDFDIT | |
| QGIRTVLQLISQPTNLCVYWLDNYNFEDETVRNNLLHQLNSPRPVILDPTDPTNNVGK | |
| DDRFWQLLAEEAQEWLNSLRLNKPHKPCWDVLPMPFFITPSHCLDKFIKDFLQPDKVF | |
| LNQIKRAVDIICSFLKETCFQNSDIKVLKIIKGGSTAKGTALQQRSDADIIVFLSSLD | |
| SYDSLETERSQYVQEIRKQLEACQKAFNLGVKFDISKWMAPRVLSFTLESKSLKQSVE | |
| FDVLPAYDALGQLRSDYTSRLKAYKKLIELYASQDSLKGGEFSVCFTELQRDFIETRP | |
| TKLKGLIRLIKHWYKQCERKMKPKASLPPKYALELLTVYAWEHGSGTDGFDTAEGFRT | |
| VLDLVIRYRQLCVFWTVNYNFEEDHMRKFLLTQIQKKRPVILDPADPTGDVGGGDRWC | |
| WHLLAKEAKEWLSSSCFQVEPKSPVQPWKVPVVQTPGSCGAQIYPVVGGVY” | |
| BASE COUNT 649a 573c 566g 523t | |
| ORIGIN | |
| 1 | agtgctctgc ctctgtcatt cacaagccca gctagcaact atgggaaact ggatgcctgg |
| 61 | ctggtcatcc agtgggtctt tgggggtgcc ccccatgcca gtgcagaagc tagaaaagtc |
| 121 | tgtccaggta aaccttgaac cagatgaaaa atgtctgagt cagaccgagg tgtcctctgt |
| 181 | accatcccag aagctagaag aatatatcca ggcaaacctc aaacccgatg aagaatctct |
| 241 | gaagcagata gaccaggctg tggatgccat ctctgacctg ctgtgcagtg aggtgatgat |
| 301 | cgatgtgctg aaagtggtta agggtggctc ctatggtcgg aaaacagtcc taagagactg |
| 361 | ctccgatggt acacttgttc tcttcaccgg tctcttcaaa cagttccaag accagaagaa |
| 421 | ataccaagat aagctccttg acttgattga acaacggctg aaaagccatg agaaatacaa |
| 481 | gaagtcagta aaacgtaaac ttagcctcct tgaagtacaa gtgtctatac cagggcagag |
| 541 | tatactcttg cagttgcttc caaccttcaa tcctctgtgc atcagtgaga atcccagcgc |
| 601 | ccaggtctat cagaatctca aaagatccat ggatcaagta aaagcatcac ctggggaatt |
| 661 | ctcagactgc ttcaccacac tgcagcagag gtttttcgag aaatatcccg ggagactgaa |
| 721 | ggatttgatt ctattggtca agcactggta taaacagttg caggataagt ggataatacc |
| 781 | ctcacctccg ccattgctat atgcactgga gctgcttact gtgtatgcct gggaacaggg |
| 841 | ctgccagact aaagactttg acatcacgca aggtatcagg actgtgctgc aactcatcag |
| 901 | tcagccgaca aacctgtgtg tctactggtt agacaattac aactttgagg atgagacagt |
| 961 | ccggaacaac cttctgcacc agctcaactc cccaagaccg gtcatcttgg atccaaccga |
| 1021 | cccaaccaac aatgtgggca aagatgacag gttctggcag ctactggcag aagaggctca |
| 1081 | ggagtggctg aactctctca gactgaataa gccacacaaa ccatgttggg atgttctgcc |
| 1141 | catgccattt ttcatcaccc caagccactg cctggacaag ttcatcaaag acttcctcca |
| 1201 | acctgacaag gtcttcctaa accaaatcaa aagagctgtt gacattatct gttcattctt |
| 1261 | aaaagagacc tgcttccaga attctgacat caaagtcctg aagatcatca agggaggatc |
| 1321 | cactgccaaa ggcacagctc tgcagcagag atcagatgct gacatcatag tgttcctcag |
| 1381 | ctcactggat agttatgact ccctagaaac tgaacgctcc cagtacgtcc aggagatccg |
| 1441 | aaagcagtta gaagcctgcc agaaggcctt taatttaggg gtgaagtttg atatttccaa |
| 1501 | atggatggcc cccagggtgc tgagttttac cctggaatcc aagagtctca agcaaagtgt |
| 1561 | ggagttcgat gtccttcccg cctatgatgc actaggtcag ctgcggtctg actacacctc |
| 1621 | caggctcaaa gcctacaaga agctcattga gctgtatgcc tcacaggaca gcctcaaagg |
| 1681 | aggggagttt tcagtctgtt ttacagagct acagagagac ttcattgaaa ccaggcccac |
| 1741 | caaactcaag ggtctgatcc gcctgatcaa gcactggtac aaacagtgtg aaaggaagat |
| 1801 | gaagccaaaa gcatctttgc ctccaaagta cgcactggag ctgctcaccg tgtatgcgtg |
| 1861 | ggagcatggc agtggcacag atggcttcga cactgctgaa ggcttccgga ccgtcctgga |
| 1921 | cttggtcata agataccggc agctctgcgt cttctggaca gtcaattaca actttgagga |
| 1981 | ggatcacatg aggaagttcc tactgaccca gatccagaaa aagaggcctg tgatcctgga |
| 2041 | tccagcagat cccacaggtg atgtgggagg aggtgaccgc tggtgttggc atcttctagc |
| 2101 | caaagaagca aaggagtggc tgtcctcctc ctgtttccaa gtggagccaa aaagccccgt |
| 2161 | gcagccgtgg aaagtaccag tagtacagac tccaggaagc tgtggagctc agatctaccc |
| 2221 | tgtggtgggt ggggtgtact aagagagtgc attcagctct ggagggaaaa tgctggaaga |
| 2281 | agcttctaga gacatctggc aaagactctg c |
| // | |
| (SEQ ID 35)LOCUS AY250706 4015 bp mRNA linear ROD 06-MAR-2003 | |
| DEFINITION Rattus norvegicus 2′-5′ oligoadenylate synthetase | |
| 3 mRNA, complete cds. | |
| ACCESSION AY250706 | |
| /translation=“MDLYHTPAGALDKLVAHSLHPAPEFTAAVRRALGSLDNVLRKNG | |
| AGGLQRPRVIRIIKGGAHARGTALRGGTDVELVIFLDCLRSFGDQKTCHTEILGAIQA | |
| LLESWGCNPGPGLTFEFSGPKASGILQFRLASVDQENWIDVSLVPAFDALGQLHSEVK | |
| PTPNVYSSLLSSHCQAGEHSACFTELRKNFVNIRPVKLKNLILLVKHWYRQVQTQVVR | |
| ATLPPSYALELLTIFAWEQGCRKDAFSLAQGLRTVLALIQRNKHLCIFWTENYGFEDP | |
| AVGEFLRRQLKRPRPVILDPADPTWDLGNGTAWCWDVLAKEAEYSFNQQCFKEASGAL | |
| VQPWEGPGLPCAGILDLGHPIQQGAKHALEDNNGHLAVQPMKESLQPSNPARGLPETA | |
| TKISAMPDPTVTETHKSLKKSVHPKTVSETVVNPSSHVWITQSTASSNTPPGHSSMST | |
| AGSQMGPDLSQIPSKELDSFIQDHLRPSSQFQQQVRQAIDTILCCLREKCVDKVLRVS | |
| KGGSFGRGTDLRGKCDVELVIFYKTLGDFKGQNSHQTEILCDMQAQLQRWCQNPAPGL | |
| SLQFIEQKSNALHLQLVPTNLSNRVDLSVLPAFDAVGPLKSGAKPLPETYSSLLSSGC | |
| QAGEHAACFAELRRNFLNTRPAKLRSLMLLVKHWYRQVAAREEGGETAGAALPPAYAL | |
| ELLTVFAWEQGCGEQKFSMAEGLRTVLRLVQQHQSLCIYWTVNYSVQDPAIRAHLLRQ | |
| LRKARPLILDPADPTWNMDQGNWKLLAQEAAALESQVCLQSRDGNLVPPWDVMPALLH | |
| QTPAQNLDKFICEFLQPDRHFLTQVKRAVDTICSFLKENCFRNSTIKVLKVVKGGSSA | |
| KGTALQGRSDADLVVFLSCFRQFSEQGSHRAEIIAEIQAQLEACQQKQRFDVKFEISK | |
| RKNPRVLSFTLTSKTLLGQSVDFDVLPAFDALGQLKSGSRPDPRVYTDLIQSYSNAGE | |
| FSTCFTELQRDFISSRPTKLKSLIRLVKHWYQQCNKTVKGKGSLPPQHGLELLTVYAW | |
| ERGSQNPQFNMAEGFRTVLELIGQYRQLCVYWTINYGAEDETIGDFLKMQLQKPRPVI | |
| LDPADPTGNLGHNARWDLLAKEAAAYTSALCCMDKDGNPIKPWPVKAAV” | |
| BASE COUNT 887a 1086c 1119g 923t | |
| ORIGIN | |
| 1 | gaaactctcc tgagggcatg ggtcaacatg gacctgtacc acacgccagc cggagctctg |
| 61 | gacaagctgg tggcccacag cctgcaccca gcccctgagt tcacagcggc tgtgcggcgt |
| 121 | gctctggggt ccctggacaa cgtcctaagg aagaacggag ccggagggtt acagagacca |
| 181 | agggtgataa ggatcatcaa gggaggagcc catgctcgag gcacagctct cagaggtggc |
| 241 | actgatgtcg aactcgtcat cttcctcgac tgcctccgga gctttggcga ccagaagacc |
| 301 | tgtcacacag agatcctggg cgccatacaa gcattgctgg agtcctgggg gtgcaaccct |
| 361 | gggcctggcc tgacttttga gttttctggg ccaaaggcgt ctggcatctt acagtttcgc |
| 421 | ctggcatcgg tggaccaaga aaactggata gatgttagcc tggtgcctgc cttcgatgcc |
| 481 | ctaggacagc tccactctga agtcaagcca acacccaatg tgtactcctc cctcctcagc |
| 541 | agccactgcc aggctgggga acactcagcc tgcttcacag agctccggaa gaactttgtg |
| 601 | aatatccgcc cagtcaaact taagaactta atcctgctgg tcaaacactg gtaccgccag |
| 661 | gtgcagacac aggttgtgag agccacactg ccccctagct acgcgctgga gctgctcacc |
| 721 | atctttgcct gggagcaggg ctgtaggaag gatgccttca gcctggccca agggctccgg |
| 781 | actgtcctgg ccttgatcca acgcaacaag catctctgca ttttctggac ggaaaactac |
| 841 | ggcttcgaag accctgcagt tggggagttc ttgcgaaggc agcttaagag acccaggccc |
| 901 | gtgatcctgg atccagctga cccaacatgg gacttgggca atgggacagc ctggtgctgg |
| 961 | gatgtgcttg ccaaggaggc tgaatacagc tttaaccagc agtgcttcaa ggaggcctca |
| 1021 | ggagcccttg tgcaaccttg ggaggggccg ggccttccat gtgctgggat cttggatttg |
| 1081 | ggtcacccta tccaacaagg agctaagcat gcccttgaag acaacaatgg ccaccttgct |
| 1141 | gttcagccaa tgaaagagag cctacaacca tcaaatccag cccgaggact cccagaaaca |
| 1201 | gccaccaaga tctccgctat gccagaccca acggtcactg aaacccacaa gagcctcaaa |
| 1261 | aaatcagtgc acccaaagac tgtcagtgaa acagtggtga atccctcaag tcatgtttgg |
| 1321 | atcacccaga gtacagcatc ctcaaacacg cctccgggcc actctagtat gtccaccgct |
| 1381 | gggtcacaga tgggcccaga tctgtcacag atccccagca aggagctgga ctccttcatc |
| 1441 | caggaccace tcaggccgag ttcccagttc cagcagcagg tgaggcaggc catcgacacc |
| 1501 | atcctgtgct gcctccggga gaagtgtgta gacaaagtct tgagagtcag caagggtggc |
| 1561 | tcttttggcc gtggcacaga cctcaggggc aaatgtgatg tggagcttgt catcttttat |
| 1621 | aaaactctcg gggacttcaa gggccagaac tcacaccaga cagagatcct gtgtgacatg |
| 1681 | caggcccagc tgcagcgctg gtgtcagaac ccagcacctg gactgagcct ccagtttatt |
| 1741 | gaacagaagt ccaatgctct gcatcttcag ctggtgccca ccaacctcag caaccgggta |
| 1801 | gacctcagtg tgctgcccgc ttttgatgca gtggggccgc tgaagtccgg cgccaaacct |
| 1861 | ctacccgaga cgtactcctc cctcctcagc agcggctgcc aggctgggga gcatgcagcc |
| 1921 | tgcttcgcag agctccgaag gaacttcata aacactcgcc ctgccaaact taggagcctg |
| 1981 | atgctactgg tcaaacactg gtaccgccag gttgccgctc gatttgaagg aggagagaca |
| 2041 | gcaggtgctg ctctgccccc agcctatgcc ctggagctcc tgacagtctt tgcctgggaa |
| 2101 | caaggctgcg gagaacaaaa gtttagcatg gctgaaggcc tgcggactgt cctgaggctg |
| 2161 | gtccagcagc accagtcact ctgtatctac tggacagtca actacagtgt gcaggaccca |
| 2221 | gccatcagag cacaccttct ccgccagctt cggaaagcca ggcctctaat cctagaccct |
| 2281 | gcagatccca cctggaacat ggaccagggc aactggaagt tgctggctca ggaggcggct |
| 2341 | gccctggagt cacaagtctg ccttcagagt agggatggga atctggtgcc accatgggat |
| 2401 | gttatgccag ccctccttca ccagaccccg gctcagaacc tggacaagtt catctgtgaa |
| 2461 | ttcctccagc ctgaccgcca tttcctgact caggtgaaga gagcagtgga caccatatgt |
| 2521 | tccttcctga aagaaaactg cttccggaat tctaccatca aggtgctcaa ggtggtcaag |
| 2581 | ggtgggtctt ctgccaaagg cactgctcta caagggcgct cggatgctga cctggtggtg |
| 2641 | ttcctcagct gcttccgcca gttctctgaa caaggcagcc atcgggcaga gatcatcgcg |
| 2701 | gagatccagg ctcagctgga ggcgtgtcag cagaagcaga ggttcgatgt caagtttgag |
| 2761 | atctccaaga ggaagaaccc ccgagttctc agcttcacgc tgacatccaa gacgctgcta |
| 2821 | ggccagagcg tggacttcga tgtgctgcca gccttcgatg ctcttggtca gctgaagtcc |
| 2881 | ggctctcggc cagatccccg ggtctacacg gacctcatcc agagctacag taatgcagga |
| 2941 | gagttctcta cctgcttcac ggagctgcag cgggacttca ttagctcccg tcccaccaaa |
| 3001 | ctcaagagtc tgatccgtct ggtgaaacac tggtaccaac agtgtaacaa gacagtcaag |
| 3061 | gggaagggtt ccttgccccc ccagcacggg ctggagctcc tgactgtgta cgcctgggag |
| 3121 | cgaggcagcc agaaccccca gttcaacatg gcggagggct tccgcacagt tctggagctg |
| 3181 | attggccagt accgtcagct gtgcgtctat tggaccatca actacggtgc agaagacgag |
| 3241 | accatcggag acttcctgaa gatgcagctt cagaagccca ggcctgtcat cctggaccca |
| 3301 | gctgacccga caggcaacct aggccacaat gcccgctggg acctgcttgc caaggaggct |
| 3361 | gcagcataca catctgccct gtgctgcatg gacaaggacg gcaaccccat caagccatgg |
| 3421 | ccagtaaagg ccgctgtgtg aagtccagaa agatcaaaaa gtgacaccag ccctcagcaa |
| 3481 | gggatactca gaatatctgg ccagatgtgt gtgtgtgtgt gtgtgtgtgt gtgttgtgta |
| 3541 | ttgtgtttat ctatatatgt ttctgtgctg tgtctgtgtg tctctgtagg tggtgtgtgt |
| 3601 | ctgtgtaccc acaggtgtct acatgtgtct gtatatatgt aagagtgtgt gtatgtatgt |
| 3661 | atgttcacgt gtctctgtgt gtgtgcatct gtgtgtttgt atgtagtgtg tctataggtg |
| 3721 | tatccttatg agtgtgtgtg tttgtggtgg agtgtgtgtg tgtctgtgta tctgtgggtg |
| 3781 | tctatatgtg tctgagtgtg tgtgattatt tgtgtgttta tgtgtctgtg tatttgtgtg |
| 3841 | tagtgagttt ataggtgcat ctgtgtgtct atatgtatat gtgtgtctat gtgtgtctat |
| 3901 | ggtgtgtgtc cgtgtatctg tgggtgtcta tgtgtgtctg tatgtacgtg tgtgtgtgtg |
| 3961 | tgacttgctt tgtccaaatg attgtatgta tgtatgtgtg ctcatgtggc tctgt |
| // | |
| (SEQ ID 36)LOCUS AY243505 1682 bp mRNA linear MAM 13-JUN-2003 | |
| DEFINITION Bos taurus 2′-5′-oligoadenylate synthetase | |
| 1 (OAS1) mRNA, complete cds. | |
| ACCESSION AY243505 | |
| VERSION AY243505 | |
| /translation=“MELRNTPAGSLDKFIEDHLLPDEEFRMQVKEAIDIICTFLKERC | |
| FRCAPHRVRVSKVVKGGSSGKGTTLRGRSDADLVVFLTNLTSFQEQLERRGEFIEEIR | |
| RQLEACQREETFEVKFEVQKRQWENPRALSFVLRSPKLNQAVEFDVLPAFDALGQLTK | |
| GYRPDSRVYVRLIQECKYLKREGEFSPCFTELQRDFLKNRPTKLKSLIRLVKHWYQLC | |
| KEQLGKPLPPQYALELLTVYAWEQGCNKTGFITAQGFQTVLKLVLKYQKLCIYWEKNY | |
| NSENPIIEEYLTKQLAKPRPVILDPADPTGNVAGKDANSWERLAQAALVWLDYPCFKK | |
| WDGSPVGSWDVSPQEHSDLMFQAYDFRQHCRPSPGIQFHGGASPQVEENWTCTIL” | |
| BASE COUNT 436a 431c 431g 384t | |
| ORIGIN | |
| 1 | gcacgagcac agattcaggc agcagctctg ccgcctctgg ctctccagtc cccagcaccg |
| 61 | tgatggagct cagaaatacc ccggccgggt ctctagacaa gttcatcgaa gaccacctcc |
| 121 | tgccagacga ggagttccgc atgcaggtca aagaagccat cgacatcatc tgcactttcc |
| 181 | tgaaggagag gtgtttccga tgtgcccctc acagagttcg ggtgtccaaa gttgtgaagg |
| 241 | gcggctcctc aggcaaaggc acgaccctca ggggacgatc agatgctgac ctcgtcgtct |
| 301 | tcctcaccaa tctcacaagt tttcaggaac agcttgagcg ccgaggagaa ttcatcgaag |
| 361 | aaatcaggag acagctggaa gcctgtcaaa gagaggaaac atttgaagtg aagtttgagg |
| 421 | tccagaaacg gcaatgggag aatccccgcg ctctcagctt tgtgctgagg tcccccaagc |
| 481 | tcaaccaggc ggtggagttc gatgtcctgc ccgcctttga tgccctaggt cagttgacca |
| 541 | aaggttacag acctgactct agagtctatg tccggctcat ccaagagtgc aagtacctga |
| 601 | agagagaagg cgagttctcc ccctgcttca cggagctgca gcgagacttc ctgaagaatc |
| 661 | gtccaaccaa gctgaagagc ctcatccgcc tggtgaagca ctggtaccaa ctgtgtaagg |
| 721 | agcagcttgg aaagccattg cccccacaat atgctctgga gcttctgacg gtctatgcct |
| 781 | gggagcaagg atgcaataaa acaggattca tcacagctca gggatttcag actgtcttga |
| 841 | aattagtcct aaagtatcag aagctttgca tctactggga aaagaactat aactctgaaa |
| 901 | accctattat tgaagaatat ctgacgaagc aacttgcaaa acccaggcct gtgattctgg |
| 961 | acccggcgga ccctacagga aatgttgctg gtaaagacgc aaatagctgg gagcggcttg |
| 1021 | cacaagcggc tttggtctgg ctggattacc cgtgctttaa gaaatgggat gggtctcccg |
| 1081 | tgggctcctg ggatgtgtcg ccccaagaac acagtgacct gatgttccag gcctatgatt |
| 1141 | ttagacagca ctgtagaccc tctccaggaa tccagttcca cggaggagcc tctccccagg |
| 1201 | tggaagagaa ctggacatgt accatcctct gaatgccaga gtatcttgga ggcaaggtct |
| 1261 | ccagagccgt ctgggccagc cctcttcact tctagggata gggggcttgg atccaaagac |
| 1321 | agctgtgaat tgatgtcaga cctgggacca gaatccaggt ctcctgaccc ccagccttcc |
| 1381 | tgctattctg tgctgtcttt tctttcatag acaatgctcc ccattggagc ctgacaatag |
| 1441 | cctctctgag ccaccaggag agactcaggc aaaagagtgg aatcccagcc ttgactttct |
| 1501 | tctgtgaacc tgaggggaaa ggtgatggtc caatttattg tcaataataa caaaaatagt |
| 1561 | agcaaatgcc atttgttggg tgttaattag cttcaaggta cagcgccaag aagtatacct |
| 1621 | gcatattatg tgtgtgtgtg catattcatt gattcaacta aagatattaa ttgggcacct |
| 1681 | gc |
| // | |
| (SEQ ID 37)LOCUS AY321355 1601 bp mRNA linear MAM 11-JUN-2003 | |
| DEFINITION Equus caballus 2′-5′ oligoadenylate synthetase | |
| 1A (Oas1a) mRNA, complete cds. | |
| ACCESSION AY321355 | |
| /translation=“MELQKTPARNLDKFIEDYLLPDTRFRRQVREAIDIICSFLKERC | |
| FRGAVPPVRVSKVVKGGSSGKGTTLRGRSDADLVVFLDYLTSFREQFERRAEFIKEIR | |
| RQLEACQREKRFDVEFEVQGQQWARPRALSFVLTSPQLNEGVEFDVLPAFDVLGQVTT | |
| SYRPDPDIYVLLIKECQSLGKEGEFSPCFTELQRAFLRQRPTKLKSLIRLVKHWYQKC | |
| KDKLGKPLPAQYALELLTVYAWEQGSRQTEFNTAQGFRTVLELVLKYQQLCIYWTKYY | |
| NFDDPVIGQYLKRQLKKPRPVILDPADPTGNVGGGDPRSWPRLAQEARAWLSYPCFKN | |
| WDGSPVGSWDVGPEEDSEDDTLTWAERAYYQCDHGRRPEFPQTGSTPQRASAPDAEEN | |
| WTCTIL” | |
| polyA_signal 1576 . . . 1581 | |
| /gene=“Oas1a” | |
| BASE COUNT 372a 452c 450g 327t | |
| ORIGIN | |
| 1 | agtttctggg agccagtccc acgagcacca gctcctctgt ccccacccgg gcgtcacgat |
| 61 | ggagctccaa aagaccccag ccaggaatct ggacaagttc attgaagact atctcttgcc |
| 121 | agacacacgg ttccgcaggc aggtccgaga agccatcgac attatctgca gtttcctgaa |
| 181 | ggagaggtgt ttccgaggtg ccgttccccc tgtgcgggtg tccaaagtgg tgaagggtgg |
| 241 | ctcctcaggc aaaggcacga ccctcagagg ccgatccgat gctgacctcg tcgtcttcct |
| 301 | tgactacctc acgagtttcc gggagcagtt tgagcgccga gcagagttca tcaaggagat |
| 361 | tcggaggcag ctggaagcct gtcaaagaga gaagaggttt gacgtggagt tcgaggtcca |
| 421 | ggggcagcag tgggcgaggc cccgcgcgct cagcttcgtg ctcacgtcgc cccagctcaa |
| 481 | tgagggggtg gaatttgatg tcctgcctgc ctttgatgtc ctaggtcagg tgactacatc |
| 541 | gtacagacct gaccctgaca tatatgtcct actcatcaaa gaatgccagt ccctggggaa |
| 601 | ggagggagag ttctccccct gcttcacgga gctgcagcga gccttcctga ggcagcggcc |
| 661 | aaccaagctc aagagcctca tccgcctggt caagcactgg taccaaaagt gcaaggataa |
| 721 | acttgggaaa ccactgccag cacagtacgc cctggagctc ctgacagtct atgcttggga |
| 781 | acagggaagc agacaaacag aattcaacac agctcaggga tttcggactg tcttggaact |
| 841 | agtcctgaag taccagcagc tttgcatcta ctggacaaag tattacaact ttgatgaccc |
| 901 | tgttattgga caatacctga aaaggcagct caagaaaccc aggcctgtga ttctggaccc |
| 961 | ggctgacccc acaggaaacg ttggtggtgg agacccacgc agctggcctc ggctggcaca |
| 1021 | ggaggcgaga gcctggctga gttacccgtg ctttaagaat tgggacgggt ctccagtggg |
| 1081 | ctcctgggac gtggggcctg aagaagacag cgaggacgac actttgacct gggctgagcg |
| 1141 | cgcatattac cagtgcgacc acggacggcg ccctgaattc ccgcagaccg gcagcacgcc |
| 1201 | ccagagggca tccgctcccg acgcggaaga gaactggacc tgcaccatcc tctgatcgcc |
| 1261 | ggcgcagcgt ggaggagagg actccagagt cgggggggcc agccccctca tttcctgggc |
| 1321 | gggatcttat cggctgtgac ttggcatcag tcctaggacc agaatccggg tctcctgacc |
| 1381 | cctcttcctg ctgttcccct ctttctcgcc ctccctaggt agcgctgccc gcagcctcat |
| 1441 | cccgccacag cctgttttct gacaatattc tctgagaggc aacagttgag gtttagacaa |
| 1501 | aagagtggaa actcagcctt gactttcttc tgtgtgcctg gtgagaaggt tatggtccaa |
| 1561 | tttattatca ataacaataa aaataatagc agataaaaaa a |

This work was supported by Public Health Service Grants: GM54896 from the National Institute of General Medical Sciences and AI45135 from the Institute of Allergy and Infection Diseases, National Institutes of Health.
References
1. Webster, L. T. (1923) J. Exp. Med. 37, 231-244.
2. Sabin, A. B. (1952) Ann. N.Y Acad. Sci. 54, 936-944.
3. Green, M. C. (1981) in Genetic variants and strains of the laboratory mice, ed. Green, M. C. (Gustav Fischer Verlag, Stuttgart), p. 84.
4. Shellam, G. R., Sangster, M. Y., & Urosevic, N. (1998) Rev. Sci. Tech. 17, 231-48.
5. Goodman, G. T., & Koprowski H. (1962) Proc. Natl. Acad. Sci. USA 48,160-165.
6. Darnell, M. B., Koprowski, H., & Lagerspetz, K. (1974) J. Inf. Dis. 129, 240-247.
6. 7. Webster, L. T., & Johnson, M. S. (1941) J. Exp. Med. 74, 489-494.
8. Vainio, T. (1963) Ann. Med. Exp. Biol. Fenn. 41, 1-24.
9. Darnell, M. B., & Koprowski, H. (1974) J. Inf. Dis. 129, 248-256.
10. Brinton, M. A. (1997) in Viral Pathogenesis, ed Nathanson, N. (Lippincott-Raven, Philadelphia), pp. 303-328.
11. Sangster, M. Y., Heliams, D. B., MacKenzie, J. S., & Shellam, G. R. (1993) J. Virol. 67, 340-347.
12. Sangster, M. Y., Mackenzie, J. S., & Shellam, G. R. (1998) Arch. Virol. 143, 697-715.
13. Groschel, D., & Koprowski, H. (1965) Arch. Ges. Virusforsch. 17, 379-391.
14. Jerrells, T. R., & Osterman, J. V. (1981) Infect. Imnun. 31, 1014-1022.
15. Shellam, G. R., Urosevic, N., Sangster, M. Y., Mansfield, J. P., & Mackenzie, J. S., & (1993) Mouse. Genome 91, 572-574.
16. Urosevic, N., Mansfield, J. P., Mackenzie, J. S., & Shellam, G. R. (1997) Arbovirus Res. Aust. 7, 296-299.
17. Lovett, M. (1994) in Current Protocols in Human Genetics, eds. Seidman, J. & Seidman, C., (Wiley Interscience), pp. 6.3.1-6.3.15.
18. Sambrook, J., Fritsch, E. F., & Maniatis, T. (1989) in Molecular Cloning. a Laboratory Manual, Second edition (Cold Spring Harbor Laboratory Press, NY), pp 7.37-7.52.
19. Hovnanian, A., Rebouillat, D., Mattei, M. G., Levy, E. R., Marie, I., Monaco, A. P., & Hovanessian A G. (1998) Genomics 52, 267-277.
20. Thompson, J. D., Gibson, T. J., Plewniak, F., Jeanmougin, F., & Higgins, D. G. (1997) Nucleic Acids Res. 25, 4876-4882.
21. Saitou, N., & Nei, M. (1987) Mol. Biol. Evol. 4, 406-425.
22. Felsenstein, J. (1985) Evolution 39, 783-791.
23. Bateman, A., Birney, E., Cerruti, L., Durbin, R., Etwiller, L., Eddy, S. R., Griffiths-Jones, S., Howe, K. L., Marshall, M., & Sonnhammer, E. L. (2002) Nucleic Acids Res. 30, 276-280.
24. Corpet, F., Servant, F., Gouzy, J., & Kahn D. (2000) Nucleic Acids Res. 28, 267-269.
25. Dougherty, J. P., Samanta, H., Farrell, P. J., & Lengyel, P. (1980) J. Biol. Chem. 255, 3813-3816.
26. St.-Laurent, G, Yoshie, O., Floyd-Smith, G., Samanta, H., Sehgal, P. B., & Lengyel, P. (1983) Cell 33, 95-102.
27. Ichii, Y., Fukunaga, R., Shiojiri, S., & Sokawa, Y. (1986) Nucleic Acids Res. 14, 10117.
28. Rutherford, M. N., Kumar, A., Nissim, A., Chebath, J., & Williams, B. R. (1991) Nucleic Acids Res. 19, 1917-1924.
29. Ghosh, S. K., Kusari, J., Bandyopadhyay, S. K., Samanta, H., Kumar, R., & Sen, G. C. (1991) J. Biol. Chem. 266, 15293-15299.
30. Ghosh, A., Desai, S. Y., Sarkar, S. N., Ramaraj, P., Ghosh, S. K., Bandyopadhyay, S., & Sen, G. C. (1997) J. Biol. Chem. 272, 15452-15458.
31. Saraste, M., Sibbald, P. R., & Wittinghofer, A. (1990)Trends Biochem. Sci. 15, 430-434.
32. Yamamoto, Y., Sono, D., & Sokawa, Y. (2000) J. Interferon Cytokine Res. 20, 337-344.
33. Ghosh, A., Sarkar, S. N., Guo, W., Bandyopadhyay, S., & Sen, G. C. (1997) J. Biol. Chem. 272, 33220-33226.
34. Benech, P., Merlin, G., Revel, M., & Chebath, J. (1985) Nucleic Acids Res. 13, 1267-1281.
35. Marie, I., & Hovanessian, A. G. (1992) J. Biol. Chem. 267, 9933-9939.
36. Rebouillat, D., Hovnanian, A., Marie, I., & Hovanessian, A. G. (1999) J. Biol. Chem. 274, 1557-1565.
36. Hartmann, R., Olsen, H. S., Widder, S., Jorgensen, R., & Justesen, J. (1998) Nucleic Acids Res. 26, 4121-4128.
37. Rebouillat, D., Marie, I., & Hovanessian, A. G. (1998) Eur. J. Biochem. 257, 319-330.
38. Tiefenthaler, M., Marksteiner, R., Neyer, S., Koch, F., Hofer, S., Schuler, G., Nussenzweig, M., Schneider, R., & Heufler, C. (1999) J. Immunol. 163, 760-765.
39. Hovanessian, A. G., Brown, R. E., & Kerr, I. M. (1977) Nature 268, 537-540.
40. Clemens, M. J., & Williams, B. R. (1978) Cell 13, 565-572.
41. Brinton, M. A., Arnheiter, H., & Haller, O. (1982) Infect. Immun. 36, 284-288.
42. Urosevic, N., Silvia, O. J., Sangster, M. Y., Mansfield, J. P., Hodgetts. S. I., & Shellam, G. R. (1999) J. Gen. Virol. 80, 897-906.
43. Poidinger, M., Hall, R. A., & Mackenzie, J. S. (1996) Virology 218, 417-421.
44. Urosevic, N., van Maanen, M., Mansfield, J. P., Mackenzie, J. S., & Shellam G. R. (1997) J. Gen. Virol. 78, 23-29.

### EXAMPLES

### Example 1

Cell Cultures and Virus Stocks

Cell lines were previously established by SV-40 transformation of embryofibroblasts obtained from congenic C3H.PRI-Flv^{r} and C3H/He (9). Baby hamster kidney (BHK-21/WI2) cells (referred to hereafter as BHK cells) were used for virus plaque assays (8).

The cDNAs corresponding to the ORFs of the C3H.PRI-Flv^{r} alleles of the Oas1b (AF328926) and Na⁺/Ca²⁺-exchanger (AF261233) genes were cloned separately into the pEF6/V5-His-TOPO mammalian expression vector (Invitrogen) and these plasmids were transfected into susceptible C3H/He cells using LipofectAMINE 2000 (Life Technologies). Stable integrants were selected using blasticidin S and cells from individual foci were isolated with cloning rings and propagated. Expression of the recombinant proteins, which contained C-terminal 6×His and V5 tags, was analyzed by Western blotting of cell lysates using V5 antibody (Invitrogen). Stable cell lines expressed a low level (1×), an intermediate level (8×) or a high level (20×) of Oas1b protein were obtained.

A stock of West Nile virus (WNV), strain Eg101, was prepared as a 10% (w/v) newborn mouse brain homogenate (titer=2×10⁸ PFU/ml). A stock of Sindbis virus, strain SAAR 339, was prepared as a 10% (w/v) newborn mouse brain homogenate (titer=7×10⁹ PFU/ml). For virus growth experiments, confluent monolayers in T25 flasks were infected with WNV or Sindbis virus at a multiplicity of infection (MOI) of 0.5. Both viruses were titered on BHK cells by plaque assay.

### Example 2

Construction of the BAC Contig

A single genomic clone, 171N24, which contained the D5Mit159 marker and was about 40 kb in length, was isolated from a mouse BAC library (Baylor College of Medicine) using a unique probe derived from D5Mit159. The terminal sequence obtained from the T7 promoter side of the 171N24 clone did not match any of the DNA sequences in the GenBank database, but the sequence located next to the SP6 promoter was part of a large interspersed repeat. Additional BAC clones were subsequently isolated from the RPCI-23 C57BL/6 mouse BAC library (Roswell Park Cancer Institute) using a probe designed from the 171N24 clone sequence adjacent to the T7 promoter. Four positive signals were detected and the clones, 244P21, 274O1, 297M4, and 359J23, were purchased and analyzed. The size of the insertion in each clone was estimated from restriction patterns observed after pulse-field gel electrophoresis. The terminal DNA sequences for each of the BAC clones were determined and used to design eight new primer pairs for PCR amplification of fragments from each end of each BAC clone. Each BAC clone DNA was then tested as a template in a PCR with each primer pair and the data obtained was used to align the clones into a single BAC contig of 300 kb.

Two sequences, 297M4T7 and 244P21SP6, which were the most distal in the initial contig were then used to re-screen the library and eight additional BAC clones were isolated. These clones were partially sequenced and aligned into the contig by screening them with other BAC clone terminal sequences. A GenBank search against the 5′ and 3′ BAC insert sequences identified of one additional BAC clone, 39M18 (AC015535).

Physical Map of the Flv Interval

To create a genomic contig, two independent mouse BAC libraries were screened and fourteen BAC clones were isolated. Alignment of these clones provided a BAC contig with an estimated length of more than 700 kb (FIG. 1). Two flanking microsatellite markers, D5Mit408 and D5Mit242, were mapped outside the contig according to the Celera mouse database. The D5Mit159 marker was located in the central part of the contig (FIG. 1).

### Example 3

Isolation of Transcription Units from the BAC Contig

Direct cDNA selection, exon trapping and searches of genes annotated in the Celera database (www.celera.com), were used to identify transcripts from the Flv interval. Direct cDNA selection was performed according to the protocol of Lovett (17) using adaptor-ligated double-stranded cDNA prepared from C3H.PRI-Flv^{r} cells. Exon trapping was performed using an Exontrap kit (MoBiTec). The cDNAs obtained after each exon trapping or cDNA selection experiment were tested by hybridization with the different BAC clone DNAs, and those that showed specific hybridization with the initial BAC clone DNA were cloned into a pCR-XL-TOPO vector (Invitrogen) and sequenced.

The length of the mRNA corresponding to a partial cDNA isolated by cDNA selection or exon trapping was estimated by Northern blotting using the method of Sambrook et al. (18). The partial cDNA sequences were extended by RACE using a Marathon cDNA amplification kit (Clontech). The expression patterns of the candidate genes were analyzed using mouse multiple tissue poly-A⁺ (Stratagene) and total RNA (Seegene) Northern blots hybridized to probes excised with endonucleases from the cDNAs and labeled with the RTS RadPrime kit (Life Technologies).

Partial and full-length cDNA sequences were used to search the Celera mouse genome database to identify additional transcripts from closely linked genes in the Flv region. Primer pairs designed from sequenced cDNAs and from gene sequences obtained from the Celera database were used to amplify cDNAs from the congenic C3H.PRI-Flv^{r} and C3H/He mouse strains. The primer sequences are listed in Table 5 (published as supporting information).

Transcript Map of the Flv Region

Direct cDNA selection and exon trapping techniques as well as searches of the GenBank and Celera mouse databases were used to identify candidate genes. Four novel transcripts, a Ca²⁺-channel (AF217002), an unknown mRNA (AF217003), an ATP-dependent helicase (AF319547), and a serine dehydratase (AF328927), were identified by direct cDNA selection. Three novel transcripts, a Na⁺/Ca²⁺-exchanger (AF261233), the Oas2 (AF418010), and the Oas3 (AF453830), were detected by exon trapping. The partial sequences obtained were extended to full-length cDNAs by 5′ and 3′ RACE techniques. Two previously identified genes, Oas1a and Oas1b (AF328926), were also cloned using the exon trapping technique (FIG. 1).

Although small (42 kDa), medium (70 kDa), and large (105 kDa) forms of 2′-5′ oligoadenylate synthetases had previously been detected in mice using various biochemical techniques (25-26), only the cDNAs of some of the mouse 42 kDa proteins had been previously cloned (27-29). Three DNA sequences of the Oas1a gene were reported in the Mouse Genome Informatics (MGI) database (www.informatics.jax.org) under the accession ID 97429. Two of these sequences, M33863 and X04958, are almost identical to each other and to the Celera transcript, mCT15312, whereas the third sequence, X58077, is similar to the Celera transcript mCT15074, which maps to a different genomic region. We designated this gene Oas1g (see below). The sequence, AF328926, cloned in this study was identical to the previously isolated partial (576 bp) Oas1b sequence, X55982 (28). Two other sequences, M63849 and M63850, deposited in MGI under the accession ID 97430 and also designated Oas1b were similar to each other, but not to X55982 and AF328926 nor to any mouse transcripts, expressed sequence tags (EST) or genomic sequences in neither the NCBI or the Celera databases. However, M63849 and M63850 showed identity with the human OAS1 sequences and it is likely that these two sequences were cloned from a mouse cDNA library contaminated with human clones.

Twelve additional genes were identified in the Flv region by searching the Celera mouse database with sequences from the nine loci detected by cDNA selection and exon trapping. The LIM homeobox 5 (Lhx5), a threonine dehydratase (BC021950), an unknown protein (AK017032), the RAS protein activator-like 1 (Rasal1), the deltex 1 (Dtx1), the Oas1c (AB067528), the rabphilin 3A (Rph3a), and the protein tyrosine phosphatase, non-receptor type 11 (Ptpn11) sequences were available in GenBank. Genomic and cDNA sequences of four novel genes annotated by the Celera database (subsequently named Oas1d, Oas1e, Oas1f, Oas1g, see Table 1) were also identified in the Flv region and their sequences were used to search the NCBI mouse EST database. The EST sequences obtained were used to generate cDNA consensus sequences, as well as to design PCR primers for the amplification of each novel gene from mRNA. One additional gene, Oas1h (AB067530), not annotated in Celera, was subsequently identified using a BLAST search of the NCBI database using the Oas1b sequence as a query. The Oas1h cDNA sequence was used to search the Celera database and this gene was mapped on the Flv interval between Oas1f and Oas1g (FIG. 1).

### Example 4

Amplification and Sequencing of the Oas1b Exons from Genomic DNA

Genomic DNAs for eight mouse strains, 129/SvJ, BALB/c, BRVR, C57BL/6, CASA/Rk, CAST/Ei, CBA/J, and MOLD/Rk, were purchased from Jackson laboratory and used for PCR amplification of Oas1b exons. The primers (Table 6, supporting information) used for amplification and direct sequencing were designed from the genomic DNA sequence of the Oas1b gene (AC015535).

### Example 5

Phylogenetic and Domain Architecture Analysis of Oas Sequences

Protein sequences of mouse and human 2′-5′ oligoadenylate synthetases 2 and 3 were divided into fragments corresponding to a single functional unit (19). Multiple sequence alignments were constructed using CLUSTAL X (20). Phylogenetic trees were built from multiple alignments using the neighbor-joining method (21). The bootstrapping procedure (22) was applied to the PHYLIP format tree output. Known and putative domains in Oas sequences were revealed by searches against Pfam (23) and ProDom (24) databases.

TABLE 4
Mouse 2′-5′ oligoadenylate synthetase genes and their orthologs.

| | GenBank | | |
|---|---|---|---|
| | accession # | | |
| Mouse | (cDNA clone | Celera | Orthologous |
| gene | name) | transcript | sequences |
| Oas1a | X04958, | mCT15312 | rat - Z18877, |
| | M33863 (L3), | | pig - AJ225090, |
| | BC013715 | | marmot - AF082498 |
| | | | OAS1*-NM_002534, |
| | | | NM_016816 |
| Oas1b | X55982 (L1), | mCT15306 | rat - AF068268 |
| | AF328926, | | OAS1*-NM_002534, |
| | AB067529, | | AF418004-AF418009 |
| | BC012877, | | |
| | NM_016816 | | |
| Oas1c | AB067528, | mCT15073 | OAS1*-NM_002334, |
| | AF459815 | | NM_016816 |
| Oas1d | AB067532, | mCT15317 | OAS1*-NM_002334, |
| | AY055829 | | NM_016816 |
| Oas1e | AB067531, | mCT15075 | OAS1*-NM_002334, |
| | AY055830, | | NM_016816 |
| | AY055831 | | |
| Oas1f | AF481733 | mCT15304 | OAS1*-NM_002334, |
| | | | NM_016816 |
| Oas1g | X58077 (L2), | mCT15074 | OAS1*-NM_002334, |
| | BC018470 | | NM_016816 |
| Oas1h | AB067530 | none | OAS1*-NM_002334, |
| | | | NM_016816 |
| Oas2 | AB067535, | mCT15077 | OAS2*-NM_002535, |
| | AF418010 | | NM_016817 |
| Oas3 | AB067534, | mCT15081 | OAS3*-NM_006187 |
| | AF453830 | | |
| Oasl1 | AB067533, | mCT18390 | OASL*-NM_003733 |
| | AY057107 | | |
| Oasl2 | AF068835 | mCT118383 | unknown |
| | | mCT18449 | |

TABLE 5

Primers used for PCR amplification
of murine genes from the Flv interval.

| | | |
|---|---|---|
| Gene | Forward Primer (5′ to 3′) | Reverse Primer (5′ to 3′) |

| | | |
|---|---|---|
| AF217002 | AAGGCTGGCGCAGCTGCCGCT | AACCCAGCTAGGTGACAGTCTGG |
| AF217003 | ACCTGCCCTCGCGATGGCGGC | ATCCTCCTGCCTCATCTTCTGAGT |
| AF261233 | ACTAGAGCAGCCAGCCCGTGAGCA | CTGGCTTACAGAGTGAGTTCCAGG |
| AF319547 | TGTTATGTCCCTCAGGTCCTGCTC | AAACTCAATGCTGGGTCAGAGGCA |
| AK017032 | TTACTGGAGGCTGTGAAATCTAGG | TAGGGCGTGGTGGAAGCTGAACA |
| BC021950 | CAGACACAATCTACTCCTCTCGCT | CATTGTCAAGTGTATCCCACCCCA |
| Dtx1 | CTAAGGGATTGAGATCATGTCCCC | GTGCTTGACTCAAGTCCTGGGAAA |
| Lhx5 | GAAGTCTTGGTTGATCCGTAACGG | TGACTTTGGTCCCGAGAAATTGCG |
| Oas1a | AGACCCAGGAAGCTCCAGACTTAG | GACAGAACCTTCCAACAGGTGGAC |
| Oas1c | AAACACTCCTGGCCTCAGGATGG | CAGCCCCAGTGCATTGTGATTTAA |
| Oas1d | GTCAGCAAACACTCCTGGCCTC | GCGTTTTGCTTTAATTTAGTGCTTC |
| Oas1e | TCAGGATGGCGAGGGAACTCTTCA | AACTGCCATAAACCCGGCCTGCAT |
| Oas1f | GTCAGCAAACACTTCCTGGCCATA | CAGGTGGAAGTCAAGCTTGAGTTC |
| Oas1g | AGACCCAGGAAGCTCCAGACTTAG | AGTTCCACGACAGCGTGTGTCACA |
| Oas1h | GGCTGCAGTCAGCAAACATTCCTG | CGGTCCTCTCTAGGTCAAAGTCTT |
| Oas2 | GACCAGCTAGCAACGATGGGAAAC | ACTGACCCAGGATCTTCTGTCCCA |
| Oas3 | GAAACTCTACTGAGAGTACCGGTC | GGGGAGGAAAGGGTTTATTCAGCT |
| Ptpn11 | AAGACGGGAGGAACATGACATCGC | AGCAGTCTCTCCTTAGCTGAGGAA |
| Rasal1 | TGAACCGGCTGACAGCGTGCTTG | AAGAGCCTGTGTCCGGCTTCGAG |
| Rph3a | AACCTTCCATGTGGAGTAGTCTGG | GGACCTGAAGATGCTTAAGGTCAG |
| Sds | TTCCTGATTCTGTCTCACGTGGCT | TTGATGACCCACCAGGTGTCCAGG |

TABLE 6

Primers used for PCR amplification
of the Oas1b exons from genomic DNA.

| | | |
|---|---|---|
| Exon | Forward Primer (5′ to 3′) | Reverse Primer (5′ to 3′) |

| | | |
|---|---|---|
| First | AATCCTAGACCTGCAAGTCCAGAG | GGTTGCAGCCTGGCTTCTGAAAGT |
| Second | TGGTGATGGACTCCAGTTCAGCAT | ACTAGAAGGGAGGATGAAGGCATG |
| Third | GGCTGAGCTGTTCCACTGAATGAA | CTCTGTACAGATGAAGCGCACAGT |
| Fourth | TGGCTATAGCACAAGAAGGCATAG | TGACATGAAGAGTGTTGGACGACC |
| Fifth | GGTCAGTTGACAGCAGCTCGTTTC | AATAAGGAGAGCCAATGGCCTCAA |
| Sixth | CTATTACAGACGGAGGTTGCAG | TGCTACTGTGAACAGACACCATGACC |

1. A method for genotyping flavivirus resistance, comprising,
a) restricting a nucleic acid sample of amplified DNA obtained from a subject with at least one endonuclease wherein the endonuclease restricts the DNA at a site so that the DNA fragments obtained differentiate between a flavivirus resistance allele and a flavivirus susceptibility allele; and
b) identifying the restricted DNA fragments.

2. The method of claim 1, wherein the nucleic acid sample is obtained from a human.

3. The method of claim 1, wherein the nucleic acid sample is derived from an animal other than humans.

4. The method of claim 1, wherein the at least one endonuclease is Alu1.

5. The method of claim 21, wherein the identifying is by gel electrophoresis.

6. The method of claim 1, wherein the identifying is by differential binding of the fragments to a microarray of known sequences.

7. The method of claim 1, wherein the amplified products comprise nucleic acids encoding SEQ ID No. 39.

8. The method of claim 1, wherein the amplified products comprise nucleic acids comprising SEQ ID No. 40.

9. The method of claim 6, wherein the microarray comprises sequences complementary to SEQ.ID No. 39.

10. The method of claim 6, wherein the microarray comprises sequences complementary to SEQ ID No. 40.

11. A composition, comprising an isolated nucleic acid construct of a human OAS 1 gene sequence comprising SEQ ID No. 39 or SEQ. ID. No. 40.

12. The composition of claim 11, wherein the composition comprises a cell comprising the isolated nucleic acid construct comprising SEQ ID No. 39.

13. The composition of claim 11, wherein the composition comprises a cell comprising the isolated nucleic acid construct comprising SEQ ID No. 40.

34. The composition of claim 11, wherein the nucleic acid construct is capable of expressing a protein.

35. The composition of claim 11, wherein the nucleic acid construct is SEQ ID No. 39.

16. The composition of claim 11, wherein the nucleic acid construct is SEQ. ID. No. 40.

17. A method of testing agents for antiviral activity, comprising
a) applying an agent to a cell line expressing a human OAS 1 gene sequence of SEQ ID No. 39 or SEQ. ID No. 40; and
b) measuring a change in the cells.

18. The method of claim 17, wherein the gene sequence is SEQ ID No. 39.

19. The method of claim 37, wherein the gene sequence is SEQ ID No. 40.


SEQUENCE LISTING (133 sequences)

SEQ ID NO: 1 - DNA, 2742 bp, Mus musculus
  CDS (280)..(2733)
aaggctggcgcagctgccgctgtggcagcggtgaggcggcggtggcggctgctgaggctccgcgctggggatattggcggcggcaactgcgggctgagctacgctgtgcagacccagtgcacagtgcgggatcccgggacggcgcgtaccttagaagatgcctctgatggaacaggctctgggaagcttccccggccccgtggctttgaacaggagctcaagccggaggcagtttaaagccctggccgttgtatcctgaggaccgcaggtcaggagaagatggctgtaagtttagatgacgatgtgccgctcatcctgaccttggacgaggctgagagtgctccgctgcctccttcgaacagcctgggccaagagcagctgcccagcaaaaatgggggcagccacagcatccacaactcccaggtccccagtctggtctccggagcggacagccccccctccagtcccaccggacacaactgggagatgaattatcaagaggcggcaatctacctccaggaaggtcagaacaacgacaagttcttcacccaccccaaggatgccagagcgctggcggcctacctcttcgtccacaaccacttcttctacatgatggagctgctcacggccctgctcctgctgctgctgtcgctgtgcgagtcccccgctgtccccgtgctcaagctgcacacttacgtccacgccacgctggaactctttgccctcatggtggtggtatttgaactctgcatgaaattgcggtggctgggcttccacacgttcgtccggcacaaacgtaccatggtcaagacgtccgtcctcgtggtgcagttcatcgaggccattgtggtgctggttcggcagacgtcccacgtgcgggtgacccgggcactacgctgcattttcctggtggactgtcgctactgtggcggtgtacggcgcaacctgcggcagatcttccagtctctcccacccttcatggacatcctcctgttgctgctcttctttatgatcatctttgccatcctcggtttctacttattctccacaaatccttccgacccctacttcagcaccctggagaacagcatcgtcaacctgttcgttctcctgaccacagccaactttccagatgtcatgatgccctcctactcccggaacccctggtcctgcgtcttcttcattgtatacctctccattgagctgtacttcatcatgaacctgctcctggccgtggtgttcgacaccttcaacgacattgaaaagcacaagttcaagtctttgctgctgcacaaacggaccgccatccagcatgcctacggcctgcttgccagccaacggaggccggctggcatctcctacaggcagttcgaaggcttaatgcgcttctacaagccccggatgagtgcaagggaacgcttcctgactttcaaggccttgaaccagagcaacacgcctctgctcagcctgaaggacttctatgatatttacgaagtcgctgctctgcagtggaaggcaaagagaaacagacagcattggtttgatgagctcccccggacagccttcctcatcttcaaagggattaacatccttgtgaattccaaggccttccagtatttcatgtacttggtggtggctgtcaacggtgtctggatcctggtggagacattcatgttgaaaggtgggaatttcacctcaaagcatgtgccctggagttacctcgtgtttcttaccatctatggagttgaactgttcatgaaggtggctggcctgggccctgtggagtacctgtcctctggatggaacctgttcgatttctcggtcacggcattcgccttcctgggactgctcgcactgacgctcaacatggaacccttctatttcattgtggtcctgcgtccccttcagctgctgaggttatttaaactgaagaaacgctaccgcaacgtgttggacaccatgtttgagctgctgccgcggatggccagccttggcctcacgctgctcaccttctactattccttcgccatcgtgggcatggagttcttcaacgggcggctgacccccaactgctgcaacaccagcacagtggccgacgcctaccggttcatcaaccacactgtgggcaataagaccaaggtagaggaaggctactactatctcaacaactttgacaacatcctcaacagcttcgtgaccttgtttgagctcaccgttgtcaacaattggtacatcatcatggaaggcgtcacctcgcagacgtcccactggagccgcctgtacttcatgaccttttacatagtgaccatggtggtgatgaccattatcgtggccttcatcctggaggccttcgtcttccgcatgaactacagccgcaagagccaggactcggaagtggacagtggcatcgtcatcgagaaggaaatgtccaaggaggagcttatggccgtcctggagctttatcgtgaggagcgaggcacctcctctgacgtgacccggctgctggacaccctctctcagatggagaaataccagcaaaattccatggtgtttctgggacggcgatcgagaaccaaaagtgacctgagtctgaagatgtaccaggaggagatccaggagtggtacgaggagcatgcccgggaacaggagcagcagaagctcaggggcagcgtgcccggccctgcagcccagcagccccctggcagtcgccagcgctcccagactgtcacctagctgggtttc

SEQ ID NO: 2 - protein, 817 aa, Mus musculus
MAVSLDDDVPLILTLDEAESAPLPPSNSLGQEQLPSKNGGSHSIHNSQVPSLVSGADSPPSSPTGHNWEMNYQEAAIYLQEGQNNDKFFTHPKDARALAAYLFVHNHFFYMMELLTALLLLLLSLCESPAVPVLKLHTYVHATLELFALMVVVFELCMKLRWLGFHTFVRHKRTMVKTSVLVVQFIEAIVVLVRQTSHVRVTRALRCIFLVDCRYCGGVRRNLRQIFQSLPPFMDILLLLLFFMIIFAILGFYLFSTNPSDPYFSTLENSIVNLFVLLTTANFPDVMMPSYSRNPWSCVFFIVYLSIELYFIMNLLLAVVFDTFNDIEKHKFKSLLLHKRTAIQHAYGLLASQRRPAGISYRQFEGLMRFYKPRMSARERFLTFKALNQSNTPLLSLKDFYDIYEVAALQWKAKRNRQHWFDELPRTAFLIFKGINILVNSKAFQYFMYLVVAVNGVWILVETFMLKGGNFTSKHVPWSYLVFLTIYGVELFMKVAGLGPVEYLSSGWNLFDFSVTAFAFLGLLALTLNMEPFYFIVVLRPLQLLRLFKLKKRYRNVLDTMFELLPRMASLGLTLLTFYYSFAIVGMEFFNGRLTPNCCNTSTVADAYRFINHTVGNKTKVEEGYYYLNNFDNILNSFVTLFELTVVNNWYIIMEGVTSQTSHWSRLYFMTFYIVTMVVMTIIVAFILEAFVFRMNYSRKSQDSEVDSGIVIEKEMSKEELMAVLELYREERGTSSDVTRLLDTLSQMEKYQQNSMVFLGRRSRTKSDLSLKMYQEEIQEWYEEHAREQEQQKLRGSVPGPAAQQPPGSRQRSQTVT

SEQ ID NO: 3 - DNA, 3569 bp, Mus musculus
  CDS (14)..(1798)
acctgccctcgcgatggcggcccccgtggatgggagctccggcggctgggcggcccgggcgctacggcgggcactggcgctgacctccctgaccacactggccttgctggcctcgctgaccgggctgttgctgagcggcccggcgggcgctctccctaccctggggcccggctggcagcgccaaaatccggacccgccggtctcccgcacccgctcgctgctgctggacgccgcgtcgggccagctgcgcctggaggatggcttccaccccgacgcggtggcctgggccaacctcaccaacgccatccgcgagaccgggtgggcctatctggacctgagcacaaatggcaggtacaatgacagcctgcaggcctatgcagctggtgtggtggaggcctctgtgtctgaggagctcatctacatgcactggatgaacacggtggtcaactactgcggccccttcgaatacgaagtcggctactgtgagaagcttaagaacttcctggaggccaacctggagtggatgcagagggaaatggagcttaacccggactctccgtactggcaccaggtgcggctgaccctcctgcagctgaaaggcctggaggacagctatgaaggccgtttaaccttcccaactgggaggttcaccatcaaacccttggggttcctcctgctgcagatctctggagacctggaagacctagagccagccctgaataagaccaacaccaagccttccctgggctccggttcatgctctgccctcatcaagctgctgcctggcgggcatgacctcctggtggcgcacaacacgtggaactcctaccagaacatgttacgcatcatcaagaagtacaggctgcagttccgggaggggccgcaagaggagtaccccctggttgctggcaacaacttggttttctcgtcctacccgggcaccatcttctccggagatgacttctacatcctgggcagtggcctggtcaccctggagaccaccattggcaacaagaacccagccctgtggaagtacgtgcagccccagggctgtgtgctggagtggatacgaaatgtcgtggccaaccgcctggccttggacggggccacctgggcagacgtcttcaagcggttcaacagcggcacgtacaatagccagtggatgattgtggactacaaggcattcctccccaacggacccagccctggaagccgggtgctcactatcctagaacagatcccgggcatggtggtggtggctgacaagactgcagagctctacaaaacgacctactgggctagctacaacatcccgtactttgagactgtattcaacgctagtgggctgcaggccctggtggcccagtatggagattggttctcttacactaagaaccctcgagccaagatcttccagagggaccagtcactggtggaggacatggacgccatggtccggctcatgaggtacaatgatttcctccatgaccctctgtcattgtgtgaagcctgcaacccaaagcctaatgcggagaatgccatctctgcccgctctgacctcaaccccgccaatggctcctacccatttcaggccctgcatcagcgcgcccatggtggcattgatgtgaaggtgaccagctttaccctggccaagtacatgagcatgctggcggccagtggccccacgtgggaccagtgtcctccgttccagtggagcaaatcgccattccacagcatgctgcacatgggccagcccgacctctggatgttctcacccatcagggtgccatgggactgagagagagtccgcctccgcctagctgccttcattttgtgtggccagtgggtcatacacctgccgtccacccctcgggcttctgtcttcactagactctggtcctagcggcttccttcgcaaggacacaacccagtgggctcagagttgcctctgtccctgagccctctgccccttcatggctcatcctccctgtccctgtcaccagcaggctggggcttatgcttggctgtgggcctggtgggatccggggcacacattctcctagtgctggtccctcagcatatgtgtgaactgacaggggacattatggttgtcactgctggcctgtgggcccatcgcctcagaaggcagccctgtgcccttctgggcagctcttctaagtgcaggagcttgagaacaaaaccaaagtttctggctgcttgtagctggagggccttgagtcttctttcagcaggaggaatggaccgtcaccccacacttctacccctactcccagccaagcctgcccctggcctcctggtaggtgtctctggctgtgtgctccggtcagcgaagcccaggctgtgcttctgttaaacaagccttgtggggcggcaccacaccctgtctgtccatggctgtctccttccatctgtcctttctctagtccacgtacctactgttcacctgcatcaccagccccctgcccgtccatcccctctcctgtccaccctaccgtcttttcactcagtcagcggtccacacacctacccgtccacttgcctgccttttcatctgaacgtcctcacctctcacccacccatccacctgcccgtccgtcagtctgtccacccagaactgcacatcccttttcattttggttttgctctgtaactcagtgtggccttgaacttgaatgccttgtggtccacagtcatcctctggttattgtatctgctggccgccttcaccctcacatgctgggtttccatgtacaggccgccgtgccttgattctgcttttcatcagcccactcatctctctgtccatttatccctgttagtccttctgctcagcatctgtctcaagtgctgtattctgggtcctagcctggcccaacctgctgaccactaagaccctaaatcctagacccagagacccacatgaaaccaagtcccctgtgtggggcccagagagctcacagcctgtgaaccagaggccctaagatgtgtgggtagctttacccgagccagtccgggcctgccatgtgttctgagtttcgttggtagtcaggggttggtttcctgtcaggttcactccaccttccctgctctgcagatgcctgcacggggttggttttccagacaccagggtggggccgctggtgtctcaggatggccttgtcctgtccctaaggacttcatattagcatgtggtggccttgcctgtgcatctcccagctgctttgtgaccccaccctagatccctgtgtggcttagctctggtcctctctcctgacttacggatgtgtgggttccaccagtcaatccactgggagacccaagaccccagaggggagcaccactcctttgagagccagggacgatgtggctgggtgtaaagggagactgaacaaggcaaggaagctaacgcctgtctgcccggcactcagaagatgaggcaggaggatctccagtttgagaccagcctgggctgcacatagaccctcatgtcaagaacaaacaaagctcaaaagatctttgctctataaatatatatttattttttattt

SEQ ID NO: 4 - protein, 594 aa, Mus musculus
MAAPVDGSSGGWAARALRRALALTSLTTLALLASLTGLLLSGPAGALPTLGPGWQRQNPDPPVSRTRSLLLDAASGQLRLEDGFHPDAVAWANLTNAIRETGWAYLDLSTNGRYNDSLQAYAAGVVEASVSEELIYMHWMNTVVNYCGPFEYEVGYCEKLKNFLEANLEWMQREMELNPDSPYWHQVRLTLLQLKGLEDSYEGRLTFPTGRFTIKPLGFLLLQISGDLEDLEPALNKTNTKPSLGSGSCSALIKLLPGGHDLLVAHNTWNSYQNMLRIIKKYRLQFREGPQEEYPLVAGNNLVFSSYPGTIFSGDDFYILGSGLVTLETTIGNKNPALWKYVQPQGCVLEWIRNVVANRLALDGATWADVFKRFNSGTYNSQWMIVDYKAFLPNGPSPGSRVLTILEQIPGMVVVADKTAELYKTTYWASYNIPYFETVFNASGLQALVAQYGDWFSYTKNPRAKIFQRDQSLVEDMDAMVRLMRYNDFLHDPLSLCEACNPKPNAENAISARSDLNPANGSYPFQALHQRAHGGIDVKVTSFTLAKYMSMLAASGPTWDQCPPFQWSKSPFHSMLHMGQPDLWMFSPIRVPWD

SEQ ID NO: 5 - DNA, 2868 bp, Mus musculus
  CDS (150)..(1907)
ctccgggccaggactctaggggcggaaggttgtggcgctggccatccggctagaggaagactccgaggtcgcggatccaggccccgcccgaggcactagagcagccagcccgtgagcagagagggctctggtcaggcctcaaggggcccatggcaagcagatggctggctctgctctgggctcctgtcttcctctgtgtggctctgttactggagacggcgtctgggaccggagacccatccacaaaagcccatggacacatccagttttcagctggaagtgtcaaccagactgccatggcggattgccgagccgtgtgtggcctgaacacatctgatcgctgtgactttgtcaggaggaatccggactgccgcagcgaggcgggctacctggactaccttgagggcatcttctgctacttcccccccaacctcctccctctggccatcaccctctacgttttctggctgctttacctctttctgatcctgggagtcaccgcggccaagttcttctgccctaacctgtcagccatctccactaacctcaaactctcccacaacgtggcaggtgtcaccttcctggcctttggaaatggcgctccagacatcttcagtgctttagtggctttctcagacccacgtactgccggcctggccatcggggctctgtttggtgcaggggtgctggtcaccactgtggtggctggaggcatcaccatcctgcatcccttcatggctgcctccaggcccttcctcagggacatcgctttctacatggtggctgtgttcctaaccttcactgcactctatcttggcaggatcacgctggcgtgggcgctgggttacctgggcctctacgtgttctacgtggtcacggtcatcatctgcacttgggtctaccaacggcagcgaagcaggtctctggtccactccatatcggagacaccagagttgctgtctgagtcagaggaggaccagatgtcttccaacaccaacagctatgactatggagatgagtaccggcctctgttgctgggtcaggagaccactgtccagatcctgatccaagccctgaatcccttggactacaggaagtggagaactcagtcgatatcctggagggtcctgaaggtagtcaagttacctgtggagttcttgttgctgctcacagtaccggttgtggaccctgacaaggacgatcggaattggaaacggccactcaactgtctgcagctggtcatcagccccctggtcctggtcctgaccctgcagtcgggggtctatggcctctatgagattggcggtctccttcctgtctgggctgtggtggtgatcgtgggcacagcgctggcttcagtgaccttctttgccacgtctaacagagaaccccctagactgcactggctctttgctttcctgggtttcctgaccagtgccctgtggatcaatgcagccgccacagaggtggtgaacatcttacggtccctgggtgtgatcctccgcctgagcaacaccgtcctagggctgaccctcctggcctggggaaacagcattggagatgccttctcagatttcacgctggcccgccaaggataccctcggatggccttctccgcctgtttcgggggcatcatcttcaacatcctggttggtgtggggctgggctgcttgctgcagatcatcaggaaccacgttgtggaggtgaagctggagccagacggattactggtgtgggtgctggccagtgccctgggcctcagcttgatcttctccctggtctccgtgccgcttcagtgtttccagctcagcaaggcttacggcctctgcctcctcctcttctacgtctgtttccttgttgtggtcctgctcacagagtttggggtgattcacctgaagaaggcgtgactgaagctgcttggcctagaggtgtgggggcgattctgctagcctcctgagggggaggtgtggggagggggaccctctgtggtccccgtggatctcctgagaagatagtcactggcagagctctgcagggtgagaaggtcctgactgccggcacctaacagccttagtgtggggatctggaggctggctttgctggggacaatcccgggtaggaatgatgggatctaaatgacaccggaggctctggggggagggcagcctttcagtcagcccccatgcctgctgggctctgggcagccctgcagttccctctctggctcttccactctctgtggggtcctgcgtacctacagggtggcctgaaaacagactccacacgtggaaacaagactgggtttctcagcttccgtgtcaagtcagccagaggaaagaggtcgagttgacagcagcaggcactgcccttcctagttggtggctgccatgttggactgtggatctaaaacacttctagagctttgtggtccaaactctggctctcccgtctgcaaaacaggagccagcatgggctctgtgcctacctcaaggggagctgggggttggggggactgaccctccccagggaggtcttacaagtagtgtgaccagtcttttgtttgtttggttggttggttgttttttggagacagggtttctctgtgtagccctggctgtcctggaactcactctgtagaccaggctggccttgaactcagaaatctgcctgcctttgcctcccaagtgctgggactaaaggtgtgtgccaccactgtccagcttgtgaccagccttttaaaagtgccactcactccctgactgccttcagctgtaattaagagacttgactgttggggtttttttttgtttgtttgttttttgtttttgttttttgttgtttccaaaaataaaagatgagttatttcac

SEQ ID NO: 6 - protein, 585 aa, Mus musculus
MASRWLALLWAPVFLCVALLLETASGTGDPSTKAHGHIQFSAGSVNQTAMADCRAVCGLNTSDRCDFVRRNPDCRSEAGYLDYLEGIFCYFPPNLLPLAITLYVFWLLYLFLILGVTAAKFFCPNLSAISTNLKLSHNVAGVTFLAFGNGAPDIFSALVAFSDPRTAGLAIGALFGAGVLVTTVVAGGITILHPFMAASRPFLRDIAFYMVAVFLTFTALYLGRITLAWALGYLGLYVFYVVTVIICTWVYQRQRSRSLVHSISETPELLSESEEDQMSSNTNSYDYGDEYRPLLLGQETTVQILIQALNPLDYRKWRTQSISWRVLKVVKLPVEFLLLLTVPVVDPDKDDRNWKRPLNCLQLVISPLVLVLTLQSGVYGLYEIGGLLPVWAVVVIVGTALASVTFFATSNREPPRLHWLFAFLGFLTSALWINAAATEVVNILRSLGVILRLSNTVLGLTLLAWGNSIGDAFSDFTLARQGYPRMAFSACFGGIIFNILVGVGLGCLLQIIRNHVVEVKLEPDGLLVWVLASALGLSLIFSLVSVPLQCFQLSKAYGLCLLLFYVCFLVVVLLTEFGVIHLKKA

SEQ ID NO: 7 - DNA, 3324 bp, Mus musculus
  CDS (367)..(2991)
agttaccacatctctcacagcttggcattcgcgcagttagtaggtcccttaagcatctatcacggttcagttgacacattccgcccccaccagttggtatgtccctcaggcctcgcctctgcccggctggtacaccacttaggccccgcccccgttctgttggttcctccttctggcttcgcccattgataaagccatgttgacgctccgcccctgcgaagttggttggtttcctcaggcccggccccgccctacgggtgaaacctggattctcgacgccgctcttgcgtctcacaggctccgcccccgcgcagtcgacgcgtcccttaggccccgccctcttccgggtctaagagcccggcccgcatggctgctggcagacgtgtgggacctggcccgccgtcgcgtcccaccatggcgccgtggaagaagaagaggctgcggaaacgccgaactggggcttcccaaggccgcgacagcgactcggatgacggcgagttcgagatccaggcggaggatgacgcccgggcgaggaagctgggccctggcagagccttgccctcatttcctacctcagagtgcgtatcagatgtggagcccgacactcgggagatggtgcgagcccagaacaagaaaaagaagaagtctggaggcttccagtccatgggcctgagttaccctgtgttcaaggggatcatgaaaaagggctacaaggtgccgacgcccatccagaggaagaccatccccgtgatcttggatggcaaggatgtggtggccatggcccggacaggcagtggcaagacggcctgcttcctcctcccgatgtttgagcggctgaaggcacgcagtgcacagacgggggctcgagccctcatcctctcacccacccgggagctggccctgcagaccatgaagttcactaaagagctaggcaagttcaccggcctcaagactgccttgatcctgggtggagacaaaatggaagaccagtttgcagccctgcacgagaaccctgacataatcattgccacccctgggcgtctggtgcatgtggctgtggagatgaacttgaagctgcagagtgtggagtatgtggtgttcgatgaagcagacaggctctttgaaatgggctttgctgagcagctacaggagatcataggccgccttcctgggggccaccagacggtgctgttctcagctacactgcccaagctgctggtggaatttgcacgggcaggcctcacagagcccgtgctcatccgcctggacgtagactccaagctcaatgagcagctcaagacctccttcctccttgtgcgcgaagacaccaaggctgccgtgctcctctacctgctgcagaatgtcgttcggccccaggaccagactgtggtgtttgtagccacaaagcaccatgcggagtacctcacagagttgctgatgggccagggtgtgagttgcgcccacatctatagtgccttggaccagacggcccgcaagatcaacttggccaagttcacacacaacaaatgttccaccctcatcgtgactgacctggccgcccggggcctggacatcccactgctggacaacgtcatcaactacagcttccctgccaagggcaagctcttcctgcaccgagtgggccgtgtggcccgagcaggccgaagtggcacagcctattctttggtggccccagacgaggtcccctacctgcttgacctacacctgttcctgggccgctctgtcaccctggcccgtccttgtgaggagccttcagtggcagatgcggttggcagggacggagtgctgggtcgcgtgccccagagtgtagtggatgatgaggacagcagcctgcagactgccatgggggcatccctggatcttcagggcctgcaccgcgtggccaacaacgctcagcagcagtatgtgcgctcacggccagcgccctcgcctgagtccatcaagagagccaaggagctggacctggcagagctgggcttgcacccactcttcagctcatgctttgaggagggagagctccaacgcctgaggctggtggacagcatcaagaactatcgcacgcgcacaaccatctttgagatcaatgcctccagcaaggacccaagcagccaaatgatgcgtgccaagcggcagagggaccggaaagctgttgccagtttccagcagcggcgccaggaaaggcaggaaggcccagctgacccagccccccagagggagctgcctcaggaggaggaggaggagatggttgagactgtagagggtgtcttcacagaagtcgtgggccagaaacggccaaggccgggacccagccaaggagccaagaggcggaggatggagacccgtcagcgagaccaggagttttatgtcccctaccggcccaaggatttcgacagtgagcgggggctgagtgtcagtggggctggaggggcctttgagcagcaggtggctggtgcagtcctggacctgatgggggatgaagcacagaacatgagccgggggcagcagcagctcaagtgggaccggaagaagaagcggtttgtggggcagtcaggccaagaagacaagaaaaagatcaagacagagagcggccggtttatcagcagctcttacaagcgggatctctaccagaagtggaagcagaagcagaaaattgatgaccgggactccgaggaagaagggccatccaaccagcgaggccctgggccccgcagaggtggaaagcgaggtcgtagtcaaggcacatcccagccccgagcttccagtgtacccgcaggccgcatgcgctcggaactcaagaccaaggagcaaatcctcaagcagcgccggcaagctcagaagcagcgcttcctgcagcgagggggcctgaagcagctttcagcacgcaaccgacgccgagcccaggagctgcgccagggcgcctttggccggggtgctccctccaggaagggcaagatgaggaaaaggatgtgaggagccagacgcagccctggggcttcctggtagccccgggtgtggacgtcagggactatgtccatgtgctgttggaagatccttccacaggcgctgctctgtgaggagtagtgccatatggccacagagcaacagctgcttttgactgggacattgggtgacctctgaaaggatgcataggagtctagctatgcaaagcaggcagacccaagtcctgaccctgcaagtcacagcggcttctggttccacaccttcaggattcagagtcagggccgtgtggatgcctctgacccagcattgagttttaatgtaataaactttactgcctctagt

SEQ ID NO: 8 - protein, 874 aa, Mus musculus
MAAGRRVGPGPPSRPTMAPWKKKRLRKRRTGASQGRDSDSDDGEFEIQAEDDARARKLGPGRALPSFPTSECVSDVEPDTREMVRAQNKKKKKSGGFQSMGLSYPVFKGIMKKGYKVPTPIQRKTIPVILDGKDVVAMARTGSGKTACFLLPMFERLKARSAQTGARALILSPTRELALQTMKFTKELGKFTGLKTALILGGDKMEDQFAALHENPDIIIATPGRLVHVAVEMNLKLQSVEYVVFDEADRLFEMGFAEQLQEIIGRLPGGHQTVLFSATLPKLLVEFARAGLTEPVLIRLDVDSKLNEQLKTSFLLVREDTKAAVLLYLLQNVVRPQDQTVVFVATKHHAEYLTELLMGQGVSCAHIYSALDQTARKINLAKFTHNKCSTLIVTDLAARGLDIPLLDNVINYSFPAKGKLFLHRVGRVARAGRSGTAYSLVAPDEVPYLLDLHLFLGRSVTLARPCEEPSVADAVGRDGVLGRVPQSVVDDEDSSLQTAMGASLDLQGLHRVANNAQQQYVRSRPAPSPESIKRAKELDLAELGLHPLFSSCFEEGELQRLRLVDSIKNYRTRTTIFEINASSKDPSSQMMRAKRQRDRKAVASFQQRRQERQEGPADPAPQRELPQEEEEEMVETVEGVFTEVVGQKRPRPGPSQGAKRRRMETRQRDQEFYVPYRPKDFDSERGLSVSGAGGAFEQQVAGAVLDLMGDEAQNMSRGQQQLKWDRKKKRFVGQSGQEDKKKIKTESGRFISSSYKRDLYQKWKQKQKIDDRDSEEEGPSNQRGPGPRRGGKRGRSQGTSQPRASSVPAGRMRSELKTKEQILKQRRQAQKQRFLQRGGLKQLSARNRRRAQELRQGAFGRGAPSRKGKMRKRM

SEQ ID NO: 9 - DNA, 1858 bp, Mus musculus
  CDS (64)..(1194)
ctgcttcagcgagcctaggagacacaggacctgctggctgcagaggtattagctggacctaggatggagcaggatctgaggagcatcccggcctcgaagcttgataagttcatagagaaccatctcccggacaccagcttctgtgctgacctcagagaagtcatagatgccctgtgtgctctcctgaaggacagatccttccggggccccgtccgccgaatgagggcctctaaaggggtcaagggcaaaggcaccacactcaagggcaggtcagacgctgacctggtggtgttccttaacaatctcaccagctttgaggatcagttaaaccaacagggagtgttgattaaggaaattaagaaacagctgtgcgaggttcagcatgagagacgttgtggagtgaagtttgaggtccacagtttaaggagtcccaactcccgggctctgagcttcaagctgagcgcccccgacctgctgaaggaggtgaagtttgatgtgctgccagcctatgatttactggatcatcttaacatcctcaagaagcctaaccaacaattctacgccaatctcatcagtgggcgtaccccgccggggaaggagggcaagttatcgatctgctttatggggcttcggaagtacttcctgaactgtcgcccaaccaagctgaagcgcctcatccgcctggtcacgcactggtaccaactgtgtaaggagaagctgggggacccgctgcccccacagtatgccctggagctgctcacagtctatgcctgggagtatgggagtcgagtaactaaattcaacacagcccagggcttccgaaccgtcttggaactggtcaccaagtacaaacagcttcgaatctactggacagtgtattatgactttcgacatcaagaggtctctgaatacctgcaccaacagctcaaaaaagacaggcctgtgatcttggaccccgctgacccaacaaggaacatagctggtttgaacccaaaggactggcggcgtctagcaggagaggctgccacctggctgcaatacccatgctttaagtacagggacggttccccagtgtgctcctgggaggtgccgacggaggttggagtgccaatgaagtatctcttttgtcgtattttctggttattgttttggtctttgtttcatttcatctttgggaagacttcatctggatagcccagagtgtcttggatattgccatcctcctgccttagcgctggcatgactgcagcgtaggcctgttatgctctgcctcccctccatcctcaagtggacaagaactgggcatgtgttttcctgtgagcccagtgggacctgtccaggaggctccagagtcaggggcatgtcctgctctgctacagggccttgacccagagaagacaggaaggtgcccaaagcccaagagagggagggtccaacctgtgatcagactccaggcttctgtcccctgtcctcaacccctgcacagacagcctttctcacagcatgctttatctgtcttgtcccccaacagtgttctctgggagacaagagattcagaaggagaatatgatggtttgtatatggttggcccagggaatggcactgttaggagatgtggccatgttggaatgggtgtggccttgtgggtgtgggctttctcttgtcttagctgcctggaagtcagtatgctgctagcagccttcaaatgaagatgtagaactctcagctcctcctgcaccatgcctgcctggacgttgccatgctcttgccttggtgataatggactgaacttctgaacctgtaagccaaccccaattaaatgttgtttttataaaaattgccttggtcatggtgtctg

SEQ ID NO: 10 - protein, 376 aa, Mus musculus
MEQDLRSIPASKLDKFIENHLPDTSFCADLREVIDALCALLKDRSFRGPVRRMRASKGVKGKGTTLKGRSDADLVVFLNNLTSFEDQLNQQGVLIKEIKKQLCEVQHERRCGVKFEVHSLRSPNSRALSFKLSAPDLLKEVKFDVLPAYDLLDHLNILKKPNQQFYANLISGRTPPGKEGKLSICFMGLRKYFLNCRPTKLKRLIRLVTHWYQLCKEKLGDPLPPQYALELLTVYAWEYGSRVTKFNTAQGFRTVLELVTKYKQLRIYWTVYYDFRHQEVSEYLHQQLKKDRPVILDPADPTRNIAGLNPKDWRRLAGEAATWLQYPCFKYRDGSPVCSWEVPTEVGVPMKYLFCRIFWLLFWSLFHFIFGKTSSG

SEQ ID NO: 11 - DNA, 1482 bp, Mus musculus
  CDS (331)..(1410)
gaagaccttgacagaaaacctcacaccccaagggcacatgcagaagaggccttcctgattctgtctcacgtggcttcgttcttagatgagcccaggtcgttcatgagccatgagctgcgtaaggaagacagagaggattgaatacccccacgggttcatcttggtcattttttgttgtgagttctcaccaggaccccagaatcaggagctgtccctttaacaaggaggaggggccaggccctggtagccggaagctgatctggtagccagtgcgtccaggttggtcctggctgggctgtccttcaggaaggtggtcagcgcgctgctggaatggagggggccttggcagaacgcgtcggggcggagcctttccacagggtcacgcccctgctggagagctgggcgctgtctcaggtggcaggcatgccggtcttcctcaaatatgagaatgtgcagatagctggctcctttaagattcggggcatcggacatttctgccagcagatggccaagaggggatgcagacatctggtgtgctcctcagggggcaatgcgggcattgcggctgcatactcggctcgtaagctgggcatccccgtcaccatcgtgctcccagagggcacctccgtgcaggtggtgaggcggctcgagggggaaggggccgaggtccagctgactgggaaagtctgggatgaagccaatgtaaaagcacaagaactggccacaagggatggctgggtgaacgtctccccgtttgaccatccccttatatgggaaggccatgccagcctagtgcgggagctgaaggagtcactagggacccctccaggtgccgtggtgctggccgtggggggcggagggctcctggcaggtgtgactgctggcctgctggaggtgggctggcagcatgtgcccatcgttgccatggagacccgcggggcgcacagtttcaattcggccttgcaggcaggcaggccggtcaccctgccagacatcaccagtgtagccaagagcctcggagccaagacggtggctgcacggaccttggagtgtgcaaaggagtgtgaggtcctctctgaggtggtagaagaccgggaggctgtcagcgctgtgcagaggttcctggacgatgagcgcatgctggtggaacctgcctgcggtgccgccctggccgccatctactcgggcatcctgtggaggcttcaggctgagggccgcctgagttctgccctagcttccgttgtggtcatcgtgtgcggtggcaacaacattagtagccaacagcttcaggagctgaaaaccagctgggctgcattgaaaatctcaggaactcccccaaagttcctggacacctggtgggtcatcaagggacctcggttccagtggtcctgccctcttccttccaggtagccctcctgggttgctctcagtggctccctgctgtccagtgaataaacctgactgagctgaaaaaaaaaaaaa

SEQ ID NO: 12 - protein, 359 aa, Mus musculus
MEGALAERVGAEPFHRVTPLLESWALSQVAGMPVFLKYENVQIAGSFKIRGIGHFCQQMAKRGCRHLVCSSGGNAGIAAAYSARKLGIPVTIVLPEGTSVQVVRRLEGEGAEVQLTGKVWDEANVKAQELATRDGWVNVSPFDHPLIWEGHASLVRELKESLGTPPGAVVLAVGGGGLLAGVTAGLLEVGWQHVPIVAMETRGAHSFNSALQAGRPVTLPDITSVAKSLGAKTVAARTLECAKECEVLSEVVEDREAVSAVQRFLDDERMLVEPACGAALAAIYSGILWRLQAEGRLSSALASVVVIVCGGNNISSQQLQELKTSWAALKISGTPPKFLDTWWVIKGPRFQWSCPLPSR

SEQ ID NO: 13 - DNA, 1837 bp, Mus musculus
  CDS (64)..(822)
ctgcttcagccagcctaggagacacaggacctgctggctgcagaggtaaaagctggacctaggatggagcaggatctgaggagcatcccggcctcgaagcttgataagttcatagagaaccatctcccggacaccagcttctgtgctgacctcagagaagtcatagatgccctgtgtgctctcctgaaggacagatccttccggggccccgtccgccgaatgagggcctctaaaggggtcaagggcaaaggcaccgcgctcaagggcaggtcagacgctgacctggtggtgttccttaacaatctcaccagctttgaggatcagttaaaccaacagggagtgttgattaaggaaattaagaaacagctgtgcgaggttcagcatgagagacgttgtggagtgaagtttgaggtccacagtttaaggagtcccaactcccgggctctgagcttcaagctgagcgcccccgacctgctgaaggaggtgaagtttgatgtgctgccagcctatgatttactggatcatcttaacatcctcaagaagcctaaccaacaattctacgccaatctcatcagtgggcgtaccccgccggggaaggagggcaagttatcgatctgctttatggggcttcagaagtacttcctgaactgtcgcccaaccaagctgaagcgcctcatccgcctggtcacgcactggtaccaactgtgtaaggagaagctgggggacccgctgcccccacagtatgccctggagctgctcacagtctatgcctgggagtatgggagtcgagtaactaaattcaacacagcccagggcttctgaaccgtcttggaactggtcaccaagtacaaacagcttcaaatctactggacagtgtattatgactttcgacatcaagaggtctctgaatacctgcaccaacagctcaaaaaagacaggcctgtgatcttggaccccactgacccaacaaggaacatagccggtttgaacccaaaggactggaggcgtctagcaggagaggctgccgcctggctgcaatacccatgctttaagtacagggacggttcctcagtgtgctcctgggaggtgccgacggaggttgcagtgccaacgaagtatctcttttgtcgtattttctggttattgttttggtctttgtttcatttcatctttgggaagacttcatctggatagcccagagtgtcttggatattgccatcctcctgccttagcgctggcatgactgcagcgtaggcctgttatgccctgcctcccttccatcctcaagtggacaagaactgggcatgtgttttcctgtgagcccagtgggacctgtccaggatgctccagagtcagacgcatgtcctgctctgctgcagggccttgacccagagaagacaggaaggtgcccaaagcccaagagagggagggtccaacctgtgatcagactccaggcttctgtcccctgccctcaacccctgcacagacagcctttctcacagcctgctttatctgccttgtcccccaacagtgttctctgggagacaagagattcagaaggagaatattatggtttgtatatggttggcccagggaatggcactgttaggaggtgtggccatgttggagtgggtgtggccttgtgggtgtgggctttctcttgtcttagctgcctggaagtcagtattctgctagcagccttcagatgaagatgtagaactctcagctcctcctgcaccatgcctgcctggacgttgccatgctcttgccttggttataatggactgaacgtctgaacctgtaagccaaccccaattaaatgttgtttttataaaaa

SEQ ID NO: 14 - protein, 252 aa, Mus musculus
MEQDLRSIPASKLDKFIENHLPDTSFCADLREVIDALCALLKDRSFRGPVRRMRASKGVKGKGTALKGRSDADLVVFLNNLTSFEDQLNQQGVLIKEIKKQLCEVQHERRCGVKFEVHSLRSPNSRALSFKLSAPDLLKEVKFDVLPAYDLLDHLNILKKPNQQFYANLISGRTPPGKEGKLSICFMGLQKYFLNCRPTKLKRLIRLVTHWYQLCKEKLGDPLPPQYALELLTVYAWEYGSRVTKFNTAQGF

SEQ ID NO: 15 - DNA, 1837 bp, Mus musculus
  CDS (64)..(1194)
ctgcttcagccagcctaggagacacaggacctgctggctgcagaggtaaaagctggacctaggatggagcaggatctgaggagcatcccggcctcgaagcttgataagttcatagagaaccatctcccggacaccagcttctgtgctgacctcagagaagtcatagatgccctgtgtgctctcctgaaggacagatccttccggggccccgtccgccgaatgagggcctctaaaggggtcaagggcaaaggcaccacactcaagggcaggtcagacgctgacctggtggtgttccttaacaatctcaccagctttgaggatcagttaaaccaacagggagtgttgattaaggaaattaagaaacagctgtgcgaggttcagcatgagagacgttgtggagtgaagtttgaggtccacagtttaaggagtcccaactcccgggctctgagcttcaagctgagcgcccccgacctgctgaaggaggtgaagtttgatgtgctgccagcctatgatttactggatcatcttaacatcctcaagaagcctaaccaacaattctacgccaatctcatcagtgggcgtaccccgccggggaaggagggcaagttatcgatctgctttatggggcttcggaagtacttcctgaactgtcgcccaaccaagctgaagcgcctcatccgcctggtcacgcactggtaccaactgtgtaaggagaagctgggggacccgctgcccccacagtatgccctggagctgctcacagtctatgcctgggagtatgggagtcgagtaactaaattcaacacagcccagggcttccgaaccgtcttggaactggtcaccaagtacaaacagcttcgaatctactggacagtgtattatgactttcgacatcaagaggtctctgaatacctgcaccaacagctcaaaaaagacaggcctgtgatcttggaccccgctgacccaacaaggaacatagctggtttgaacccaaaggactggcggcgtctagcaggagaggctgccacctggctgcaatacccatgctttaagtacagggacggttccccagtgtgctcctgggaggtgccgacggaggttggagtgccaatgaagtatctcttttgtcgtattttctggttattgttttggtctttgtttcatttcatctttgggaagacttcatctggatagcccagagtgtcttggatattgccatcctcctgccttagcgctggcatgactgcagcgtaggcctgttatgctctgcctcccctccatcctcaagtggacaagaactgggcatgtgttttcctgtgagcccagtgggacctgtccaggaggctccagagtcaggggcatgtcctgctctgctacagggccttgacccagagaagacaggaaggtgcccaaagcccaagagagggagggtccaacctgtgatcagactccaggcttctgtcccctgtcctcaacccctgcacagacagcctttctcacagcatgctttatctgtcttgtcccccaacagtgttctctgggagacaagagattcagaaggagaatatgatggtttgtatatggttggcccagggaatggcactgttaggagatgtggccatgttggaatgggtgtggccttgtgggtgtgggctttctcttgtcttagctgcctggaagtcagtatgctgctagcagccttcaaatgaagatgtagaactctcagctcctcctgcaccatgcctgcctggacgttgccatgctcttgccttggtgataatggactgaacttctgaacctgtaagccaaccccaattaaatgttgtttttataaaaa

SEQ ID NO: 16 - protein, 376 aa, Mus musculus; identical to SEQ ID NO: 10

SEQ ID NO: 17 - DNA, 1837 bp, Mus musculus
  CDS (64)..(1194)
ctgcttcagccagcctaggagacacaggacctgctggctgcagaggtaaaagctggacctaggatggagcaggatctgaggagcatcccggcctcgaagcttgataagttcatagagaatcatctcccggacaccagcttgtgtgctgacctcagagaagtcatagatgccctgtgtgctctcctgaaggacagattcttccggggccccgtccgccgaatgagggcctctaagggggtcaagggcaaatgcaccgcgctcaagggcaggtcagacgctgacctggtggtgttccttaacaatctcacctactttgaggatcaattaaaccaacagggagtgttgattaaggaaattaagaaacagctgtacgaggttcagcatgagagacgttttggagtcaagtttgaggtccagagtttaaggagtcccaactcccgggctctgagcttcaagctgagcgcccccgacctgctgaaggaggtgaagtttgacgtgctgccagcctatgatttactggatcatcttaacatcctcaagaagcctaaccaacaattctacgccaatctcatcagtgggcgtaccccgctggggaaggagggcaagttattgacctgctttatggggcttcggaagtacttcctgaactgtcgcccaaccaagctgaagcgcctcatccgcctggtcacgcactggtaccaactgtgtaaggagaagctgggggacccgctgcccccacagtatgccctggagctgctcacagtctatgcctgggagtatgggagtcgagtaactaaattcaacacagcccagggcttccgaaccgtcttggaactggttaccaagtacaaacagcttcgaatctactggacagtgtattatgactttcgacatcaagaggtctctgaatacctgcaccaacagctcaaaaaagacaggcctgtgatcttggaccccgctgatccaacaaggaatatagctggtttgaacccaaaggactggcggcgtctagcaggagaggctgccgcctggctgcaatacccatgctttaagtacagggacggttccccagtgtgctcctgggaggtgccgacggaggttgcagtgccaacgaagtatctcttttgtcgtattttctggttattgttttggtctttgtttcatttcatctttgggaagacttcatctggatagcccagagtgtcttggatattgccatcctcctgccttagcgctggcatgactgcagtgtaggcctgttatgccctgcctcccctccatcctcaagtggacaagaactgggcatgtgttttcctgtgagcccagtgggacctgtccaggatgctccagagtcagacgcatgtcctgctctgctgcagggccttgacccagagaagacaggaaggtgcccaaagcccaagagagggaggttccaacctgtgatcagactccaggcttctgtcccctgccctcaacccctgcacagacagcctttctcacagcctgctttatctgtcttgtcccccaacagtgttctctgggagacaagagattcagaaggagaatatgatggtttgtatatggttggcccagggaatggcactgttaggaggtgtggccatgttggagtgggtgtggccttgtgtgtgtgggctttctcttgtcttagctgcctggaagtcagtatgctgctagcagccttcagatgaagatgtagaactctcagctcctcctgcaccatgcctgcctggacgttgccatgctcttgccttggtgataatggactgaacttctgaacctgtaagccaactccaattaaatgttgtttttataaaaa

SEQ ID NO: 18 - protein, 376 aa, Mus musculus
MEQDLRSIPASKLDKFIENHLPDTSLCADLREVIDALCALLKDRFFRGPVRRMRASKGVKGKCTALKGRSDADLVVFLNNLTYFEDQLNQQGVLIKEIKKQLYEVQHERRFGVKFEVQSLRSPNSRALSFKLSAPDLLKEVKFDVLPAYDLLDHLNILKKPNQQFYANLISGRTPLGKEGKLLTCFMGLRKYFLNCRPTKLKRLIRLVTHWYQLCKEKLGDPLPPQYALELLTVYAWEYGSRVTKFNTAQGFRTVLELVTKYKQLRIYWTVYYDFRHQEVSEYLHQQLKKDRPVILDPADPTRNIAGLNPKDWRRLAGEAAAWLQYPCFKYRDGSPVCSWEVPTEVAVPTKYLFCRIFWLLFWSLFHFIFGKTSSG

SEQ ID NO: 19 - DNA, 1587 bp, Mus musculus
  CDS (64)..(1194)
ctgcttcagccagcctaggagacacaggacctgctggctgcagaggtaaaagctggacctaggatggagcaggatctgaggagcatcccggcctcgaagcttgataagttcatagagaatcatctcccggacaccagcttctgtgctgacctcagagaagtcatagatgccctgtgtgctctcctgaaggacagattcttccggggccccgtccgccgaatgagggcctctaagggggtcaagggcaaatgcaccgcgctcaagggcaggtcagacgctgacctggtggtgttccttaacaatctcacctactttgaggatcaattaaaccaacagggagtgttgattaaggaaattaagaaacagctgtacgaggttcagcatgagagacgttttggagtcaagtttgaggtccagagtttaaggagtcccaactcccgggctctgagcttcaagctgagcgcccccgacctgctgaaggaggtgaagtttgacgtgctgccagcctatgatttactggatcatcttaacatcctcaagaagcctaaccaacaattttacgccaatctcatcagtgggcgtaccccgccggggaaggagggcaagttattgatctgctttatggggcttcggaagtacttcctgaactgtcgcccaaccaagctgaagcgcctcatccgcctggtcacgcactggtaccaactgtgtaaggagaagctgggggacccgctgcccccacagtacgccctggagctgctcacagtgtacgcctgggaaagtgggagtcgagactgtgaattcaacacagcccagggcttccgaactgtcttggaactggtcaccaagtacaagcggcttcgaatctactggacagtgtattatgactttagaaagacgaaggtctctgaatacctgcacaaactgctccaaaaagacaggcctgtgatcttggaccccgctgatccaacaaggaatatagctggtttgaacccaaaggactggcggcgtctagcaggagaggctgccgcctggctgcaatacccatgctttaagtacagggacggttccccagtgtgctcctgggaggtgccgacggaggttgcagtgccaacgaagtatctcttttgtcgtattttctggttattgttttggtctttgtttcatttcatctttgggaagacttcatctggatagcccagagtgtcttggatattgccatcctcctgccttagcgctggcatgactgcagtgtaggcctgttatgccctgcctcccctccatcctcaagtggacaagaactgggcatgtgttttcctgtgagcccagtgggacctgtccaggatgctccagagtcagacgcatgtcctgctctgctgcagggccttgacccagagaagacaggaaggtgcccaaagcccaagagagggaggttccaacctgtgatcagactccaggcttctgtcccctgccctcaacccctgcacagacagcctttctcacagcctgctttatctgtcttgtcccccaacagtgttctctgggagacaagagattcagaaggagaatatgatggtttgtatatggttg

SEQ ID NO: 20 - protein, 376 aa, Mus musculus
MEQDLRSIPASKLDKFIENHLPDTSFCADLREVIDALCALLKDRFFRGPVRRMRASKGVKGKCTALKGRSDADLVVFLNNLTYFEDQLNQQGVLIKEIKKQLYEVQHERRFGVKFEVQSLRSPNSRALSFKLSAPDLLKEVKFDVLPAYDLLDHLNILKKPNQQFYANLISGRTPPGKEGKLLICFMGLRKYFLNCRPTKLKRLIRLVTHWYQLCKEKLGDPLPPQYALELLTVYAWESGSRDCEFNTAQGFRTVLELVTKYKRLRIYWTVYYDFRKTKVSEYLHKLLQKDRPVILDPADPTRNIAGLNPKDWRRLAGEAAAWLQYPCFKYRDGSPVCSWEVPTEVAVPTKYLFCRIFWLLFWSLFHFIFGKTSSG

SEQ ID NO: 21 - DNA, 1587 bp, Mus musculus
  CDS (64)..(1194)
ctgcttcagccagcctaggagacacaggacctgctggctgcagaggtaaaagctggacctaggatggagcaggatctgaggagcatcccggcctcgaagcttgataagttcatagagaatcatctcccggacaccagcttctgtgctgacctcagagaagtcatagatgccctgtgtgctctcctgaaggacagattcttccggggccccgtccgccgaatgagggcctctaagggggtcaagggcaaatgcaccgcgctcaagggcaggtcagacgctgacctggtggtgttccttaacaatctcacctactttgaggatcaattaaaccaacagggagtgttgattaaggaaattaagaaacagctgtacgaggttcagcatgagagacgttttggagtcaagtttgaggtccagagtttaaggagtcccaactcccgggctctgagcttcaagctgagcgcccccgacctgctgaaggaggtgaagtttgacgtgctgccagcctatgatttactggatcatcttagcatcctcaagaagcctaaccgacaattatacgccaatctcatcagtgggcgtaccccgccggggaaggaccccaagttatcgatctgctttatggggcttcggaagtacttcctgaactgtcgcccaaccaagctgaagcgcctcatccgcctggtcacgcaatggtaccaactgtgtaaggagaagctgggggacccgctgcccccacagtacgccctggagctgctcacagtgtacgcctgggaaagtgggagtcgagactgtgaattcaacacagcccagggcttccgaactgtcttggaactggtcaccaagtacaagcggcttcgaatctactggacagtgtattatgactttagaaagacgaaggtctctgaatacctgcacaaactgctccaaaaagacaggcctgtgatcttggaccccgctgatccaacaaggaatatagctggtttgaacccaaaggactggcggcgtctagcaggagaggctgccgcctggctgcaatacccatgctttaagtacagggacggttccccagtgtgctcctgggaggtgccgacggaggttgcagtgccaacgaagtatctcttttgtcgtattttctggttattgttttggtctttgtttcatttcatctttgggaagacttcatctggatagcccagagtgtcttggatattgccatcctcctgccttagcgctggcatgactgcagtgtaggcctgttatgccctgcctcccctccatcctcaagtggacaagaactgggcatgtgttttcctgtgagcccagtgggacctgtccaggatgctccagagtcagacgcatgtcctgctctgctgcagggccttgacccagagaagacaggaaggtgcccaaagcccaagagagggaggttccaacctgtgatcagactccaggcttctgtcccctgccctcaacccctgcacagacagcctttctcacagcctgctttatctgtcttgtcccccaacagtgttctctgggagacaagagattcagaaggagaatatgatggtttgtatatggttg

SEQ ID NO: 22 - protein, 376 aa, Mus musculus
MEQDLRSIPASKLDKFIENHLPDTSFCADLREVIDALCALLKDRFFRGPVRRMRASKGVKGKCTALKGRSDADLVVFLNNLTYFEDQLNQQGVLIKEIKKQLYEVQHERRFGVKFEVQSLRSPNSRALSFKLSAPDLLKEVKFDVLPAYDLLDHLSILKKPNRQLYANLISGRTPPGKDPKLSICFMGLRKYFLNCRPTKLKRLIRLVTQWYQLCKEKLGDPLPPQYALELLTVYAWESGSRDCEFNTAQGFRTVLELVTKYKRLRIYWTVYYDFRKTKVSEYLHKLLQKDRPVILDPADPTRNIAGLNPKDWRRLAGEAAAWLQYPCFKYRDGSPVCSWEVPTEVAVPTKYLFCRIFWLLFWSLFHFIFGKTSSG

SEQ ID NO: 23 - DNA, 1587 bp, Mus musculus
  CDS (64)..(1194)
ctgcttcagccagcctaggagacacaggacctgctggctgcagaggtaaaagctggacctaggatggagcaggatctgaggagcatcccggcctcgaagcttgataagttcatagagaatcatctcccggacaccagcttctgtgctgacctcagagaagtcatagatgccctgtgtgctctcctgaaggacagattcttccggggccccgtccgccgaatgagggcctctaagggggtcaagggcaaatgcaccgcgctcaagggcaggtcagacgctgacctggtggtgttccttaacaatctcacctactttgaggatcaattaaaccaacagggagtgttgattaaggaaattaagaaacagctgtacgaggttcagcatgagagacgttttggagtcaagtttgaggtccagagtttaaggagtcccaactcccgggctctgagcttcaagctgagcgcccccgacctgctgaaggaggtgaagtttgacgtgctgccagcctatgatttactggatcatcttaacatcctcaagaagcctaaccaacaattttacgccaatctcatcagtgggcgtaccccgccggggaaggagggcaagttatcgatctgctttatggggcttcggaagtacttcctgaactgtcgcccaaccaagctgaagcgcctcatccgcctggtcacgcactggtaccaactgtgtaaggagaagctgggggacccgctgcccccacagtacgccctggagctgctcacagtgtacgcctgggaaagtgggagtcgagactgtgaattcaacacagcccagggcttccgaactgtcttggaactggtcaccaagtacaagcggcttcgaatctactggacagtgtattatgactttagaaagacgaaggtctctgaatacctgcacaaactgctccaaaaagacaggcctgtgatcttggaccccgctgatccaacaaggaatatagctggtttgaacccaaaggactggcggcgtctagcaggagaggctgccgcctggctgcaatacccatgctttaagtacagggacggttccccagtgtgctcctgggaggtgccgacggaggttgcagtgccaacgaagtatctcttttgtcgtattttctggttattgttttggtctttgtttcatttcatctttgggaagacttcatctggatagcccagagtgtcttggatattgccatcctcctgccttagcgctggcatgactgcagtgtaggcctgttatgccctgcctcccctccatcctcaagtggacaagaactgggcatgtgttttcctgtgagcccagtgggacctgtccaggatgctccagagtcagacgcatgtcctgctctgctgcagggccttgacccagagaagacaggaaggtgcccaaagcccaagagagggaggttccaacctgtgatcagactccaggcttctgtcccctgccctcaacccctgcacagacagcctttctcacagcctgctttatctgtcttgtcccccaacagtgttctctgggagacaagagattcagaaggagaatatgatggtttgtatatggttg

SEQ ID NO: 24 - protein, 376 aa, Mus musculus
MEQDLRSIPASKLDKFIENHLPDTSFCADLREVIDALCALLKDRFFRGPVRRMRASKGVKGKCTALKGRSDADLVVFLNNLTYFEDQLNQQGVLIKEIKKQLYEVQHERRFGVKFEVQSLRSPNSRALSFKLSAPDLLKEVKFDVLPAYDLLDHLNILKKPNQQFYANLISGRTPPGKEGKLSICFMGLRKYFLNCRPTKLKRLIRLVTHWYQLCKEKLGDPLPPQYALELLTVYAWESGSRDCEFNTAQGFRTVLELVTKYKRLRIYWTVYYDFRKTKVSEYLHKLLQKDRPVILDPADPTRNIAGLNPKDWRRLAGEAAAWLQYPCFKYRDGSPVCSWEVPTEVAVPTKYLFCRIFWLLFWSLFHFIFGKTSSG

SEQ ID NO: 25 - DNA, 3897 bp, Mus musculus
  CDS (16)..(2271)
gaccagctagcaacgatgggaaactggctgactggaaactggtcatctgacaggtcatctggctattcatctggctggtcacctggtgggtcttcaggggtgccctccgggccagtgcacaagttagaaaagtctatccaggcaaacctcacacccaacgaaaactgtctgaagcagattgcggtgtcctcggtgccatcgcagaagctagaagggtatatccaggaaaacctcaaacctaacagagaatctctgaagcagatagaccaggccgtggatgccatctgggacctgctgcgcagtcagatccctgtgaaggaagtggctaagggtggctcctatggccgggaaacagccctaagaggctgctccgatggtacccttgttctcttcatggactgcttccaacagttccaggatcagataaaataccaagatgcataccttgacgtcattgaactgtggctgaaaatccatgagaagaagtcagtaaagcatgaacatgcccttgtagtacaagtgtctgtaccagggcagagaatactcctgcaattacttccagtcttcaatcctctacgctccaatgagaatcccagctcctgtgtctatgtggatctcaaaaaatccatggatcaagtaagagcctcaccaggggagttctcagactgcttcaccacactgcagcagcggtttttcaagaaatatccccgaagactgaaggatttgatcctattggtcaagcactggtatgaacagtgccaggagaagtggaaaacacccccacctcagccattgctgtacgcactggaactgctcactgtgtatgcctgggaacagggctgccaagctgaagacttcgacatggcacaaggcgtcaggaccgtgctgcgacttatccagcggccgacagagctgtgtgtctactggacagtcaattacaactttgaggatgagacagtccggaacatccttctgcaccagctcaggtcccaaagaccagtcatcttggatccaactgacccaaccaataatgtgggcaaagatgatgggttctgggagctactgacagaggaagctatggcctggctgtactctcccagcctgaatactgagtcacctgcaccatattgggatgttctgcccatgccacttttcgtcactccaagccacttactgaacaagttcatcaaggactttctccagcccaacaagctcttcctaaagcagatcaaggaagctgttgacattatatgttccttccttaaaaatgtctgcttcttgaattctgacaccaaagtcctgaagaccgtcaagggaggatccactgccaaaggcacagctctgaagcggggatcagatgctgacattgttgtgttcctctcctcgctggagagttacgactctctaaaaaccaaccgctcccagttcgtccaggagatccagaagcagttagaagaattcgtgcaggcgcaggagtgggaggtgacgtttgagatttcaaaatggaaggctcccagagtgctgagttttaccttgaaatccaagactctcaatgaaagtgtcgagttcgatgtccttcccgcctatgatgcactaggtcaactgcggtctgacttcaccctcaggcccgaagcctacaaggatctcattgagctgtgtgcatcacaggacatcaaagaaggagagttttctatctgttttactgagctgcagagaaacttcattcaaacccggcccaccaaactgaagagtctactccgcctgatcaagcactggtacaaacagtatgaaaggaagatgaagccaaaagcatctttacccccaaagtacgccctggagctgctcaccgtgtatgcctgggagcagggcagtggcacagatgactttgacattgctgaaggcttccggaccgtcctggacctggttataaaataccggcagctctgcatcttctggacagtcaattacaactttgaagaggaatacatgcggaagttcctactgacccagatccagaaaaagaggcctgtaatcctggatccagcagatcccacaggcgatgtgggaggaggtgaccgctggtgctggcatcttctagctgaagaagcgaaggagtggctgtcctccccttgtttccaagtggagcaaaaaggcctggtacagccttggaaagtgccagtacctagagatctaaagacaagtgacatggtgggagtgttcactacaggagggatcttgtggcaggaccagggctttttgtcatttgtctaggtaatgcagacccccggaagctgtggaggtcagatctaccccactgtgggtggagttactaagtaggagtccattcagctctggaagacgcttctggagtgatctggcaaagactcagactgtgttagaaaagggagcctggttcagtcctctctggcaggctcgcacctctattcttccttcttggaatcaagacatgggattatccttcctcctcccccagggtctcacagcacaggccctgctctgtgtgagtgacctccttcagagacacttgccccatgcagctcgatgggttctggttttgtctgtattctgtgcagttattttcctgcctcctgctctgttagtctctagtcagcagctccagactcaccctgtgtcactaaggttaaggccctccctagcccttcagcattgtcaatcccaactagccctcggagtcttccattgtgcgtctttgcctgtctctttccctgtccctgtggatacagagatgtaccatccatccagcagctagccaactcccctccctccacctctgctgttaaaaccctttctcttggggaaatgtaaacaatatctacctctcttaatgtcccaggacaaactaagctgcatttctcccttccctgagaagccaaagcttccctgattgagcttrgctgctcacaggagaggggttacaggcctttgaagctggccacactagaagatctgcacccagctagatgggtgcagatggcttccctggggctgcataaagagaacccctcccctcatctttcctcctgtatcctctagcccctctcagagatcctgtgcaatcagggcagaatagcatgcagctggttgaaaccacttgctaaataactcaggtgagggtcccataaccttcccagcccacctcccttccaagagtgaagataacagtcaacaagcccagctgtgatgttcattgataagcaggctctggtggactcctaaagatggtgccagtgtggctcagtgaatagccctgcataacattttacacacaccaaatgctggttgatatctcttgctggctgcccagggagccttcaccccagggctttaactgcacagagacatgaggtctaagcccttcgcatccccaagtaaggctgagccttttttctgcctgtgcttgctctgatgcattgaggatcatgcctggccactgtgcaacttttaagcagagccgtgcaacatcccagggagttgacttctatgtaaacaccttcatccatttctgatgtatgctttgaggtggctcaggctgggctagcccagcccagacagaaatcctaggcatgtgattagaggatcagaacccttctggcccttcttcaggggagagatggggctgaaggtggggttcaaatctcatgccgagtgatggaacccgacatccctaggtgctaaggccccaccaaattctctggataaggaagttccaggaatctttactgataaacatcccaatgtatcaacaaggtagactctgacctccatgggacagaagatcctgggtcagtcccctccctggggactctgcagttggctgttcatttatatgcttcataataaatggtttctttgtgt

SEQ ID NO: 26 - protein, 751 aa, Mus musculus
MGNWLTGNWSSDRSSGYSSGWSPGGSSGVPSGPVHKLEKSIQANLTPNENCLKQIAVSSVPSQKLEGYIQENLKPNRESLKQIDQAVDAIWDLLRSQIPVKEVAKGGSYGRETALRGCSDGTLVLFMDCFQQFQDQIKYQDAYLDVIELWLKIHEKKSVKHEHALVVQVSVPGQRILLQLLPVFNPLRSNENPSSCVYVDLKKSMDQVRASPGEFSDCFTTLQQRFFKKYPRRLKDLILLVKHWYEQCQEKWKTPPPQPLLYALELLTVYAWEQGCQAEDFDMAQGVRTVLRLIQRPTELCVYWTVNYNFEDETVRNILLHQLRSQRPVILDPTDPTNNVGKDDGFWELLTEEAMAWLYSPSLNTESPAPYWDVLPMPLFVTPSHLLNKFIKDFLQPNKLFLKQIKEAVDIICSFLKNVCFLNSDTKVLKTVKGGSTAKGTALKRGSDADIVVFLSSLESYDSLKTNRSQFVQEIQKQLEEFVQAQEWEVTFEISKWKAPRVLSFTLKSKTLNESVEFDVLPAYDALGQLRSDFTLRPEAYKDLIELCASQDIKEGEFSICFTELQRNFIQTRPTKLKSLLRLIKHWYKQYERKMKPKASLPPKYALELLTVYAWEQGSGTDDFDIAEGFRTVLDLVIKYRQLCIFWTVNYNFEEEYMRKFLLTQIQKKRPVILDPADPTGDVGGGDRWCWHLLAEEAKEWLSSPCFQVEQKGLVQPWKVPVPRDLKTSDMVGVFTTGGILWQDQGFLSFV

SEQ ID NO: 27 - DNA, 4708 bp, Mus musculus
  CDS (28)..(3444)
gaaactctactgagagtaccggtcaacatggacctgttccacacgccagccggagctctggataagctggtggcccacaacctgcacccagcccctgagttcacagcagccgtacggggtgctctggggtcgctaaacatcaccctacagcagcacagagcccgagggtcacagagaccaagagtgataaggattgccaagggaggagcctatgcccggggcacagctctcagaggtggcaccgatgtcgaactcgtcatcttcctcgactgcttccagagctttggtgaccagaagacctgtcactcagagaccctgggtgccatgcgaatgttgctggagtcctgggggggccaccccgggcctggcctgacttttgagttttctcagtcaaaggcgtccaggatcttacagtttcgtctggcatcggcagacggagaacactggatagatgttagcctggtgcctgcctttgatgtcctaggacagccccgctctggagtcaagccgacacccaacgtgtactcctccctccttagcagccactgccaggccggggagtactcagcctgcttcactgagccccgaaagaactttgtgaacactcgcccagccaagcttaagaacttaatcctgctggtcaaacactggtaccaccaggtgcagacacaggccgtgagggccacactgccccccagctacgccctagagctgcttaccatctttgcctgggagcagggctgtgggaaggacagcttcagcctggcccaagggctccggaccgtcctggccttgatccaacacagcaagtacctctgcattttctggacggaaaactatggcttcgaggaccctgcagttggagagttcttgcgaaggcagcttaagagacccaggcccgtgatcctggatccagctgatccaacgtgggacgtgggcaacgggacagcctggcgctgggatgtgctggcccaggaggctgagtccagctttagccagcagtgcttcaagcaggcctcaggagtccttgtgcagccttgggaggggccgggcctgccacgggctgggatcttggatttgggccacccaatctatcaagggcctaaccaggcccttgaagacaacaaaggccaccttgctgttcagtcaaaggaaaggagccaaaaaccttccaattcagctccaggatttccagaagcagccaccaagatccctgctatgcccaacccaagtgccaataaaacccgcaagatccgcaagaaagcagctcacccaaagactgtccaggaagcagcattggatagtatctcaagtcatgttcggatcacccagagcacagcatcctcacacatgcctcctgaccgctctagcatctccaccgctgggtcacggatgagcccagatctgtcacagatccccagcaaggatctagactgcttcatccaggaccaccttaggccgagtccccagttccagcagcaggtgaagcaggccatcgacgccatcttgtgctgcctccgggagaagagtgtatacaaagtcttgagggtcagcaagggcggctctttcggccgtggcacagacctcaggggcagctgcgatgtggaacttgtcatcttttataaaaccctcggggacttcaagggccagaagcctcaccaggcagagatcctgcgtgacatgcaggcccagctacgacactggtgtcagaaccccgtgcctggactgagcctccagtttattgaacagaagcccaacgctctgcaactccagctggcgtccaccgacctcagcaaccgggtggacctcagtgtgctgcctgcttttgatgctgtggggccgctgaagtccggcaccaaacctcagccccaggtgtactcctcgctcctcagcagcggctgccaggctggggagcacgcagcctgcttcgcagagcttcgaaggaacttcataaacacttgccctcccaaacttaagagcctgatgctactggtcaaacactggtaccgccaggttgtcactcgatataaaggaggagaggcggcaggtgatgctccgcccccagcctacgccctggagctcctgaccatctttgcctgggaacaaggctgtggagagcaaaagttcagcctggctgaaggcctgcggaccatcctgaggctgatccaacagcaccagtcgctttgtatctactggacggtcaactacagtgtgcaggacccgaccatcagagcacatcttctctgccagcttcggaaagccaggcctctagtcctggaccctgcagatcccacctggaacgtgggccagggcgactggaagctattagctcaggaggcagctgcccttgggtcacaagtctgccttcagagtggggatgggactctggtgccaccctgggatgtgacgccagccctccttcaccagaccctagctgaggacctggacaaattcatcagtgaattcctccagcccaaccgccacttcctgactcaagtgaagagagccgtggacaccatatgttccttcctgaaagaaaactgcttccggaactctaccatcaaggtgctcaaggtggtcaagggtgggtcttctgccaaaggcacggctctacaaggacgctcagatgccgacctggtggtgtttctcagctgcttccgccagttctctgagcaaggcagccatcgggcagagatcatctcggagatccaggctcagctggaggcgtgtcagcagacgcacagcttcgatgtcaagtttgaggtctccaagaggaagaacccccgagtgctcagcttcacgctgacatcccagacgctgctggaccaaagcgtggactttgacgtcctgccagcctttgatgctctcggccagctgaggtccggctctcggcctgatccccgggtctacacagacctcatccacagctgcagtaatgcaggagagttctctacctgcttcacagagctgcagagggacttcattacctcccgtcccaccaaactcaagagcctgatccggctggtgaaatactggtaccaacagtgtaacaagaccatcaaggggaagggttccttgcctccccagcacgggctggagctcctaactgtgtacgcctgggagcaaggtggccagaatccccagttcaacatggcggagggcttccgcactgttctggagctgattgtccagtaccggcagctctgcgtctattggaccatcaactacagcgcagaagacaagaccatcggtgacttcctgaagatgcagcttcggaagcccaggcctgtcatcctggacccagctgacccgacaggcaacctgggccacaacgctcgctgggatctgcttgccaaggaggctaccgtgtacgcatctgccctgtgctgcgtggacagggatggcaatcccatcaagccatggccggtaaaggccgctgtgtgaagtctagagagatcagtggtcaccattgatagaaagtgacaccagccctcagcaagtgatactcagagtatctgagtgtgtgtgtgtgtgtgttgtatttatctgtatgtgtgtatttgtggtatgtctgtgtgcctatatgagggtgtgtctatgtgcgtgtctgtgtatctgtgggtatctatatgtgtctgtatatatgtatgtgtgtgtgtgtgtgtgtgtgtattcatgtatgtgtgtctgtttgtgtatagtgtgtctataggtgactctgtgtgtctgtgtatctgtgagtatctatatgtgtctgtctgtatgtaaatgtgtgtatgtatgtgtgttcatatgtctgtgtgtgtgtctatatctgtgtatctttgggtatctatatgtgtctatctatatgtaaatgtatgtatgtacttatgttcatgtgtatctgtgtgaatgtctgtgtgtttatgtgtagtgtatctgtaagtgtatctgtatgtctatagatgtattatgtctttgtgtgtcgacatgtctgtgtgtatgtatgtttgtatgtgtatgttatatatgtatatatgcatgtatgtgcttcctcacaccatctcccttctgcccacctgcccacccatagccctccctttcttcccactgtttacccacctggtggggcttcattgacctcaaccatgatcatcccggtgtccctgactcccacactagacaccctaggaaccagacatctctagatcttctagtctgctgttcatctaccatgggctctgccccaacttccacagccccacccaggagtgcctcagccctgccaagaagccatactcctccctggcatctctctgccccttgagcctgtgtatatccctctgcctacagagacccaccagctgaggtccaactatgttcctgtactggctggttttgtgtgtcaacttgacataggctggagttatcagagaaaggagcttcagttggggaaatgcctccatgagatccagctgtggggcattttctcagttggtgatcaaagaggagggcccattgtgggtggtgccatccctgggctggtagtcttgagttctataagagatcaagctgagcaagccaggggaagcaagccaataagaaacatccctccatggcctctgcatcagctcctgcttcctgacctgtttgagttccagttctgacttcctttagtgatgaacagcaatgtggaagtgtaagctgaataaaccctttcctcccc

SEQ ID NO: 28 - protein, 1138 aa, Mus musculus
MDLFHTPAGALDKLVAHNLHPAPEFTAAVRGALGSLNITLQQHRARGSQRPRVIRIAKGGAYARGTALRGGTDVELVIFLDCFQSFGDQKTCHSETLGAMRMLLESWGGHPGPGLTFEFSQSKASRILQFRLASADGEHWIDVSLVPAFDVLGQPRSGVKPTPNVYSSLLSSHCQAGEYSACFTEPRKNFVNTRPAKLKNLILLVKHWYHQVQTQAVRATLPPSYALELLTIFAWEQGCGKDSFSLAQGLRTVLALIQHSKYLCIFWTENYGFEDPAVGEFLRRQLKRPRPVILDPADPTWDVGNGTAWRWDVLAQEAESSFSQQCFKQASGVLVQPWEGPGLPRAGILDLGHPIYQGPNQALEDNKGHLAVQSKERSQKPSNSAPGFPEAATKIPAMPNPSANKTRKIRKKAAHPKTVQEAALDSISSHVRITQSTASSHMPPDRSSISTAGSRMSPDLSQIPSKDLDCFIQDHLRPSPQFQQQVKQAIDAILCCLREKSVYKVLRVSKGGSFGRGTDLRGSCDVELVIFYKTLGDFKGQKPHQAEILRDMQAQLRHWCQNPVPGLSLQFIEQKPNALQLQLASTDLSNRVDLSVLPAFDAVGPLKSGTKPQPQVYSSLLSSGCQAGEHAACFAELRRNFINTCPPKLKSLMLLVKHWYRQVVTRYKGGEAAGDAPPPAYALELLTIFAWEQGCGEQKFSLAEGLRTILRLIQQHQSLCIYWTVNYSVQDPTIRAHLLCQLRKARPLVLDPADPTWNVGQGDWKLLAQEAAALGSQVCLQSGDGTLVPPWDVTPALLHQTLAEDLDKFISEFLQPNRHFLTQVKRAVDTICSFLKENCFRNSTIKVLKVVKGGSSAKGTALQGRSDADLVVFLSCFRQFSEQGSHRAEIISEIQAQLEACQQTHSFDVKFEVSKRKNPRVLSFTLTSQTLLDQSVDFDVLPAFDALGQLRSGSRPDPRVYTDLIHSCSNAGEFSTCFTELQRDFITSRPTKLKSLIRLVKYWYQQCNKTIKGKGSLPPQHGLELLTVYAWEQGGQNPQFNMAEGFRTVLELIVQYRQLCVYWTINYSAEDKTIGDFLKMQLRKPRPVILDPADPTGNLGHNARWDLLAKEATVYASALCCVDRDGNPIKPWPVKAAV

SEQ ID NO: 29 - DNA, 1914 bp, Mus musculus
  CDS (20)..(1108)
aaacactcctggcctcaggatggagaatggtctctgcagcatccaagccagggagctggacgagttcatatgtgattacctctttcctgacaccaccttccttactgagctcagagcagacatcgactccataagtgctttcctgaaggagagatgcttccaaggtgccgcccatcctgtgagggtctccagggttgtgatgggcggctcctatgatgaacacactgcactcaagggcaagtcagaggccaaaatggtgttgttctttaacaatctcaccagctttgaggagcagttaaagcgacggggagagttcgttgaggaaattcagaaacacctgtgtcagctgcagcaagagaaaccatttaaagtgaagtttgaagtgcagagctcagaggagcccaactccaggtctctgagcttcaagctgagctcccccgagctccagcaggaggtggaatttgatgtgcagccagcctatgatgtcctatatgaactgagaaacaacacgtatgctgaaccccaattctacaacaaagtctacgcccaactcatccatgagtgcaccaccctggagaaggagggcgatttctccatctgcttcaccgacctccatcagaacttcatgaggtatcgtgcgcccaagctctggaacctcatccgtctggtcaagcactggtatcaactgtgtaaggagaagctgagggagccgctgcccccacagtacgccctggagctgctcactgtctatgtatgggaacattcgaataaaaatcaagaaaaagtaaccacagccaagaacttccggaccttcttagaactggtcgcctattacaagaatcttcgaatctactggacatggtattatgacttccgacatcaagaggtctgtgcctacctgtgcagacagctcaaaaaagccaggcctctgatcctggatccagcagacccaacaaggaacgtggctggttcagacttacaggcatgggacctgctggcaaaggaggctcagacctggatgcagtcctcttgctttagaaactgtgatatgtcctttgtgcccacctgggatttgtcgccagagagacaagaatgtgccttccagtgagcagtgcagcgcttgctctgaaggctccagagtcaggggcataccttcctctgctgcaagaccttgacctagagaggacaggatggtgctaaaggctccagtgaggggcatccagcctgtgatcagactccaggcttctgatccctgactgcccatggatagccttcctcacaggctgcttcgtctgccttagcttccaacagtgttctctgggagtcagactgtgatggacagagaagaacgcaagctcgacttccatctgtccacctgttgggaggttctgtccaacagtggctgattgtcatcaacaaaccacagcaagccatgggggagggtgcactctgagagaaggaacctttaagtacacttgtgtgtctgtgtgtttaaggatgtggtgtgtccatatgcaactagaaaccttgagcacgtgttacaagctccacatgggcccaggtaattgccagaaaggggtggacagagcaaaaccaaactgttacacgtattgatgttgggtagcttgggatccttctagatctctgatgcaagaaacccagactagaatccatggctcctgctgtccattctcctgtgacaaaattttaggccttccccatcccacacagaaactgttctccaaccacacatgaccctggagccctgggaatctggccagcgtgcatcgtggtgcactgattctgcagcatgcaggctgaggtccacagcagtgtgggaaagtaaaactatgtgcaatttgtgaccagtgatgacttgaaagcttagctgtctgtgtgagggtgagatttgaa

SEQ ID NO: 30 - protein, 362 aa, Mus musculus
MENGLCSIQARELDEFICDYLFPDTTFLTELRADIDSISAFLKERCFQGAAHPVRVSRVVMGGSYDEHTALKGKSEAKMVLFFNNLTSFEEQLKRRGEFVEEIQKHLCQLQQEKPFKVKFEVQSSEEPNSRSLSFKLSSPELQQEVEFDVQPAYDVLYELRNNTYAEPQFYNKVYAQLIHECTTLEKEGDFSICFTDLHQNFMRYRAPKLWNLIRLVKHWYQLCKEKLREPLPPQYALELLTVYVWEHSNKNQEKVTTAKNFRTFLELVAYYKNLRIYWTWYYDFRHQEVCAYLCRQLKKARPLILDPADPTRNVAGSDLQAWDLLAKEAQTWMQSSCFRNCDMSFVPTWDLSPERQECAFQ

SEQ ID NO: 31 - DNA, 1911 bp, Mus musculus
  CDS (20)..(1108)
aaacactcctggcctcaggatggagaatggtctctgcagcatccaagccagggagctggacgagttcatatgtgattacctctttcctgacaccaccttccttactgagctcagagcagacatcgactccataagtgctttcctgaaggagagatgcttccaaggtgccgcccatcctgtgagggtctccagggttgtgatgggcggctcctatgatgaacacactgcactcaagggcaagtcagaggccaaaatggtgttgttctttaacaatctcaccagctttgaggagcagttaaagcgacggggagagttcgttgaggaaattcagaaacacctgtgtcagctgcagcaagagaaaccatttaaagtgaagtttgaggtgcagagctcagaggagcccaactccaggtctctgagcttcaagctgagctcccccgagctccagcaggaggtggaatttgatgtgcagccagcctatgatgtcctatatgaactgagaaacaacacgtatgctgaaccccaattctacaacaaagtctacgcccaactcatccatgagtgcaccaccctggagaaggagggcgatttctccatctgcttcaccgacctccatcagaactttatgaggtatcgtgcgcccaagctctggaacctcatccgtctggtcaagcactggtatcaactgtgtaaggagaaagctgaggagccgctgcccccacagtacgccctggagctgctcactgtctatgtatgggaacattcgaataaaaatcaagaaaaagtaaccacagccaagaacttccggaccttcttagaactggtcgcctattacaagaatcttcgaatctactggacatggtattatgacttccgacatcaagaggtctgtgcctacctgtgcagacagctcaaaaaagccaggcctctgatcctggatccagcagacccaacaaggaacgtggctggttcagacttacaggcatgggacctgctggcaaaggaggctcagacctggatgcagtcctcttgctttagaaactgtgatatgtcctttgtgcccacctgggatttgtcgccagagagacaagaatgtgccttccagtgagcagtgcagcgcttgctctgaaggctccagagtcaggggcataccttcctctgctgcaagaccttgacctagagaggacaggatggcactcaaggctccagtgaggggcatccagcctgtgatcagactccaggcttctgatccctgactgcccatggatagccttcctcacaggctgcttcatctgccttagcttccaacagtgttctctgggagtcagactgtgatggacagagaagaacgcaagctcgacttccatctgtccacctgttgggaggttctgtccaacagtggctgattgtcatcaacaaaccacagcaagccatgggggagggtgcactctgagagaaggaacctttaagtacacttgtgtgtctgtgtgtttaaggatgtggtgtgtccatatgcaactagaaaccttgagcacgtgttacaagctccacatgggcccaggtaattgccagaaaggggtggacagagaaaaaccaaactgttacacgtattgatgttgggtagcttgggatccttctagatctctgatgcaagaaacccagactagaatccatggctcctgctgtccattctcctgtgacaaaattttaggccttccccatcccacacagaaactgttctccaaccacacatgaccctggagccctgggaatctggccagcgtgcatcgtggtgcactgattctgcagcatgcaggctgaggtccacagcagtgtgggaaactatgtgcaatttgtgaccagtgatgacttgaaagcttagctgtctgtgtgagggtgagatttgaagca

SEQ ID NO: 32 - protein, 362 aa, Mus musculus
MENGLCSIQARELDEFICDYLFPDTTFLTELRADIDSISAFLKERCFQGAAHPVRVSRVVMGGSYDEHTALKGKSEAKMVLFFNNLTSFEEQLKRRGEFVEEIQKHLCQLQQEKPFKVKFEVQSSEEPNSRSLSFKLSSPELQQEVEFDVQPAYDVLYELRNNTYAEPQFYNKVYAQLIHECTTLEKEGDFSICFTDLHQNFMRYRAPKLWNLIRLVKHWYQLCKEKAEEPLPPQYALELLTVYVWEHSNKNQEKVTTAKNFRTFLELVAYYKNLRIYWTWYYDFRHQEVCAYLCRQLKKARPLILDPADPTRNVAGSDLQAWDLLAKEAQTWMQSSCFRNCDMSFVPTWDLSPERQECAFQ

SEQ ID NO: 33 - DNA, 4380 bp, Homo sapiens
  CDS (29)..(2677)
ccttctgcgttcccagcgcgcggcccgaatggcggccgacaagggcccggcggctggacctcggtcgcgagctgccatggcccagtggaggaagaagaaagggctccggaagcgccgaggcgcggcctcccaggcccgcggcagcgactcggaggacggcgagtttgagatccaggcggaagatgacgcccgggcccggaagctgggacctggaagacccctgcccaccttccccacctcggaatgcacctcggatgtggagccggacacccgggagatggtgcgtgcccagaacaagaagaagaagaagtctggaggcttccagtccatgggcctgagctacccggtgttcaaaggcatcatgaagaaggggtacaaggtgccaacacccatccagaggaagaccatcccggtgatcttggatggcaaggacgtggtggccatggcccggacgggcagtggcaagacagcctgcttcctcctcccaatgttcgagcggctcaagacccacagtgcccagaccggggcccgcgccctcatcctctcgccgacccgagagctggccctgcagaccctgaagttcactaaggagctaggcaagttcactggcctcaagactgccctgatcctgggtggagacaggatggaagaccagtttgcagccctgcacgaaaatcccgacataattattgccacgcccggacggttggtgcatgtggctgtggaaatgagcctgaagctgcagagtgtggaatacgtggtgttcgatgaagctgaccggctttttgaaatgggtttcgcagagcagctgcaggagatcatcgcccgcctccccgggggccaccagacggtgctgttctccgccacgctgcccaaactgctggtggaatttgcccgggctggcctcacggagcccgtgctcatccggcttgacgtggataccaagctcaacgagcagctgaagacctccttcttcctcgtgcgggaggacaccaaggctgccgtgctgctccacctgctgcacaacgtggtgcggccccaggaccagaccgtggtgtttgtggccacgaagcaccacgccgagtacctcactgagctgctgacgacccagcgggtgagctgcgcccacatctacagtgccctagacccgacagcccgcaagatcaatctcgccaaattcacgcttggcaagtgctccactctcattgtgactgacctggccgcccgaggcctggacatcccgctgctggacaatgtcatcaactacagcttccccgccaagggcaaactcttcctgcaccgcgtgggccgtgtggctcgggctggccgaagtggcacagcctactccttggtggcccctgatgaaatcccctacctgctggatctgcacctgttcctgggccgctccctcaccctcgcccgacccctcaaggagccctcaggtgtggccggtgtggatggcatgctgggtcgggtgccacagagtgtggtggacgaggaggacagtggtctgcagagcaccctggaggcatcgctggagctacggggcctggcccgcgttgctgataacgcccagcagcagtatgtgcgctcacgcccggcgccctcgcctgagtccatcaagagggccaaggagatggaccttgtggggctgggcctgcaccccctcttcagctcgcgttttgaggaggaggagctgcagcggctgaggctggtggacagcataaagaactaccgctcccgggcgactatctttgagatcaacgcctccagccgagacctgtgcagccaggtgatgcgcgccaagcggcagaaggaccgcaaggccatcgcccgcttccagcagggacagcaggggcggcaggagcagcaggagggcccagtgggcccagccccgagccgcccagcactgcaggagaagcagcctgagaaggaggaggaggaggaggcgggagagagtgtggaggacattttctcagaggtcgtgggccggaagcggcagcggtcaggacccaacaggggagccaagaggcggagggaggaggcccggcagcgggaccaggaattctacatcccctaccggcccaaggactttgacagcgagcggggcctgagcatcagcggggaagggggagcctttgagcagcaggcagctggcgctgtcctggacttgatgggggatgaagcccagaacctgacgaggggccggcagcagctcaagtgggaccgtaagaagaagcggtttgtgggacagtcaggacaggaagacaagaagaagattaagacagagagcggccgctacatcagcagctcctacaagcgagacctgtatcagaagtggaaacagaaacagaaaattgatgatcgtgactcggacgaagaaggggcatctgaccggcgaggcccagagcgaagaggtgggaagcgagaccgtggccaagcaggtgcatcccggccccacgccccaggcacccctgcaggccgagtccgcccggaactcaagaccaagcagcagatcctgaagcagcggcgccgggcccagaagctgcacttcctgcagcgtggtggcctcaagcagctctctgcccgcaaccgccgccgcgtccaggagctgcagcagggcgccttcggccggggtgcccgctccaagaagggcaagatgcggaagaggatgtgaggaccaggacccagccccgtggctccttgattggccttagggtgggcatcagcagacgttcccgtgcaccactgtgtgcctggccctgtgctgggcactgggggcactccctgcaggagccatcatctgtgaaaaggagcactgtatggccacagaagggcagcagctgcgtcagcctaagacagagacatttgaacagggccttgaagggtgtgcaggagttcgccagcaaagccaggcaggccaagacttgagttggcaactcagctgctgctgcttccatgtgttctgggttcagaggtcatggctgcaccggtcagagccctgagtgcctcagggtttggcaatggaatttttaatgtaataaatctttattgagcactgctggtggccaggagtgcggtctacttggggaactggaatggagagacccaggtactaaaatcccagctaacgtggcagaggagttgcgggtctcctgagggtgagttctgctgccttgtccatttagcgatgaggaaagtgaagctcagagcacaaaccaggtgccagaggcgggagttggtcccccttcctcccactggacatggttgcagctgggagtgggctgggggaggggaacaggatgcccagcccagggggcaaggacacagctgctcccctctggctatgaagaggttaacgcggcccctccacacctggaggtcagaacctggcctgtcctctgtcttcttgccacccaccccctgtttgaggttctgagaaggtcaagggcagccccagcagctggattctcaggctgggcccctcacctggcagagtccatagtggagggggccttggtgatctctcatctagcatggaccctgttctgagacctgacaaagagttttttttcatgccccaaccaccctggcagggagggcctggtctgatctcattttagaggcagttgccccacacatagccccttgaccttcccatcacctcctcatcagggcctgcatttatggagtgcttgctatgtgcccctcatggcaggcccacagcactctgaacaggcacagccctcccatcttcccaatgaggaaacggattcaggaagagccactcagtccacaccccattggaaatgtgggccctcctatctcagggtctctcaggggttccctctcccgctgctcacctgtgtcagggctaggaggcaggggctgcaggctcagcctgacccaggccggccagcatggcagagaggtcctgcatgaacagcttcacctgggggggcccagcacgaagtcacatcccagccccagagtcactgtccagcccccacccctcaacacgcgggggaggccgaaaggtagggcggggactggagatcccctcattaaaagaacagtgatgatggtggttcccagaggtggtgactgagatcctaaaccgttctgggttttgaaagcctcaggccaaccttcccaactgctgcgtgagcagacaccttcacagcttcctcgctgctgtcacctgcactatccaattagtattttcatttacatcaatcagctttattttcttgtaactggatcagtcatattcattggtttgtgacctactcttatctccgtgggtggctctccttttgttttaattaacttctttatgaatatgaacttaataaataccatggatccattgtaaaaact

SEQ ID NO: 34 - protein, 882 aa, Homo sapiens
MAADKGPAAGPRSRAAMAQWRKKKGLRKRRGAASQARGSDSEDGEFEIQAEDDARARKLGPGRPLPTFPTSECTSDVEPDTREMVRAQNKKKKKSGGFQSMGLSYPVFKGIMKKGYKVPTPIQRKTIPVILDGKDVVAMARTGSGKTACFLLPMFERLKTHSAQTGARALILSPTRELALQTLKFTKELGKFTGLKTALILGGDRMEDQFAALHENPDIIIATPGRLVHVAVEMSLKLQSVEYVVFDEADRLFEMGFAEQLQEIIARLPGGHQTVLFSATLPKLLVEFARAGLTEPVLIRLDVDTKLNEQLKTSFFLVREDTKAAVLLHLLHNVVRPQDQTVVFVATKHHAEYLTELLTTQRVSCAHIYSALDPTARKINLAKFTLGKCSTLIVTDLAARGLDIPLLDNVINYSFPAKGKLFLHRVGRVARAGRSGTAYSLVAPDEIPYLLDLHLFLGRSLTLARPLKEPSGVAGVDGMLGRVPQSVVDEEDSGLQSTLEASLELRGLARVADNAQQQYVRSRPAPSPESIKRAKEMDLVGLGLHPLFSSRFEEEELQRLRLVDSIKNYRSRATIFEINASSRDLCSQVMRAKRQKDRKAIARFQQGQQGRQEQQEGPVGPAPSRPALQEKQPEKEEEEEAGESVEDIFSEVVGRKRQRSGPNRGAKRRREEARQRDQEFYIPYRPKDFDSERGLSISGEGGAFEQQAAGAVLDLMGDEAQNLTRGRQQLKWDRKKKRFVGQSGQEDKKKIKTESGRYISSSYKRDLYQKWKQKQKIDDRDSDEEGASDRRGPERRGGKRDRGQAGASRPHAPGTPAGRVRPELKTKQQILKQRRRAQKLHFLQRGGLKQLSARNRRRVQELQQGAFGRGARSKKGKMRKRM

SEQ ID NO: 35 - DNA, 1993 bp, Mus musculus
  CDS (37)..(1140)
gccaggctgggagacccaggaagctccagacttagcatggagcacggactcaggagcatcccagcctggacgctggacaagttcatagaggattacctccttcccgacaccacctttggtgctgatgtcaaatcagccgtcaatgtcgtgtgtgatttcctgaaggagagatgcttccaaggtgctgcccacccagtgagggtctccaaggtggtgaagggtggctcctcaggcaaaggcaccacactcaagggcaggtcagacgctgacctggtggtgttccttaacaatctcaccagctttgaggatcagttaaaccgacggggagagttcatcaaggaaattaagaaacagctgtacgaggttcagcatgagagacgttttagagtcaagtttgaggtccagagttcatggtggcccaacgcccggtctctgagcttcaagctgagcgccccccatctgcatcaggaggtggagtttgatgtgcttccagcctttgatgtcctgggtcatggtagtatcaataagaagcctaatcccttaatctacaccatcctcatctgggaatgtacctccctggggaaggatggcgagttctctacctgcttcacggagctccagcggaacttcctgaagcagcgcccaaccaagctgaagagtctcatccgcctggtcaaacactggtaccaactgtgtaaggagaagctggggaagccactgcccccacagtatgccctggagctactcactgtctatgcctgggaacaggggaatggatgtaatgagttcaacacagcccagggcttccggaccgtcttggaactggtcatcaattatcagcatcttcgaatctactggacaaagtattatgactttcaacacaaggaggtctccaaatacctgcacagacagctcagaaaagccaggcctgtgatcctggacccagctgacccgacagggaatgtggctggtgggaacccagagggctggaggcggttggctgaagaggctgatgtgtggctgtggtacccatgttttatgaaaaatgatggttcccgagtgagctcctgggatgtgccgacggtggttcctgtaccttttgagcaggtggaggagaactggacatgtatcctgctgtgagcacagcagcacctgcccaggagactgctggtcaggggcatttgctgctctgctgcaggcccatgacccagtgagggagggccccacctggcatcagactccgtgcttctgatgcctgccagccatgtttgactcctgtccaatcacagccagccttcctcaacagattcagaaggagaggaaagaacacacgcttggtgtccatctgtccacctgttggaaggttctgtctgacaaagtctgatcaacaataaaccacagcaggtgccgtcatggtgtgtgaactctgaggagtgggccatacaagaacagtgcaggtgtgtgagcgtgtgtgtgcccatgcacatgcgtgtgtgtcttcacggttcaactagatgcatttagtgagcacttactacatatgctacatgattcagatgttcagcagtggttagagcaaagcctaactgctaggctttttgatgcaagttggattgggatccttccaggtctcttcttacacatacacacaagagaggaacccttggtttcttctgcccatgaccccaagacaagattctagccctgccctatctgacacagaaacagttccctggccacacatggacatggaacactgagactgtggcctgtgctctcagggtgcccttgagtggctacaacatgcaggctgggggcccataggtatgatgaaaataaaaggtacctggaatttttgacacatgtaactttgaaacagggtcattggtagcaacgatcagctttatcacatttagttaaatcacaatgattgtggtttcctttctgagacatgaatttgatgtgacacacgctgtcgtggaactcacaggaa

SEQ ID NO: 36 - protein, 367 aa, Mus musculus
MEHGLRSIPAWTLDKFIEDYLLPDTTFGADVKSAVNVVCDFLKERCFQGAAHPVRVSKVVKGGSSGKGTTLKGRSDADLVVFLNNLTSFEDQLNRRGEFIKEIKKQLYEVQHERRFRVKFEVQSSWWPNARSLSFKLSAPHLHQEVEFDVLPAFDVLGHGSINKKPNPLIYTILIWECTSLGKDGEFSTCFTELQRNFLKQRPTKLKSLIRLVKHWYQLCKEKLGKPLPPQYALELLTVYAWEQGNGCNEFNTAQGFRTVLELVINYQHLRIYWTKYYDFQHKEVSKYLHRQLRKARPVILDPADPTGNVAGGNPEGWRRLAEEADVWLWYPCFMKNDGSRVSSWDVPTVVPVPFEQVEENWTCILL

SEQ ID NO: 37 - DNA, 1476 bp, Mus musculus
  CDS (26)..(1120)
tcagcaaacacttcctggccataaaatggtgaaggatcttagcagcaccccagcctgtgagctggacaagttcatacgtgatcatctccttcccgattccagcttccatgctgaggccagagcagacgtggacttcataggtgctttcctgaaggagagatgcttccaaggtgccacccaccctgtgagggtctccagggttgtgatgggcggctcctacgacgaacacactgcactcaagagcaagtcagaggctaaaatggtggtgttccttaacaatctcaccagcttcgaggagcagttaaagcgacggggagagttcattgaggaaattcggaaacacctgtgtcagctgcaggatgagaaaccatttaaagtgaagtttgaggtgcagagctcagaggagcccaactccaggtctctgagcttcaagctgagctcccctgagctccagcaggaggtggaatttgatgtgcagccagcctatgatgtcctgtatgaactgagaaacaacaagtatgctgaactctacttgtacaacaaaatctacgcccaactcatccatgagtgcaccacactaaagaaggagggcgagttctccatctgcttcaccgacctccatcagagcttcctggaggatcgtgcacccaagctgaagaacctcatccgtttggtcaagcactggtatcaactgtgtaaggagaagctggggaagccgctgcccccacagtatgccctggagctgctcacagtgtacgcctgggaaagtgggagtagagactgcgaattcaacacagcccagggcttccgaactgtcttggaactggtcaccaagtacaagtggcttcgaatctactggacagtgtattatgactttagaaagacgaaggtctctgaatacctgcacaaaatgctccaaaaagtcaggcctgtgatcctggaccctgctgacccaacaaggaacgtggctggtaccaacctactaggctgggggctgttggcaaaagaagctgccatctggctgcagtcctcctgctttaggaactgtgatacgtgcctcgtgggcccctggggtgtgccggtgaaggtcgagattccacaggactgtgtccttctatgagcaccaaagcacctgccaggatgctcaagagtcagggatatgagatccttgctctgctgcaggcccttgaaccagagaagggggaaagctgctcacggccccaatcagggagggtccaacctgtgatcagactccaggcttctgacccctgccttctcacccctgcatccggtcctatcacagatagccttcttcgaagcctgctttatctgccttatccaccaacagtgtcctccgggagatgagagattcagaattcagaaggggaggcaggaactcaagcttgacttccacctgtccacctgttgggaggttctgtccaatgtctgatgcacaataataaatcacagagagcc

SEQ ID NO: 38 - protein, 364 aa, Mus musculus
MVKDLSSTPACELDKFIRDHLLPDSSFHAEARADVDFIGAFLKERCFQGATHPVRVSRVVMGGSYDEHTALKSKSEAKMVVFLNNLTSFEEQLKRRGEFIEEIRKHLCQLQDEKPFKVKFEVQSSEEPNSRSLSFKLSSPELQQEVEFDVQPAYDVLYELRNNKYAELYLYNKIYAQLIHECTTLKKEGEFSICFTDLHQSFLEDRAPKLKNLIRLVKHWYQLCKEKLGKPLPPQYALELLTVYAWESGSRDCEFNTAQGFRTVLELVTKYKWLRIYWTVYYDFRKTKVSEYLHKMLQKVRPVILDPADPTRNVAGTNLLGWGLLAKEAAIWLQSSCFRNCDTCLVGPWGVPVKVEIPQDCVLL

SEQ ID NO: 39 - DNA, 13943 bp, Mus musculus
  CDS (1514)..(1693)
  CDS (3240)..(3516)
  CDS (6428)..(6612)
  CDS (10018)..(10250)
  CDS (10901)..(11054)
  CDS (12252)..(12350)
catcctttttttttttttccattcatttcttcatctgttttagggttttttccccctcacactctggagtgaatccagctgtagtgtagcccaggatggaccagcctccactatccatcctcctgcatcagcctctgcctgctgaactgctagggccacccacaatgctgtttttcttccctgttttcctgcatgtgaagggctaatgacctagaagccccaggcagtctgagtgactccctgcaggaggagatatggtgctgatgtcagtgtgcagtgaggatcagggaggccaacggggtaagcaccccaccaaaatccaccagcttggggaagcaggactaggtctgcagacccccaaaggtcccatttccaccctaatcccctggagctccctgtgcccggtgagcagtctccactagcaatggcttccttcctggagatgcataaaggccaatcctgctcttgcagaaggccagactctttatttcctcatcgaagtacatcctggttaatgctcgggtgtgcacatgtgagtatacacttgtttgtgtatttgtgtgcatatgtgcacaggcaagctcaggagtgtatttagtgtgtgcatgccctgtgcgtgtgcatgtgaatgtgtgtgtgtgtgtgtgtgtgtgtgtgtttgggacctgccatgtcagttctcagaattggaactggaagcacagcccgagctcttaaactctgagccctctctctgccctctaaacaagagatgagaagatggaggcaggggttggagggcatcaaactgttttgatgaggccttgggtatgtcacccaaaggtgattttcaaccttgccagggccctgaaagacagctcattttaatagtcactgctgacctgtactgtccccagcttggtggtacggcctgtacttcacccttggggatgcccagaccccaccctcacccagaccccctgcaccacaggggaaaatgtagggtgtgagtaggagagggggcttctgggaagtctgagctttctgttagaattgtctgtaatcctagacctgcaagtccagaggtaaaggaaaaacaaaacctggggaggtgctgttcttaaaaagaaaaaaaaaaaaaaaaagcagttccgcctaaaacgtttgcaggagatggaagccgagctcaggagccaccagccaccttctgtggtttcctgattccgtttcccttcttctacgtgcacagattgttcctaaaacgtaccgtgtaacagccttttttccagaagaaatcccgagaaagccaggtcagtttccttacacattcttccctgggccggatcttaagaaagctcaggcttggatggggaggtacctgttcagaagccctaacgccattggctgctcgggcctggatgatttgcatatccgcgcccttcccgggaaatggaaactgaaagtcccatttctgcttcagcgagcctaggagacacaggacctgctggctgcagaggtattagctggacctaggatggagcaggatctgaggagcatcccggcctcgaagcttgataagttcatagagaaccatctcccggacaccagcttctgtgctgacctcagagaagtcatagatgccctgtgtgctctcctgaaggacagatccttccggggccccgtccgccgaatgagggcctctaaaggggtcaaggtgagccttcctcagcctgagctggccgagatgaggtgggacaggactttcagaagccaggctgcaaccctgatccctcctcttaattctgatcacagctggcgatgggttcttccccccaagtcccacatctgtattggagaaggagcctcagctacagtttatgttccccactcccaggccatgtccatttcagagtcggggaaactgaggcccagaatggcaaagcagttttctggaaagtggaggggcaggtggtggggcagtggtaatacaacctttgcactgctgtgtgacttatggtaacttaactaccttctccatgtccagtgttccacgccccaggaatgataaggtcacatagactaagtcaaatgtgacacggaggacagaccaggactcgcctgctatgtgactggattgatggaccctctctggactcggttcccgggctgtctgtcaaatcacatttcatgacactggtacttaactctcagactctgtgaagggtgcagaaatgaagttgttttcttgttttcagttgactgtatttctacatttttagttgtgagtggggctctgtatgtgttagtgcaggtttggtcagaattgaaaagggggtgtcagatcccttagagctgcagttacataggtggctctgagctggcctgcttgggtgctgggaactaaactgaggtcatcagcaggaacagcaggcagttttagctactgacccacctctccatccggagatgtgacaatttgtaatgccctctctcaacagtagctgatgcttatgtgtgttagtgatgattctaagcattttatttagggaggctttgttcagcatgaattcttctttttatgatatttgttttacagacttggatcacctttattgtttttttcagtttctctatgatgttatatgttattctatatcacacaaggccatcttctctttaatgatgtcacttgcgcatatttcctcccagctaacaatgccaaccatgggactatggctacattctcatatatctgtattaatttagtcatagtctccttccaatctctactatcctttacctcccaccagaccctccccagagattcactctctgatggtatgtcacatgtgtgcagctaatgtgggcactgcatgagtgctactgtacccttcccaaactcttctgtgcttgtttcatttagacctctcccccactcccatacacagcccccccccacacacacacacacttgaacacacagagactccacagatgaacatggatattttccttcacggtgtggtttatcttctgtatcaagaggagatccagcaccctctattttcctacatagattctgatgtcactcttcagggctgactaatattctcctgcacatctagcagcttgctccatccatccaaccatccatccatccatccatccatctatcaccttggtgatggactccagttcagcatctggacctcccagcatggctctccctgtcctttgtcattctctttcttgtctttcagggcaaaggcaccacactcaagggcaggtcagacgctgacctggtggtgttccttaacaatctcaccagctttgaggatcagttaaaccaacagggagtgttgattaaggaaattaagaaacagctgtgcgaggttcagcatgagagacgttgtggagtgaagtttgaggtccacagtttaaggagtcccaactcccgggctctgagcttcaagctgagcgcccccgacctgctgaaggaggtgaagtttgatgtgctgccagcctatgatttactgggtaaggcagcctgccagagagcctcagctcacccttctgcatgccttcatcctcccttctagttccacctctgtgtgtgtgcacatgtgtgttgcgtaggtgttctcttgtgtgtgggtgtgatccggaaccatggctaactcatctcaagagtcatccatcttttttgagaccttgtctctccctggttcagaattcacacttgagcctgactggctgggaagagccccagggaccctcagagtctcccccttctcagttctgagattacaatcctacaccaccaacactggctttttctctgagttctgggattccgacgggggtcctcatgcttgcgagccaagcacgttacagactgagctatctccccagcccacaccttccatccctgaactgaaaaccattatcatttgagaaattcaactaaagagaatgtctctttaaagctgcgctggggaggagggattttggaaatttggtagcggggagacagaatccatcccacagggacattgagcagcctgctggctcacttaggggagggagtcacactagatgctccctcagcataccagtcctactcacggctagctgtgggttttgtcttcttaaacatgagacagtgtagacttcactgaaaataacaaactcgcgaattttctccctcagagctgttcagagacctggggtgtctgcaggagagtttgcacaggacgcagatctcatcggagctctggcccctcagtaaaactgcagatgcagacacccaactcacacatgcacacacattttataacattacacattaaaagatcttatcttttgacaaaaatctcaattattcaattttaccaatgaagacttgcaaagccagatactgtggtaaaatcctgctagctcagggaggcagagaaagcacctagctgaccttcctcctgagccagaatgttccagaatgcatccccttctcatgctatctcataacctctttcaaatggaatgtccctcccctctacttcctgtgggtctctctatccctccttctgactccctcttactctctgtggttccttcttagtaatcccatgttcgcttctggtcagctggttgcttgctctgcctcttgatctatgattggctttatttaatcctgtttacaatattcaagcagaaagctcctggattaagggtgtgtgctaaggctgagccaccacacctaaaaccagttttttccaataaacaactcaatctcagggttcacagtgtgatcaaatatcctgcaacaatcttttaccaggcctgatacagcatgcgtcgccaaaattacagattgtccaactcaggaaacactcctgagagcacagtgggatcttgggggagcttagagaatacttgaggctttacatttgggggaagcacagcaccccctgttggccactgtgtgaacttctgctgttaacttagtgacaaggccactagcccttggcttaggagagccgtggaaacagtccagagtctcaggcagcagggactgagagggccagcctgggagtaggaccagggcagcctgctgtgggaagctgcaagaggcttagaaccttgttcagtggagggggatcagagtgggtgaccagaaatgatctgtgacagcaggtcccacagaagcctgtctgggtcatggatttcctcagcctcagcagctttccagtggtgaggggctctaccacagaaggactgcctgtgggcaacagccaaagagaggatcatagccacagccctatagaagatgacataaagcaatgagccctattgtaactagaccctgacactaatctagactagcccataacctccatggtcactgagtgacaggcttgtcgaagctccatccgtgagtgactttgccctgtgtcagcatcacagtctgatggtgtggtctgctgcatggcacacatgtgcacacacctaaggcacagcctgcctagactactcaaatgtgcacacacacctatacacacatctattccatagtctgctgctcttctactctataagtgcacaggacacacacacacaaacacacacacaccctaacactgccatatcttgttccatctggacaaacaaaaccacatgatattaacttatcactacaagaaatcacccaccgaaaacacacagtggctggggagacagctcagtggataagggcgattgctgctcaagcatgagtacctgagttcaaatccccaatatccacctaaacttaggaagtgccacacattgacctggatacctggtcactatgggggcaaaagacaggagattctgtaagacctgcaggattccagccaagctccacatttacagacagactctgacgcaaagaaacatactgacaaactataaagaagagacaccttaggtcctctcctgtctctgaacatatcaggaacactcgccatgcatacacaatatagagcagagactctgtcagtgtgagacgtgatatgtgaatgcagccgactgctccacagtgaactttgacacatacatgagtgcacaactttgacaagaactatgcagaagatactggaaccagacgtggttggccattatctgatgatcacgtgtgtgtgaccacagtgtgctggttcctcccacctctgtctccaggcctgcaagaaagaccctcactagagcaaactgggagcagggactttgtagtgcacagcatgtgtgcagtcctaaggagtggtggggcagatgggtatatgcggagcgatgcctgctggggaaaaacgctgcctgttcgctgaaccccataggctgtgattctggacctggggagactgaggaacctcagagaggggctgagctgttccactgaatgaaatcttgtttacagatcatcttaacatcctcaagaagcctaaccaacaattctacgccaatctcatcagtgggcgtaccccgccggggaaggagggcaagttatcgatctgctttatggggcttcggaagtacttcctgaactgtcgcccaaccaagctgaagcgcctcatccgcctggtcacgcactggtaccaactggtaaggcattggcctggtcactgtgcgcttcatctgtacagagtgtagacagggggcagagaaaatgtgtactctagttcggcgtctattgaggtaataactctatcacatgaccaaaacgctcctgggaaggaagggtattcttatgtttacacatctaaggcacactccatcattgagggaagtcagaggaataactgaagcagaggccatggaggaattctgctcaccgtttgctgtccatgacgcactcagcggccatttctacacagcccaaggccacattccattcatggctccaaccacagtggtctggtcccacccacatcaatcagtgattagtaacatgcccataaactacccaagccagtcagatggatctgcattttactgagcaagatggactcctaccagaggaggctagctggtgtttagtggacaaaaagtaagcagcacaattgaccccttttcaacatggcacacaaacaaaccacagttgaaccataacctttcccttcttgcttgtccctaagatctcatgtcgctatcacaccatagaacacaacttaggtttaaagtccccaaattaaaaatcaaacctaaagaaactcccaacacataaaaagactagtctctcaatgctggcctcatataaaattaaaacacaagttaaatactttcttactgcaagagggaagaaccaagtcactgtcacaatctaattaaaacacaaccaaaattcaacagaacaaaaggctcagtatctgatatctgggactcatagttctctgagctttttgggctctgccatccacagcacacacagcaggtctcaggctcaggccagctttgttctcacagtgctctgttcctgacagtcatctcctgctcagggcaaggccagtatcttggggtttccactgccattgaggctgcactttcactaatggcttctcctggcctctcgcagtgccaggcctcagctgctctccaaggccccttcagtccagcagttctcatgggacttgaaatctgggagacatctctgcagttccaggtgtgattgacacttggagatgcagacttgggacccctggactacagctgatagagtccatggtctgccaaagaatgatgtcctgcactcaaattgtatgcaaaagcaaagagcgtttttattctgtggagtccagcatgctgggatctatcattaaccaagatggagatgcccagatgagatcacaggctcaatttaaagcacattagtggaattccatggaagagtaggtgacctttttcttgattggttggctctattctagggacattcctgaatcattactggggggctagaaactgtcacttggggaagcctggaaactgttgctgactctgttctttccctaggccaggtggcagggaagcttctgatggctgggcagtttctgattggatgctctaggcctggggtttttccaataaatgacttgcctggacttgtacagttctgagaaacacaaattcaggcctagtctcctgatatgccagtttgaagcccattatggaatcaccctggctcagtccacttacacctactcttgctgactatgaagaaactctttccaaaagatttgcacccccccccccacacacacccatggtgcaggtttcttctttatcagagcagattttcagccccaactgatgagaaattactgactcttaatgtaaacacagcacacaaacagcccagatgggtttttgcttttctctgaaacttcactccagcctctgtcacctacctgcctctcagcactcctgtcttccaagttcccacaaacagcctggtaggctctcagctctcaacagctctcccacccaaagttccataatcctttctcaatcctcccaaggagcaacatggcaggtcagtcacagcagcgcccctcctccctaggaccagtttctgtcctagtttgctctgactgctgtggtaaagatgctgcaaagtaaattgaggtggaaatggattgtctggctcacgttcctgcatcacactccatcactgagggaagcagaggcaggaactgaagctgaggccatggaggaactctgctagctgccggagacccttgggcgatcagtctgatttttacaaacaactcaggaacacctgcccagggcaggcaatgcccacaatgggcggggcccttctacactgatcaaggaaatgccccacaggcttgactccaggccaggctgatggaggcattttctcagttgtgggtccatcttctccagatgattctgtgtcactttggcaaaaagaatcaaaattagcaaaagcagtgagaaaaacaatgtaaaaaaaaaaaaaaagtgggtggaggggaatcagaggggaggggatgagagagaagcaggggtgagtggagatggggggagatgatgctgtaggaggggcaaggagtggggtgggggatctgagaggagggaggattttccttgcctgttctaggtgatttcttctagtccactttccaatccgctaactctctcttcagctgtgtctgattcactgttccatggcttctgcttcatttgtctttggatttttctcccattgttggcaaatccttcatgtcacttttcatactttgattttcctgtcttgtcaagctatgtttttaaatgtgtgatttcttattatatcctgagtgtttttatgtgtatgtgtttatacctattcttggaaacacacgtgtatgtgtaggcatgtgtgtccattaggatttgagggcagagtgtgagagcaggttctgagtcacgccccaccttgtgtaatcagtcagggtttcacacgtgacactccgtgtgctagattagcagttctggacagtcatacagcccgggggagccccatctttatccccttgaacagggatgaggagtcagcttcctcaagagcctgacaacacatagatgctggggatctgacctccagttctcatccctgcatggggagggaggcgctttacctctgagccctttatctccctggccccttcccagttacttcagagaccacaagcgaacttctacagtgagtactccttatgtcctgccccaccctcactcctagctgtcactcatggtgtcctgattcctcctgggcctgttagttacgtgctcagtctgtggagcacacagtgtccatctcaaccgctcagctcttggctatagcacaagaaggcatagactgaattcaagtgactgggtgtgacagtgtgacaataaccctgtcatgacatcatcaggtggcagctgaattgagcctgtgagaagcagccccacacacaaccaccaggaaccccgttttttttctcattgcctttctcttctcagtgtaaggagaagctgggggacccgctgcccccacagtatgccctggagctgctcacagtctatgcctgggagtatgggagtcgagtaactaaattcaacacagcccagggcttccgaaccgtcttggaactggtcaccaagtacaaacagcttcgaatctactggacagtgtattatgactttcgacatcaagaggtctctgaatacctgcaccaacagctcaaaaaagacaggtagtctgtccccacatgcctgtgatccccgtggagtgtcaggctgcacttgtgtttaaagggggaggtgggagctctgtgtctatgcagcatcccctgtactgagcactgtcctgctgtcacctgattgacatgaggtcctacagccctgggaggcaggggtgggctctcctgtctgcagatgaggagaccaaggctctgacattgtcccaacccaatcagcaaaggaaaggggggggggacatggagtggggactgagattttccaacagtgacagcatcataaaggctgtgaggcaccaaagacctgaggtggacaaattacctgtaaataatacagtgctatgtcaccctagggagtgacatgtcttcattctgtgttgtgttcagtttccacaccctattctttgagcctggctggtccaacactcttcatgtcacagatcagagaccctgagggatctgtcccaggacacacagctactaaatcatgcccaaggatccctccaggcttctatatgctgtggggcagggacaaaaaaaaaatctttgaaaaggagctctctgctgggctcctcccccacccctgtgcagtggttcctgggtcagttgacagcagctcgtttccctctcttcatgttgctctgcaggcctgtgatcttggaccccgctgacccaacaaggaacatagctggtttgaacccaaaggactggcggcgtctagcaggagaggctgccacctggctgcaatacccatgctttaagtacagggacggttccccagtgtgctcctgggaggtgccggtgagaacgtgtcaccatcatcttcattccacaccccccccccttctcttcatctacctggagaatcttcccacacagatgtggtctccgcactcctgtggaggccattggctctccttattccacagccatcttagcgctattcacagatttcatggactcacaggatggctcagtgggtaaaggtgcttgctgccaacacagatgacctgggtttcatcccaggatctcctatggtagaaggagagaatcaaatccccaaagttatcctctgaccttcatctctgctgttacacacagggacatgagtgtgtgcatacacacacatacacatacacacacacatgtcactacacccacccacccacctatccacaggttgcccccaaccaccaccacatagacagacacacacaaaaatacacaaacacagaggttgcccacacccattcacacaaagacacacacacagacacacacacacagacacacctacacacacacacaccatcatcatcatcatcatcatcatcatccacaaatagataacacacatacatagacttggagacctgaagccactggctcggggacactcagatagagtgaagaacggctgggaccccgtggactctgaagcccctgtcaggtccatgaagcagcctgtcttccctgtctctatcatgtggccaatgggactcagctgggctctatcacagcacagaggcacagagggacctgcacttggacctttctcctcctggggcagggcctgcctgatgctagggacccagtgatgccatgtgtgccctccagatggcgtttagcatggctgtagtaatgctggaaccataccccagggcagaatgggagtttgatgggtgtggggatggtgatttgggaggtgggaggggcgagtcagactgcagtggcctttagagctgtccctctggctatgcccactctcaattgatggatctgtcagtggatgagaggggacaagtgggaggcatggcttcagggaagagctgtatatatcctcaagggcctaggccctgtttagcctactgttgaccttctcagcagcctgaccaaggggcccaggtggcctgtcacagaactcatcttcagggctcagttgcatccccttcccagtaagaatgtaggctcctgacatgatcgtgttttctattacagacggaggttggagtgccaatgaagtatctcttttgtcgtattttctggttattgttttggtctttgtttcatttcatctttgggaagacttcatctggatagcccagagtgtcttggatattgccatcctcctgccttagcgctggcatgactgcagcgtaggcctgttatgctctgcctcccctccatcctcaagtggacaagaactgggcatgtgttttcctgtgagcccagtgggacctgtccaggaggctccagagtcaggggcatgtcctgctctgctacagggccttgacccagagaagacaggaaggtgcccaaagcccaagagagggagggtccaacctgtgatcagactccaggcttctgtcccctgtcctcaacccctgcacagacagcctttctcacagcatgctttatctgtcttgtcccccaacagtgttctctgggagacaagagattcagaaggagaatatgatggtttgtatatggttggcccagggaatggcactgttaggagatgtggccatgttggaatgggtgtggccttgtgggtgtgggctttctcttgtcttagctgcctggaagtcagtatgctgctagcagccttcaaatgaagatgtagaactctcagctcctcctgcaccatgcctgcctggacgttgccatgctcttgccttggtgataatggactgaacttctgaacctgtaagccaaccccaattaaatgttgtttttataaaaattgccttggtcatggtgtctgttcacagcagtaaaaccctaagacagggagggaggaactcaagcttgaattgcatatgttcgcacgtcgggaggttctgtatatcaataacaacaaaccacagcaagccatgagtgtgtgtgtatgtgtgtgggtgtgggtctctctttgtgtctgtacctgtgtgtgtataaggatgtgtgtgtgtatctctgtgtctgcatttgtgtataagaatgtgtgtgtatctgtgcatgtgtgtgtatgtggaattgtgtgtgtttgtatctctctgtgtctgcatgtgtgtgtaggagggtgtatgtgtgtgcatctgtgtatgtatatgtgtttgtctttctgtgtgtgtgtctctgtgtgtgtgtgtctgcagattcatggtcaagtagctgcacttattacattctccatgtgggtcccagcaccaagtcctcagtggtggacagagcagaaccaaactgatacaacttttgatgtcaggtagcctgggatccttccaggtctcttcttgcacataaacttgagacaggaattcttagttccttctgtgttcccaagcaacaataatttctcttgggaacaacccccttttagctatcttaaagcacatagtgtcttttcctggtctgttttgttttgctctctctttctggcttagagtcagatgaccgtttgatctgagatagagatttgaggagactaaacaggctgcttaggttgggagaggagggtcataatccaactccagagccagctttataataatgctgaacagcataaaataatgcttcagtgttattcatgcccaaaagacagcatagctgagtacctgcaaaatggactcaaggactgcagggagtgactagagcaaacagagatcaaaggacactcaaagatctgagatttagcaggacttaaacatcagg

SEQ ID NO: 40 - protein, 376 aa, Mus musculus; identical to SEQ ID NO: 10

SEQ ID NO: 41 - DNA, 1695 bp, Mus musculus
  CDS (29)..(1114)
gcagtcagcaaacactcctggcctcaggatggcgagggaactcttcagaaccccaatctggaggctggataagttcatagaggatcaactccttcctgacaccaccttccttactgagctcagagcagacatcgactccataagtgctttcctgatggagagatgcttccagggtgccgcccatcctgtgagggtctccagggttgtgatgggtggctgctacaatgaatacactgtgctcaagggcaggtctgaggccaacatggtggtgttccttatcaatctcacaagctttgaggatcagttcaacggacaggtagtgttcattgaggaaatttggagacacctactccagttgcagcaagagaaactatgtaaactcaagtttgaggtccagagcccaaaggagcccaactccaggtttctgagcttcaagctgagctgccccgagcgccagcatgagttggaatttgatgtgcagccagcctatgatgccctgtatgaagtaagacacttcaagccctttgactccagtaactacaacaaagtctacgcccaactcacccatgagtgcaccacactggagaaggagggcgagttctccatctgcttcaccgacctccatcagagcttcctgaggtatcgtgcgcccaagctctggaacctcatccgtttggtcaagcactggtatcaactgtgtaaggagaagctgagggggccgctgcctccacagtacgccctggagctgctcacagtctacgtctgggaatacgggatccacgaaaaccctggactccatacagcccagtgcttccgcactgtcttagaacttgtcaccaagtacaaacggcttcgaatctactggacatggtgttatgactttcaacacgagatctctgactacctgcagggacagatcaaaaaagccaggcctctgatcctggatccagcagacccaacaaggaatgtggctggttcagacttacaggcatgggacctgctggcaaaggaggctcagatctggatagattcgactttctttacgaaccatgatatgtccattgtggaagcctgggaagtgatgccagagagacaagaatgtgtcttcctgtgagcacccccagcatctgcctaggagactccagagtcaggggcatgtcctcctcttctgtaagaccttgacctagagaggacagacaggatggcactcaaggctccagcgaggggcatccaacctgtgatcagactccaggcttctgatccctgcctgcccatggacagccttcctcacaggctgcttcgtctgccttagcttccaacagtgttctctgggagtcagactgtgatggacagagaagaacgcaagctcgacttccatctgttcacctgttgggaggttatgtccaatagtggctgatcatcatcaacaaaccacagcaagccatgagggggggtgcactctgagggaggagtcctcagaccacacagaaacttttcagcagtgcatgtggccctggagccctgggaatctggccagtgttcatcaaggtgcactgtttctgcaacatgcaggctgggtttatggtagtgcaggaaaataaaattgcatgcattttaaaatttatgattttaaaacttaggggtgtgtgtgtatgagatttgaagcactaaattaaagcaaaacgcattgaatta

SEQ ID NO: 42 - protein, 361 aa, Mus musculus
MARELFRTPIWRLDKFIEDQLLPDTTFLTELRADIDSISAFLMERCFQGAAHPVRVSRVVMGGCYNEYTVLKGRSEANMVVFLINLTSFEDQFNGQVVFIEEIWRHLLQLQQEKLCKLKFEVQSPKEPNSRFLSFKLSCPERQHELEFDVQPAYDALYEVRHFKPFDSSNYNKVYAQLTHECTTLEKEGEFSICFTDLHQSFLRYRAPKLWNLIRLVKHWYQLCKEKLRGPLPPQYALELLTVYVWEYGIHENPGLHTAQCFRTVLELVTKYKRLRIYWTWCYDFQHEISDYLQGQIKKARPLILDPADPTRNVAGSDLQAWDLLAKEAQIWIDSTFFTNHDMSIVEAWEVMPERQECVFL

SEQ ID NO: 43 - DNA, 1336 bp, Mus musculus
  CDS (6)..(1166)
tcaggatggcgagggaactcttcagaaccccaatctggaggctggataacttcatagaggatcaactccttcctgacaccaccttccttactgagctcagagcagacatcgactccataagtgctttcctgaaggagagatgcttccaaggtgccacccaccctatgagggtctccagggttgtgatgggaggctcctatgatgaacacaatgcactcaagggaaggtcagaggccaacatggtggtgttctttaatgatctcaccagctttgaggaccagttaaagtggcagcaagtgttcattgaagaaattcggaaacacctgctccagttgcagcaagagaagccatgtaaactcaagtttgaggtgcagagctcagaggagcccaacaccaggtctctgaccttcaagctgtgctcccccgagcgccagcaggaggtggaatttgatgtgcagccagcctatgatgctctgtatgaagggggatactgcaagtcctttgaatccattaactacaacaaagtctacgcccaactcatccatgagtgcaccaccctggagaaggagggcgagttctccatctgcttcaccgaccttcatcagagcttcctgaggtatcgtgcgcccaagctctggaacctcatccgtctggtcaagcactggtatcaactgtgtaaggagaagctgaggggaccgctgcctccacagtatgccctggagctgctcacagtctacgtctgggaatttggggtccaagacagctttggactccatgcagcccagtgcttccgaacggtcttagaactggtcaccaagtacaaatgccttctaatctactggacatggttttatgactttcgacctgagatctctgactacctgcacggacagatcaaaaaagccaggcctctgatcctggatccggcagacccaacaaggaacgtggctggttcagacttacaggcatgggacctgctggcaaaggaggctcagacctggatacattcaaatttttttaggaactgtgatatgtcccttgtgaatggctgggaagtgtcgcttccaacagtgttctctgggagtcaggctgtgatggacagagaagaacgcaagctcgacttccatctgtccacctgttgggaggttctgtccaatagtggctgatcgtcatcatcaaatcacagcaagccatgggggagggtgcactctgagggagtcctcagaccacacagaaacttttcagcagtgcatgtggccctggcaccctgggaatctggccagtgttcatcaaggtgcactgtttctacaacatgcaggccgggtttatggcagtt

SEQ ID NO: 44 - protein, 386 aa, Mus musculus
MARELFRTPIWRLDNFIEDQLLPDTTFLTELRADIDSISAFLKERCFQGATHPMRVSRVVMGGSYDEHNALKGRSEANMVVFFNDLTSFEDQLKWQQVFIEEIRKHLLQLQQEKPCKLKFEVQSSEEPNTRSLTFKLCSPERQQEVEFDVQPAYDALYEGGYCKSFESINYNKVYAQLIHECTTLEKEGEFSICFTDLHQSFLRYRAPKLWNLIRLVKHWYQLCKEKLRGPLPPQYALELLTVYVWEFGVQDSFGLHAAQCFRTVLELVTKYKCLLIYWTWFYDFRPEISDYLHGQIKKARPLILDPADPTRNVAGSDLQAWDLLAKEAQTWIHSNFFRNCDMSLVNGWEVSLPTVFSGSQAVMDREERKLDFHLSTCWEVLSNSG

SEQ ID NO: 45 - DNA, 1559 bp, Mus musculus
  CDS (6)..(1076)
tcaggatggcgagggaactcttcagaaccccaatctggaggctggataacttcatagaggatcaactccttcctgacaccaccttccttactgagctcagagcagacatcgactccataagtgctttcctgaaggagagatgcttccaaggtgccacccaccctatgagggtctccagggttgtgatgggaggctcctatgatgaacacaatgcactcaagggaaggtcagaggccaacatggtggtgttctttaatgatctcaccagctttgaggaccagttaaagtggcagcaagtgttcattgaagaaattcggaaacacctgctccagttgcagcaagagaagccatgtaaactcaagtttgaggtgcagagctcagaggagcccaacaccaggtctctgaccttcaagctgtgctcccccgagcgccagcaggaggtggaatttgatgtgcagccagcctatgatgctctgtatgaagggggatactgcaagtcctttgaatccattaactacaacaaagtctacgcccaactcatccatgagtgcaccaccctggagaaggagggcgagttctccatctgcttcaccgaccttcatcagagcttcctgaggtatcgtgcgcccaagctctggaacctcatccgtctggtcaagcactggtatcaactgtgtaaggagaagctgaggggaccgctgcctccacagtatgccctggagctgctcacagtctacgtctgggaatttggggtccaagacagctttggactccatgcagcccagtgcttccgaacggtcttagaactggtcaccaagtacaaatgccttctaatctactggacatggttttatgactttcgacctgagatctctgactacctgcacggacagatcaaaaaagccaggcctctgatcctggatccggcagacccaacaaggaacgtggctggttcagacttacaggcatgggacctgctggcaaaggaggctcagacctggatacattcaaatttttttaggaactgtgatatgtcccttgtgaatggctgggaagtgtcgccagagaaacaataatgtgtcttccagtgagcagtgtagcacttgcctagaaggctccagagtcaggatcatgtgctcctccgctgtaagactttgacctagagaggacaggatggtgctcatgtctccagcgaggggtatccaacctgtgattagactccaggcttctgatccctgcctgcccatggatagccttcctcacaggctgcttcatctgccttagcttccaacagtgttctctgggagtcaggctgtgatggacagagaagaacgcaagctcgacttccatctgtccacctgttgggaggttctgtccaatagtggctgatcgtcatcatcaaatcacagcaagccatgggggagggtgcactctgagggagtcctcagaccacacagaaacttttcagcagtgcatgtggccctggcaccctgggaatctggccagtgttcatcaaggtgcactgtttctacaacatgcaggccgggtttatggcagtt

SEQ ID NO: 46 - protein, 356 aa, Mus musculus
MARELFRTPIWRLDNFIEDQLLPDTTFLTELRADIDSISAFLKERCFQGATHPMRVSRVVMGGSYDEHNALKGRSEANMVVFFNDLTSFEDQLKWQQVFIEEIRKHLLQLQQEKPCKLKFEVQSSEEPNTRSLTFKLCSPERQQEVEFDVQPAYDALYEGGYCKSFESINYNKVYAQLIHECTTLEKEGEFSICFTDLHQSFLRYRAPKLWNLIRLVKHWYQLCKEKLRGPLPPQYALELLTVYVWEFGVQDSFGLHAAQCFRTVLELVTKYKCLLIYWTWFYDFRPEISDYLHGQIKKARPLILDPADPTRNVAGSDLQAWDLLAKEAQTWIHSNFFRNCDMSLVNGWEVSPEKQ

SEQ ID NO: 47 - DNA, 2041 bp, Mus musculus
  CDS (9)..(1544)
gtgtagccatggcagtcgcccaggagctgtacggcttcccggcctccaagctggactcctttgtggctcagtggctgcagccaaccagagagtggaaagaagaggtcctggagaccgtgcagacagtggagcagttcctgaggcaggagaatttccgtgaagatcgtggcccggctcgggatgtgcgcgtgctcaaggtactcaaggtaggctgctttgggaatggcaccgtgctcaggagcactacagacgtggagctggtcgtgttcctgagctgtttccacagcttccaggaagaagccaagcaccatcaggctgtcctgagactgatacagaaaaggatgtactactgccaggagctgatggaccttgggctcagtaacctgagtgtgactaacagagtacccagtagtctcatcttcacgatccagaccagggagacctgggagaccatcactgtcaccgttgtgcccgcctacagagccctgggcccttcctgtcccagctccgaggtctacgcaaatctgatcaaggctaatgggtacccaggaaatttctctccatccttcagcgagctgcagcgaaacttcgtgaagcatcggccgacgaagctgaagagcctccttcggttggtcaaacactggtaccagcagtatgtgagagacaagtgcccccgggccaacctgccccctctctatgccctggagctgctcactgtctatgcctgggaagcgggcacccgggaggatgccaacttcaggctggatgaaggcctcgccacggtgatggagctgctccaggatcatgagctcctctgtatctactggaccaagcactacacgctgcagcacccggtcatcgaggcctgtgtcaggagacagctcaggggacaaaggcctatcatcctggacccagcagaccccaccaacaatgtggcagaaggctacagatgggacatagtggctcagcgggccaaccagtgtctgaaacaggactgttgctatgacaacagggacagccccgtccccagctggagggtgaagagagcacccgatatccaggtgaccgtgcaggagtgggggcactcggatttaaccttctgggtgaacccttatgaacccataaagaaactgaaagagaaaatccaactgagccagggctacttgggcctgcagcgtctgtcctttcaggagcccggcggagaacgtcagctcatcagaagccattgcacgctcgcctactacggaatcttctgcgacacccacatctgcctgctggacaccatctcccctgagatccaggtctttgtgaaaaacccggatggcaggagccacgcctatgcgatccacccgcttgattatgtcctgaacctgaagcagcagatagaagacaggcagggccttcgatgccaggagcagcgcctggagttccagggccacatcctggaggactggtttgactttaaatcctatggcatccaagacagtgtcacagtcatcctgtccaagacgacggagggggcagctccatttgtgcccagctagcttcctctgtcggtggctctgcctgttttattgtctcatcctagactcagcctagttgcctctccttcccgtcctctgcccggatggtccacgtcttcagtaccttgccagcagggagtcagagggggtgtgagaagtcgtgtacagccagacactcttgtgtgacaatggaattctgcagtcccctgggaagtcatgccaggacctctgccttcctcgtggcctcactgtcaagactgtgtcagtgaatagctggcctcacagactattctcacatgttcagagaaagccaaaccatcttcctaaccaattacagggaccctgcttgaggttgtcccacctccaaattcttcccagtgacctccatcagggcggctctgaagccttcccctgtgtccccaaccacttctgcctgccttcgactatccaaggcaaggtaggaggggatcaagttcctttcaaatggagaataaaaaagccattgtttcttcccagat

SEQ ID NO: 48 - protein, 511 aa, Mus musculus
MAVAQELYGFPASKLDSFVAQWLQPTREWKEEVLETVQTVEQFLRQENFREDRGPARDVRVLKVLKVGCFGNGTVLRSTTDVELVVFLSCFHSFQEEAKHHQAVLRLIQKRMYYCQELMDLGLSNLSVTNRVPSSLIFTIQTRETWETITVTVVPAYRALGPSCPSSEVYANLIKANGYPGNFSPSFSELQRNFVKHRPTKLKSLLRLVKHWYQQYVRDKCPRANLPPLYALELLTVYAWEAGTREDANFRLDEGLATVMELLQDHELLCIYWTKHYTLQHPVIEACVRRQLRGQRPIILDPADPTNNVAEGYRWDIVAQRANQCLKQDCCYDNRDSPVPSWRVKRAPDIQVTVQEWGHSDLTFWVNPYEPIKKLKEKIQLSQGYLGLQRLSFQEPGGERQLIRSHCTLAYYGIFCDTHICLLDTISPEIQVFVKNPDGRSHAYAIHPLDYVLNLKQQIEDRQGLRCQEQRLEFQGHILEDWFDFKSYGIQDSVTVILSKTTEGAAPFVPS

SEQ ID NO: 49 - DNA, 1442 bp, Rattus norvegicus
  CDS (68)..(1129)
gctatggatataagggcagccaactccagaggcaaggctgcagtcagcaaacatttgtggcctcaggatggagaaggatctcaagagtactccagcctcggagctggacaagttcatacaggatcaccttcttcctgacaccacattccgtgatgaggttagagcagacattgacttcatatgtactttcctgaaggagaaatgcttccacggtgccgccttgaaggtctctaaggttgtgaagggtggctcctcaggcaaaggaaccacgctacagggcaagtcagatgcagacctggtggtgttccttaacaatctcaccagctttgaggatcagttaaagcgacggggagaggtcattgagaaaattcggaaacacctgtgccagttgcagcaagagaaacagtttaaactgaagtttgagttccagaccccagagcaggccaactccaggtctctgagcttcaagctgagctcgccccagctccagcatgtggtgaagtttgatgtgctaccagcctatgatgtcctgggtcatgttaacatcaacagcaagcctaacgcccaaatctatgccagtctcatcaggaagtgcaccgacctgaataaagagggcgcgttttctacctgcttcacggagctccagaggaacttcctgaagcggcgcccaaccaagctgaagagtctcatccgcctggtcaagcactggtaccaactgtgtaaggagaagctgggggattcgctgcccccacagtatgccctggagctgctcacggtccatgcctgggaacgtggaaatggacttactgagttcaacacagctcagggcttccggacagtcttggaactggtcacaaagtaccagcagcttcgaatctactggactatgtattatgactttcaacacctagatgtctccaaatacctatacagacagctcaaaaaacccaggcctgtgatcctggaccctgctgacccaacagggaacgtggctggtgggaaccaagaaggctggcggcggttggcctcagaggcgaggctgtggctgcagtgcccatgttttatgaacagggatggttccccagtgagctcctgggaagtgcagacggaggttcctgtatgtttctagcaggtggatgaggcctggtcatgcatcctgctgtgaacccagcagcaccagcccaggaggctccggagtcaggggcacgtgctgctctgctgcaggaccttgacacagtgagggagggccccactcgggatcacagtccatgcttctgatgcccgcccgccatgtttgaatactgtccaatcacagatagccttcctcaacagattcagaaggggcggaaagaactcaagcttgacttccatctgaccgtccacctgttgggaggttctgtccaaccatgtctgtcaacaacaataaagtacaccaggtgcca

SEQ ID NO: 50 - protein, 353 aa, Rattus norvegicus
MEKDLKSTPASELDKFIQDHLLPDTTFRDEVRADIDFICTFLKEKCFHGAALKVSKVVKGGSSGKGTTLQGKSDADLVVFLNNLTSFEDQLKRRGEVIEKIRKHLCQLQQEKQFKLKFEFQTPEQANSRSLSFKLSSPQLQHVVKFDVLPAYDVLGHVNINSKPNAQIYASLIRKCTDLNKEGAFSTCFTELQRNFLKRRPTKLKSLIRLVKHWYQLCKEKLGDSLPPQYALELLTVHAWERGNGLTEFNTAQGFRTVLELVTKYQQLRIYWTMYYDFQHLDVSKYLYRQLKKPRPVILDPADPTGNVAGGNQEGWRRLASEARLWLQCPCFMNRDGSPVSSWEVQTEVPVCF

SEQ ID NO: 51 - DNA, 1680 bp, Rattus norvegicus
  CDS (32)..(1180)
aggctgcagaagcaaatgctccggaccaatcatggggcacggactcagcagtatctcagcctcggagctggacaagttcatagaggtttacctccttccaaacaccagctttggtgctgacgtcaaattagcgatcaatgtcgtgtgtgatttcctgaaggagagatgcttccgaggtgctgcccacccagtgagggtctccaaggtggtgaagggtggctcctcaggcaaaggcaccacactcaagggcaagtcagacgctgacctggtggtgttccttaacaatctcaccagctttgaggatcagttaaacagacggggagagttcatcaaggaaattaagaaacagctgtatgaggttcaacgtgaaagacattttggagtgaagtttgaggtccagagttcatggtggcccaacccccgggctctgagcttcaagctgagtgcaccacacctccaacaggaggtggagtttgatgtgcttccagcctatgatgtcctaggtcatgtaagcatctacagcatgcctgacccccaaatctatgccagtctcatcaggaagtgcatgtacctggggaaggagggcgagttctctacctgcttcacggagctccagaggaacttcctgaagcggcgcccaaccaagctgaagagtctcatccgcctggtcaagtactggtaccatctgtgtaaggagaagctggggaagccgctgcccccacagtacgccctggagctgctcacggtctatgcctgggagcgtggaaatggatttgtcgattttgagacagcccagggcttccgggcagtcttggaactgatcataaagtaccaggagcttagaatctactggacaacctattataactttcagcaccaagaggtctccaactacctgcacacacagctcacaagaatcaggcctgtgatcctggacccggctgacccaacaggaaacattgctggttcgaacccagagggctggaggcgactagcaggagaggctgctgcctggctgcggtacccatgctttaagtacaaggacggttccccagtgtgtccctgggatgtgccgatggaggttgacgtgccgtaccaggaggatcacttttttcgtaatttttgtctattttttttgtttttgttccttttcatattttggagggtttcttgtgtatagtgcaggctctcgtgtatatcgtcatcctcctgcctcggtgctggcatgactgcagagtccgcctgatgtgccctggattccctccatcctcaagtggacaagactgtgcatctgtcgtcctgtgagcccagcaggacctgcccaggaggctccagagtcagtcatggctttctgtgctgcaggcccttgacccagagagggaaggaaggttcccaagaccccagtgagggagggtccaacctgtgatcagactctggtcttctgacccctgccttcctactcctgcatcctgtcccatcacagacagccctcctcacagcctgcttcatctgccttgtcctccaacagtgctctcttgggagacaagagattcagaaggggaggcaggaactcgagcttgacttccacctgtccacctgttgggagttctgtccaatgtgtgaccaacgacaataaaccatagcaagcaaaaaaaaaaaaaaaaaaaaaaaaaaaaaaaa

SEQ ID NO: 52 - protein, 382 aa, Rattus norvegicus
MGHGLSSISASELDKFIEVYLLPNTSFGADVKLAINVVCDFLKERCFRGAAHPVRVSKVVKGGSSGKGTTLKGKSDADLVVFLNNLTSFEDQLNRRGEFIKEIKKQLYEVQRERHFGVKFEVQSSWWPNPRALSFKLSAPHLQQEVEFDVLPAYDVLGHVSIYSMPDPQIYASLIRKCMYLGKEGEFSTCFTELQRNFLKRRPTKLKSLIRLVKYWYHLCKEKLGKPLPPQYALELLTVYAWERGNGFVDFETAQGFRAVLELIIKYQELRIYWTTYYNFQHQEVSNYLHTQLTRIRPVILDPADPTGNIAGSNPEGWRRLAGEAAAWLRYPCFKYKDGSPVCPWDVPMEVDVPYQEDHFFRNFCLFFLFLFLFIFWRVSCV

SEQ ID NO: 53 - DNA, 1712 bp, Rattus norvegicus
  CDS (29)..(1114)
gcagtcagcacacattcctgacctcaggatgtcgaggcgactcgccagaaccccaatctggaggctggacagattcatagaggatcacctccttcctgacaccaccttccttactgaggtcagagcagacattgactctatatgtgctttcctgactgagagatgcttccaaggtgccacccaccctgtgagggtctctaggattgtgaagggtggctggttcaataaatacaccatgcttaagggaaggtcagaggccaccctggtggtgttccttaacaatctcacctgctttgaggatcagttaaagcgacgagaagagttcactgaggaaatttggaaacacctgtgccagttgcagcaagagaaacagtttaaactgaattttgaggtccagagcacagaacagcccgactccaggactctgagcatcaagctgagctcgccccagctccagcaggaggtggagtttgatgtgcagccagcctatgatgtcctgtatgagctaagagacaaggacgactttaacccggataactacagggaaatctatgcccgccttatccgtgagagcaccaccctggagatggagggcgagttctccgtctgcttcacggaccttcatcagaacttcctgaagtatcgtgcacccaagctgtggaatatcatccgtctggtcaagcactggtatcaactgtgtaagaagaggctgaagtatccgctgcccccacagtacgccctggagctgctcacggtctatgcctgggagcgtgggctggaagaccgccctgcactacatacaggccactgtttccgaagtgtcttagatctgatcacagagtacaagaagcttcgaatctactggacatggtgctatgaattcaaaaaagagacctttgactatctgagcagacagataaaaaaagacaggcccgtgatcctggaccctgctgacccaacaaggaacgtggctggttcaaacatccaatcctggcacgtgctggcaaaagaagctgcggtctgggcgcggtcgtctttctttaggaacagtgatatgacctttgtcaatggctggaaagtgtcggcacagaaacaagaatgtgtctaccagtgagcaccacagcccttgcccaggaggcttctgaagtcagggccatatgctcttctgctgcaagaccttgtcctgaagagaacagtttggcgcaccagggtccagtgaggggcatccaacctgtgatcagactccaggcttctattccgtcctgcccatgggacagccttcctcacagcctgcttcacctgccttatcccccacagtgttctttgggagtcagactccgaaggaaagagaagagctcaaacgtggcttctgtctgtccacctgtggggaggctctgtccacaaacatgtctgatcatgaacaatcgaccacagcaatccctgagggagtgtgcactcggaggggtgagtcctcagcccacataggaacggctcagcagaggagcgtggcccgggaacgctggggatctggccagtgtgcatcgaggtgcactgttgggcaaagtgtgagctggggttacggcagtgcagggaaataaaattgtatgcactttaaaacttaggaccttaaaacttagatgtgtgtgcgtgtgaggatgagatttgaagccctgaattaaagtaaaatgcaaaaaaaaaaaaaaaaaaaaaaa

SEQ ID NO: 54 - protein, 361 aa, Rattus norvegicus
MSRRLARTPIWRLDRFIEDHLLPDTTFLTEVRADIDSICAFLTERCFQGATHPVRVSRIVKGGWFNKYTMLKGRSEATLVVFLNNLTCFEDQLKRREEFTEEIWKHLCQLQQEKQFKLNFEVQSTEQPDSRTLSIKLSSPQLQQEVEFDVQPAYDVLYELRDKDDFNPDNYREIYARLIRESTTLEMEGEFSVCFTDLHQNFLKYRAPKLWNIIRLVKHWYQLCKKRLKYPLPPQYALELLTVYAWERGLEDRPALHTGHCFRSVLDLITEYKKLRIYWTWCYEFKKETFDYLSRQIKKDRPVILDPADPTRNVAGSNIQSWHVLAKEAAVWARSSFFRNSDMTFVNGWKVSAQKQECVYQ

SEQ ID NO: 55 - DNA, 1518 bp, Rattus norvegicus
  CDS (6)..(1094)
tcaggatgccgtggaaacttgccaatacccccatctggaggctggacagattcatacaggatcacctccttcctgacaccaccttccttactgaggtcagagcagacattgactttatatgtgctttcctgactgagagatgcttccaaggtgccacccaccctgtgagggtctctaggattgtgatgggcggctgctacgatgaatactccatgctcaagggaaggtcagaggccaccctggtggtgttccttaacaatctcacctgctttgaggatcagttaaagcgacgagaagagttcattgaggaaatttggaaacacctgtgccagttgcagcaagagaaacagtttaaactgaattttgagttccagacctcagagcaggccaactccaggtctctgagcatcaagctgagctcgccccagctccagcaggaggtggagtttgatgtgcagccagcctatgatgtcctgtatgcttggagatacaacaagtaccttgatcccagaatctacaacaaaatctacgccagcctcatccgtgagagcaccaacctggataaggagggcgagttctccgtctgcttcacagagctccaacaacacttcctgaagcatcctggacataagatgtggaatctcatccgcctggtcaagtactggtatcaactgtggaaggagaagctgggggatttgctgcccccacagtacgccctggagctgctcacggtccatgcctgggaactggggattgaaaacacctgtgagctatacatagcccggggcttccgaagtgtcttagaactgatcatcaaatatcggtgtctcctaatcttctggacattgtgttatgactttaatcacaatgaggtctctgagtacctgaacaaacaactccaaaaagacaggcccgtgatcctggaccctgctgacccaacaaggaacgtggctggttcaaacctccaggcctggcacctgctggcagaagaggctggggcctgggtgcagtcgtctttctttaggaacagcgatatgtcccttgttcacagttggaaagtgccgccagagaaacaaacatgtgtcatcctgtgagcccagcaggacctgcccaagaggcttcggagtcagggccatgtgctcttctgctgcaagaccttgccctggagagaacagttcggtgcaccaggctccagtaaggggcatccaacctgtgatcagactccaggcttctgatcccgtcctgccctgcccagggacagccttcctcacagcctgcttcacccgccttatcccccacagtgttctctgggagtcggactctgaaggaaagggacaaacttaaccttgacttccaccttttcacctgttgtgaagctcagtccaacaatgtctgattatctatgataagccacagcaagccattgcgggggggtgtgcactctgagggatggggccttagaagtacgtgtatgacttttcatgctctactagaaacattgagtacttgttgcaagct

SEQ ID NO: 56 - protein, 362 aa, Rattus norvegicus
MPWKLANTPIWRLDRFIQDHLLPDTTFLTEVRADIDFICAFLTERCFQGATHPVRVSRIVMGGCYDEYSMLKGRSEATLVVFLNNLTCFEDQLKRREEFIEEIWKHLCQLQQEKQFKLNFEFQTSEQANSRSLSIKLSSPQLQQEVEFDVQPAYDVLYAWRYNKYLDPRIYNKIYASLIRESTNLDKEGEFSVCFTELQQHFLKHPGHKMWNLIRLVKYWYQLWKEKLGDLLPPQYALELLTVHAWELGIENTCELYIARGFRSVLELIIKYRCLLIFWTLCYDFNHNEVSEYLNKQLQKDRPVILDPADPTRNVAGSNLQAWHLLAEEAGAWVQSSFFRNSDMSLVHSWKVPPEKQTCVIL

SEQ ID NO: 57 - DNA, 1619 bp, Rattus norvegicus
  CDS (33)..(1112)
  misc_feature (1559)..(1559): n is a, c, g, or t
ggctgcagtcagcaaacactcccggccttaaaatggtgaacctcagcagcaccccagcctgtgagctggacaggttcataaaggatcacctccctgctgacaccagcttccatgctgagctcagagcagacattgacttcatatgtgctttcctgaaggagagatgcttccaaggtgccgctcaccctgtgagggtctccagggttgtgatgggcgaacacaccatgcttaagggtaggtcagaagccaacctagtggtgttccttaacgatctccccagctttgaggatcagttgaatctacagggagagttcattgaggaaattcggaaacgactgtgtcagctgcagcaagagaaaacgttacaagtgaagcttgaggtccagagctcagagcagcccagctccaagtctctgagcttcacgctgagctcgccccagctccagcaggaggtggagtttgatgtgcagccagcctatgatgtcctgtttgccctaagaaacaaccacaagcccgacccccaaatctacaccaaaacctacgcctacctcatcagtgtgtgcaccactctgaagaaggagggcgagttctccacctgcttcatggagctccggcaaaacttcctgaagcatcgggaacccaagctgaagagcctcatccgtctggtcaagcactggtatcaactgtgtaaggagaagctggggaagccgctgcccccacagtacgccctggagctgctcacggtctatgcctgggaaagtgggagtagagactgtgaattcaacacagcccagggcttccgaaccgtcttggaactggtcaccaagtatcagtggcttcgaatctactggacattgtattatgactttagaaacaagaaggtctctgattacctacacaaacagctcaaaaaaaccaggcctgtgatcctggacccggctgacccgacaagaaatgtggctggttcaaaccccctctgctggcgactgttggcaaaagaagctgctagctggctgcagtgcccatgctttaggacctgtgatatgtccctcgtgcactcctgggaagtgctgacaaaggtcgagtttccacaggaatgtgtccttctatgagcaccaaagcacctgcccaggatgctcaagagtcaggggcatgacctctgctgcaggcccttgacctagagaggagaggaagctccccaaaaccccaacaagggagggtccaacctgtgatcagactctggtcttctgacccctgccttcctactcctgcatcctgtcccatcacagacagccctcctcacagcctgcttcatctgccttgtcctccaacagtgctctcttgggagacaagagattcagaaggggaggcaggaactcgagcttgacttccacctgtccacctgttgggagttctgtccaatgtgtgaccaacgacaataaaccatagcaagccatgaggatatgtgaacattctgaaggatgtgtgttctcctccctctccctctccccttccctacctaccctgtcctgcctcacatacgttttcttacctctacntgcatatgacatgatagtatatttaagtgatcccaaaagttccaccagagaactactaaa

SEQ ID NO: 58 - protein, 359 aa, Rattus norvegicus
MVNLSSTPACELDRFIKDHLPADTSFHAELRADIDFICAFLKERCFQGAAHPVRVSRVVMGEHTMLKGRSEANLVVFLNDLPSFEDQLNLQGEFIEEIRKRLCQLQQEKTLQVKLEVQSSEQPSSKSLSFTLSSPQLQQEVEFDVQPAYDVLFALRNNHKPDPQIYTKTYAYLISVCTTLKKEGEFSTCFMELRQNFLKHREPKLKSLIRLVKHWYQLCKEKLGKPLPPQYALELLTVYAWESGSRDCEFNTAQGFRTVLELVTKYQWLRIYWTLYYDFRNKKVSDYLHKQLKKTRPVILDPADPTRNVAGSNPLCWRLLAKEAASWLQCPCFRTCDMSLVHSWEVLTKVEFPQECVLL

SEQ ID NO: 59 - DNA, 1469 bp, Rattus norvegicus
  CDS (68)..(1171)
gttccaagttcaaccaggctgcgagacacaggacctgcaggctgcagaggcaaaagctccggaggtcatggagcaggaactcaggagcaccccgtcctggaagctggacaagttcatagaggtttacctccttccaaacaccagcttccgtgatgatgtcaaatcagctatcaatgtcctgtgtgatttcctgaaggagagatgcttccgagatactgtccacccagtgagggtctccaaggtggtgaagggcggctcctcaggcaaaggcaccacactcaagggcaagtcagacgctgacctggtggtgttccttaacaatttcaccagctttgaggatcagttaaacagacggggagagttcatcaaggaaattaagaaacagctgtatgaggttcagcgtgaaaaacattttagagtgaagtttgaggtccagagttcatggtggcccaacccccgggctctgagcttcaagctgagtgcaccacacctccaacaggaggtggagtttgatgtgcttccagcctatgatgtcctaggtcatgttagcctctacagcaatcctgatcccaagatctacaccatcctcatctccgaatgtatctccctggggaaggagggcgagttctctacctgcttcacggagctccagaggaacttcctgaagcagcgcccaaccaagctgaagagtctcatccgcctggtcaagcactggtaccaactgtgtaaggagaagctggggaagccgctgcccccacagtacgccctggagctgctcacggtctatgcctgggaacgtggaaatggaattactgagttcaacacagctcagggcttccggacaatcttggaactggtcacaaagtaccagcagcttcgaatctactggacaaagtattatgactttcaacacccagatgtctccaaatacctacacagacagctcagaaaatccaggcctgtgatcctggaccctgctgacccaacagggaacgtggctggtgggaaccaagaaggctggcggcggttggcctcagaggcgaggctgtggctgcagtacccatgttttatgaacaggggtggttccccagtgagctcctgggaagtgccgacagaggttcctgtgccttcagagcaggtggatgaggcctggtcatgcatcctgctgtgaacccagcagcaccagcccaggaggctccggagtcaggggcacgtgctgctttgctgcaggaccttgacacagtgagggagggccccactcgggatcacagtccatgcttttgatgcccgcccgccatgtttgaatactgtccaatcacagacagccttcctcaacagattcagaaggggcggaaagaactcaagcttgacttccatctgaccgtccacctgttgggaggttctgtccaaccatgtctgtcaacaacaataaagtacagcaggtgccaaaaaaaaaaaaaaaaaaaaaa

SEQ ID NO: 60 - protein, 367 aa, Rattus norvegicus
MEQELRSTPSWKLDKFIEVYLLPNTSFRDDVKSAINVLCDFLKERCFRDTVHPVRVSKVVKGGSSGKGTTLKGKSDADLVVFLNNFTSFEDQLNRRGEFIKEIKKQLYEVQREKHFRVKFEVQSSWWPNPRALSFKLSAPHLQQEVEFDVLPAYDVLGHVSLYSNPDPKIYTILISECISLGKEGEFSTCFTELQRNFLKQRPTKLKSLIRLVKHWYQLCKEKLGKPLPPQYALELLTVYAWERGNGITEFNTAQGFRTILELVTKYQQLRIYWTKYYDFQHPDVSKYLHRQLRKSRPVILDPADPTGNVAGGNQEGWRRLASEARLWLQYPCFMNRGGSPVSSWEVPTEVPVPSEQVDEAWSCILL

SEQ ID NO: 61 - DNA, 1701 bp, Rattus norvegicus
  CDS (29)..(1117)
gcagtcagcaaacattcctggcctcaggatggagaaggatctcaagagtactccagcttggaagctggacaagttcatacagaatcacctccttgctgacaccaccttcgttactgaggccaaggcagacatagagttcctatgtgatttcctgactgagagatgcttccaagatgcctcccaccctgtgagggtctctaggattgtgatgggcggctgctatgatgaatactccatgctcaagggaaggtcagaggccaccctggtggtgttctttaacaatctcaccagatttgaggatcagttaaagcgacgggaagagctcattgaagaaatttggaaacacctgtgccagttgcagcacgagaaacagtttaaactgaagtttgaggtccagagctcagagcaggacaactacagctctctgagcatcaagctgagctcgccccagctccagcaggaggtggagtttgatgtgcagccagcctatgatgtcctgtatgaactgaaagaaaaactggagcttgactgtgagttctacaacaaaatctatgcccggctcatccgtgagtgcatcaccctgaggaaggagggcgagttctccgtctgcttcatggagctccagcaaaagttcctgtggaatcgtccagaagacctgaggaatctcctcgtactggtcaagcactggtatcaactgtgtaaggagaagctgggggattcgctgcccccacagtacgccctggagctgctcacggtccatgcctgggaaaacgaaattccagccaaatatggagcacagacagctcggggtttccagagtgtcttagaactgatcattaagtacacctgtcttcgagtctactggacattttattacgacattctacaccaggatgtctccagctacctgcacaaacagctccgtaaagaaaggcctgtgatcctggaccctgctgacccaacaaggaacgtggctggtttgaacatagatggctggtgtgagctggcaaaagaggcagaagcctggctgaagtacccgtgctttaggcacatcgatgagacctttgtgggctcctgggaagtgccgccagagaaacaaggaggtgtcttcctgtgagcaccacagcccttgcccaggaggctctggagtcaggggcattcactcctctgctgcaagaccttgtcctgcagagaacagtttggtacaccaggctccagtgaggggcatccaacctgtgatcagactctaggcttctgatcccgtcctgcccatgggacagccttcctcacagcctgcttcacctgccttatcctccacagtgctctctgggagtcagactccgaaggaaagagaagagctcaaacttggcttctgtctgtccacctgtggggaggctctgtccacaaacatgtctgatcatgaacaattgaccacagcaatccctgagggagtgtgcactcggaggggtgagtcctcagcccacataggaacggctcagcagaggagcgtggcccggaacgctggggatctggccagtgtgcatcaaggtgcactgttgggcaaagtgtgagctggggttacggcagtgcagggaaataagattatgtacactttaaaacttaggaccttaaaacgtatgtgtgtgcatgtgaggatgagatttgaagccctgaattaaagtaaaatgcaaaggactaaac

SEQ ID NO: 62 - protein, 362 aa, Rattus norvegicus
MEKDLKSTPAWKLDKFIQNHLLADTTFVTEAKADIEFLCDFLTERCFQDASHPVRVSRIVMGGCYDEYSMLKGRSEATLVVFFNNLTRFEDQLKRREELIEEIWKHLCQLQHEKQFKLKFEVQSSEQDNYSSLSIKLSSPQLQQEVEFDVQPAYDVLYELKEKLELDCEFYNKIYARLIRECITLRKEGEFSVCFMELQQKFLWNRPEDLRNLLVLVKHWYQLCKEKLGDSLPPQYALELLTVHAWENEIPAKYGAQTARGFQSVLELIIKYTCLRVYWTFYYDILHQDVSSYLHKQLRKERPVILDPADPTRNVAGLNIDGWCELAKEAEAWLKYPCFRHIDETFVGSWEVPPEKQGGVFL

SEQ ID NO: 63 - DNA, 1980 bp, Rattus norvegicus
  CDS (9)..(1547)
gcgcagacatggcagtagcccaggagctttacagcttcccagcctccaagctggactcctttgtggctcagtggctgcagccaaccagagaatggaaggaggaggtcctggagacggtgcagacagtggagcagttcctgaggcaggagaacttccgtggagaacgtggcccggcccaggatgtacgagtgctcaaggtactcaaggtaggctgctttgggaatggcacagtactcaggagtaccacagacgtggagctggtggtgttcctgagctgtttccacagcttccaggaagaggccaaacaccaccaggctgttctgagactgatacagaaaaggatgtcttactgccgggacctgctggatctcgggctcagtaacctgagtgtgattgaagaagtgcccagtagtctcatcttcactatccagaccagggagacctgggagcccatcactgtcaccatcgtgcccgccttcagagccctgggaccttcctgtcccaactccgccgaggtctatgtgaatctgatcaaggctaacggctaccccggaaatttctctccttccttcagcgagctacagaggagcttcgtgaagcataggccgactaagctgaagagcctcctacggttggtcaaacactggtaccagcagtacgtgagagacaagtgcccccgggccaacctgccccccctctatgccctggagctgctaactgtctacgcgtgggaagcaggtacgcaggaggattcgaacttcaggctggatgaaggtctcgccactgtcatggagttgctccaggatcatgaactcctgtgcatctactggaccaagtactacaccctgcaacacccagtcattgagcgcttcgtcaggagacagctcaaaggagaaaggcccattatcctggacccagcagaccccacccacaacgtggcgcaaggctacaggtgggatatagttgctcagcgcgccagccagtgtctgaaacaggactgttgctatgacgacagggacgcccccgtccccagctggactgtgaagagagcaccatatatccaggtgaccgtgcagcagtggggtcacccggatttaatcctctgggtgaacccttatgaacccataaagaagctgaaagagaaaatccgactgagccggggctactccggcctgcagcgcctgtcctttcaggagcccggcggccaacggcagctcatcagaagccaatgctcgcttgcctactacggaatcttctgcgacactcagatctgcctgctggacaccatctcccccgagatccaggtctttgtgaaaaacccggatggtggaagccacgcctacgccatccacccacttgacttcgtcctgagcctgaagcagcagatcgaagataggcagggccttcaaagccaggagcagcagctggagttccagggccgcgtcctggaagactggtttgactttaaatcctatggcatccaagatagtatcacgatcatcctatccaggaagagggaggggaaagccccatctgcgcccagctagcttcctctgcctcttttgctatctcatcctaaagtcagcctagtcacccctccttccggtcctcagccgggatgatcccagcagggagccagaagggaatactgccagacgctcttgtgtgacaatgaaactctgcagtcacctgtgaaatcacaccaggacctctacgctctcaagactgggtcagtgaatggccgtcccacaaataaactattctcgcttgttcttgggaagccaaacgatcttcctaaccaatcaaatggaccctgcttcaggttgttcccccacacacaccagcaacctccatcaggttggatctgaagccttcccctgtgctcccaaccacttctgcctgcctcagcctatccaaggcaaggtagggtatcaagttcctttcaaatggagaataaacaacctttgtttcctcccagat

SEQ ID NO: 64 - protein, 512 aa, Rattus norvegicus
MAVAQELYSFPASKLDSFVAQWLQPTREWKEEVLETVQTVEQFLRQENFRGERGPAQDVRVLKVLKVGCFGNGTVLRSTTDVELVVFLSCFHSFQEEAKHHQAVLRLIQKRMSYCRDLLDLGLSNLSVIEEVPSSLIFTIQTRETWEPITVTIVPAFRALGPSCPNSAEVYVNLIKANGYPGNFSPSFSELQRSFVKHRPTKLKSLLRLVKHWYQQYVRDKCPRANLPPLYALELLTVYAWEAGTQEDSNFRLDEGLATVMELLQDHELLCIYWTKYYTLQHPVIERFVRRQLKGERPIILDPADPTHNVAQGYRWDIVAQRASQCLKQDCCYDDRDAPVPSWTVKRAPYIQVTVQQWGHPDLILWVNPYEPIKKLKEKIRLSRGYSGLQRLSFQEPGGQRQLIRSQCSLAYYGIFCDTQICLLDTISPEIQVFVKNPDGGSHAYAIHPLDFVLSLKQQIEDRQGLQSQEQQLEFQGRVLEDWFDFKSYGIQDSITIILSRKREGKAPSAPS

SEQ ID NO: 65 - DNA, 1750 bp, Rattus norvegicus
  CDS (67)..(1602)
ttctcccagcattgctgagcagaagcacagaagattcaatccagaggacaggctgatccagcagagatggatacattacccgacctgtatgggaccgttggggacagtctagactacttcctggaacacagccttcagccccaaagggactggaaagaggaaggaaaggatgcctgggagagaattgagaggttctttcgggaaaagtgcttctgtgatgagctgctcctggaccaagaagtcagggtgcttaaagtggtaaagggaggctcctcaggaaaggggacggcgctgaaccacagatctgaccaggacatgattctgttcctgagctgtttttccagtttcaaacagcaggcaagagaccggaaggccgtcatagacttcattaagagcaagctgattcattgtaggaaaagcctggcctacaacatcactgtccgtcaacacaaagaaggcaaaaggacccctcgctccctgaccctagagatccagtccaggaagagcaatgacataatttgcatggatattctccctgcttacaatgccttgggatccttttccagagactgcaaaccagaacctgaaatctacgagaatctgataaggtgtaagggctaccctggcgacttctcgccaagtttcgcagagttacagagacattttgtgaaaagtcgcccggttaaactgaaaaacctcctacggttggtgaagttctggcacctgaagtacctgaggcataaatatagaagagcagtgttgccctcaaaatatgcactggagttgctgaccatctatgcttgggagatgggtacagacagcagtgataatttcaatctggatgaagggtttgtagccgtcatggagctcctcagggactaccaagacatctgcatctactggaccaagtactatgatttccaaaatgaggtcgtcaggaacttcctgaaggaacagcttaagggcgaccggcctatcattctagacccagctgaccccaccaacaacctaggaagaagaggaaaatgggaactggtggccaaagaagctacttactgcctgctacagttgtgttgtgtgactgcagaccgctggaatgttcaggtatctatcgcgcactacctccgaggagcgagggacgttcaggtgacagtgaaacaaacaggaagggaagagtggattctcttgacaaacccccacagccccatcaggaagttgaaggcaaagatcaagaagagaatgaacctctgtggggagctgcgtatctccttccaggagccgggaggggagaggcagccgctgagtggccggaaaaccctgtcggattatggaattttctctaaggtgaacatccgggtgatggagacctttcctcctgagatccaggtctttgtgaggtatcccggtggccagaacaagccttttgccatcgaccctgatgctaccatcttaagcctgtgggagaaaattgaggaagatggaggcccatgtacggaggattgggtactactgtttgagggtgaggagctggacgatgatgacaaccttgcagagcttcagatcaaggactgtgacaccatccagctcagcagggtctcctagtctgcctccccacatcacccctttactctgacatattcctcctgtagcttaaacatcatcatatccggttggggatttggctcagtggtagagcgcttgcctagcaaccgcaaggccctgggttcggtccccagctccgaaaaaaaaa

SEQ ID NO: 66 - protein, 511 aa, Rattus norvegicus
MDTLPDLYGTVGDSLDYFLEHSLQPQRDWKEEGKDAWERIERFFREKCFCDELLLDQEVRVLKVVKGGSSGKGTALNHRSDQDMILFLSCFSSFKQQARDRKAVIDFIKSKLIHCRKSLAYNITVRQHKEGKRTPRSLTLEIQSRKSNDIICMDILPAYNALGSFSRDCKPEPEIYENLIRCKGYPGDFSPSFAELQRHFVKSRPVKLKNLLRLVKFWHLKYLRHKYRRAVLPSKYALELLTIYAWEMGTDSSDNFNLDEGFVAVMELLRDYQDICIYWTKYYDFQNEVVRNFLKEQLKGDRPIILDPADPTNNLGRRGKWELVAKEATYCLLQLCCVTADRWNVQVSIAHYLRGARDVQVTVKQTGREEWILLTNPHSPIRKLKAKIKKRMNLCGELRISFQEPGGERQPLSGRKTLSDYGIFSKVNIRVMETFPPEIQVFVRYPGGQNKPFAIDPDATILSLWEKIEEDGGPCTEDWVLLFEGEELDDDDNLAELQIKDCDTIQLSRVS

SEQ ID NO: 67 - DNA, 2311 bp, Rattus norvegicus
  CDS (41)..(2242)
agtgctctgcctctgtcattcacaagcccagctagcaactatgggaaactggatgcctggctggtcatccagtgggtctttgggggtgccccccatgccagtgcagaagctagaaaagtctgtccaggtaaaccttgaaccagatgaaaaatgtctgagtcagaccgaggtgtcctctgtaccatcccagaagctagaagaatatatccaggcaaacctcaaacccgatgaagaatctctgaagcagatagaccaggctgtggatgccatctctgacctgctgtgcagtgaggtgatgatcgatgtgctgaaagtggttaagggtggctcctatggtcggaaaacagtcctaagagactgctccgatggtacacttgttctcttcaccggtctcttcaaacagttccaagaccagaagaaataccaagataagctccttgacttgattgaacaacggctgaaaagccatgagaaatacaagaagtcagtaaaacgtaaacttagcctccttgaagtacaagtgtctataccagggcagagtatactcttgcagttgcttccaaccttcaatcctctgtgcatcagtgagaatcccagcgcccaggtctatcagaatctcaaaagatccatggatcaagtaaaagcatcacctggggaattctcagactgcttcaccacactgcagcagaggtttttcgagaaatatcccgggagactgaaggatttgattctattggtcaagcactggtataaacagttgcaggataagtggataataccctcacctccgccattgctatatgcactggagctgcttactgtgtatgcctgggaacagggctgccagactaaagactttgacatcacgcaaggtatcaggactgtgctgcaactcatcagtcagccgacaaacctgtgtgtctactggttagacaattacaactttgaggatgagacagtccggaacaaccttctgcaccagctcaactccccaagaccggtcatcttggatccaaccgacccaaccaacaatgtgggcaaagatgacaggttctggcagctactggcagaagaggctcaggagtggctgaactctctcagactgaataagccacacaaaccatgttgggatgttctgcccatgccatttttcatcaccccaagccactgcctggacaagttcatcaaagacttcctccaacctgacaaggtcttcctaaaccaaatcaaaagagctgttgacattatctgttcattcttaaaagagacctgcttccagaattctgacatcaaagtcctgaagatcatcaagggaggatccactgccaaaggcacagctctgcagcagagatcagatgctgacatcatagtgttcctcagctcactggatagttatgactccctagaaactgaacgctcccagtacgtccaggagatccgaaagcagttagaagcctgccagaaggcctttaatttaggggtgaagtttgatatttccaaatggatggcccccagggtgctgagttttaccctggaatccaagagtctcaagcaaagtgtggagttcgatgtccttcccgcctatgatgcactaggtcagctgcggtctgactacacctccaggctcaaagcctacaagaagctcattgagctgtatgcctcacaggacagcctcaaaggaggggagttttcagtctgttttacagagctacagagagacttcattgaaaccaggcccaccaaactcaagggtctgatccgcctgatcaagcactggtacaaacagtgtgaaaggaagatgaagccaaaagcatctttgcctccaaagtacgcactggagctgctcaccgtgtatgcgtgggagcatggcagtggcacagatggcttcgacactgctgaaggcttccggaccgtcctggacttggtcataagataccggcagctctgcgtcttctggacagtcaattacaactttgaggaggatcacatgaggaagttcctactgacccagatccagaaaaagaggcctgtgatcctggatccagcagatcccacaggtgatgtgggaggaggtgaccgctggtgttggcatcttctagccaaagaagcaaaggagtggctgtcctcctcctgtttccaagtggagccaaaaagccccgtgcagccgtggaaagtaccagtagtacagactccaggaagctgtggagctcagatctaccctgtggtgggtggggtgtactaagagagtgcattcagctctggagggaaaatgctggaagaagcttctagagacatctggcaaagactctgc

SEQ ID NO: 68 - protein, 733 aa, Rattus norvegicus
MGNWMPGWSSSGSLGVPPMPVQKLEKSVQVNLEPDEKCLSQTEVSSVPSQKLEEYIQANLKPDEESLKQIDQAVDAISDLLCSEVMIDVLKVVKGGSYGRKTVLRDCSDGTLVLFTGLFKQFQDQKKYQDKLLDLIEQRLKSHEKYKKSVKRKLSLLEVQVSIPGQSILLQLLPTFNPLCISENPSAQVYQNLKRSMDQVKASPGEFSDCFTTLQQRFFEKYPGRLKDLILLVKHWYKQLQDKWIIPSPPPLLYALELLTVYAWEQGCQTKDFDITQGIRTVLQLISQPTNLCVYWLDNYNFEDETVRNNLLHQLNSPRPVILDPTDPTNNVGKDDRFWQLLAEEAQEWLNSLRLNKPHKPCWDVLPMPFFITPSHCLDKFIKDFLQPDKVFLNQIKRAVDIICSFLKETCFQNSDIKVLKIIKGGSTAKGTALQQRSDADIIVFLSSLDSYDSLETERSQYVQEIRKQLEACQKAFNLGVKFDISKWMAPRVLSFTLESKSLKQSVEFDVLPAYDALGQLRSDYTSRLKAYKKLIELYASQDSLKGGEFSVCFTELQRDFIETRPTKLKGLIRLIKHWYKQCERKMKPKASLPPKYALELLTVYAWEHGSGTDGFDTAEGFRTVLDLVIRYRQLCVFWTVNYNFEEDHMRKFLLTQIQKKRPVILDPADPTGDVGGGDRWCWHLLAKEAKEWLSSSCFQVEPKSPVQPWKVPVVQTPGSCGAQIYPVVGGVY

SEQ ID NO: 69 - DNA, 4015 bp, Rattus norvegicus
  CDS (28)..(3441)
gaaactctcctgagggcatgggtcaacatggacctgtaccacacgccagccggagctctggacaagctggtggcccacagcctgcacccagcccctgagttcacagcggctgtgcggcgtgctctggggtccctggacaacgtcctaaggaagaacggagccggagggttacagagaccaagggtgataaggatcatcaagggaggagcccatgctcgaggcacagctctcagaggtggcactgatgtcgaactcgtcatcttcctcgactgcctccggagctttggcgaccagaagacctgtcacacagagatcctgggcgccatacaagcattgctggagtcctgggggtgcaaccctgggcctggcctgacttttgagttttctgggccaaaggcgtctggcatcttacagtttcgcctggcatcggtggaccaagaaaactggatagatgttagcctggtgcctgccttcgatgccctaggacagctccactctgaagtcaagccaacacccaatgtgtactcctccctcctcagcagccactgccaggctggggaacactcagcctgcttcacagagctccggaagaactttgtgaatatccgcccagtcaaacttaagaacttaatcctgctggtcaaacactggtaccgccaggtgcagacacaggttgtgagagccacactgccccctagctacgcgctggagctgctcaccatctttgcctgggagcagggctgtaggaaggatgccttcagcctggcccaagggctccggactgtcctggccttgatccaacgcaacaagcatctctgcattttctggacggaaaactacggcttcgaagaccctgcagttggggagttcttgcgaaggcagcttaagagacccaggcccgtgatcctggatccagctgacccaacatgggacttgggcaatgggacagcctggtgctgggatgtgcttgccaaggaggctgaatacagctttaaccagcagtgcttcaaggaggcctcaggagcccttgtgcaaccttgggaggggccgggccttccatgtgctgggatcttggatttgggtcaccctatccaacaaggagctaagcatgcccttgaagacaacaatggccaccttgctgttcagccaatgaaagagagcctacaaccatcaaatccagcccgaggactcccagaaacagccaccaagatctccgctatgccagacccaacggtcactgaaacccacaagagcctcaaaaaatcagtgcacccaaagactgtcagtgaaacagtggtgaatccctcaagtcatgtttggatcacccagagtacagcatcctcaaacacgcctccgggccactctagtatgtccaccgctgggtcacagatgggcccagatctgtcacagatccccagcaaggagctggactccttcatccaggaccacctcaggccgagttcccagttccagcagcaggtgaggcaggccatcgacaccatcctgtgctgcctccgggagaagtgtgtagacaaagtcttgagagtcagcaagggtggctcttttggccgtggcacagacctcaggggcaaatgtgatgtggagcttgtcatcttttataaaactctcggggacttcaagggccagaactcacaccagacagagatcctgtgtgacatgcaggcccagctgcagcgctggtgtcagaacccagcacctggactgagcctccagtttattgaacagaagtccaatgctctgcatcttcagctggtgcccaccaacctcagcaaccgggtagacctcagtgtgctgcccgcttttgatgcagtggggccgctgaagtccggcgccaaacctctacccgagacgtactcctccctcctcagcagcggctgccaggctggggagcatgcagcctgcttcgcagagctccgaaggaacttcataaacactcgccctgccaaacttaggagcctgatgctactggtcaaacactggtaccgccaggttgccgctcgatttgaaggaggagagacagcaggtgctgctctgcccccagcctatgccctggagctcctgacagtctttgcctgggaacaaggctgcggagaacaaaagtttagcatggctgaaggcctgcggactgtcctgaggctggtccagcagcaccagtcactctgtatctactggacagtcaactacagtgtgcaggacccagccatcagagcacaccttctccgccagcttcggaaagccaggcctctaatcctagaccctgcagatcccacctggaacatggaccagggcaactggaagttgctggctcaggaggcggctgccctggagtcacaagtctgccttcagagtagggatgggaatctggtgccaccatgggatgttatgccagccctccttcaccagaccccggctcagaacctggacaagttcatctgtgaattcctccagcctgaccgccatttcctgactcaggtgaagagagcagtggacaccatatgttccttcctgaaagaaaactgcttccggaattctaccatcaaggtgctcaaggtggtcaagggtgggtcttctgccaaaggcactgctctacaagggcgctcggatgctgacctggtggtgttcctcagctgcttccgccagttctctgaacaaggcagccatcgggcagagatcatcgcggagatccaggctcagctggaggcgtgtcagcagaagcagaggttcgatgtcaagtttgagatctccaagaggaagaacccccgagttctcagcttcacgctgacatccaagacgctgctaggccagagcgtggacttcgatgtgctgccagccttcgatgctcttggtcagctgaagtccggctctcggccagatccccgggtctacacggacctcatccagagctacagtaatgcaggagagttctctacctgcttcacggagctgcagcgggacttcattagctcccgtcccaccaaactcaagagtctgatccgtctggtgaaacactggtaccaacagtgtaacaagacagtcaaggggaagggttccttgcccccccagcacgggctggagctcctgactgtgtacgcctgggagcgaggcagccagaacccccagttcaacatggcggagggcttccgcacagttctggagctgattggccagtaccgtcagctgtgcgtctattggaccatcaactacggtgcagaagacgagaccatcggagacttcctgaagatgcagcttcagaagcccaggcctgtcatcctggacccagctgacccgacaggcaacctaggccacaatgcccgctgggacctgcttgccaaggaggctgcagcatacacatctgccctgtgctgcatggacaaggacggcaaccccatcaagccatggccagtaaaggccgctgtgtgaagtccagaaagatcaaaaagtgacaccagccctcagcaagggatactcagaatatctggccagatgtgtgtgtgtgtgtgtgtgtgtgtgtgttgtgtattgtgtttatctatatatgtttctgtgctgtgtctgtgtgtctctgtaggtggtgtgtgtctgtgtacccacaggtgtctacatgtgtctgtatatatgtaagagtgtgtgtatgtatgtatgttcacgtgtctctgtgtgtgtgcatctgtgtgtttgtatgtagtgtgtctataggtgtatccttatgagtgtgtgtgtttgtggtggagtgtgtgtgtgtctgtgtatctgtgggtgtctatatgtgtctgagtgtgtgtgattatttgtgtgtttatgtgtctgtgtatttgtgtgtagtgagtttataggtgcatctgtgtgtctatatgtatatgtgtgtctatgtgtgtctatggtgtgtgtccgtgtatctgtgggtgtctatgtgtgtctgtatgtacgtgtgtgtgtgtgtgacttgctttgtccaaatgattgtatgtatgtatgtgtgctcatgtggctctgt

SEQ ID NO: 70 - protein, 1137 aa, Rattus norvegicus
MDLYHTPAGALDKLVAHSLHPAPEFTAAVRRALGSLDNVLRKNGAGGLQRPRVIRIIKGGAHARGTALRGGTDVELVIFLDCLRSFGDQKTCHTEILGAIQALLESWGCNPGPGLTFEFSGPKASGILQFRLASVDQENWIDVSLVPAFDALGQLHSEVKPTPNVYSSLLSSHCQAGEHSACFTELRKNFVNIRPVKLKNLILLVKHWYRQVQTQVVRATLPPSYALELLTIFAWEQGCRKDAFSLAQGLRTVLALIQRNKHLCIFWTENYGFEDPAVGEFLRRQLKRPRPVILDPADPTWDLGNGTAWCWDVLAKEAEYSFNQQCFKEASGALVQPWEGPGLPCAGILDLGHPIQQGAKHALEDNNGHLAVQPMKESLQPSNPARGLPETATKISAMPDPTVTETHKSLKKSVHPKTVSETVVNPSSHVWITQSTASSNTPPGHSSMSTAGSQMGPDLSQIPSKELDSFIQDHLRPSSQFQQQVRQAIDTILCCLREKCVDKVLRVSKGGSFGRGTDLRGKCDVELVIFYKTLGDFKGQNSHQTEILCDMQAQLQRWCQNPAPGLSLQFIEQKSNALHLQLVPTNLSNRVDLSVLPAFDAVGPLKSGAKPLPETYSSLLSSGCQAGEHAACFAELRRNFINTRPAKLRSLMLLVKHWYRQVAARFEGGETAGAALPPAYALELLTVFAWEQGCGEQKFSMAEGLRTVLRLVQQHQSLCIYWTVNYSVQDPAIRAHLLRQLRKARPLILDPADPTWNMDQGNWKLLAQEAAALESQVCLQSRDGNLVPPWDVMPALLHQTPAQNLDKFICEFLQPDRHFLTQVKRAVDTICSFLKENCFRNSTIKVLKVVKGGSSAKGTALQGRSDADLVVFLSCFRQFSEQGSHRAEIIAEIQAQLEACQQKQRFDVKFEISKRKNPRVLSFTLTSKTLLGQSVDFDVLPAFDALGQLKSGSRPDPRVYTDLIQSYSNAGEFSTCFTELQRDFISSRPTKLKSLIRLVKHWYQQCNKTVKGKGSLPPQHGLELLTVYAWERGSQNPQFNMAEGFRTVLELIGQYRQLCVYWTINYGAEDETIGDFLKMQLQKPRPVILDPADPTGNLGHNARWDLLAKEAAAYTSALCCMDKDGNPIKPWPVKAAV

SEQ ID NO: 71 - DNA, 1682 bp, Bos taurus
  CDS (63)..(1232)
gcacgagcacagattcaggcagcagctctgccgcctctggctctccagtccccagcaccgtgatggagctcagaaataccccggccgggtctctagacaagttcatcgaagaccacctcctgccagacgaggagttccgcatgcaggtcaaagaagccatcgacatcatctgcactttcctgaaggagaggtgtttccgatgtgcccctcacagagttcgggtgtccaaagttgtgaagggcggctcctcaggcaaaggcacgaccctcaggggacgatcagatgctgacctcgtcgtcttcctcaccaatctcacaagttttcaggaacagcttgagcgccgaggagaattcatcgaagaaatcaggagacagctggaagcctgtcaaagagaggaaacatttgaagtgaagtttgaggtccagaaacggcaatgggagaatccccgcgctctcagctttgtgctgaggtcccccaagctcaaccaggcggtggagttcgatgtcctgcccgcctttgatgccctaggtcagttgaccaaaggttacagacctgactctagagtctatgtccggctcatccaagagtgcaagtacctgaagagagaaggcgagttctccccctgcttcacggagctgcagcgagacttcctgaagaatcgtccaaccaagctgaagagcctcatccgcctggtgaagcactggtaccaactgtgtaaggagcagcttggaaagccattgcccccacaatatgctctggagcttctgacggtctatgcctgggagcaaggatgcaataaaacaggattcatcacagctcagggatttcagactgtcttgaaattagtcctaaagtatcagaagctttgcatctactgggaaaagaactataactctgaaaaccctattattgaagaatatctgacgaagcaacttgcaaaacccaggcctgtgattctggacccggcggaccctacaggaaatgttgctggtaaagacgcaaatagctgggagcggcttgcacaagcggctttggtctggctggattacccgtgctttaagaaatgggatgggtctcccgtgggctcctgggatgtgtcgccccaagaacacagtgacctgatgttccaggcctatgattttagacagcactgtagaccctctccaggaatccagttccacggaggagcctctccccaggtggaagagaactggacatgtaccatcctctgaatgccagagtatcttggaggcaaggtctccagagccgtctgggccagccctcttcacttctagggatagggggcttggatccaaagacagctgtgaattgatgtcagacctgggaccagaatccaggtctcctgacccccagccttcctgctattctgtgctgtcttttctttcatagacaatgctccccattggagcctgacaatagcctctctgagccaccaggagagactcaggcaaaagagtggaatcccagccttgactttcttctgtgaacctgaggggaaaggtgatggtccaatttattgtcaataataacaaaaatagtagcaaatgccatttgttgggtgttaattagcttcaaggtacagcgccaagaagtatacctgcatattatgtgtgtgtgtgcatattcattgattcaactaaagatattaattgggcacctgc

SEQ ID NO: 72 - protein, 389 aa, Bos taurus
MELRNTPAGSLDKFIEDHLLPDEEFRMQVKEAIDIICTFLKERCFRCAPHRVRVSKVVKGGSSGKGTTLRGRSDADLVVFLTNLTSFQEQLERRGEFIEEIRRQLEACQREETFEVKFEVQKRQWENPRALSFVLRSPKLNQAVEFDVLPAFDALGQLTKGYRPDSRVYVRLIQECKYLKREGEFSPCFTELQRDFLKNRPTKLKSLIRLVKHWYQLCKEQLGKPLPPQYALELLTVYAWEQGCNKTGFITAQGFQTVLKLVLKYQKLCIYWEKNYNSENPIIEEYLTKQLAKPRPVILDPADPTGNVAGKDANSWERLAQAALVWLDYPCFKKWDGSPVGSWDVSPQEHSDLMFQAYDFRQHCRPSPGIQFHGGASPQVEENWTCTIL

SEQ ID NO: 73 - DNA, 1601 bp, Equus caballus
  CDS (59)..(1255)
agtttctgggagccagtcccacgagcaccagctcctctgtccccacccgggcgtcacgatggagctccaaaagaccccagccaggaatctggacaagttcattgaagactatctcttgccagacacacggttccgcaggcaggtccgagaagccatcgacattatctgcagtttcctgaaggagaggtgtttccgaggtgccgttccccctgtgcgggtgtccaaagtggtgaagggtggctcctcaggcaaaggcacgaccctcagaggccgatccgatgctgacctcgtcgtcttccttgactacctcacgagtttccgggagcagtttgagcgccgagcagagttcatcaaggagattcggaggcagctggaagcctgtcaaagagagaagaggtttgacgtggagttcgaggtccaggggcagcagtgggcgaggccccgcgcgctcagcttcgtgctcacgtcgccccagctcaatgagggggtggaatttgatgtcctgcctgcctttgatgtcctaggtcaggtgactacatcgtacagacctgaccctgacatatatgtcctactcatcaaagaatgccagtccctggggaaggagggagagttctccccctgcttcacggagctgcagcgagccttcctgaggcagcggccaaccaagctcaagagcctcatccgcctggtcaagcactggtaccaaaagtgcaaggataaacttgggaaaccactgccagcacagtacgccctggagctcctgacagtctatgcttgggaacagggaagcagacaaacagaattcaacacagctcagggatttcggactgtcttggaactagtcctgaagtaccagcagctttgcatctactggacaaagtattacaactttgatgaccctgttattggacaatacctgaaaaggcagctcaagaaacccaggcctgtgattctggacccggctgaccccacaggaaacgttggtggtggagacccacgcagctggcctcggctggcacaggaggcgagagcctggctgagttacccgtgctttaagaattgggacgggtctccagtgggctcctgggacgtggggcctgaagaagacagcgaggacgacactttgacctgggctgagcgcgcatattaccagtgcgaccacggacggcgccctgaattcccgcagaccggcagcacgccccagagggcatccgctcccgacgcggaagagaactggacctgcaccatcctctgatcgccggcgcagcgtggaggagaggactccagagtcgggggggccagccccctcatttcctgggcgggatcttatcggctgtgacttggcatcagtcctaggaccagaatccgggtctcctgacccctcttcctgctgttcccctctttctcgccctccctaggtagcgctgcccgcagcctcatcccgccacagcctgttttctgacaatattctctgagaggcaacagttgaggtttagacaaaagagtggaaactcagccttgactttcttctgtgtgcctggtgagaaggttatggtccaatttattatcaataacaataaaaataatagcagataaaaaaa

SEQ ID NO: 74 - protein, 398 aa, Equus caballus
MELQKTPARNLDKFIEDYLLPDTRFRRQVREAIDIICSFLKERCFRGAVPPVRVSKVVKGGSSGKGTTLRGRSDADLVVFLDYLTSFREQFERRAEFIKEIRRQLEACQREKRFDVEFEVQGQQWARPRALSFVLTSPQLNEGVEFDVLPAFDVLGQVTTSYRPDPDIYVLLIKECQSLGKEGEFSPCFTELQRAFLRQRPTKLKSLIRLVKHWYQKCKDKLGKPLPAQYALELLTVYAWEQGSRQTEFNTAQGFRTVLELVLKYQQLCIYWTKYYNFDDPVIGQYLKRQLKKPRPVILDPADPTGNVGGGDPRSWPRLAQEARAWLSYPCFKNWDGSPVGSWDVGPEEDSEDDTLTWAERAYYQCDHGRRPEFPQTGSTPQRASAPDAEENWTCTIL

SEQ ID NO: 75 - DNA, 12956 bp, Homo sapiens
aaccaacagcagtccaagctcagtcagcagaagagataaaagcaaacaggtctgggaggcagttctgttgccactctctctcctgtcaatgatggatctcagaaataccccagccaaatctctggacaagttcattgaagactatctcttgccagacacgtgtttccgcatgcaaatcaaccatgccattgacatcatctgtgggttcctgaaggaaaggtgcttccgaggtagctcctaccctgtgtgtgtgtccaaggtggtaaaggtgagtccaggcctgcctggccaggggaggggtggctgaatgtgcaagagttgagattgagaatgagagagagagagagagagaagcaaaaacctagaacccagggtgcaaatgtgagtacagagagctgagatcttctgggatggtggtttcttatttatccacacagcatgttaaaatagattctggggtgaaatcctacatccctattattaacaagtgaccctcccccctacttcccgctgaagtttatgaaccactgtcctgggcgatgcccatttcagaaatagggaactgaatcccagctctggtaaacagtttgctaattcgtggccaggctaggggctcaccatttctgcagtgaagaatcatatgttttgaaagcaaatagcacctgctggctgcaagaccttgagcaagtcacttaactactctgtgttccaatttcctcagccataatccccaatactgttgcagtcttgccagtgcaccttaatgtagcagcttctcactgaattagtacccaaggttctttgtcctgcatccaagaaaattaaggaacatggacacaaacgtgagcttggagcaaaagttcagtaagcaaaagaagaaagctgtctccactgtggagagggaagtctgagtggattgccagattgcagctgaatgcaaaaaacttttataagaaaccactctcctccctgtaactgtttgagaaactttttatcagtaaagctgtgcaacttcccttaccttatgcagctgtgggtatatctctaggcaagcataaagcgctgcttctcttgtatgtataactgtggatttgttttaggtaagtcccactccctgcgccagtttcaggcaggccgctcctccagggcccagccttgaccatttacctaactgatttttcctctactttccctcaatacctcatagggccgtgtagattaagtaaaatagtaagtgtgaaccacccagcataagctagtcctgggcatcgtaaaggacaatgggaaaagaacacagatcctggaagaaggcccccaggtttgaattgtatttgccacctactagctgggtgatggggctgatatattatctcactgagcatccattttcccatctgtaaaatgggaactaatgataatggcatccaaatcatagcatcattgtgagcattataggagtttaagacatgcaatgccttcagaacagtggctagtgctccataatgttagtgattgctcctgtcattttatttagggaggtttgcctcactaagcatcaattattatttttgtcgtctttttcagggtggctcctcaggcaagggcaccaccctcagaggccgatctgacgctgacctggttgtcttcctcagtcctctcaccacttttcaggatcagttaaatcgccggggagagttcatccaggaaattaggagacagctggaagcctgtcaaagagagagagcattttccgtgaagtttgaggtccaggctccacgctggggcaacccccgtgcgctcagcttcgtactgagttcgctccagctcggggagggggtggagttcgatgtgctgcctgcctttgatgccctgggtgagagctcccagcttctttttctccctcttcccatttctgagcagaaatctcccacagtttgagagctttttgccccaacagggcatctctctaaagcagggtgggaggagatcttaggatctgtcccggggcaagaatgaatacggtcatgatctatcacaggagagacattaaacagcaaattggcataatgtggggacaaagacatttcttacagaacatctgcaaggcttactggttctgtttaaggcaaaatgtgtgaattttatctttctaaaatcaggcagcaaagatgtggcttaaagttcatgttactctcatctttgtcccaacatgagatctcatcaaacgtatgcagcacgttgggagatagatatttataatttgcaggaacatttggacaggaagtgtaacctctcagaggctcccttgccacatcaggagaattggtaaaaccacactacctgtatcatatcattattttaagtgataaatgatcatctacattcagctctgatgagtaataggtgttcaaaaataggaacttccagccaagtgtggtggctcatgcttgtaattccaacacttttggaggctgaggcaggagggtcgcttgagcccaggagttcaagaccagcctgggcagcaaagtgaaacctcatctctactaaaaattttaaaacattagccaagtgtggtggtacatgcctgtggtcgcagttattcaggacgctgagactgaacgatcacatgaggccagccaaggattcgaggtgtcagtgagccacgaatgtaccactgcactccatcctaggcacagagcaagagcaagaccctgtctcaatcaatcagtcaatcagtcaaaactatgaatttcccagctgtatatgaaggcacctcaaaacaccacagtgaactcacagagggacacggaatagtttagattttaattttttgagggaaatgcgatgacatctgtcacacaccgcacaaacggctactattaaactgaacttactgattagtggctactaattaatagttggtcattaagcagtaattagtgattaattatcaagtaattaggacttaattaaaggaactgtcacagtttcctttagtcctagggcagccatgaaaaaaaaaatgctgactctccaaagacaccagggtatgagaaagttttggattctctcctttgtgccatctcctgtgttgggggctgaagtacaatggttgtaaaagacaagagggagaaggctggtcacagtggctcacgcctgtaatctcagcactttgggaggccaaagtggggggatcacttgaagtcaggaattcaagaccagcctggccaacatggtgaaatctcacatctactaaaaatacaaaaattagctgggcgtggtggtgtgtgcctgtaatcacagctactcgggaggctgaggcaggagaattgcttgaacccaggagatggaggttgcaatgagccaagatcatgccattgcactccagcctgggcaacagagtgagactccatctcgaaaaaaagaaaaaagaaaagaatataaggagtgattaaaaaagaaaagaaaagaaaactaagtagggtgaaacaatagatagccatgggggttagggagcttttttagacagggtcgtgagggagggtccctgagcctgagtggcgagaaggagtgagccttggggagatctggaggttctgggaagaggaatggcaagtgcagaggccctgaagcagcaatgaccatggcacatttgaggaagagagaaaaagtcagagaagtagaaagtgggcaaaggaagcaagacaggaggtgaggtgggagaggttccagagaccagatcacaccagacatcattggccaccataagatctttgggttttaaaattccagatgttatgggatgcaggaagcagcatgatcagcagcattctctaggtgccaggttgagaacaggctgtgggggaacctgtaaagaggttgctgccatagttccggcgagtgacggtggtggcttggatggggtgatggcagtggagagggcaggagggaggatcaggaatggacctcaagacttcccagccctgggtctgctgcacttttcaatcaaaccccatggccagggagattgtcccctcagagtgactgaaggaaattcagagaagagctgacacctaagttgtagattttgcccgaacaggtcagttgactggcggctataaacctaacccccaaatctatgtcaagctcatcgaggagtgcaccgacctgcagaaagagggcgagttctccacctgcttcacagaactacagagagacttcctgaagcagcgccccaccaagctcaagagcctcatccgcctagtcaagcactggtaccaaaatgtatggccctcccaccaggcctggtgggtcctgtctcgactgggagcagaggaggggtggggggaggagagaaagaagggagtgaagggaagaggagggggagtggtggagggaaatagagggatggaaaaaggagagaaaggaaaaagaggtggagagaggagcctgcaacagaagggagaatgaaagggaaggaagagagaaaggaagggattttggtgttctgttcactgctgtatccccagaacttaaaacagagcctggtgcataataggtgtaaataactgttgaataaatgaatcaatgctacatacacacacgcacgcacacacacacagagagagagtcaaccacactcttcagaaggtggataagttaaaacaagagtttcaaacaaatatatgttcagatgccctttcctcccacttactggctggctggccttaagtaagcaacttaacctttctgttctttctgctttcttatctgcaacgagtagcatgccatagctagagtaacacggcatatagttggtcctgataaatgtagcatattttagccaccataggagtacacataataaaagctaacatgtagtatgtgcttagcttatctatgttttgtggatgtgatacaattttctgttcacttttaaatgccctgcatcttagtcaattttaacagtgattctgtaagttagataaggttaggcattattattaaatccattttacaccaagagaaacttgggtcaaaaagagaaactcctgggtcacatggctcattcggccaataagtagcagaagtaaaatttgaatttggctgggcgcggtagctcacaccagtaatcccagcactttgggaagccaaggcaggtagattgcttgagcccaggagttcaagactagcctgagcaacatggcaaaacctcgtctctacaaaataaactaaaaatttagccaggtgtgatggtgagcacctgtagccccagctactgggtaggctgaggtgggaggatcgcttgagcctgggaggaggaggttgcagtaagtcaggattgcactactgccctccagcctgtgagacagagcaagatcttctctcaaacaaacaaacaaacaaacaaacaaaaactcgaatttgggtctattgacttaagagtttgcctgataataataggcattcaatgtatatttcttgaatgaacgaatgaatgaaaataatcaggaataaactttccaatttaaaagtaacacctctaggtaaaaaaaagacaatcatttagttgccagacttctaagtgtttgctgttctatgaattgtaatcatggagcctgagcattgtagaatttacaaaagcagttcctgacaaaagcagcactgcccccagggacatattgaaaattaatgagggtgtttttggtaaccatggtgatgggaggacatgggtgctacttatatttagtggaaagaagacaagaatgctagttattgtacaatgatcaagagagtcctgcacagccaagaattgtctttttctttctttcttgatgctgttctcctttaaaacaagacaagattaacaataatttaactccactaaccaccatcatcaccacctccaacttatatgctacatttcttgtatatttcaagtctgtttatattttcaagtgcctcgaagtattattgttttatagccaaatgtttagttaatctgctcacagatttaccactttcttcactattcattctgtcttacacctctaacattccatctggggtaattttcctaaatgatcatgcatcctttgggatttcttttgatgatggtctattggtagtaaactctctcagttattgtttgtctgaaaatgtcatgcttttgccttcattgttgaagggtgcttttgctgggtggtcatttcagtatattgaatatatcattccatcttccagtgtcatcattaaaaagtcagttgccagtctaactgcagctcttttataagtaacctgtcttattcttctggctgcatgtaaaagttttctctttgtctttgattttgtttagcttcaatctgctgtgtcttaatgatgggttcctattgtttgtcctgattgggattccgttaagattcctgaatctgtgggtagatatctttaatcagttttgaaacttctcagccattcttctaaaatattgattctccttcattctctcctcaccttctagaattccaattaaatgtatgttagaccctgctctatctttcatatctctatactctcttctgtgtttttcatccttttgtctatttttccatgctttattctgaatagttccttctaatctaccttccaattaactaattttctctttagctatatctaatttgctgtaattaattacagttgccatttttatcctaaaatttctatttcatatttttgtatctgccatggtacttcttatggcttttaattccctgctaactatttaaagttcttattttatcctgtgaatatgatattcctagttattttatttttaatttttattatttgttaatcttatgttttatttacacttcttttctgtgacatgagcacacacagattcatgtgtatacatatatggctctgatacctctcctttcctgtcctcattcaaaccactgatcacagagagaggactatttttttttatttttaatttttctatttcaataggtttttgggggaacaggtggtgtttggttacatgaataagttctttagtggtgattttggtgcacccatcacccaaacagtgtacattgtacccaatgtgtaatcttttaacccttgccacaccccaccctttccccgcagtccgcaaagtcccatgtatcattcttatgcctttgcttcctcatagcttagctcccacatatgagtgagaacatacaatgtttggttttccattcctgagttatttaattaaaataatagtatccaattccatccaggttgctgtgaatgccattattttgttcctttttatggttgagtagtattccatggtgtgtttgtgtgtgtataacatttttctttatccactcattgattgatgggcatttgggctggttccatatttttgcaattgcaaattgtgctgttataaacatgtgtgtgcaagtatcttttttgtataatgacttcttttcctctgggtagatacctagtagtgggattgctggatcaaatggtagatctacttttagttctataaggaatctccacactgttttccatagtggttgtatgagtttacattcccaccaatggtgtaaaagtgttcccttttcaccacatccacaccaacatctattattgtttgattttttattatgaccattcttgcaggagtgaggtggtatcacattgtggttttgatttgcatttccctgataattagggatgttgagcatttttccatatgcttgttggtatttgttttttttttttttttttcattattatactttaagttttagggtacatgtgcacaatgtgcaggttagttacatatgtatacatgtgccatgctggtgtgctgcacccattaacccgtcatttagcattaggtatatctcctaatgctatccctccccaattccccccaccccgcttgttggtatttgtatatcttcatttgagaattctctgttcatgtccttagcccactttttgatgagattttttttttcttgctgattcgtttgagttctttgtagattctggatattagttggatgtatagattgtgaagattttctcccattctgtgggttgtctgttaactctgctaattatttcttttgctttgcagaagctttttagtttaattaagtcccatctatttatctttgtttttgttgcatttgcttttgggttcttggtcatgaagtctttgcctaagccaatgtgtaggagggtttttccaatattatcttctagaatctttatggtttcaggtcttagatttaagtatttgatcgattttgagttgaattttgtataaggggagagagaaggattcagtttcattcttctacatgcaacttgccaattatcctaggaccatttgttgaatagggtgtcctttccccattttatgtttttgtttggtttgtcaaagatcagttggctgtaagtgtttggctttatttctgggttatctattctgttccatttgtctacgtgactatttttataccagtaccatgttgttttggtgactatggccttacagtatagtttgaagtctgataatgtaatgcctccagatttgttctttttacttagtcttgctttggctatgtgagctcttttttggtgccatatgaattttaggattgttttttctagttctgtgaagaatgatggtggtattttgatgggaattgcattgaatttgtagattgtttttgggagtatggtcattttcacaatattgattctacccattcatgagcatgggatgtgtttccatttgtttgtgtcatctatgattttctttcagcaatgttttgtagttttccttgtagagttcctagttattttaaagtctgtgttcggtctttcagcatttaaagtttgtaggtttattactatttctcttctttctgttggtcataactcttagtgttttgtttccttgtgtgcctggttacatatgtgctggtcattgtatttgaaaattatgtgtgaaataatttgaggttttggattatgtatattcctccagaaagaatttcatttgcttctgtgcatttcttaggaacattacaagtccttcttctcagttaattttcgtagtatctttatcagataggtgctattacaaccactcacttagcagatgaaaatcatgaggctctgagagtctaagtcatctacttagaattggacaatggtgaagccaggattcaaacccacatcaataagaatccagcgctcttaacaaggggccagtacacttttttaaaaaataaaaggctagatagtaaatattttagactttgtggactgcacagcctctgttgcaactactcaaccctgcctttgtagcatgaatgcagtcataaactatacataaatgaatgagcctggattcgttccaaggaaactttataaaaacaggtggcaggctggatttggcccatgagaagtgtagtttacacaaaagttgagcaaaccaatttttttctgattgtttttcctcttctcagtgtaagaagaagcttgggaagctgccacctcagtatgccctggagctcctgacggtctatgcttgggagcgagggagcatgaaaacacatttcaacacagcccagggatttcggacggtcttggaattagtcataaactaccagcaactctgcatctactggacaaagtattatgactttaaaaaccccattattgaaaagtacctgagaaggcagctcacgaaacccaggtatgctatccccacatggcttagctcccctatgtaaatgaacacctggatacaggtacagtgccttggaaatggaggaggtgggagggctccccacttagtgagaatctcctgttgcccatcattgtactgggcattttactactgccatctgttttaaacacctacctccaaccctgtgaggcaggcactatgccaattattttacaggtgagtaaactgaggttctgagaggtaaggagcttgtccaacccttaacagaaaatgagtaaaatagctgcagtttgaactgaaataagaacagcagcaacaacaatgatagtaattgctcccaggtattgaaagcttgttgtaagactaacacatgctaatataatagtaaaaattattagcaatattactgatatgtatgttatgttctagtcgctgtgctgagcatttcatataactgggctttttctatcctcacagcatagcctttgagataggtatgtggaactattcccattttacagataagaatcctgaggcttagagagttcaagtgacctacccaagggcacatcactgataaagggcagaggtgggattcaaacccacatctgtcaggtgcaagtgcaaggctccttctcctcatgctcactgcctgctggggaatagggcactggggacataccccagggagcccttcctcatgttctgagtcccagttcatcccatgctgctattttgctctcccaggagcatctggactccctagacagagccccagcttctcacctgtccctctctaaatgctgctctgcaggcctgtgatcctggacccggcggaccctacaggaaacttgggtggtggagacccaaagggttggaggcagctggcacaagaggctgaggcctggctgaattacccatgctttaagaattgggatgggtccccagtgagctcctggattctgctggtgagacctcctgcttcctccctgccattcatccctgcccctctccatgaagcttgagacatatagctggagaccattctttccaaagaacttacctcttgccaaaggccatttatattcatatagtgacaggctgtgctccatattttacagtcattttggtcacaatcgagggtttctggaattttcacatcccttgtccagaattcattcccctaagagtaataataaataatctctaacaccatttattgactgtctgcttcgggctcaggttctgtcctaagccctttaatatgcactctctcattaaatagtcacaacaatcccatgaggcatttttaaaaattttttattattttagattcagagggcacatgtgccatttgttacacagctatattgtgtaatggtggggtttgggcctctattgatcctgtcgcccaaatagtgaacagagtacccaaaaagaattttttcaacctttgcctttctcccttcctcctccctgttggagtccctagtgtctattgttcccatctttagcagatgttaagtatttgattttctgtttctgggttaattcacttcggataatggcctccagctgcaaccatgatttcattctttcttatggctgcataatactccatggtgtagatataccacactttctttatccagttcacactgatgggcacttaagttgattccatgactttgctattgtgaatcgtactgcgataaacatacgagtgccggtgtcttttgatagaatgatttctttacctttgggtagataccgagtagtgggattgctgggttgaatggacattctacttttagttatttgaaaagtcccatgaggcatgttttctatcattcccatcttacagatgagacaaaggctcagagaggtgaggtcacttgctcaaggacatcagctaacaagtggtggaaatggaattcaagctcagtggactctaaagccagtgctcatgtcactgtgctaaacagcctgccttgtcacatccccacctctcatctgaccaatgggagactctgagcagctgagtgacttgggttgtcacacagctaaacaggggcaaaggacccagtcttggatctttccacctccaagcaggaatctgtctgattccaggggattgatgatgttgcagatggctaggaagcagactccaggatggaatttagtatgcaggatgttctgggggagagccactggaaccagcactcagggaaaggggggaagaaaggataggaaggaagcatgaaagagaatagggagaagtgaacagggatgcagagcgaatgccagtttcagccaactccaaggacagccctggagctggaatggcctttagagctgccccatggtgacagaggtggccaggcttctatacccctacgtggatcactcactgtgcttgggcaccttgggaaagggcatggctttgagcaaaaggctctctgcagctgaggcaacccctaaaagggctgacggctgaagtctgtctgctgaccactgtcccagcagctggggcttgttagtccttcctcaaagggggatccagatggcatgtcacagtgtctaccgtaaatgctcactgaatccagctgcaatgcaggaagactccctgatgtgatcatgtgtctcaccctttcaggctgaaagcaacagtgcagacgatgagaccgacgatcccaggaggtatcagaaatatggttacattggaacacatgagtaccctcatttctctcatagacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaggacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccattta

SEQ ID NO: 76 - DNA, 748 bp, Homo sapiens
cagatggcatgtcacagtgtctaccgtaaatgctcactgaatccagctgcaatgcaggaagactccctgatgtgatcatgtgtctcaccctttcaggctgaaagcaacagtgcagacgatgagaccgacgatcccaggaggtatcagaaatatggttacattggaacacatgagtaccctcatttctctcatagacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaggacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttattgggtgtttattaacttcaaggcacagagccaagaagtacagatgcatatctaggggtattgtgtgtgtatatacattgattcaacaagaaatatttattgagcacttactatgtgccaagcatagctctgg

SEQ ID NO: 77 - DNA, 748 bp, Homo sapiens
cagatggcatgtcacagtgtctaccgtaaatgctcactgaatccagctgcaatgcaggaagactccctgatgtgatcatgtgtctcaccctttcaagctgaaagcaacagtgcagacgatgagaccgacgatcccaggaggtatcagaaatatggttacattggaacacatgagtaccctcatttctctcatagacccagcacactccaggcagcatccaccccacaggcagaagaggactggacctgcaccatcctctgaatgccagtgcatcttgggggaaagggctccagtgttatctggaccagttccttcattttcaggtgggactcttgatccagagaggacaaagctcctcagtgagctggtgtataatccaggacagaacccaggtctcctgactcctggccttctatgccctctatcctatcatagataacattctccacagcctcacttcattccacctattctctgaaaatattccctgagagagaacagagagatttagataagagaatgaaattccagccttgactttcttctgtgcacctgatgggagggtaatgtctaatgtattatcaataacaataaaaataaagcaaataccatttattgggtgtttattaacttcaaggcacagagccaagaagtacagatgcatatctaggggtattgtgtgtgtatatacattgattcaacaagaaatatttattgagcacttactatgtgccaagcatagctctgg

SEQ ID NO: 78 - DNA, 24 bp, Artificial Sequence
  Synthetic
cagatggcatgtcacagtgtctac

SEQ ID NO: 79 - DNA, 24 bp, Artificial Sequence
  Synthetic
ccagagctatgcttggcacatagt

SEQ ID NO: 80 - DNA, 21 bp, Artificial Sequence
  AF217002 Forward Primer
aaggctggcgcagctgccgct

SEQ ID NO: 81 - DNA, 23 bp, Artificial Sequence
  AF217002 Reverse Primer
aacccagctaggtgacagtctgg

SEQ ID NO: 82 - DNA, 21 bp, Artificial Sequence
  AF217003 Forward Primer
acctgccctcgcgatggcggc

SEQ ID NO: 83 - DNA, 24 bp, Artificial Sequence
  AF217003 Reverse Primer
atcctcctgcctcatcttctgagt

SEQ ID NO: 84 - DNA, 24 bp, Artificial Sequence
  AF261233 Forward Primer
actagagcagccagcccgtgagca

SEQ ID NO: 85 - DNA, 24 bp, Artificial Sequence
  AF261233 Reverse Primer
ctggcttacagagtgagttccagg

SEQ ID NO: 86 - DNA, 24 bp, Artificial Sequence
  AF319547 Forward Primer
tgttatgtccctcaggtcctgctc

SEQ ID NO: 87 - DNA, 24 bp, Artificial Sequence
  AF319547 Reverse Primer
aaactcaatgctgggtcagaggca

SEQ ID NO: 88 - DNA, 24 bp, Artificial Sequence
  AK017032 Forward Primer
ttactggaggctgtgaaatctagg

SEQ ID NO: 89 - DNA, 23 bp, Artificial Sequence
  AK017032 Reverse Primer
tagggcgtggtggaagctgaaca

SEQ ID NO: 90 - DNA, 24 bp, Artificial Sequence
  BC021950 Forward Primer
cagacacaatctactcctctcgct

SEQ ID NO: 91 - DNA, 24 bp, Artificial Sequence
  BC021950 Reverse Primer
cattgtcaagtgtatcccacccca

SEQ ID NO: 92 - DNA, 24 bp, Artificial Sequence
  Dtx1 Forward Primer
ctaagggattgagatcatgtcccc

SEQ ID NO: 93 - DNA, 24 bp, Artificial Sequence
  Dtx1 Reverse Primer
gtgcttgactcaagtcctgggaaa

SEQ ID NO: 94 - DNA, 24 bp, Artificial Sequence
  Lhx5 Forward Primer
gaagtcttggttgatccgtaacgg

SEQ ID NO: 95 - DNA, 24 bp, Artificial Sequence
  Lhx5 Reverse Primer
tgactttggtcccgagaaattgcg

SEQ ID NO: 96 - DNA, 24 bp, Artificial Sequence
  Oas1a Forward Primer
agacccaggaagctccagacttag

SEQ ID NO: 97 - DNA, 24 bp, Artificial Sequence
  Oas1a Reverse Primer
gacagaaccttccaacaggtggac

SEQ ID NO: 98 - DNA, 23 bp, Artificial Sequence
  Oas1c Forward Primer
aaacactcctggcctcaggatgg

SEQ ID NO: 99 - DNA, 24 bp, Artificial Sequence
  Oas1c Reverse Primer
cagccccagtgcattgtgatttaa

SEQ ID NO: 100 - DNA, 22 bp, Artificial Sequence
  Oas1d Forward Primer
gtcagcaaacactcctggcctc

SEQ ID NO: 101 - DNA, 25 bp, Artificial Sequence
  Oas1d Reverse Primer
gcgttttgctttaatttagtgcttc

SEQ ID NO: 102 - DNA, 24 bp, Artificial Sequence
  Oas1e Forward Primer
tcaggatggcgagggaactcttca

SEQ ID NO: 103 - DNA, 24 bp, Artificial Sequence
  Oas1e Reverse Primer
aactgccataaacccggcctgcat

SEQ ID NO: 104 - DNA, 24 bp, Artificial Sequence
  Oas1f Forward Primer
gtcagcaaacacttcctggccata

SEQ ID NO: 105 - DNA, 24 bp, Artificial Sequence
  Oas1f Reverse Primer
caggtggaagtcaagcttgagttc

SEQ ID NO: 106 - DNA, 24 bp, Artificial Sequence
  Oas1g Forward Primer
agacccaggaagctccagacttag

SEQ ID NO: 107 - DNA, 24 bp, Artificial Sequence
  Oas1g Reverse Primer
agttccacgacagcgtgtgtcaca

SEQ ID NO: 108 - DNA, 24 bp, Artificial Sequence
  Oas1h Forward Primer
ggctgcagtcagcaaacattcctg

SEQ ID NO: 109 - DNA, 24 bp, Artificial Sequence
  Oas1h Reverse Primer
cggtcctctctaggtcaaagtctt

SEQ ID NO: 110 - DNA, 24 bp, Artificial Sequence
  Oas2 Forward Primer
gaccagctagcaacgatgggaaac

SEQ ID NO: 111 - DNA, 24 bp, Artificial Sequence
  Oas2 Reverse Primer
actgacccaggatcttctgtccca

SEQ ID NO: 112 - DNA, 24 bp, Artificial Sequence
  Oas3 Forward Primer
gaaactctactgagagtaccggtc

SEQ ID NO: 113 - DNA, 24 bp, Artificial Sequence
  Oas3 Reverse Primer
ggggaggaaagggtttattcagct

SEQ ID NO: 114 - DNA, 24 bp, Artificial Sequence
  Ptpn11 Forward Primer
aagacgggaggaacatgacatcgc

SEQ ID NO: 115 - DNA, 24 bp, Artificial Sequence
  Ptpn11 Reverse Primer
agcagtctctccttagctgaggaa

SEQ ID NO: 116 - DNA, 23 bp, Artificial Sequence
  Rasa11 Forward Primer
tgaaccggctgacagcgtgcttg

SEQ ID NO: 117 - DNA, 23 bp, Artificial Sequence
  Rasa11 Reverse Primer
aagagcctgtgtccggcttcgag

SEQ ID NO: 118 - DNA, 24 bp, Artificial Sequence
  Rph3a Forward Primer
aaccttccatgtggagtagtctgg

SEQ ID NO: 119 - DNA, 24 bp, Artificial Sequence
  Rph3a Reverse Primer
ggacctgaagatgcttaaggtcag

SEQ ID NO: 120 - DNA, 24 bp, Artificial Sequence
  Sds Forward Primer
ttcctgattctgtctcacgtggct

SEQ ID NO: 121 - DNA, 24 bp, Artificial Sequence
  Sds Reverse Primer
ttgatgacccaccaggtgtccagg

SEQ ID NO: 122 - DNA, 24 bp, Artificial Sequence
  First Exon Forward Primer for Oas1b
aatcctagacctgcaagtccagag

SEQ ID NO: 123 - DNA, 24 bp, Artificial Sequence
  Frist Exon Reverse Primer for Oas1b
ggttgcagcctggcttctgaaagt

SEQ ID NO: 124 - DNA, 24 bp, Artificial Sequence
  Second Exon Forward Primer for Oas1b
tggtgatggactccagttcagcat

SEQ ID NO: 125 - DNA, 24 bp, Artificial Sequence
  Second Exon Reverse Primer for Oas1b
actagaagggaggatgaaggcatg

SEQ ID NO: 126 - DNA, 24 bp, Artificial Sequence
  Third Exon Forward Primer for Oas1b
ggctgagctgttccactgaatgaa

SEQ ID NO: 127 - DNA, 24 bp, Artificial Sequence
  Third Exon Reverse Primer for Oas1b
ctctgtacagatgaagcgcacagt

SEQ ID NO: 128 - DNA, 24 bp, Artificial Sequence
  Fourth Exon Forward Primer for Oas1b
tggctatagcacaagaaggcatag

SEQ ID NO: 129 - DNA, 24 bp, Artificial Sequence
  Fourth Exon Reverse Primer for Oas1b
tgacatgaagagtgttggacgacc

SEQ ID NO: 130 - DNA, 24 bp, Artificial Sequence
  Fifth Exon Forward Primer for Oas1b
ggtcagttgacagcagctcgtttc

SEQ ID NO: 131 - DNA, 24 bp, Artificial Sequence
  Fifth Exon Reverse Primer for Oas1b
aataaggagagccaatggcctcaa

SEQ ID NO: 132 - DNA, 22 bp, Artificial Sequence
  Sixth Exon Forward Primer for Oas1b
ctattacagacggaggttgcag

SEQ ID NO: 133 - DNA, 26 bp, Artificial Sequence
  Sixth Exon Reverse Primer for Oas1b
tgctactgtgaacagacaccatgacc